(19) United States
(12) Patent Application Publication
(10) Pub. No.: US 2026/0009064 A1
(43) Pub. Date: Jan. 8, 2026

(54) STEROL PRODUCTION IN YEAST

(71) Applicant: APIX BIOSCIENCES, Wingene (BE)

(72) Inventors: Geraldine WRIGHT, Oxford (GB); Elynor MOORE, Oxford (GB); Irina BORODINA, Kongens Lyngby (DK); Jane Dannow DYEKJAER, Kongens Lyngby (DK); Jonathan Asmund ARNESEN, Kongens Lyngby (DK)

(73) Assignee: APIX BIOSCIENCES, Wingene (BE)

(21) Appl. No.: 18/710,789

(22) PCT Filed: Nov. 17, 2022
(86) PCT No.: PCT/GB2022/052917

§ 371 Date: May 16, 2024

(30) Foreign Application Priority Data
Nov. 19, 2021 (GB) 2116694.7

(51) Int. Cl. C12P 33/00; A23K 10/16; A23K 20/105; A23K 50/90; C12N 1/16; C12N 9/02; C12N 9/10; C12N 9/88; C12N 9/90; C12R 1/645

(52) U.S. Cl. CPC C12P 33/00; A23K 10/16; A23K 20/105; A23K 50/90; C12N 1/16; C12N 9/001; C12N 9/0071; C12N 9/1007; C12N 9/88; C12N 9/90; C12R 2001/645; C12Y 103/01021; C12Y 103/01071; C12Y 103/01072; C12Y 114/19; C12Y 201/01041; C12Y 402/01123; C12Y 504/99017

(57) ABSTRACT

The present invention relates to genetically-modified oleaginous yeasts for producing non-native sterols at commercially useful levels, especially for example in providing sterols individually or as a mixture in an artificial dietary composition for honeybees or other insects or animals. For this purpose, an oleaginous yeast, e.g. Yarrowia lipolytica, may be employed wherein the yeast has reduced production of ergosterol compared with a wild-type oleaginous yeast or is incapable of producing ergosterol and is provided with a sterol surrogate to aid growth. From such yeast, however, other yeast may be engineered which retain useful sterol production without need for a sterol surrogate, e.g. production of sterol mixtures in which 24-methylenecholesterol or campesterol is the dominant sterol.

### FIELD OF INVENTION

The invention relates to methods for producing sterols and compounds derived therefrom. In particular, the invention relates to genetically modified yeasts, in particular oleaginous yeasts for producing non-native sterols endogenously and methods of using such yeasts. Non-native sterols and non-native sterol mixtures produced as now disclosed have uses in a wide variety of compositions including artificial dietary compositions, food products and pharmaceutical compositions. One application of sterols and sterol mixtures produced as now taught and which is of especial interest is in provision of artificial dietary compositions for honeybees. Honeybees are sterol auxotrophs and hence phytosterols such as 24-methylenecholesterol are essential nutrients for such bees. However, up to now no method has been available for readily commercially producing sterols for this purpose by culturing cells such as yeast cells using a cheap and simple carbon source.

### BACKGROUND

Sterols are a subset of triterpenoids ubiquitous among eukaryotes. Their biological functions are diverse. Notably, sterols are structural components of phospholipid membranes, steroidal hormone precursors and signalling molecules. Terminal intermediates in the phytosterol biosynthesis pathway, including desmosterol, cholesterol, 24-methylenecholesterol, campesterol, isofucosterol, beta-sitosterol and stigmasterol, are often the most prevalent sterols in plants. For plants, sterols may be used for growth, development regulation and hormone synthesis as well as pest and pollinator control. Sterols may be used in the formation of artificial food compositions that replace natural sterol sources, such as a pollen replacement for pollinators such as bees and other insects.

For example, the domesticated insect, the Western honeybee (Apis mellifera) requires a mixture of sterols. Svoboda at al. (1980) J. Insect Phys. 28, 291-294 and Svoboda et al. (1988) Insect Biochem. 16(3): 479-482) teach that honeybees' tissues are comprised of a mixture of sterols derived from floral pollen that include 24-methylenecholesterol (51.2%), β-sitosterol (23.7%), isofucosterol (13.2%), campesterol (8.7%), cholesterol (2.2%), and desmosterol (1.0%); the relative proportions of each sterol can vary from 5-10%. Svoboda et al. (1979) J. Insect Phys. 28: 287-289 further teaches that when bees are deprived of any of these sterols, the whole colony will die within 8-12 weeks, thus demonstrating the essentiality of these particular sterols in diet and the essentiality of their plurality.

Published International Application WO20171085477 (University of Newcastle-Upon-Tyne) relates to provision of artificial bee feed comprising 24-methylenecholesterol. e.g. bee feed comprising 24-methylenecholesterol and one or more other phytosterols such as campesterol, β-sitosterol and cholesterol. However, some phytosterols. Including 24-methylenecholesterol and isofucosterol are not produced industrially. For this reason, they cannot be readily provided to bees or other domesticated insects in feed at the present time. As indicated above, there has remained a need for preparing such a composition by means of convenient yeast cell culture, especially using an oleaginous yeast.

Oleaginous yeasts such as Yarrowia lipolytica have high capacity for sterol ester storage in lipid particles and are more suitable for non-native sterol production than for example Saccharomyces cerevisiae. Nevertheless, S. cerevisiae has been much used in investigation of enzymic pathways in both yeast and plants for sterol production. Such studies however, as further discussed below, have not enabled production of sterols and sterol compositions as now taught and desired for many applications, including bee feeds.

Sterols, such as phytosterols, are of further industrial interest, as precursors to brassinosteroids. Brassinosteroids have agricultural application as plant growth hormones, as well as broad pharmaceutical applications.

Sterols, such as phytosterols, have a cholesterol-lowering effect when present in the diet of mice and humans, via inhibitory effects on cholesterol absorption in the liver. Plant sterols are advised as part of a healthy diet for the management of dyslipidemia. They may be desirably added to a variety of food products.

Phytosterols have also been associated with reduced risk of cancer, cardiovascular disease, arthritis, hepatic inflammation, and may have anti-oxidant function. Phytosterols are of further pharmaceutical interest, as precursors to withanolides. These bioactive steroidal lactones are of broad pharmacological interest, including the treatment of cancers, inflammation and ageing. Similarly, sterols and phytosterols are precursors to important steroidal compounds. For example, cholesterol can be used for the synthesis of vitamin D3.

24-methylenecholesterol is not presently available commercially. Several other phytosterols (e.g. β-sitosterol) can currently be isolated from vegetable oils such as wheatgerm oil. However, phytosterols are present at very low abundance in plant tissue, making isolation of phytosterols by extraction non-viable on a commercial scale. Plant sources also contain a mixture of phytosterols which are difficult to separate due to their highly similar structures and chemical properties. Cholesterol is commonly obtained by isolation from lanolin and cattle spinal cord. As for plant tissues, yield is low, and supply is further limited by extraction efficiency. Total chemical synthesis of cholesterol has been described, for example from hydroquinone. However, this is complex and inefficient for industrial production of phytosterols. Further, stereospecificity, particularly of C-24 ethyl sterols, is difficult to achieve.

Consequently, modified yeast strains have been of interest for producing non-native phytosterols. Endogenous sterol pathway flux in yeast is such that only the end-product sterol, ergosterol, accumulates. All preceding intermediates are transient and represent a small percentage of the overall sterol component of the cell. Since the fungal sterol biosynthesis pathway shares intermediates with those of plants and mammals, yeast cells have been engineered to divert sterol pathway flux away from ergosterol toward an alternative, non-native sterol. Production of desmosterol, cholesterol, 24-methylenecholesterol, campesterol, isofucosterol and β-sitosterol has been achieved by introducing varying combinations of heterologous sterol reductases and sterol methyltransferases, whilst inactivating endogenous ergosterol pathway genes encoding Erg4p, Erg5p or Erg6p.

The sterol composition of Saccharomyces cerevisiae has been modified by altering the activity of enzymes involved in ergosterol synthesis; enzymes in the latter half of the pathway are non-essential. Reduced oxygen or heme availability inhibit ergosterol synthesis, since the pathway is metabolically demanding and oxygen-dependent. Under such conditions, S. cerevisae will take up diverse sterols from the growth medium to substitute ergosterol function. This ability is not common in other yeast genera: most yeasts lack sterol uptake transporters and therefore, the genes encoding enzymes necessary in the final steps of sterol production may be considered essential.

A method for the production of cholesterol in Saccharomyces cerevisiae has been described in Souza et al. (2011) Metabolic Eng. A stable yeast strain efficiently producing cholesterol instead of ergosterol is functional for tryptophan uptake, but not weak organic acid resistance. The strategy to create the cholesterol-producing strain involved disrupting the ERG5 and ERG5 genes and replacing them with genes encoding dehydrocholesterol reductases DHCR24 and DHCR7 from fish.

Sawai et al. (2014) The Plant Cell, Sterol side chain reductase 2 is a key enzyme in the biosynthesis of cholesterol, the common precursor of toxic steroidal glycoalkaloids in potato, discloses an engineered S. cerevisiae yeast strain in which both the ERG4 and ERG5 genes have been knocked out and in which a potato (Solanum tuberosum) gene has been introduced for expression of the delta-7 sterol reductase, StDWF5. However, the sole interest of the authors is elucidating the biosynthetic pathway of cholesterol and related steroidal glycoalkaloids and the content or purity of 24-methylenecholesterol was not quantified. They do not address producing 24-methylenecholesterol for commercial use with a cheap carbon source or indeed teach whether this is feasible.

A method for the production of campesterol in S. cerevisiae has been described in Tsukagoshi et al. (2016) J. Biol. Chem. Ajuga delta 24-sterol reductase catalyzes the direct reductive conversion of 24-methylenecholesterol to campesterol. They employed the above-noted engineered S. cerevisiae strain of Sawai et al. solely to characterise the ArDWF1 gene from an expression sequence tag library of Ajuga reptans var. atropurpurea and compare this with Oryza sativa DWF1. They established that ArDWF1, like OsDWF1, is functionally equivalent to yeast Erg4p and resulted in production of campesterol. However, again the studies did not address producing 24-methylenecholesterol nor suggest how 24-methylenecholesterol might be usefully produced in any engineered yeast strain in useful quantity for commercial use.

Oleaginous yeasts such as Yarrowia lipolytica and Rhodotorula sp. have the capacity to grow on lipidic substrates such as vegetable oils. This provides the potential for growth on waste vegetable oils: a cheap and abundant carbon source with a positive environmental impact.

Campesterol production in Yarrowia lipolytica is described in CN107083338A. Campesterol production in Y. lipolytica using glucose or sunflower seed oil as the carbon source has also been described in Du et al. (2016) PLOS ONE, Engineering Yarrowia lipolytica for campesterol overproduction. Zhang at al. (2017) Biotechnol. Lett. Improved campesterol production in engineered Yarrowia lipolytica strains, and Qian at al. (2020) Appl. Microbiol. Biotech. Increased campesterol synthesis by improving lipid content in engineered Yarrowia lipolytica. For such campesterol production, it was found possible to knock out the ERG5 gene of the Y. lipolytica cells employed and express in the same cels a codon-optimised coding sequence for a 7-dehydrocholesterol reductase (DHCR7). The DHCR7 gene from Xenopus laevis or Danio rerio was favoured.

Such studies do not consider, however, the stereochemistry around the C-24 which can be expected to be affected by reliance on endogenous yeast ERG4 for conversion of 24-methylenecholesterol to campesterol; it can be expected that an epimer of plant campesterol will be produced [24β configuration (S) rather than the correct plant 24α configuration (R)]. As discussed in Xu et al. (2020) ACS Synth. Biol. Engineering of phytosterol-producing yeast platforms for functional reconstitution of downstream biosynthetic pathways, for many applications, including downstream synthesis, it will be desirable to retain the stereochemistry at the C-24 of campesterol to be expected from use of a plant DWF1 delta-24 sterol reductase (see FIG. 16 of the present application).

Xu et al. hence used only plant enzymes in engineered S. cerevisiae to produce campesterol, Their above-noted paper sets out comparison of the ergosterol biosynthetic pathway in yeast, the phytosterol biosynthetic pathway in plants and engineered plant steroid pathways in yeast for example to synthesize β-sitosterol, 24-methylenecholesterol is thereby merely referred to as an engineered biosynthetic pathway intermediate and there is no suggestion to produce 24-methylenecholesterol. Whilst Xu et al. suggest that phytosterol synthesis established in S. cerevisiae should also be considered for reconstitution in Y. lipolytica for manufacturing purpose, they even hypothesize that 24-methylenecholesterol is toxic for yeast and may be a major cause of growth deficiency in S. cerevisiae strain YYL67 (dw1/5/7.MVA1,Δare1/are2/erg4, evolved).

Yang et al (2021) Biomolecules, Engineering of Saccharomyces cerevisiae for 24-Methylene-Cholesterol Production nevertheless teaches that S. cerevisae can be engineered to produce 24-methylenecholesterol by disrupting both the ERG4 and ERG5 genes and expressing a 7-dehydrocholesterol (DHCR7) enzyme. However, no suggestion is made for transfer of this approach to Y. lipolytica. Indeed, previous campesterol production in engineered Y. lipolytica is merely noted by Yang et al. as low yield and not satisfying large scale fermentation production. Rather they point to three approaches for addressing commercial production of 24-methylenecholesterol: searching for new potential microbial chassis strains, rational design of biosynthetic pathways and thirdly characterising further DHCR7 enzymes. The reported studies focus on testing the DHCR7 of Physalis angulate in S. cerevisiae with disrupted ERG4 and ERG5 genes for this purpose. However, on the basis of their studies the same authors merely go on to favour instead expression of duplicated codon-optimised sequences encoding Xenopus laevis DHCR7 and suggest still significant room for improvement of 24-methylenecholesterol yield.

Thus, a commercially viable method for producing 24-methylenecholesterol is still required either alone or as part of a sterol mixture.

The inventors in this instance set out to provide improved phytosterol production in yeast with one aim being to address this problem and provide engineered yeast synthesising 24-methylenecholesterol, either as a sole purified sterol, or as part of a mixture, e.g. including campesterol preferably as the 24α epimer, e.g. for use in preparing artificial bee nutrition, by firstly selecting an oleaginous yeast.

In so doing, it has been established that in such a yeast required disruption of ergosterol synthesis may not be compatible with adequate growth for useful heterologous gene expression, or indeed any colony growth, on a conventional yeast growth medium, e.g. at 30° C. on yeast extract peptone dextrose (YPD) medium containing 10 g/l yeast extract, 20 g/l peptone and 20 g/l glucose, supplemented with 20 g/l agar for preparation of solid medium. In other words, knock-out of any of the ERG4, ERG5 and ERG6 genes may simply lead to undesirable growth deficiency or no growth as illustrated by the studies reported herein in Example 2 on unsuccessful ERG4, ERG5 and ERG6 gene knock out in the Y. lipolytica strain ST9100. Undesirable growth deficiency in this context may be equated with a growth rate which is economically unviable on an industrial scale, such as a reduction in the maximum achievable biomass accumulation below 10 g cel dry weight per litre of culture. e.g. when cells are grown at 30° C. on yeast peptone dextrose (YPD) medium containing glucose as the carbon source.

This was an unexpected problem given the earlier studies noted above on campesterol production by different ERG5-knock out Y. lipolytica strains. Previously, ERG4 was identified as essential by transposon mutagenesis but only in the wild-type strain W29 (CLIB89/ATCC20460™, Patterson et al. (2018) Metab. Eng. 48, 184-196). Gene essentially has also been examined by CRISPR-Cas9 mediated gene disruption in Y. lipolytica PO1f (MatA, leu2-270, ura3-302.xpr2-322, axp-2, Schwartz et al. (2019) Metab. Eng. 55, 102-110). Classification of ERG4 essentiality was inconclusive and varied according to the criterion used. However, the ST9100 strain is a modified strain for increased squalene synthesis, first reported in Arnesen et al. (2020) Front. Bioeng. Biotech. Yarrowia lipolytica strains engineered for production of terpenoids. It has been hypothesized by the inventors that inability to knock out ERG4, ERG5 or ERG6 in this strain without further engineering as taught herein may be related to the resulting accumulation of sterol intermediates which are toxic in high concentrations, for example due to detrimental effects to the membrane. Nevertheless, the Y. lipolytica strain ST9100 and other similar Y. lipolytica strains with increased squalene synthesis as a precursor to the sterol pathway are now enabled by teaching herein as highly desirable host cells for engineering to produce high levels of non-native sterols with appropriate gene knock-out.

As further discussed below, this gene knock out issue has been addressed by intracellular provision of a sterol surrogate as illustrated in the exemplification by introduction of a squalene-tetrahymanol cyclase coding sequence. e.g. Tetrahymena thermophila squalene-tetrahymanol cyclase sequence (TtSTC coding sequence) under the control of a weak yeast promoter. Introduction of such a coding sequence into yeast has previously been suggested in Published International Application W2021/133171 (Technische Universiteit Delft) but only in Saccharomyces species, e.g. S. cerevisiae and Kluyveromyces species. e.g. Kluyveromyces marxianus to enable growth anaerobically without sterol supplements. However, WO2012/133171 does not address the problem of gene knock out addressed herein and need to balance expression of a sterol surrogate coding sequence such as a TtSTC coding sequence with required flux through a biosynthetic pathway for high production of a non-native sterol in an engineered Y. lipolytica strain.

The prior art does not provide for creating an industrially viable source of non-native sterol through a yeast with a high sterol content relative to wild type strains. In addition, the prior art does not address the issue of essentiality of certain genes involved in ergosterol production in any oleaginous yeast. Furthermore, the prior art fails to provide scalable methods for the production of a range of sterols at suitable titres for commercial use of such sterols.

As such, there is a need for improved methods of producing sterols. For example, methods that provide increased production of a range of sterols by engineered yeast. There is also a need for improved genetically modified yeast for use in such methods that have improved feasibility.

It is an aim of certain embodiments of the invention to provide yeast for the improved production of sterols and compounds derived therefrom, for example, with increased titre of sterols as well as a broader range of sterols than that produced in the prior art.

It is an aim of certain embodiments to provide improved methods for producing sterols.

It is an aim of certain embodiments to provide improved methods for producing and extracting sterols from the yeast for use in feeds and pharmaceuticals.

It is an aim of certain embodiments to provide products such as artificial dietary compositions, food products and pharmaceutical compositions that include sterols produced by the methods and yeast described herein.

It is an aim of certain embodiments to provide products such as artificial dietary compositions, food products and pharmaceutical compositions that include the yeast as described herein.

### SUMMARY OF INVENTION

The invention provides genetically modified oleaginous yeast capable of producing a sterol surrogate such as tetrahymanol, such that normal ergosterol biosynthesis is no longer essential. As a result, certain genes involved in ergosterol biosynthesis are no longer essential and their expression can be partially or totally reduced. The sterol composition of such modified strains can be dramatically altered to achieve production of desired non-native sterols with high purity and content.

Thus provided in a first aspect of the invention is an oleaginous yeast for expression of one or more heterologous genes for production of one or more desired non-native sterols or compounds derived therefrom, wherein
(i) the yeast has reduced production of ergosterol compared with a wild-type oleaginous yeast or is incapable of producing ergosterol; and
(ii) wherein the yeast is provided with a sterol surrogate to aid cell growth.

A suitable reference wild-type yeast may be a corresponding wild type of the chosen host cells or conveniently for any oleaginous host cells, and especially such engineered Y. lipolytica cells, e.g. Y. lipolytica W29 strain Y-63746 as commercially available from the ATCC as ATCC20460™ or from the ARS culture collection, NCAUR, United States

As indicated above, such provision of a sterol surrogate will be desirable whenever the oleaginous yeast ahead of such engineering to modify sterol production is resistant to knock-out of any of the ERG4. ERG5 and ERG6 genes required for ergosterol synthesis via the native yeast ergosterol pathway (see FIG. 2) such that such knock out results in undesirable growth deficiency or no growth. This may be associated with, for example, increased flux toward the sterol pathway such as increased squalene synthesis compared with wild-type as observed for Y. lipolytica ST9100 Thus, it may be associated with any of increased synthesis of squalene or another sterol precursor or sterol pathway intermediate compared to a suitable reference strain. As indicated above, a growth rate is desired which is economically viable on an industrial scale. Hence undesirable growth deficiency may be equated with a reduction in the maximum achievable biomass accumulation below 10 g cell dry weight per litre of culture, e.g. when employing conventional YPD medium containing glucose as the carbon source at 30° C.

Y. lipolytica ST9100 is an example of a preferred Y. lipolytica strain for engineering to provide a yeast in accordance with the invention. i.e. wherein a sterol surrogate is provided to compensate for any native gene for yeast ergosterol production which cannot otherwise be deleted without undesirable growth deficiency or no growth and permit expression of one or more required heterologous genes for desired alternative sterol production. This strain as indicated above is a prior described strain in Arnesen et al. (2020) Front. Bioeng. Biotech. It can be obtained starting from the commercially available Y. lipolytica W29 (MatA) strain Y-63746 as available from the ATCC as ATCC20460™ or from the ARS culture collection. NCAUR, in accordance with the strain development set forth by Arnesen et al., i.e. via initial production of the strain ST6512 strain as described in Marella et al. (2020) Metabolic Eng. 61, 427-436 (also available from Euroscarf as accession no. Y41408). Details for such strain development are also provided in the exemplification herein. It will be appreciated that similar strains to ST9100 with increased squalene synthesis may be developed starting from Y. lipolytica Y-63746 and may be similarly employed as the platform strain for provision of a yeast in accordance with the invention suitable for producing one or more desired non-native sterols with simultaneous provision of a sterol surrogate.

It will be appreciated such sterol surrogate provision may be achieved by intracellular expression of a nucleic acid sequence encoding an enzyme for indirect or preferably direct provision of the sterol surrogate. e.g., such a coding sequence optimised for the particular yeast host. This may be especially preferred where the oleaginous yeast cells are incapable of taking up exogeneous sterols or sterol surrogates from the environment. Further, it will be recognised that the expression of the sterol surrogate will be controlled with a view to preventing diversion of carbon flux from impeding desired non-native sterol production, i.e. the engineered oleaginous yeast must remain suitable for production of the desired one or more non-native sterols, e.g. campesterol and/or 24-methylenecholesterol. Hence, for example, it may be found preferable for expression of the sterol surrogate to be under the control of a weak promoter in the chosen yeast cells. Such a promoter may, for example, be the PrGPAT promoter or PrDGA1 promoter as referred to in Holkenbuink et al. (2018) Biotech. J. EasyCloneYALI: CRISPR/Cas9-Based Synthetic Toolbox for Engineering of the yeast Yarrowia lipolytica, but may be any yeast promoter (synthetic or natural) which provides similar function as determined by conventional fluorescence assay of control of GFP expression. A suitable assay for this purpose is also described in the above-noted paper of Holkenbrink et al. It will be appreciated, however, that other known means for achieving low level expression may alternatively be employed, e.g. promoter truncation, feedback regulation etc.

Preferably, the sterol surrogate may be tetrahymanol. This may be especially preferred where the oleaginous yeast cells, e.g. Y. lipolytica cells, have increased squalene synthesis compared with wild type. Thus the oleaginous yeast may comprise a heterologous nucleic acid encoding a squalene-tetrahymanol cyclase, preferably such a coding sequence optimised for expression in the yeast. For example, the squalene-tetrahymanol cyclase may be the Tetrahymanol thermophila squalene-tetrahymanol cyclase or a functional variant thereof for providing the sterol surrogate.

It will be appreciated however that an alternative sterol surrogate may be provided. For example, the sterol surrogate may be a hopanoid. Again, this may be especially preferred where the oleaginous yeast cells, e.g. Y. lipolytica cells, have increased squalene synthesis compared to wild type. Thus, the oleaginous yeast may comprise a heterologous nucleic acid encoding a squalene-hopene cyclase, preferably codon-optimised for expression in the yeast. For example, the squalene-hopene cyclase may be a Schizosaccharomyces Japonicus squalene-hopene cyclase or a functional variant thereof for providing the sterol surrogate.

As indicated above, the sole purpose of the sterol surrogate is to permit deletion of one or more genes for ergosterol synthesis which otherwise is precluded by growth deficiency or no growth. i.e. compensate for essentiality of a gene. e.g. ERG4 where production of 24-methylenecholesterol is desired or ERG6 where production of cholesterol is desired. It will be understood therefore that as described herein, the term “sterol surrogate” relates to a non-native sterol or functionally equivalent triterpenoid but as implied by the term “surrogate” has a distinct purpose which is distinct from the one or more non-native sterols which it is desired to produce for further application. For example, non-native sterol refers to any plant phytosterol such as desmosterol, cholesterol, 24-methylenecholesterol, campesterol, isofucosterol, beta-sitosterol or stigmasterol which may be desired alone or as part of a mixture of such sterols.

It will also be understood that the yeast provided herein may provide a commercial or industrially relevant amount of non-native sterol as described herein. For example, as further illustrated by the exemplification, a Y. lipolytica yeast in accordance with the invention, derived from Y. lipolytica strain ST9100 by ERG5 knock out and intracellular expression of a heterologous delta-7 sterol reductase, can enable a campesterol yield of 40-42 mg/g dry cell weight (DCW). As also illustrated herein, a Y. lipolytica strain in accordance with the invention derived from Y. lipolytica strain ST9100 with both the native ERG4 and ERG5 genes knocked out and expressing a heterologous delta-7 sterol reductase can enable a 24-methylenecholesterol yield of more than 40 mg/g DCW, e.g. about 48 mg/g DCW. As additionally illustrated by the exemplification, a Y. lipolytica yeast in accordance with the invention and derived from Y. lipolytica strain ST9100 to express intracellularly a sterol surrogate can provide mixtures of non-native sterols of industrial use wherein the yield of 24-methylenecholesterol is still industrially significant, e.g. as much as 9-10 mg/g DCW, for example, in a mixture also including campesterol and cholesterol. Indeed, engineered Y. lipolytica strains have been achieved which provide still higher 24-methylenecholesterol production, above 15 mg/g DCW e.g. about 18-23 mg/g DCW, e.g. between about 22-23 mg/g DCW, as the dominant non-native sterol in a non-native sterol mixture including measurable campesterol and importantly campesterol as the plant (24R) epimer, e.g. a sterol mixture providing 24-methylenecholesterol together with all of campesterol, cholesterol, isofucosterol, desmosterol in quantifiable amount and detectable β-sitosterol as illustrated herein by genetically engineered Y. lipolytica strain ST12178. Similar non-native sterol production might be expected with other oleaginous yeast engineered in accordance with the invention which also exhibit increased squalene synthesis, especially other such engineered Y. lipolytica strains. Such production of 24-methylenecholesterol, either alone or in a mixture, may be especially useful for example for provision of phytosterols in a food composition where a domesticated insect like the honeybee requires several sterols in its diet.

Optionally, the oleaginous yeast of the first aspect comprises an attenuated or deleted endogenous sterol C-22 desaturase (ERG5). Optionally, the oleaginous yeast further comprises an attenuated or deleted endogenous delta-24 sterol reductase (ERG4). Optionally, the oleaginous yeast comprises an attenuated or deleted endogenous sterol C-24 methyltransferase (ERG5). Optionally, the oleaginous yeast comprises an attenuated or deleted endogenous sterol C-22 desaturase (ERG5) and further comprises an attenuated or deleted endogenous delta-24 sterol reductase (ERG4), and/or sterol C-24 methyltransferase (ERG6).

Preferably, the oleaginous yeast of the first aspect is further engineered to comprise one or more heterologous nucleic acid sequences capable of expression to provide one or more of:
a. a delta-7 sterol reductase enzyme;
b. a delta-24(28) sterol reductase enzyme;
c. a delta-24(25) sterol reductase enzyme;
d. a sterol C-28 sterol methyltransferase enzyme and
e. a sterol C-22 desaturase enzyme
   whereby production of the desired one or more non-native sterols or compounds derived therefrom can be achieved. One or more heterologous delta-24(28) sterol reductase and/or delta-24(25) sterol reductase may be provided as further discussed below depending on the non-native sterol or sterols required.

Thus, an oleaginous yeast of the invention expressing a sterol surrogate, e.g. tetrahymanol, may have any combination of (i) endogenous gene deletion or attenuation(s) with (ii) expressible heterologous gene(s) as set out in Table 2 with a view to producing a specific sterol, e.g. campesterol or 24-methylenecholesterol or a mixed sterol composition.

### More Specifically, Such a Yeast May be an Oleaginous Yeast as Follows:

a. wherein the oleaginous yeast further comprises:
(i) an attenuated or deleted endogenous sterol C-22 desaturase enzyme (ERG5);
(i) a heterologous nucleic acid sequence encoding a delta-7 sterol reductase enzyme, whereby one or more non-native sterols can be produced comprising campesterol (see FIG. 4);
b. wherein the oleaginous yeast further comprises:
(i) an attenuated or deleted endogenous sterol C-22 desaturase enzyme (ERG5) and an attenuated or deleted delta-24 sterol reductase enzyme (ERG4):
(ii) a heterologous nucleic acid sequence encoding a delta-7 sterol reductase enzyme, whereby one or more non-native sterols can be produced comprising 24-methylenecholesterol (see FIG. 5);
c. wherein the oleaginous yeast further comprises:
(i) an attenuated or deleted endogenous sterol C-24 methyltransferase (ERG6), optionally an attenuated or deleted endogenous sterol C-22 desaturase enzyme (ERG5) and/or optionally an attenuated or deleted delta-24 sterol reductase enzyme (ERG4) and;
(ii) a heterologous nucleic acid sequence encoding a delta-7 sterol reductase enzyme, whereby one or more non-native sterols can be produced comprising desmosterol (see FIG. 6);
d. wherein the oleaginous yeast further comprises:
(i) an attenuated or deleted endogenous sterol C-22 desaturase enzyme (ERG5) and an attenuated or deleted sterol C-24 methyltransferase (ERG8);
(ii) a heterologous nucleic acid sequence encoding a delta-7 sterol reductase enzyme; and
(lii) a heterologous nucleic acid sequence encoding a delta-24 sterol reductase enzyme, whereby one or more non-native sterols are produced comprising cholesterol (see FIG. 7);
e. wherein the oleaginous yeast further comprises:
(i) an attenuated or deleted endogenous sterol C-22 desaturase enzyme (ERG5) and an attenuated or deleted delta-24 sterol reductase enzyme (ERG4);
(ii) a heterologous nucleic acid sequence encoding a delta-7 sterol reductase enzyme and
(iii) a heterologous nucleic acid sequence encoding a sterol C-28 methyltransferase enzyme,
whereby one or more non-native sterols can be produced comprising isofucosterol (delta-24(28)-Z isomer) or fucosterol (delta-24(28)-E isomer) (see FIG. 8 and FIG. 16b);
f. wherein the oleaginous yeast further comprises:
(i) an attenuated or deleted endogenous sterol C-22 desaturase enzyme (ERG5):
(ii) a heterologous nucleic acid sequence encoding a delta-7 sterol reductase enzyme and
(iii) a heterologous nucleic acid sequence encoding a sterol C-28 methyltransferase enzyme.
wherein one or more non-native sterols can be produced comprising beta-sitosterol (see FIG. 9)
g. wherein the oleaginous yeast further comprises:
(i) a heterologous nucleic acid sequence encoding a delta-7 sterol reductase enzyme and
(ii) a heterologous nucleic acid sequence encoding a sterol C-28 methyltransferase enzyme
whereby one or more non-native sterols are produced comprising stigmasterol; optionally,
wherein the oleaginous yeast has an attenuated or deleted endogenous sterol C-22 desaturase enzyme (ERG5) and/or delta-24 sterol reductase enzyme (ERG4) and optionally additionally one or more further heterologous nucleic acid sequences are provided to express a DWF1 enzyme and/or sterol C-22 desaturase enzyme (see FIG. 10)
h. wherein the oleaginous yeast further comprises:
(i) an attenuated or deleted endogenous sterol C-22 desaturase enzyme (ERG5) and an attenuated delta-24 sterol reductase enzyme (ERG4), preferably where the ERG5 gene is deleted and the activity of ERG4 is attenuated by provision of the same encoding sequence, or a corresponding plant delta-24(28) sterol reductase (DWF1) coding sequence, under the control of a weak promoter selected from PrDGA1 and functionally equivalent weak yeast promoters;
(ii) a heterologous nucleic acid sequence encoding a delta-7 sterol reductase enzyme, optionally a heterologous nucleic acid sequence encoding a delta-24(25) sterol reductase and/or optionally a heterologous nucleic acid sequence encoding a C-28 sterol methyl transferase, whereby a non-native sterol mixture can be produced, preferably such that a sterol mixture can be produced comprising both 24-methylenecholesterol and campesterol, optionally together with one or more further non-native sterols in detectable amount, e.g. cholesterol.

Where it desired to produce a sterol mixture as in (h) above in which campesterol is present as the plant (24R) epimer, it will be recognised that normal yeast delta-24 sterol reductase activity will in effect be attenuated with additional consequential change of stereochemistry to ensure production of campesterol as the 24R epimer by replacement of the ERG4 gene by a coding sequence for an enzyme functioning as a plant delta-24(28) reductase (i.e. DWF1 enzyme) under the control of a weak yeast promoter as defined. “Plant DWF1 coding sequence” in this context will be understood as a coding sequence providing any enzyme with the characteristics of a plant delta-24(28) reductase capable of converting some 24-methylenecholesterol to campesterol as the 24R epimer. Conveniently, the DWF1 encoded may be one of many naturally occurring such enzymes, e.g. the delta-24(28) of Solanum tuberosum. Where production of the plant 24R epimer of campesterol is not an overriding consideration, then attenuation of yeast ERG4 may be by simply providing an alternative to the native ERG4 gene in which the native ERG4 coding sequence is under the control of a weak yeast promoter.

Both Solanum tuberosum and Solanum lycopersicum possess two delta-24 sterol reductase variants, one catalyses delta-24(25) reduction and the other catalyses delta-24(28) reduction. While such a delta-24(28) sterol reductase can be employed as a substitute for yeast ERG4 activity, it will be recognised that for cholesterol production as descried above a heterologous coding sequence will be provided encoding a delta-24(25) sterol reductase. Preferably for example the delta-24(25) sterol reductase of Solanum lycopersicum may be chosen for this purpose as illustrated by the exemplification provided below.

Where a heterologous coding sequence is referred to above it will be appreciated that generally this will be a coding sequence codon-optimised for the yeast host species. Heterologous nucleic acid sequences may be provided, for example, encoding all of a delta-7 sterol reductase with deletion of the ERG5 gene, a plant DWF1 as discussed above to in effect substitute for the native ERG4 gene and additionally both a delta-24(25) sterol reductase and a C-28 sterol methyltransferase whereby a sterol mixture can be obtained comprising 24-methylenecholesterol, the plant (24R) epimer of campesterol and measurable cholesterol together with other non-native sterols. 24-methylenecholesterol may preferably be the dominant sterol in such sterol mixture which may contain additional non-native sterols in at least detectable amount, e.g. all of isofucosterol, desmosterol and beta-sitosterol.

As indicated above as an alternative to PrDGA1 as a weak yeast promoter may be chosen PrGPAT as disclosed in Holkenbrink et al (2018) ibid. However, as also indicated above functionally equivalent weak yeast promoters may be readily determined in well-known manner. Any promoter may be employed that gives expression in the same genomic context capable of fulfilling the same purpose. Such determination may for example include conventional fluorescence assay of control of GFP expression in appropriate yeast host cells, e.g. Y. lipolytica yeast cells. The term ‘weak yeast promoter’ as used herein will be understood to include promoters that by such assay in Y. lipolytica yeast cells, e.g. the reference wild-type Y. lipolytica W29 strain Y-63746, show lower activity than the well-known PrTEFintron promoter as first employed for oil production in Y. lipolytica (Tai and Stephanopoulos, Metab. Eng. (2013) 1-9). For the sequence of the PrTEFintron reference may again be made to the supplementary information of the Holkenbrink et al (2018) paper. The term ‘PrGPAT promoter’ as used herein will be understood to always equate with the sequence given for that promoter in the referenced 2018 paper of Holkenbrink et al. (i.e. the sequence corresponding to the promoter region of gene YALI1_C00209 g in Y. lipolytica W29 strain Y-83746, with genomic location of the promoter being Chromosome 1C:20927-22056, and genomic location of the downstream gene being Chromosome 1C: 18,422-20,926)

With regard to a yeast of the invention as above for production of stigmasterol, although no endogenous genes are deleted in this case, an optimal activity of C-28 sterol methyltransferase can be expected to divert much, if not all, pathway flux away from ergosterol biosynthesis towards stigmasterol production. In this case, ergosterol would be absent or too low to fulfil cellular sterol requirements such as membrane requirements. Stigmasterol may not adequately substitute for ergosterol. Presence of tetrahymanol or another sterol surrogate would therefore be beneficial to such a strain in order to fulfil the membrane requirements and improve cell fitness thereby aiding growth.

In some instances of engineered yeast strains of the invention, it may prove beneficial to employ more than one heterologous coding sequence to provide a required activity, for example where C-28 sterol methyltransferase activity is required one or more C-28 sterol methyltransferase coding sequences may employed. Mere more than one heterologous coding sequence is provided for a required enzyme activity such coding sequences may encode the same enzyme or different enzymes, e.g. 2 or 3 different enzymes. For example, beta-sitosterol and isofucosterol-producing engineered Y. lipolytica strains may be provided as described herein with up to 3 different C-28 sterol methyltransferase genes. Increasing the copy number of heterologous genes (same or different variants) for any required activity may be advantageous to increase pathway flux to a desired product.

Just as campesterol may be produced as one of two epimers in a yeast of the invention, the same applies for isofucosterol. It is recognised that isofucosterol can be produced, for example, in green plants as the delta-24(28)-Z isomer or can be found alternatively as the delta-24(28)-E isomer, for example in red algae and as such is commonly referred to as fucosterol. This arises from the nature of the C-28 methyltransferase (SMT; EC2.1.1.143) responsible as shown in FIG. 18b. Depending on the C-28 sterol methyltransferase provided for isofucosterol production in a yeast of the invention, either the Z isomer or E isomer may thus be attained. Hereinafter the term ‘isofucosterol’ should be taken to embrace production of either isomer, unless a specific plant C-28 sterol methyltransferase is specified as in the exemplification which ensures the Z-isomer.

The oleaginous yeast may be any oleaginous yeast where provision of the sterol surrogate is required to facilitate cell growth in the face of the specified gene alterations including any deletion or deletions in the ergosterol biosynthesis pathway. Preferably for example, the oleaginous yeast may be an engineered Y. lipolytica production strain, for example such a strain engineered from Y. lipolytica ST9100 or another Y. lipolytica which shares all or some of the same modified genotype features of Y. lipolytica ST9100 compared to Y. lipolytica W29 strain Y-63746 as the reference strain with increased synthesis of squalene, or possibly another sterol precursor or sterol pathway intermediate, compared to that reference strain.

It will be understood that “deleted” equates with non-functional and thus alternatively a deleted gene may be described as knocked-out. This may be achieved in any known manner, e.g. using a CRISPR-Cas/gRNA or marker-mediated gene deletion.

As regards (a) above, it will be recognised that preferably the endogenous ERG4 gene may be additionally substituted by a plant delta-24(28) sterol reductase (DWF1; e.g. the DWF1 of Arabidopsis thaliana or more preferably the delta-24(28) sterol reductase of Solanum tuberosum) such that the 24α configuration (R) of campesterol is attained. Again it will be recognised that the designation ‘plant delta-24(28) sterol reductase’ can be applied to any delta-24 reductase which ensures the correct plant stereochemistry at the C24 position when converting 24-methylenecholesterol to campesterol as shown in FIG. 16. As indicated above, this may be highly preferred for downstream uses of campesterol, e.g., for provision in honeybee feed. For example, the moulting hormone Makisterone A is produced from campesterol in bees. Provision of the 24β epimer of campesterol would lead to production of epi-Makisterone A, which may not function in the same way. The same ERG4 gene substitution may be additionally favoured in producing beta-sitosterol and stigmasterol; see FIGS. 9 and 10. As already discussed above, it may also be favoured in producing a sterol mixture in accordance with (h) above comprising 24-methylenecholesterol and campesterol, e.g. particularly such a sterol mixture to be used as a source of sterols in an artificial feed for honeybees or other insects or animals.

Plant DWF1 enzymes that may be employed for substitution of ERG4 as above include by way of example any of Solanum tuberosum delta-24(28) sterol reductase (NCBI seq. ref: BAQ55274.1), Arabidopsis thaliana delta-24 sterol reductase (NP_850618.1), Arachis duranensis delta-24 sterol reductase (XP_015952627.2), Selaginella moellendorffii delta-24 sterol reductase (XP_002960921.1), Capsicum chinense delta-24 sterol reductase (PHU28881.1), Artemisia annua delta-24 sterol reductase (PWA66182.1), Helianthus annuus delta-24 sterol reductase (XP_022012299.1), Cocos nucifera delta-24 sterol reductase (EHA8587492.1), Triticum urartu delta-24 sterol reductase (EMS57493.1), Gracilariopsis chorda delta-24 sterol reductase (PXF44537.1), Capsella rubella delta-24 sterol reductase (XP_006297345.1) and Ajuga reptans var. atropurpurea delta-24 sterol reductase (BAS68578.1)

Yeast in accordance with the invention as discussed above which are provided with a sterol surrogate may be specifically engineered to produce sterol mixtures of interest with provision of heterologous genes in combination with endogenous ERG5 gene deletion or attenuation, preferably deletion, and attenuation of ERG4 activity, for example preferably whereby 24-methylenecholesterol is produced in combination with campesterol, possibly with one or more further phytosterols, e.g. cholesterol. Preferably, 24-methylenecholesterol may be the major sterol of the mixture, e.g. at a level of about 2-3 mg/g or higher DCW, possibly as high as 9-10 mg/g DCW or as indicated above even considerably higher. e.g. at least 15 mg/g DCW, preferably at least about 18 or even at least 20 mg/g DCW. In this case, the endogenous ERG5 gene may be deleted in conventional manner, e.g. using CRISPR-Cas9/gRNA. Attenuation of endogenous ERG4 activity may be achieved by changing the promoter of the native ERG4 gene to a weaker promoter e.g. deleting the native ERG4 gene and re-introducing the ERG4 coding sequence under the control of a weaker yeast promoter, e.g. a weak yeast promoter as discussed above such as a PrDGA1 promoter. Alternatively, the endogenous ERG4 gene may be substituted by a plant DWF1 coding sequence under the control of such a promoter. Indeed, as noted above, this may be favoured so that the plant-produced epimer of campesterol is attained in the resulting non-native sterol mixture. As indicated above, this is combined with expression of a heterologous delta-7 sterol reductase, optionally with a delta-24(25) sterol reductase or C-28 sterol methyltransferase or both a delta-24(25) sterol reductase and a C-28 sterol methyltransferase. Expression of a heterologous delta-7 sterol reductase with just a delta-24(25) sterol reductase may be preferred. This is so since the delta-24(25) sterol reductase will permit conversion of 24-methylenecholesterol to cholesterol. Expression may be controlled so that all of 24-methylenecholesterol, campesterol and cholesterol is attained at measurable amount. See Example 4. In this way, a Y. lipolytica derived from strain ST9100 has been attained which produces nearly 10 mg/g DCW of 24-methylenecholesterol, together with both significant campesterol and cholesterol and unexpectedly some desmosterol.

It will be appreciated that such an advantageous sterol mixture in which 24-methylenecholesterol is combined with campesterol and cholesterol, preferably with yeast expression of a DWF1 sequence to attain the correct plant epimer of campesterol, may also be attained by equivalent engineering of alternative oleaginous yeast strains, e.g. Y. lipolytica strains which like ST9100 have increased squalene synthesis compared with the corresponding wild type.

It will be recognised that advantageous sterol mixtures which can be attained by engineering of yeast strains of the invention also include such sterol mixtures in which 24-methylenecholesterol is combined with isofucosterol, preferably such strains with deletion of ERG4 and ERG5 and in which expression of a SMT variant (i.e. C-28 sterol methyltransferase variant) ensures production of isofucosterol as the delta-24(28) Z-isomer. Suitable such C-28 methyltransferase variants include, for example, any of the C-28 methyltransferase variants of Chenopodium quinoa, Arabidopsis thaliana and Amborella trichopoa. Expression of one or more heterologous coding sequences for a C-28 methyltransferase will be combined with expression of one or more heterologous coding sequences for a delta-7 sterol reductase enzyme. Such production of isofucosterol is illustrated in the exemplification by engineering of Y. lipolytica strains derived from ST9100. Equivalent engineering of alternative oleaginous yeast strains, e.g. Y. lipolytica strains which like ST9100 have increased squalene synthesis compared with the corresponding wild type, is however again contemplated.

It will be appreciated that such an advantageous sterol mixture in which isofucosterol as the delta-24(28) Z-isomer is combined with 24-methylenecholesterol may additionally include cholesterol arising from additional expression of a delta-24(25) sterol reductase. Again such sterol mixture production may be attained by equivalent engineering of various alternative oleaginous yeast strains, e.g. Y. lipolytica ST9100 and other Y. lipolytica strains which like ST9100 have increased squalene synthesis compared with the corresponding wild type.

As indicated above, neither 24-methylenecholesterol nor isofucosterol has previously been available industrially. Significantly, by means of yeast of the invention isofucosterol can be obtained, preferably as the delta-24(28)-Z isomer, for incorporation into compositions and hence artificial dietary compositions for bees and other insects or animals can now be disclosed for the first time which incorporate this sterol. It will be appreciated that such incorporation may be preferably as part of a sterol mixture attained in accordance with the invention additionally comprising 24-methylenecholesterol, for example comprising both 24-methylenecholesterol and campesterol as the 24R plant epimer.

As now further highlighted, especially preferred may be expression of a heterologous delta-7 sterol reductase with a delta-24(28) sterol reductase (DWF1), a delta-24(25) sterol reductase and C-28 sterol methyltransferase accompanied by deletion of the endogenous ERG5 gene and attenuation of ERG4 activity to again attain a sterol mixture comprising all of 24-methylenecholesterol, campesterol, preferably as the plant epimer in view of expression of a plant DWF1 coding sequence in substitution for yeast ERG4, and cholesterol plus additional sterols in detectable amount. In this way, a Y. lipolytica derived from strain ST9100, or a similar Y. lipolytica strain as discussed above, may be attained capable of producing 24-methylenecholesterol as the dominant sterol plus other sterols including all of campesterol as the plant (24R) epimer, cholesterol, isofucosterol and desmosterol and possibly also beta-sitosterol in at least detectable amount, for example where the production of 24-methylenecholesterol is at least 20 mg/g DCW, e.g. about 22-23 mg/g DCW, but other sterols are also attained at measurable amount, including all of campesterol, cholesterol, isofucosterol and desmosterol. It has been shown that such a sterol mixture may be achieved in which campesterol as the plant epimer is present in an amount of at least 2-3 mg/g DCW and cholesterol is also present at higher amount, e.g. at least 8-9 mg/g DCW, together with isofucosterol and desmosterol. Such a strain is illustrated by Y. lipolytica strain ST12178, the engineering of which is described in Example 4. Production of a sterol mixture by an oleaginous yeast of the invention in which 24-methylenecholesterol is present with at least campesterol as the plant epimer but preferably also an of at least cholesterol, isofucosterol and desmosterol, preferably additionally with beta-sitosterol, is especially of interest in relation to provision of a sterol mixture in a dietary composition for honeybees. For this purpose, sterols may be isolated from yeast cells of the invention. However, alternatively yeast cels of the invention may be recovered from culture as a yeast biomass which is then inactivated, e.g. heat-inactivated, and dried, e.g. by heating at no more than 60° C. The dried yeast biomass may then be preferably converted to a powder. Such a dried yeast biomass can be stored frozen, e.g. at −20° C., prior to use, e.g. incorporation into a composition such as a dietary composition. Mile such a dietary composition is of especial interest to the inventors for provision as a feed to honeybees, it will be appreciated that such compositions may have wider use, e.g. in the healthcare field for humans and animals. The sterol composition could be adjusted as required in each case. In some instances, a non-yeast produced sterol supplement may be added. e.g., such a supplement comprising beta-sitosterol.

In the case of oleaginous yeast strains of the invention expressing both a delta-24(25) sterol reductase and a C-28 sterol methyltransferase so as to produce a mixture of phytosterols, it has additionally surprisingly been found that omission of the sterol surrogate gene from such a sterol mixture producing strain may be carded out with retention of useful phytosterol mixture production including campesterol, 24-methylenecholesterol and cholesterol, making up the majority of the mixture. Again by using a DWF1 plant enzyme coding sequence under the control of a weak promoter, campesterol may be attained in industrially significant amount with the plant stereochemistry at the C-24. The delta-24(25) sterol reductase and C-28 sterol methyltransferase may also be expressed employing a weak yeast promoter such as a PrGPAT promoter as disclosed in Holkenbrink et al (2018) ibid.

The studies reported herein enable other genetic modifications of oleaginous yeasts taught above to be extended for the first time to such yeast without a sterol surrogate gene to attain desired non-native sterol or non-native sterol mixture production. The sterol composition of such modified strains may nevertheless be dramatically altered to achieve commercially useful production of exogenous sterols with high purity and titre.

In relation to such embodiments of the invention as now discussed below, it is not deemed feasible for any sterol pathway engineering in a facultative anaerobic yeast such as S. cerevisiae to be assumed to be immediately transferable to an obligate aerobic yeast such as Y. lipolytica. In this connection, it is for example, especially noteworthy that ERG8 has been found to be essential in Y. lipolytica ST9100, as will be returned to below in relation to cholesterol production, as well as in the wild-type Y. lipolytica strains W29 (CLIB89/ATCC20460™, Patterson et al. (2018) Metab. Eng. 48, 184-196) and PO1f (MatA, leu2-270, ura3-302,xpr2-322, axp-2, Schwartz et al. (2019) Metab. Eng. 55, 102-110).

Thus in a further aspect of the invention, there is provided an oleaginous yeast for production of at least one non-native sterol, wherein the at least one native sterol comprises 24-methylenecholesterol or a derivative thereof, the yeast comprising:
(i) an attenuated or deleted endogenous sterol C-22 desaturase enzyme (ERG5) and an attenuated or deleted delta-24 sterol reductase enzyme (ERG4); and
(ii) a heterologous nucleic acid sequence encoding a delta-7 sterol reductase enzyme.

In another aspect, there is provided an oleaginous yeast for production of at least one non-native sterol, wherein the at least one non-native sterol comprises desmosterol or a derivative thereof, the yeast comprising:
(i) an attenuated or deleted endogenous sterol C-24 methyl-transferase (ERG6), optionally an attenuated or deleted endogenous sterol C-22 desaturase enzyme (ERG5) and/or optionally an attenuated or deleted delta-24 sterol reductase enzyme (ERG4) and; (ii) a heterologous nucleic acid sequence encoding a delta-7 sterol reductase enzyme.

In a still further aspect, there is provided an oleaginous yeast for production of at least one non-native sterol, wherein the at least one non-native sterol comprises isofucosterol (delta-24(28)-Z isomer) and/or fucosterol (delta-24(28)-E isomer) or a derivative thereof, the yeast comprising:
(i) an attenuated or deleted endogenous sterol C-22 desaturase enzyme (ERG5) and an attenuated or deleted delta-24 sterol reductase enzyme (ERG4);
(ii) a heterologous nucleic acid sequence encoding a delta-7 sterol reductase enzyme; and
(iii) a heterologous nucleic acid sequence encoding a sterol C-28 methyl-transferase enzyme.

In another aspect, there is provided an oleaginous yeast for production of at least one non-native sterol, wherein the at least one native sterol comprises cholesterol or a derivative thereof the oleaginous yeast comprising:
(i) an attenuated or deleted endogenous sterol C-22 desaturase enzyme (ERG5) and an attenuated or deleted sterol C-24 methyl-transferase (ERG6);
(ii) a heterologous nucleic acid sequence encoding a delta-7 sterol reductase enzyme;
(iii) a heterologous nucleic acid sequence encoding a delta-24 sterol reductase enzyme.

As noted above. ERG6 is essential in a Y. lipolytica such as Y. lipolytica ST9100 so a cholesterol-producing engineered such strain could not be produced by the same methods described to create cholesterol-producing strains of S. cerevisiae or P. pastoris. In contrast, a cholesterol-producing oleaginous yeast of the invention expressing a sterol surrogate such as tetrahymanol might be evolved to remove sterol surrogate dependency. The same extrapolation can reasonably be made for any sterol surrogate dependent yeast of the invention described above.

In another aspect, there is provided an oleaginous yeast for production of at least one non-native sterol, wherein the least one non-native sterol comprises beta-sitosterol or a derivative thereof, the oleaginous yeast comprising:
(i) an attenuated or deleted endogenous sterol C-22 desaturase enzyme (ERG5);
(ii) a heterologous nucleic acid sequence encoding a delta-7 sterol reductase enzyme and
(iii) a heterologous nucleic acid sequence encoding a sterol C-28 methyltransferase enzyme.

In a still further aspect, there is provided an oleaginous yeast for production of at least one non-native sterol where the at least one non-native sterol comprises stigmasterol or a derivative thereof, the yeast comprising:
(i) a heterologous nucleic acid sequence encoding a delta-7 sterol reductase enzyme, e.g. a plant DWF5 and
(ii) a heterologous nucleic acid sequence encoding a sterol C-28 methyltransferase enzyme, e.g. a plant SMT2; optionally.
wherein the oleaginous yeast has an attenuated or deleted endogenous sterol C-22 desaturase enzyme (ERG5) and/or delta-24 sterol reductase enzyme (ERG4) and optionally additionally one or more further heterologous nucleic acid sequences are provided to express a plant delta-24(28) sterol reductase (DWF1) enzyme and/or sterol C-22 desaturase enzyme.

When the yeast includes an attenuated or deleted endogenous ERG5, the yeast will include a heterologous nucleic acid encoding a sterol C-22 desaturase enzyme, e.g. plant CYP710A. The yeast may include an attenuated or deleted endogenous ERG4 and have in substitution a heterologous nucleic acid sequence encoding a plant delta-24(28) sterol reductase enzyme (DWF1). This provides a yeast that produces stigmasterol in the same configuration (epimer) as found in plants. See FIG. 10.

In yet another aspect, there is provided an oleaginous yeast for production of a non-native sterol mixture, the yeast comprising:
(i) an attenuated or deleted endogenous sterol C-22 desaturase enzyme (ERG5) and an attenuated delta-24 sterol reductase enzyme (ERG4), preferably where the ERG5 gene is deleted and the activity of ERG4 is attenuated by provision of the same encoding sequence, or a corresponding plant delta-24(28) sterol reductase (DWF1) coding sequence, under the control of a weak promoter selected from PrDGA1 and functionally equivalent weak yeast promoters;
(i) a heterologous nucleic acid encoding a delta-7 sterol reductase enzyme, optionally a heterologous nucleic acid encoding a delta-24(25) sterol reductase and/or optionally additionally a heterologous nucleic acid encoding a C-28 sterol methyl transferase, whereby a non-native sterol mixture is produced, preferably such that a sterol mixture is produced comprising both 24-methylenecholesterol and campesterol, optionally together with one or more further non-native sterols, e.g. cholesterol.

Preferably, the oleaginous yeast may be the oleaginous yeast Yarrowia lipolytica, for example a yeast derived from Yarrowia lipolytica W29 strain Y-63746, As indicated above, especially preferred is a yeast derived from the Y. lipolytica strain ST9100 in which case a sterol surrogate may be provided intracellularly to enable required native gene deletion or attenuation but then removed from the strain after further engineering to provide heterologous coding sequences. As indicated above, this may for example be achieved and preferred where the required heterologous coding sequences enable a mixture of non-native sterols to be produced, e.g. all of campesterol, 24-methylenecholesterol, cholesterol and desmosterol, possibly supplemented by isofucosterol and/or beta sitosterol, as exemplified herein.

Genetically modified yeast of the invention may be cultured in conventional manner for production of the one or more desired sterols or derivatives thereof, preferably with a simple carbon source such as glucose. The one or more sterols thus synthesized may be converted to one or more further sterol derivatives in the same cells or once isolated. The one or more sterols or derivatives thus produced may be further incorporated into a composition for dietary, cosmetic or pharmaceutical use (e.g. any of an artificial dietary composition for animals or insects such as bees, a food product, an agricultural composition, a cosmetic composition and pharmaceutical composition). As indicated above, yeast of the invention may for example be genetically-modified with a view to specifically addressing the desire for sterols for incorporation into artificial bee feed, for example, production from a single genetically-modified yeast strain of a mixed sterol composition comprising 24-methylenecholesterol and campesterol, possibly with one or more other phytosterols. It may be chosen to simply incorporate yeast cells of the invention into an artificial dietary composition, e.g. for bees, without a sterol extraction step. In this case as discussed above such yeast cell incorporation may be in the form of a dried yeast cell biomass attained following recovery of yeast cells from a culture medium, e.g. culture medium following growth of yeast cells by conventional fed-batch fermentation.

Embodiments of the invention are further described herein with reference to the accompanying drawings as set out below.

### BRIEF DESCRIPTION OF FIGURES

FIG. 1 shows the chemical formula and nomenclature used for various sterols including, desmosterol, cholesterol, 24-methylenecholesterol, campesterol, isofucosterol, beta-sitosterol, and stigmasterol.

FIG. 2 shows a schematic of the later stage of the biosynthetic pathway for sterol production in yeast.

FIG. 3 shows a schematic of the production of a sterol surrogate in yeast. TtSTC1 (squalene-tetrahymanol cyclase) is introduced.

FIG. 4 shows a schematic of genetic modifications made to the later stage of the biosynthetic pathway for sterol production in yeast to produce campesterol. The gene ERG5 (sterol C-22 desaturase enzyme) has reduced activity (for example is knocked out (KO)) and a heterologous DWF5 (delta-7 sterol reductase enzyme) is introduced.

FIG. 5 shows a schematic of genetic modifications made to the later stage of the biosynthetic pathway for sterol production in yeast to produce 24-methylenecholesterol. The genes ERG5 (sterol C-22 desaturase enzyme) and ERG4 (delta-24 sterol reductase enzyme) have reduced activity (for example are knocked out (KO)) and heterologous DWF5 (delta-7 sterol reductase enzyme) is introduced.

FIG. 6 shows a schematic of genetic modifications made to the later stage of the biosynthetic pathway for sterol production in yeast to produce desmosterol. The gene ERG6 (sterol C-24 methyl-transferase) has reduced activity (for example is knocked out (KO)) and heterologous DHCR7 (delta-7 sterol reductase enzyme) is introduced. Optionally, the genes ERG5 (sterol C-22 desaturase enzyme) and ERG4 (delta-24 sterol reductase enzyme) also have reduced activity (for example are knocked out (KO)).

FIG. 7 shows a schematic of genetic modifications made to the later stage of the biosynthetic pathway for sterol production in yeast to produce cholesterol. The genes ERG6 (sterol C-24 methyl-transferase) and ERG5 (sterol C-22 desaturase enzyme) have reduced activity (for example are knocked out (KO)) and heterologous DHCR7 (delta-7 sterol reductase enzyme) and DHCR24 (delta-24 sterol reductase enzyme) are introduced.

FIG. 8 shows a schematic of genetic modifications made to the later stage of the biosynthetic pathway for sterol production in yeast to produce isofucosterol. The genes ERG5 (sterol C-22 desaturase enzyme) and ERG4 (delta-24 sterol reductase enzyme) have reduced activity (for example are knocked out (KO)) and heterologous DWF5 (delta-7 sterol reductase enzyme) and heterologous SMT2 (sterol C-28 methyltransferase enzyme) are introduced.

FIG. 9 shows a schematic of genetic modifications made to the later stage of the biosynthetic pathway for sterol production in yeast to produce beta-sitosterol. The gene ERG5 (sterol C-22 desaturase enzyme) has reduced activity (for example is knocked out (KO)) and heterologous DWF5 (delta-7 sterol reductase enzyme) and heterologous SMT2 (sterol C-28 methyl-transferase enzyme) are introduced.

FIG. 10 shows a schematic of genetic modifications made to the later stage of the biosynthetic pathway for sterol production in yeast to produce stigmasterol. Heterologous DWF5 (delta-7 sterol reductase enzyme) and heterologous SMT2 (sterol C-28 methyltransferase enzyme) are introduced. Optionally the gene ERG5 (sterol C-22 desaturase enzyme) has reduced activity (for example is knocked out (KO)) and CYP710A1 (sterol C-22 desaturase enzyme) is introduced.

FIGS. 11A and B: A. compares sterol and tetrahymanol production by Y. lipolytica strains as referred to in Example 3. Values represent the mean of three technical replicates. Error bars represent ±1 standard deviation. B. compares sterol and tetrahymanol production in engineered Y. lipolytica strains according to the invention for the production of campesterol and 24-methylenecholesterol (ST11071 and ST11064) and in parental strains used in their construction (ST6512, ST9100 and ST1105. ST11027 an ST11040). STC=expressing a squalene-tetrahymanol cyclase. Deletion of the ERG4 and ERG5 genes is indicated by—erg4 and—erg5 respectively. DHCR7=a delta-7 sterol reductase enzyme. Values again represent the mean of three technical replicates. Error bars represent ±1 standard deviation.

FIG. 12 shows campesterol production by strains ST11086 to ST11075. A different delta-7 sterol reductase source was used for each strain. Codon optimised coding sequences for each such variant enzyme were employed under the control of the PrTEFintron promoter. Values represent the mean of three technical replicates. Error bars represent ±1 standard deviation.

FIG. 13 shows campesterol production by strains expressing Ectocarpus silculosus delta-7 sterol reductase under the control of the PrTEFintron promoter in different genetic backgrounds. Values represent the mean of three technical replicates. Error bars represent ±1 standard deviation.

FIG. 14 shows 24-methylenecholesterol production by strains ST11056 to ST11065. Values represent the mean of three technical replicates. Error bars represent ±1 standard deviation.

FIG. 15 shows sterol and tetrahymanol production by various engineered Y. lipolytica strains as referred to in the exemplification. Values represent the mean of three technical replicates. Error bars represent ±1 standard deviation. Asterix (*) represents trace amount where value is below limit of detection for quantification.

FIG. 16A shows a schematic of comparison of action of ERG4 (i.e. yeast sterol C-24 reductase) and DWF1 (i.e. plant delta-24(28) sterol reductase) leading to different stereochemistry at the C-24 position of a sterol such as campesterol.

FIG. 16B shows a schematic comparison of production of isofucosterol (delta-24(28)-Z isomer) or fucosterol (delta-24(28)-E isomer) relying on C-28 methyltransferases of different source.

FIG. 17A shows (1) production of β-sitosterol, campesterol and tetrahymanol by engineered Y. lipolytica strains ST11804 and ST12139 as described in Example 4 and (1) production of isofucosterol, 24-methylenecholesterol and tetrahymanol by engineered Y. lipolytica strains ST11803 and ST21208 as also described in Example 4. Values represent the mean of three technical replicates. Error bars represent ±1 standard deviation.

FIG. 17B shows (I) desmosterol and tetrahymanol production by engineered Y. lipolytica strain ST11346 a described in Example 4 and (i) production of cholesterol and tetrahymanol by engineered Y. lipolytica strains ST11829 and ST11830 as additionally described in Example 4. The only difference between strains ST11829 and ST11830 is the heterologous delta-24 sterol reductase provided in the cells via expression of a coding sequence codon-optimised for Y. lipolytica, the delta-24 sterol reductase from Danio rerio or the delta-24 sterol reductase from Mus musculus respectively. Strain ST11829 expressing the Danio rerio delta-24 sterol reductase produced far higher cholesterol, almost twice as much at 26.6 mg/g DCW. Values represent the mean of three technical replicates. Error bars represent ±1 standard deviation.

FIG. 18A shows production of 24-methylenecholesterol and tetrahymanol in the ΔERG4ΔERG5 Y. lipolytica strain ST11064 expressing the delta-7 sterol reductase of Tetraselmis sp. GSL018 under the control of the PrTEFintron promoter and non-native sterol production of strains further engineered therefrom: (i) strain ST11943 additionally expressing the delta-24(28) sterol reductase of Solanum tuberosum under the control of a weaker promoter to enable production of campesterol as the plant epimer and (ii) strain ST12140 engineered from strain ST11943 to additionally express a C-28 sterol methyltransferase under the control of a weak yeast promoter. Error bars represent ±1 standard deviation Asterix (*) represents trace amount of R-sitosterol where value is below limit of detection for quantification:

FIG. 188 shows sterol mixture production of a Y. lipolytica strain ST12178, engineered from strain ST12140 by additional introduction of expression of the delta-24(25) sterol reductase of S. lycopersicum from a codon-optimise coding sequence under the control of the PrGPAT promoter. Again error bars represent ±1 standard deviation. Asterix (*) represents trace amount of β-sitosterol below the limit of detection for quantification.

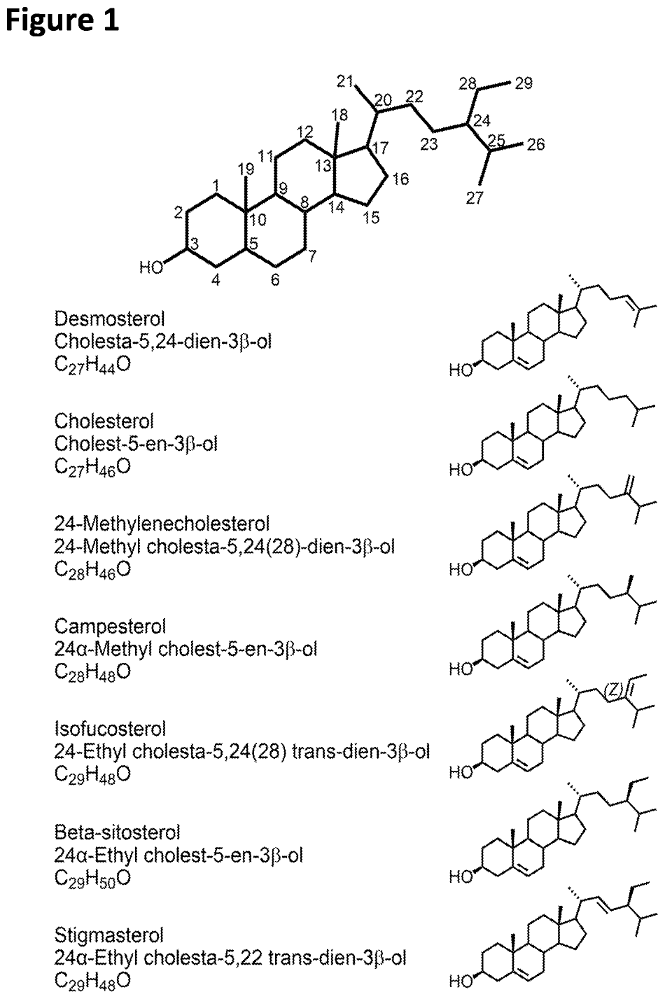

FIG. 19 shows time course of total sterol production, DCW and glycerol addition during fed-batch fermentation of strain ST11064 in a 250 ml bioreactor.

FIG. 20 shows the time course of sterol production, DCW, OD600, glucose concentration and glucose addition during fed-batch fermentation of ST4842 in a 5-L bioreactor. Error bars represent ±1 standard deviation.

FIG. 21: shows the time course of sterol production. DCW, OD600, glucose concentration and glucose addition during fed-batch fermentation of ST11005 in a 5-L bioreactor. Error bars represent ±1 standard deviation.

FIG. 22: shows the time course of sterol production, DCW, OD600, glucose concentration and glucose addition during fed-batch fermentation of ST121785 in a 5-L bioreactor. Error bars represent ±1 standard deviation.

FIG. 23 shows brood production over the course of three months in managed honeybee colonies provided with artificial diets supplemented with engineered yeast biomass in the form of a dried powder.

FIG. 24 shows provision and consumption of artificial diets provided to the same honeybee colonies.

### INCORPORATION BY REFERENCE

The sequence listings in SterolUSASeqUracil.xml created on Mar. 28, 2025, and having a size of 398,907 bytes, are incorporated herein by reference in its entirety.

### DETAILED DESCRIPTION

### Yeast Strains for Use in Providing Yeast of the Invention

“Oleaginous yeast” refers to yeast that can naturally accumulate more than 20% of their dry cell weight (DCW) as lipid and are of the Dikarya subkingdom of fungi. Oleaginous yeast includes organisms such as Yarrowia lipolytica, Cryptococcus albidus, Lipomyces lipofera, Lipomyces starkeyi, Rhodosporidium toruloides, Rhodotorula glutinis, Trichosporon pullulan and Cutaneotrichosporon oleaginosus.

Yeast engineered in accordance with the invention may be selected from any of the above-noted oleaginous yeast species. Preferably, the yeast cells are Yarrowia lipolytica or Rhodotorula glutinis. Most preferably the yeast is of the Yarrowia lipolytica species.

Yarrowia lipolytica is dimorphic yeast and belongs to the Hemiascomycetes. The entire genome of Yarrowia lipolytica is known. Yarrowia species is aerobic and considered to be non-pathogenic. Yarrowia is efficient in using hydrophobic substrates (e.g. alkanes, fatty acids, oils) and can grow on sugars. It has a high potential for industrial applications. Yarrowia lipolytica can accumulate lipid content to approximately 40% of its dry cell weight and is a model organism for lipid accumulation and remobilization.

For engineering of an oleaginous yeast strain of the invention where provision of a sterol surrogate is employed to ease required gene knock out in the ergosterol pathway, as indicated above, the oleaginous yeast ahead of engineering to modify sterol production (sometimes referred to as the platform strain) may be any oleaginous yeast where such knock out results in undesirable growth deficiency or no growth on a standard growth medium as defined above. As also noted above, such a Y. lipolytica yeast as preferred may be derived, for example, from the Y. lipolytica W29 strain Y63746 (designated in Table 5 as ST4842 and available from the ARS culture collection) and is exemplified herein by the strain ST9100. ST9100 has a genotype:
MATa ku70,6::PrTEF1→Cas9-TTe112::PrGPD→DsdA-TLip2 IntC_2-HMG1←PrGPD-PrTeflnt→ERG12 IntC_3-SeACS←PrGPDPrTeflnt→YIACL1 IntD_1-IDI1←PrGPD-PrTeflnt→ERG20 (Arnesen at al (2020) ibid).

It exhibits increased squalene biosynthesis and has other genotype characteristics which rendered it of particular interest for engineering for non-native sterol production.

Ku70p is implicated in DNA double-stranded break repair by non-homologous end-joining in Y. lipolytica. Thus deletion of the ku70 gene in ST9100 promotes DNA double-stranded break repair by homologous recombination, allowing for easier genomic manipulation. A Y. lipolytica codon-optimized Cas9 gene from Streptococcus pyogenes is integrated into the ku70 locus under control of the Tef promoter and terminator, using a dsdA marker cassette which allows growth on D-serine and can be used for selection. The integrated Cas9 gene enables efficient CRISPR/Cas9-mediated genome editing. Upregulation of a number of enzymes has been shown to increase terpenoid production in Y. lipolytica. The primary precursor of sterols is acetyl-CoA. The acetyl CoA pool can be increased by overexpression of the native ATP citrate lyase 1 (ACL) and the Salmonella enterica acetyl-CoA synthetase (SeACS). The enzyme 3-hydroxy-3-methylglutaryl-CoA reductase (HMG) catalyses a key rate-limiting step of the mevalonate pathway and HMG overexpression upregulates this pathway. The mevalonate pathway feeds into the farnesyl pyrophosphate biosynthesis pathway. Overexpression of mevalonate kinase (ERG12), isopentyl diphosphate isomerase (IDI) and farnesyl diphosphate synthase (ERG20) increase flux toward farnesyl pyrophosphate (FPP). FPP is converted to squalene, which is the first intermediate of the sterol biosynthesis pathway.

It will be appreciated however that other oleaginous yeast may be alternatively similarly desirably engineered to provide yeast according to the invention expressing a sterol surrogate, e.g. other oleaginous yeast which exhibit increased squalene production above wild-type, especially other such Y. lipolytica which may be engineered also from Y. lipolytica W29 strain Y-63746 (ST4842) or any of ST6512, ST8980, ST9027 and ST9100. The starting platform yeast for introduction of the required heterologous genes(s) may desirably have all the modified genotype features of ST9100 compared with Y. lipolytica W29 strain Y-63746 as set out above. Some or all of these may desirably be retained in the final engineered yeast strain consistent with maintaining the desired non-native sterol production as further discussed below.

### Gene Deletion

Yeast of the invention may have a reduced ability to produce ergosterol or are incapable of producing ergosterol. Ergosterol, a 5,7,22-triene sterol, is the most abundant sterol in fungal cell membranes, where it regulates permeability and fluidity. Yeast ergosterol is synthesized through a highly conserved and complex pathway that can be divided into three modules. The first module is conserved across all eukaryotes and results in the formation of mevalonate from acetyl-coenzyme A (acetyl-CoA) by acetyl-CoA C-acetyltransferase (ERG10), formation of 3-hydroxy-3-methylglutaryl-coenzyme A (HMG-CoA) by HMG-CoA synthase (ERG13) and reduction of HMG-CoA by HMG-CoA reductases (HMGR) Hmg1 and Hmg2 (Hmg1/2). The second module is carried out in the vacuole and involves the formation of farnesyl pyrophosphate (farnesyl-PP) by mevalonate kinase (ERG12), phosphomevalonate kinase (ERG6), mevalonate pyrophosphate decarboxylase (Mvd1/ERG19), isopentenyl diphosphate isomerase (NH) and farnesyl pyrophosphate synthetase (ERG20).

The third module or late pathway involves ergosterol synthesis itself through consecutive reactions that mainly occur in the endoplasmic reticulum (ER) membrane. Firstly, two molecules of farnesyl-PP are used by squalene synthase (ERG9) to form squalene, which is the precursor of all steroids.

Secondly, squalene is converted into lanosterol by the consecutive action of the squalene epoxidase (ERG1) and lanosterol synthase (ERG7). Lanosterol is transformed to zymosterol through a complex process involving various demethylation, reduction and desaturation reactions catalyzed by lanosterol 14-a-demethylase (ERG11, also known as Cyp51), C-14 reductase (ERG24) and C-4 demethylation complex which includes sterol C-4 methyl oxidase (ERG25), sterol C-3 dehydrogenase (ERG26) and sterol C-3 ketoreductase (ERG27). ERG28 and ERG29 are likely to function in the C-4 demethylation complex reaction.

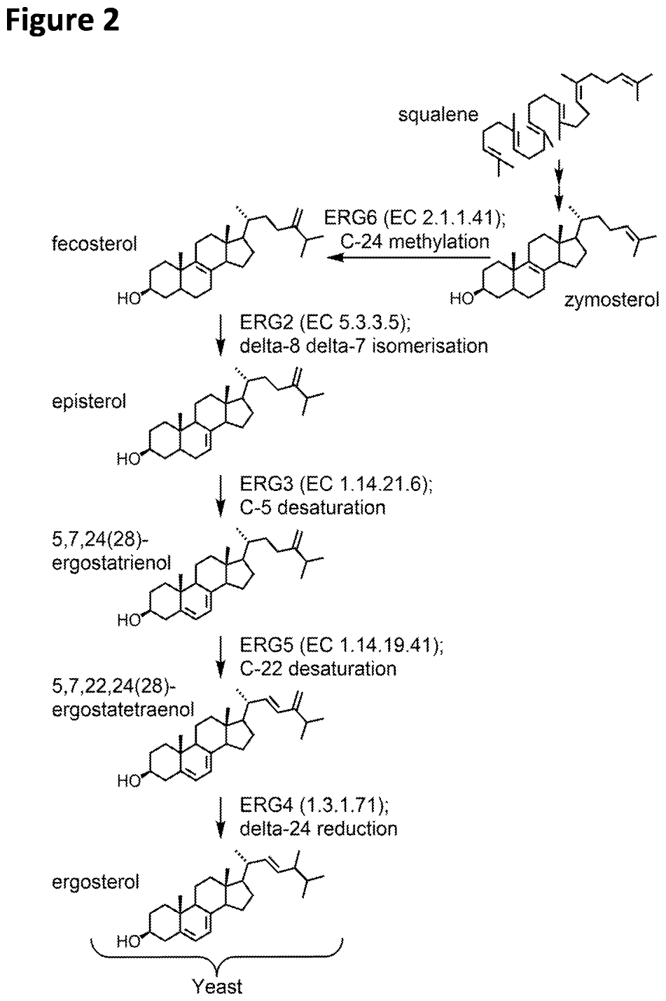

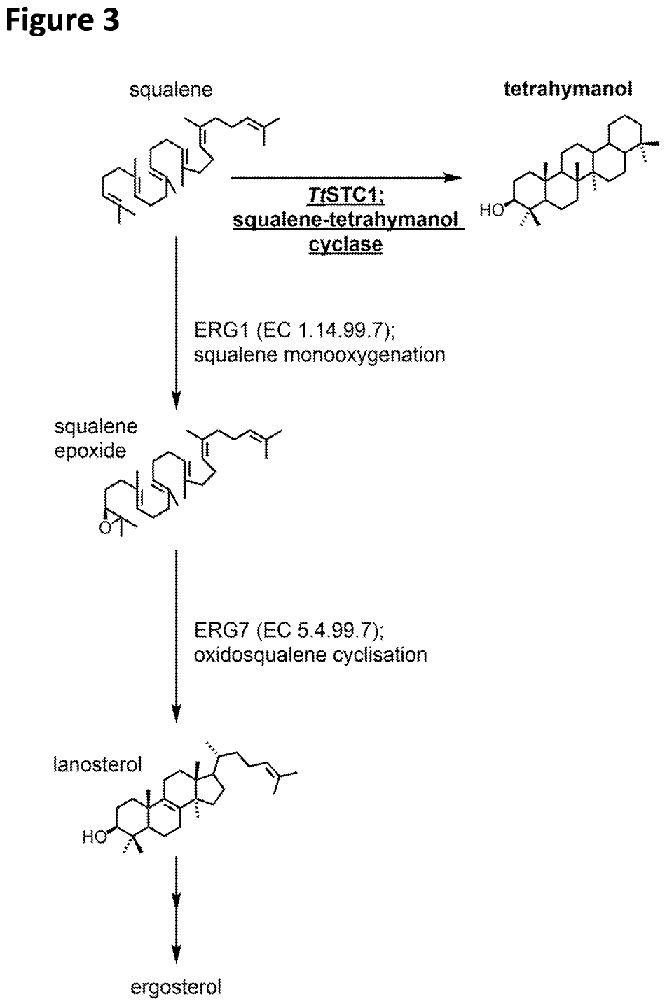

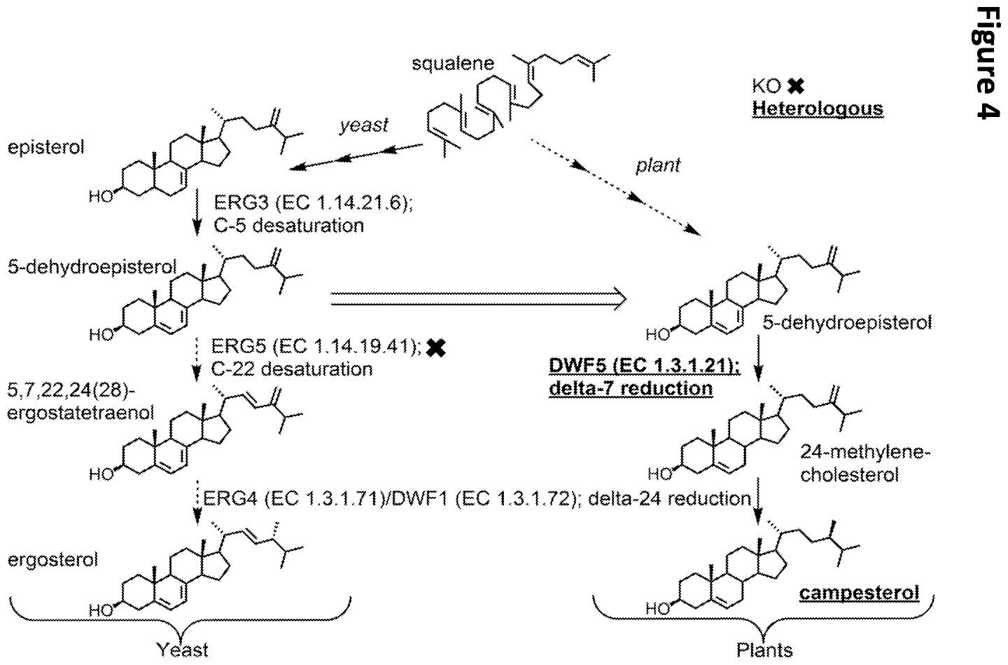

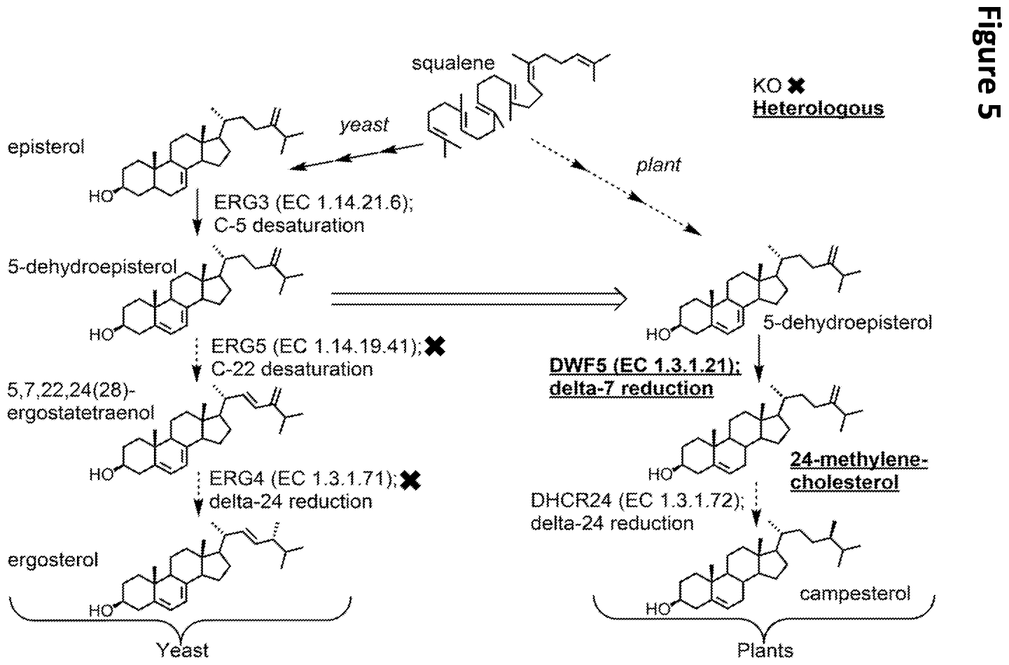

As seen in FIG. 2, sterol C-24 methyltransferase (ERG6) converts zymosterol into fecosterol, followed by the formation of epiestrol by sterol C-8 isomerase (ERG2), which is desaturated and reduced by sterol C-5 desaturase (ERG3) to form 5, 7, 24(28)-ergostatrienol. This is then desaturated by sterol C-22 desaturase (ERG5) to 5, 7, 22, 24(28)-ergostatetraenol which is finally reduced by sterol C-24 reductase (ERG4) to ergosterol.

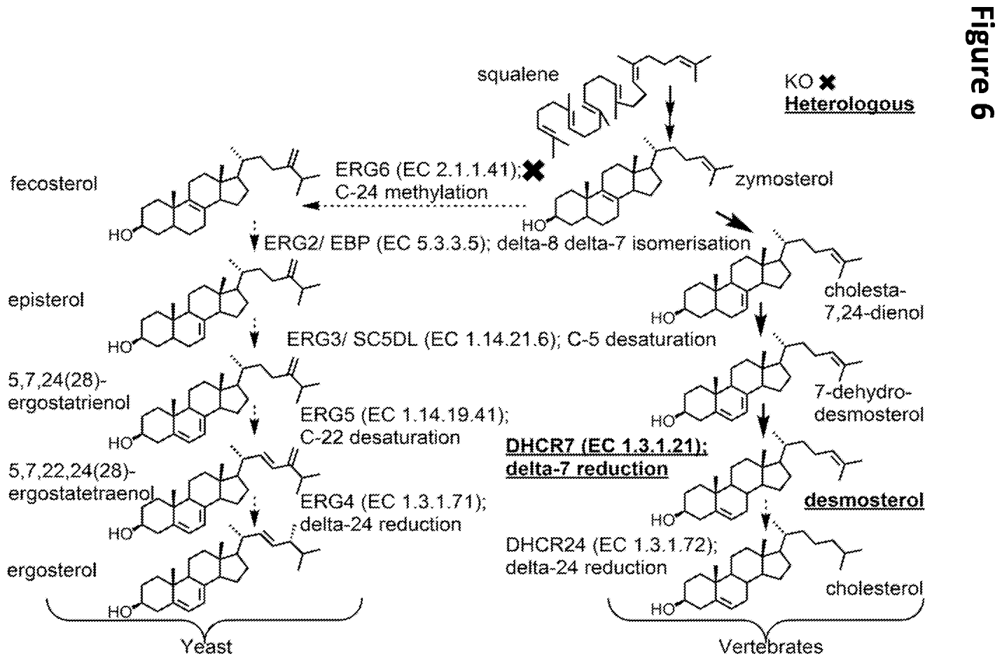

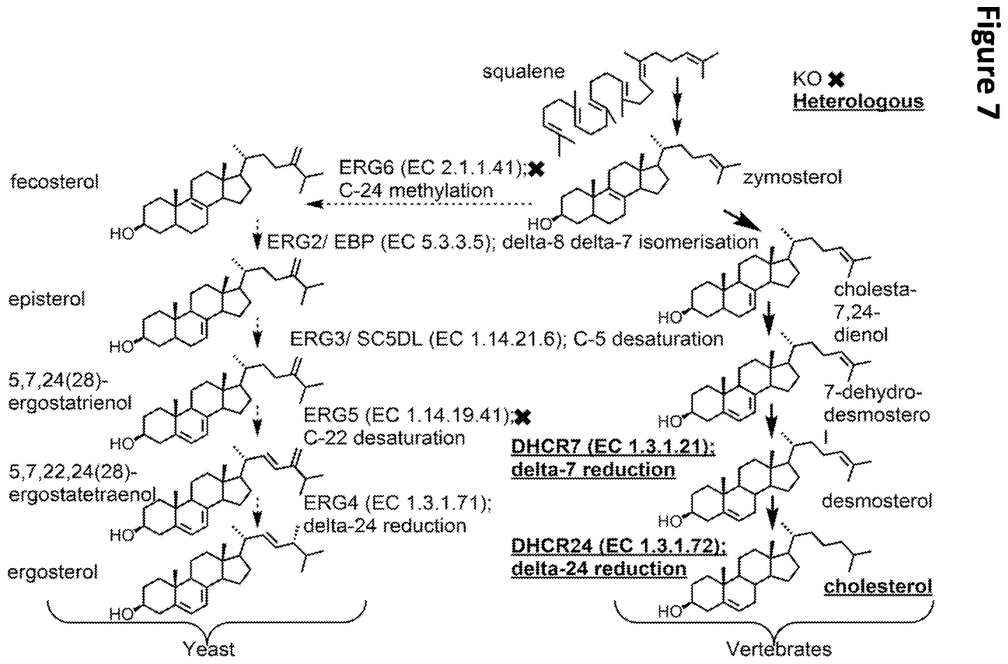

A yeast of the invention may be made incapable of producing ergosterol by attenuating activity of, or deleting, any one of the enzymes utilised in the third module or late pathway of ergosterol production. For example by attenuating activity of, or deleting, any one or more of sterol C-24 methyltransferase (ERG6), sterol C-8 isomerase (ERG2), sterol C-5 desaturase (ERG3), sterol C-22 desaturase (ERG5), and/or sterol C-24 reductase (ERG4). Commonly however, disruption of ergosterol production will be by attenuating activity of, or deleting, endogenous sterol C-22 desaturase enzyme (ERG5), delta-24 sterol reductase enzyme (ERG4) and/or sterol C-24 methyltransferase (ERG6). One or more of ERG4, ERG5 and ERG5 expression may be attenuated or deleted depending on the desired non-native sterol production desired as exemplified herein and illustrated by FIGS. 4 to 9.

As indicated above, a yeast of the invention may include replacement of ERG4 with a plant delta-24 sterol reductase (i.e. DWF1). That is to say that the yeast provided herein may include an attenuated or deleted ERG4 and include a heterologous nucleic acid encoding a plant delta-24(28)
reductase (DWF1). The use of a plant delta-24 sterol reductase may be chosen to enable production of plant epimers of non-native sterols which may be beneficial for certain uses and applications such as insect (e.g. bee) foods.

The term attenuating or reducing activity are used to refer to a change in activity of an enzyme that leads to a level of the enzyme product below detectable levels or to levels lower than that seen in a reference or corresponding wild type strain. Attenuation or reduction of activity of an enzyme may be achieved by the use of genetic modification in order to inactivate or delete a gene or part of a gene encoding the protein. Suitable methods for reducing or preventing activity of a protein are well-known.

The terms “deletion,” deleted, “knockout.” and knocked our can be used interchangeably to refer to an endogenous gene that has been manipulated to no longer be expressed in an yeast of the invention.

A deletion can mean that at least part of the subject nucleic acid sequence is lost, but a deletion can also be accomplished by disrupting a gene through, for example, the insertion of another sequence (e.g. a selection marker), or a combination of deletion and insertion, but a deletion can also be performed by other genetic modifications. A deletion can mean that the gene no longer produces its functional gene product or. In various embodiments, that the gene produces less than 20% or less than 10% or less than 5% or less than 1% of its functional gene product versus production without the deletion under standard culturing conditions. The terms deletion cassette or vector and disruption cassette or vector are used interchangeably and refer to nucleic acid constructs that are inserted into a yeast of the invention to delete to attenuate a gene and therefore the gene product thereof such as the encoded enzyme

Required gene deletion or attenuation may be achieved using CRISPR-Cas gRNA based technology. Such technology may be used to replace the target gene with a selectable marker such as an antibiotic resistance gene. Deletion of the target gene then can be confirmed by exposing the modified yeast to the corresponding antibiotic.

After confirmation that the target gene has been deleted the selectable marker can be subsequently removed for example by use of a Cre-recombinase episomal system.

Other suitable methods include site directed mutagenesis, site specific nuclease based methods and/or homologous recombination.

Yeast of the invention may at least exhibit attenuated activity or deletion of an endogenous sterol C22 desaturase enzyme (ERG5). Yeast of the invention may exhibit attenuated activity or deletion of endogenous delta-24 sterol reductase enzyme (ERG4). Yeast of the invention may exhibit attenuated activity or deletion of endogenous sterol C-24 methyltransferase (ERG6). That is to say that yeast of the invention may include attenuated activity or deletion of endogenous sterol C-22 desaturase enzyme (ERG5), attenuated or deleted endogenous delta-24 sterol reductase enzyme (ERG4), and/or attenuated or deleted endogenous sterol C-24 methyltransferase enzyme (ERG6). A yeast of the invention as illustrated by embodiments exemplified herein may have deletion or attenuation of ERG5 and deletion or attenuation of one of ERG4 and ERG6.

The detrimental effects of attenuation or deletion of one or more of the above mentioned enzymes or genes may be increased in oleaginous yeast due to aerobic yeasts lacking sterol transporters that anaerobic yeast such as Saccharomyces and other yeasts possess which enable them to acquire sterols from their culture media.

### Provision of a Sterol Surrogate

As indicated above, it has been found by the inventors that to enable appropriate diversion of sterol biosynthetic flux from normal ergosterol synthesis in an oleaginous yeast to enable desired non-native sterol production, it may be necessary to provide intracellularly a sterol surrogate. Such provision of a sterol surrogate is called for whenever a parent strain for desired gene knock out in the ergosterol pathway is incompatible with adequate cell growth for useful non-native sterol production. This has first been observed with the known Y. lipolytica strain ST9100 which has a genotype that might otherwise be considered well-designed for non-native sterol production but as noted above can be expected to be a problem with other engineered oleaginous yeast strains.

As used herein, the term “sterol surrogate” refers to a heterologous compound that is utilised by a yeast to compensate for detrimental growth effect of disruption of the normal sterol biosynthesis pathway to ergosterol. It should enable deletion of any of the ERG4 gene, ERG5 gene and ERG6 genes which otherwise precludes adequate cell growth. It will be provided intracellularly, possibly at low level, for its required compensatory purpose. The inclusion of a sterol surrogate will help improve growth of a yeast that has reduced production of ergosterol or is incapable of producing ergosterol. In addition, the sterol surrogate may increase the production of exogenous (non-native) desired sterols.

A sterol surrogate may preferably be provided by expression of a heterologous nucleic acid encoding an enzyme for its direct production and as noted above will be controlled so that production of the desired non-native sterols can be achieved. For this reason, expression of the sterol surrogate may be under the control of a weak promoter in the chosen yeast cells such as the PrGPAT promoter or a functionally equivalent weak yeast promoter.

The sterol surrogate may preferably be tetrahymanol. Tetrahymanol is a pentacyclic triterpenoid having a 3beta-(21alpha-)hydroxy-substituted germacrane structure. Other suitable compounds having similar structure and properties may also be used.

Without being bound by theory, tetrahymanol has the advantage of being better tolerated in the yeast membrane than other sterol surrogates and therefore may reduce toxicity of heterologous sterol overproduction or toxic intermediate production.

Tetrahymanol can be produced from squalene in a single, oxygen-independent cyclization reaction catalysed by a tetrahymanol synthase referred to by the Enzyme Commission (EC) number 4.2.1.123. For example, the tetrahymanol synthase may be a squalene-tetrahymanol cyclase.

Thus, a yeast of the invention may be provided with a heterologous nucleic acid sequence encoding a tetrahymanol synthase such as a squalene-tetrahymanol cyclase. The squalene-tetrahymanol cyclase may be the Tetrahymena thermophila squalene-tetrahymanol cyclase (GenBank ascension number XP_001026696.2) or a functional variant thereof, preferably encoded by a codon-optimised sequence and preferably under the control of the PrGPAT promoter or a functionally equivalent weak yeast promoter.

Alternatively, the sterol surrogate may be a hopanoid. Hopanoids are a diverse group of pentacyclic triterpenoid lipids mainly produced by bacteria. For example, the sterol surrogate may be hopene. Hopene can be produced from squalene by a squalene-hopene cyclase.

Thus, a yeast of the invention may be provided with a heterologous nucleic acid sequence encoding a squalene-hopene cyclase. For example the squalene-hopene cyclase may be the Schizosaccharomyces japonicus squalene-hopene cyclase or a functional variant thereof

Thus, by way of example, there is provided an oleaginous yeast comprising an attenuated or deleted endogenous sterol C-22 desaturase (ERG5) and at least one heterologous nucleic acid sequence encoding a squalene-tetrahymanol cyclase or a squalene-hopene cyclase.

Also provided is an oleaginous yeast comprising an attenuated or deleted endogenous sterol C-22 desaturase (ERG5) and an attenuated or deleted endogenous delta-24 sterol reductase (ERG4) and at least one heterologous nucleic acid sequence encoding a squalene-tetrahymanol cyclase or a squalene-hopene cyclase.

Also provided is an oleaginous yeast comprising an attenuated or deleted endogenous sterol C-22 desaturase (ERG5), preferably a deleted ERG5, an attenuated endogenous delta-24 sterol reductase which is either an attenuated ERG4 or an attenuated plant DWF1 provided by expression of a coding sequence, and at least one heterologous nucleic acid encoding a squalene-tetrahymanol cyclase or a squalene-hopene cyclase.

Also provided is an oleaginous yeast comprising an inactivated or deleted endogenous sterol C-22 desaturase (ERG5), an attenuated or deleted endogenous sterol C-24 methyl-transferase (ERG6) and at least one heterologous nucleic acid encoding a squalene-tetrahymanol cyclase or a squalene-hopene cyclase.

Also provided is an oleaginous yeast comprising an attenuated or deleted endogenous sterol C-24 methyl-transferase (ERG6) and at least one heterologous nucleic acid encoding a squalene-tetrahymanol cyclase or a squalene-hopene cyclase.

Such use of a sterol surrogate may allow for the yeast to maintain a growth rate similar to that of a wild type or reference yeast ahead of gene deletion.

As now further discussed below a yeast as above which (a) has reduced production of ergosterol production compared with a wild-type oleaginous yeast or is incapable of producing ergosterol and (b) is capable of expressing a sterol surrogate may additionally have incorporated one or more expressible heterogeneous genes whereby it is capable of producing one or more desired non-native sterols selected from one or more of campesterol, 24-methylenecholesterol, cholesterol, desmosterol, 13-sitosterol, isofucosterol and stigmasterol. As indicated above, of especial interest, for example, is production of 24-methylenecholesterol either alone or in combination with one or more other sterols, e.g. campesterol and cholesterol.

### Heterologous Sterol Encoding Nucleic Acid Sequences

Yeast of the invention may include one or more heterologous nucleic acids encoding one or more of a delta-7 sterol reductase enzyme, a delta-24 sterol reductase enzyme, sterol C-28 methyltransferase enzyme and a sterol C-22 desaturase enzyme (see Table 1).

Heterologous enzymes which may be introduced into an oleaginous yeast, e.g. a Y. lipolytica yeast, to provide an engineered yeast according to the invention may include, for example one or more of the following:
a. a delta-7 sterol reductase enzyme selected from the group consisting of:
   Legionella drancourtii delta-7 sterol reductase;
   Ectocarpus siliculosus delta-7 sterol reductase;
   Candidatus Protoch/amydia amoebophila delta-7 sterol reductase;
   Coccomyxa subellipsoidea delta-7 sterol reductase;
   Glycine sofa delta-7 sterol reductase;
   Tetrase/mis sp GS1018 delta-7 sterol reductase
   Solanum tuberosum delta-7 sterol reductase;
   Danio rerio delta-7 sterol reductase
   Mortierella verticillata delta-7 sterol reductase
   Waddlia chondrophila delta-7 sterol reductase and functional variants thereof; and/or
b. a delta-24 sterol reductase selected from the group consisting of:
   Danio rerio delta-24 sterol reductase;
   Bombyx mori delta-24 sterol reductase;
   Penaeus vannamei delta-24 sterol reductase;
   Aedes aegypti delta-24 sterol reductase;
   Gallus gallus delta-24 sterol reductase;
   Mus muscu/us delta-24 sterol reductase;
   Xenopus tropicalis delta-24 sterol reductase
   Solanum lycopersicum delta-24 sterol reductase;
   Notechis scutatus delta-24 sterol reductase; and/or
   Amblyraja radiata delta-24 sterol reductase;
   Arabidopsis thaliana delta-24 sterol reductase;
   Solanum tuberosum delta-24 sterol reductase;
   Arachis duranensis delta-24 sterol reductase;
   Selaginella moellendorffii delta-24 sterol reductase;
   Capsicum chinense delta-24 sterol reductase;
   Artemisia annua delta-24 sterol reductase;
   Helianthus annuus delta-24 sterol reductase;
   Cocos nucifera delta-24 sterol reductase;
   Triticum urartu delta-24 sterol reductase;
   Gracilariopsis chorda delta-24 sterol reductase;
   Capsella rubella delta-24 sterol reductase
   Afuga reptans delta-24 sterol reductase and functional variants thereof; and/or
c. a sterol C-28 methyltransferase enzyme selected from the group consisting thereof:
   Cucurbita pepo sterol C-28 methyltransferase;
   Eutrema sa/sugineum sterol C-28 methyltransferase;
   Arabidopsis thaliana sterol C-28 methyltransferase;
   Morus notabilis sterol C-28 methyltransferase;
   Amborella trichopoda sterol C-28 methyltransferase;
   Creolimax fragrantissima sterol C-28 methyltransferase;
   U/va mutabilis sterol C-28 methyltransferase; Rhodamnia argentea sterol C-28 methyltransferase;
   Chenopodium quinoa sterol C-28 methyltransferase;
   Glycine sofa sterol C-28 methyl-transferase and functional variants thereof; and/or
d. a sterol C-22 desaturase enzyme which is:
   Cytochrome P450 710A1 or a functional variant thereof.

As noted above, both Solanum tuberosum and Solanum lycopersicum possess two delta-24 sterol reductase variants, one catalyses delta-24(25) reduction and the other catalyses delta-24(28) reduction (see Table 1). One or more heterologous coding sequences for a delta-24(28) sterol reductase and/or one or more heterologous coding sequences for a delta-24(25) sterol reductase may be expressed in a yeast of the invention.

As indicated above, where it is desired to maintain the same stereochemistry at a C-24 position of a sterol as observed in plants. e.g. in production of campesterol for some applications, then the yeast ERG4 gene encoding the yeast delta-24 sterol reductase will be substituted by a plant delta-24(28) sterol reductase, i.e. a DWF1 enzyme such as the Arabidopsis thaliana delta-24(28) sterol reductase (see Table 1). For production of the plant epimer of campesterol together with 24-methylenecholesterol in a Y. lipolytica of the invention lacking ERG5 and ERG4, expression of the Solanum tuberosum delta-24(28) sterol reductase may, for example be preferred as illustrated herein by engineered Y. lipolytica strain ST11064 and strains derived therefrom.

Desirably, each heterologous enzyme incorporated in a yeast of the invention will be encoded by a codon-optimised sequence for expression in yeast, more desirably for use in the selected host species, e.g. Y. lipolytica. Appropriate codon-optimised sequences for all the above heterologous gene categories for use in engineering a Y. lipolytica strain are provided in Table 4 below.

For strong expression, the known PrTEFintron promoter may for example preferably be selected or a functionally equivalent promoter; i.e. a promoter that provides at least substantially the same expression or higher. However, as previously noted above, in some instances heterologous enzyme expression will be required or desirable under the control of a weaker promoter, e.g. the PrDGA1 or PrGPAT promoter as disclosed in Holkenbrink et al (2018) ibid or a functionally equivalent weak yeast promoter.

Delta-7 sterol reductase enzymes (EC 1.3.1.21) are involved in the production of cholesterol by reduction of the C7-C8 double bond of 7-dehydrocholesterol (7-OHC) and are capable of converting:
7-dehydrodesmosterol to desmosterol;
5-dehydroepisterol to 24-methylenecholesterol; and
5-dehydroavenasterol to isofucosterol.

The delta-7 sterol reductase may be an animal delta-7 sterol reductase such as DHCR7 or a plant delta-7 sterol reductase such as DWF5.

As noted above, the delta-7 sterol reductase enzyme may be selected from: Legionella drancourtii delta-7 sterol reductase; Ectocarpus siliculosus delta-7 sterol reductase; Candidatus Protochiamydia amoebophila delta-7 sterol reductase; Coccomyxa subellipsoidea delta-7 sterol reductase; Glycine sofa delta-7 sterol reductase; Tetraselmis sp GS/018 delta-7 sterol reductase; Solanum tuberosum delta-7 sterol reductase, Danio rerio delta-7 sterol reductase. Mortierella verticillata delta-7 sterol reductase. Waddlia chondrophila delta-7 sterol reductase and functional variants thereof. For expression in an engineered Y. lipolytica of the invention, expression of the delta-7 reductase of
Tetraselmis sp. GS1080 or Legionella drancourtii may for example be favoured. However, it will be recognised that use of other delta-7 sterol reductases is not excluded. Expression in an engineered Y. lipolytica of the invention may desirably be under the control of the PrTEFintron promoter or a functionally equivalent strong promoter.
Delta-24 sterol reductase enzymes act on a range of steroids with a 24(25)-double bond or 24(28)-double bond and are capable of converting:
   desmosterol to cholesterol;
   24-methylenecholesterol to campesterol; and
   isofucosterol to sitosterol.

Where expression of a delta-24(25) sterol reductase is required this may be an animal delta-24(25) sterol reductase such as DHCR24 or a plant delta-24(25) reductase such as SSR2. As previously discussed, a plant delta-24(28) sterol reductase (DWF1) will be utilised to substitute for yeast ERG4.

As noted above, a delta-24 sterol reductase for expression in a yeast of the invention may be selected from or be a functional variant of: Danio rerio delta-24 sterol reductase; Bombyx mori delta-24 sterol reductase; Penaeus vannamei delta-24 sterol reductase; Aedes aegypti delta-24 sterol reductase; Gallus gallus delta-24 sterol reductase Mus musculus delta-24 sterol reductase; Xenopus tropicalis delta-24 sterol reductase; a Solanum lycopersicum delta-24 sterol reductase; Notechis scutatus delta-24 sterol reductase; Amblyraja radiata delta-24 sterol reductase; Arabidopsis thaliana delta-24 sterol reductase; a Solanum tuberosum delta-24 sterol reductase; Arachis duranensis delta-24 sterol reductase; Selaginella moellendorffii delta-24 sterol reductase; Capsicum chinense delta-24 sterol reductase; Artemisia annua delta-24 sterol reductase; Helianthus annuus delta-24 sterol reductase; Cocos nucifera delta-24 sterol reductase; Triticum urartu delta-24 sterol reductase; Gracilariopsis chorda delta-24 sterol reductase; Capsella rubella delta-24 sterol reductase; or Ajuga reptans delta-24 sterol reductase.

However, it is further emphasised that for production of plant sterols, such as campesterol, beta-sitosterol and stigmasterol, a plant delta-24(28) sterol reductase is required to replace yeast ERG4 and thereby preferably achieve the correct stereochemistry. Stereochemistry around C-24 in ergosterol is different to that of plant sterols, due to differences in the respective delta-24 sterol reductase enzymes. As such, sterols such as campesterol produced in previous work using animal or yeast delta-24 sterol reductases are the C-24₁₃ epimer forms of the sterols. For example, ERG4 produces campesterol in the C-2413 configuration (S). The epimers may be indistinguishable by gas chromatography-mass spectroscopy but the differences may not be trivial. For example, as previously noted, the moulting hormone Makisterone A is produced from campesterol in honeybees. Provision of the 24₁₃ epimer of campesterol would lead to the production of epi-Makisterone A, which may not function in the same way (e.g. some insects use only the plant epimer form). By deleting the native

ERG4 and expressing a heterologous plant delta-24(28) sterol reductase, the correct plant stereochemistry at C-24 (24a configuration (R)) of for example campesterol can be achieved (see FIG. 16). As previously noted, by way of example of a preferred enzyme for this purpose in an engineered Y. lipolytica of the invention is the delta-24(28) sterol reductase of Solanum tuberosum. Expression will generally be under the control of a weak yeast promoter such as the PrDGA1 promoter or a functionally equivalent weak yeast promoter. Sterol C-28 methyltransferase enzymes (EC 2.1.1.143) also known as 24-methylenesterol C-methyltransferase acts in the second methylation step of plant sterol biosynthesis and is capable of converting:
epiestrol to delta 7-avenasterol; and
5-dehydroepisterol to 5-dehydroavenasterol.

The sterol C-28 methyltransferase enzyme employed in yeast of the invention may be a plant sterol C-28 methyltransferase enzyme such as SMT2.

As noted above, the sterol C-28 methyltransferase enzyme may be selected from or a functional variant of: Cucurbita pepo C-28 methyltransferase; Eutrema salsugineum C-28 methyltransferase; Arabidopsis thaliana C-28 methyltransferase; Morus notabilis C-28 methyltransferase; Amborella trichopoda C-28 methyltransferase; Creolimax fragrantissima C-28 methyltransferase; U/va mutabilis C-28 methyl-transferase; Rhodamnia argentea C-28 methyl-transferase; Chenopodium quinoa C-28 methyl-transferase or Glycine sofa C-28 methyltransferase. Use of more than one such enzyme may be favoured, e.g. one, two or three selected from the C-28 sterol methyltransferases of Chenopodium quinoa, Arabidopsis thaliana and Amborella trichopoda.

Sterol C-22 desaturase enzymes (EC1.14.19.41) act on sitosterol and 24-epi-campesterol, producing stigmasterol and brassicasterol respectively. The sterol C-22 desaturase enzyme may be Cytochrome P450 710A1.

Table 1 provides details of the enzymes mentioned above as well as the names of the enzymes in yeast, plants and animals.

### Optional Additional Genotype Features

Optionally, an oleaginous yeast of the invention may exhibit reduced activity of at least one or more of endogenous:
PAH1 (Mg2+-dependent phosphatidate phosphatase), Bts1 (geranylgeranyl pyrophosphate synthase), GDH1 (NADP-specific glutamate dehydrogenase 1), Are1 (sterol O-acyltransferase). Say1 (stearyl acetyl hydrolase), Sds23, and/or Ins1. For example, PAH1 gene knock-out may be deemed desirable to increase the amount of membranes and capacity for sterol accumulation as illustrated by Y. lipolytica strain ST11197 referred to herein (an ERG5/PAH1 knock out derived from ST9100 and expressing a squalene-tetrahymanol cyclase gene and Ectocarpus silculosus delta-7 sterol reductase). This strain enabled production of campesterol at as high as 41.5 mg/g DCW with 72 hrs culture with glucose as the sole carbon source.
Optionally, an oleaginous yeast of the invention may exhibit increased activity of at least one or more of:

HMG1 (3-hydroxy-3-methylglutaryl-coenzyme A reductase 1), truncated HMG1, UPC2 (sterol uptake control protein 2), Ecm22 (sterol regulatory element binding protein), Erg1 (squalene epoxidase), Erg3 (sterol C-5 desaturase), Erg7 (lanosterol synthase), Erg8 (phosphomevalonate kinase), Erg9 (squalene synthase), Erg10 (acetyl-CoA C-acetyltransferase), Erg11 (lanosterol 14-a-demethylase), Erg12 (mevalonate kinase), Erg13 (HMG-CoA synthase). Erg19 (mevalonate pyrophosphate decarboxylase), Erg20 (farnesyl pyrophosphate synthetase), Erg25 (sterol C-4 methyloxydase). Erg26 (sterol C-3 dehydrogenase). Erg27 (sterol C-3 ketoreductase), IDI (isopentenyl diphosphate isomerase), Acl (ATP-citrate lyase). Pot1 (3-ketoacyl-CoA thiolase), Pat1 (peroxisomal acetoacetyl-CoA thiolase), Pex10 (peroxisomal membrane E3 ubiquitin ligase), GDH2 (NAD(+)-dependent glutamate dehydrogenase), G6PD1 (Glucose-6-phosphate 1-dehydrogenase 1), Are1 (sterol O-acyltransferase), Are2 (acyl-CoA:sterol acyltransferase), Atf2 (alcohol acetyltransferase), Acs1 (acetyl-coA synthetase). SSD1 (protein SSD1), YBP1 (YAP1-binding protein), Sre1 (sterol regulatory element-binding protein 1) and/or Css1 (secreted protein CSS1) or variants thereof.

As noted above, stereochemistry around C-24 in ergosterol is different to that in plant sterols. Thus, where it is desired to obtain a non-native sterol with plant stereochemistry at C-24, e. g. the 24α epimer of campesterol, deletion of the endogenous delta-24 sterol reductase enzyme (ERG4) will be accompanied by expression of a heterologous nucleic acid encoding a plant delta-24 sterol reductase enzyme (DWF1). Thus whilst production of campesterol may be achieved by just deletion or attenuation of the ERG5 gene and expression of a heterologous gene for a delta-7 sterol reductase, e.g. a plant DWF5, preferably in some instances, the yeast ERG4 gene may additionally be knocked out and a DWF1 expressed whereby campesterol production is maintained but as the plant epimer.

### Culturing for Non-Native Sterol Production

For production of the one or more desired sterols by a yeast of the invention, culturing may be carried out in any culture medium under conditions suitable for expressing the one or more of the heterologous nucleic acids as described herein. However, preferably a single simple carbon feed source will be provided. e.g. glucose. Thus for example culturing may be in conventional yeast extract peptone dextrose (YPD) medium supplemented with glucose at 30° C. By such culturing of a yeast of the invention, as exemplified herein, a desired sterol or mixture of sterols may be attained at a production level of at least 9-10 mg/g of dry cell weight (DCW), for example at least 25 mg/g DCW, possibly at least 30 or 40 mg/g DCW or even higher, e.g., at least 45-50 mg/g.

By way of example, the invention provides an oleaginous yeast that is capable of producing commercially viable levels of 24-methylenecholesterol having:
an attenuated or deleted endogenous sterol C-22 desaturase enzyme (ERG5) and delta-24 sterol reductase enzyme (ERG4);
a heterologous nucleic acid for provision intracellularly of a sterol surrogate, e.g. encoding a squalene-tetrahymanol cyclase and
a heterologous nucleic acid encoding a delta-7 sterol reductase enzyme as described herein;
wherein the yeast is capable of producing 24-methylenecholesterol at a concentration of at least 9-10 mg/g DCW, possibly together with one or more additional non-native sterols, e.g. campesterol and cholesterol at measurable amounts, or greater than 25 mg/g DCW. By means of such a yeast 24-methylenecholesterol concentrations of at least 30 mg/g, 35 mg/g, 40 mg/g or 45-50 mg/g may for example be achieved.

Preferred embodiments of engineered yeast of the invention are further discussed below with reference to Table 2 and the FIGURES.

### Preferred Embodiments of Yeast of the Invention Expressing a Sterol Surrogate

Table 2 provides a summary of different genetic modifications for producing specific sterols.

Now described are various preferred embodiments of yeast of the invention as discussed above where the yeast has reduced production of ergosterol compared with a wild-type oleaginous yeast or is incapable of producing ergosterol and this is coupled with intracellular provision of a sterol surrogate whereby the disruption of ergosterol biosynthesis, e.g. by gene deletion, is compensated for to aid growth. By way of example, for such preferred embodiments, the platform strain for such engineering, as illustrated by the exemplification, may be Y. lipolytica ST9100. However, as indicated above, it is envisaged that similar non-native sterol production may be achieved by the same strategy with other oleaginous yeast, e.g. other Y. lipolytica strains, which share with ST9100 knock out of any of the ERG4, ERG5 and ERG6 genes being incompatible with adequate cell growth, e.g. other such engineered yeast which have been modified to increase squalene synthesis compared to wild-type. Without being bound by theory, it has been postulated that this may be related to harmful accumulation of sterol intermediates but equally increased squalene synthesis may contribute to good non-native sterol production in final engineered strains of the invention.

A. Thus, in one preferred embodiment a yeast as described above expressing a sterol surrogate and engineered for production of one more non-native sterols comprising campesterol has:
(i) an attenuated or deleted endogenous sterol C-22 desaturase enzyme (ERG5); and
(ii) a heterologous nucleic acid sequence encoding a delta-7 sterol reductase enzyme as described herein.

Preferably, the sterol surrogate may be provided by expression of a squalene-tetrahymanol cyclase under the control of the PrGPAT promoter or a functionally equivalent weak yeast promoter.

The yeast may further include an attenuated or deleted endogenous delta-24 sterol reductase enzyme (ERG4) and a heterologous nucleic acid encoding a plant delta-24(28) sterol reductase enzyme (DWF1). That is to say the yeast may have endogenous delta-24 sterol reductase enzyme (ERG4) replaced by a heterologous nucleic acid encoding a plant delta-24(28) sterol reductase enzyme (DWF1).

As seen in FIG. 4, the yeast converts squalene to 5-dehydroepisterol using endogenous sterol C-24 methyltransferase (ERG6), sterol C-8 isomerase (ERG2) and sterol C-5 desaturase (ERG3). As the yeast lacks endogenous sterol C-22 desaturase (ERG5), the 5-dehydroepisterol is converted by the heterologous delta-7 reductase (DWF5) to 24-methylenecholesterol. Endogenous sterol C-24 reductase (ERG4) converts the 24-methylenecholesterol to campesterol. Alternatively, the final step is performed by a plant DWF1 substituting for the endogenous ERG4 to attain the plant 24α epimer (24(R)-campesterol).

By incorporating into a platform strain as above, e.g. Y. lipolytica ST9100, (i) ERG5 gene deletion facilitated by expression of a codon-optimised coding sequence for squalene-tetrahymanol cyclase of Tetrahymena thermophila (TtSTC) under the control of a weak yeast promoter, e.g. PrGPAT and (ii) a codon-optimised coding sequence for expression of a delta-7 sterol reductase, e.g. Coccomyxa subellipsoidea delta-7 sterol reductase, Ectocarpus silculosus delta-7 sterol reductase or the delta-7 sterol reductase gene variant of Tetraselmis sp. GSL08, under the control of the PrTEFintron promoter or a functionally equivalent yeast promoter, production of campesterol has been shown to be achievable at a level of at least 30 mg/g DCW, e.g. about 40 mg/g DCW (culturing at 30° C. in conventional YPD medium with glucose). Such engineering may be combined with PAH1 gene knock-out to further boost campesterol production. See Example 3 and FIGS. 12 and 13. As indicated above, it may also be combined with substitution of yeast ERG4 by a plant DWF1.

Campesterol is an important precursor for steroid drugs such as progesterone, pregnenolone, and hydrocortisone. Campesterol is also a major precursor for the production of brassinosteroids which may have antiviral, antifungal, antiproliferative, antibacterial, neuroprotective and immunomodulatory properties in animals. In addition, campesterol may be used as a dietary additive. Campesterol may also be used in artificial dietary compositions for example for insects such as bees. Particularly, it is again noted that campesterol produced by a plant delta-24(28) sterol reductase enzyme may be useful in an artificial dietary composition for bees as the campesterol produced will be the plant epimer.

B. In another preferred embodiment a yeast as described above expressing a sterol surrogate and engineered for production of one more non-native sterols comprising desmosterol has:
(i) an attenuated or deleted endogenous sterol C-24 methyltransferase (ERG6); and
(ii) a heterologous nucleic acid sequence encoding a delta-7 sterol reductase enzyme as described herein.

The yeast may further include an attenuated or deleted endogenous sterol C-22 desaturase enzyme (ERG5) and/or delta-24 sterol reductase enzyme (ERG4).

Preferably, again the sterol surrogate may be provided by expression of a squalene-tetrahymanol cyclase under the control of the PrGPAT promoter or a functionally equivalent weak yeast promoter

Preferably, the delta-7 sterol reductase encoded by the heterologous nucleic acid is an animal delta-7 sterol reductase, for example DHCR7. The chosen delta-7 sterol reductase, e.g. the delta-7 sterol reductase of Legionella drancourtii or another delta-7 sterol reductase as listed above, will preferably be expressed by a codon-optimised coding sequence e.g. a codon-optimised sequence for Y. lipolytica again under the control of the PrTEFintron promoter or a functionally equivalent yeast promoter.

As seen in FIG. 6, as the yeast lacks an endogenous C-24 methyltransferase (ERG6) it is incapable of converting zymosterol to fecosterol. Instead zymosterol is converted by endogenous sterol C-8 isomerase (ERG2) to cholesta-7,24-dienol, which is then converted to 7-dehydrodesmosterol by endogenous sterol C-5 desaturase (ERG3). This is then converted to desmosterol by the heterologous delta-7 reductase (DHCR7). Attenuation or deletion of endogenous sterol C-22 desaturase (ERG5) and endogenous sterol C-24 reductase (ERG4) activity may help to prevent or reduce the production of by-products as the yeast is incapable of acting on the 7-dehydrodesmosterol or desmosterol as well as incapable of producing ergosterol leading to production of desmosterol.

Desmosterol, also known as cholesta-5,24-dien-313-ol, is an immediate precursor of cholesterol in the Bloch pathway of sterol synthesis and an abundant membrane lipid in specific types of organisms.

C. In another preferred embodiment a yeast as described above expressing a sterol surrogate and engineered for production of one more non-native sterols comprising cholesterol has:
(i) an attenuated or deleted endogenous sterol C-22 desaturase enzyme (ERG5) and an attenuated or deleted sterol C-24 methyltransferase (ERG6):
(ii) a heterologous nucleic acid sequence encoding a delta-7 sterol reductase enzyme as described herein and
(iii) a heterologous nucleic acid sequence encoding a delta-24 sterol reductase enzyme as described herein.

The delta-7 sterol reductase encoded by a heterologous nucleic acid may be an animal delta-7 sterol reductase, for example DHCR7, but a wide variety of enzymes with delta-7 sterol reductase activity may alternatively be employed. Preferably, the delta-24 sterol reductase encoded by a heterologous nucleic acid is an animal delta-24 sterol reductase, for example DHCR24.

By way of example, expression of the delta-7 sterol reductase of Legionella drancourtii may be combined with expression of the delta-24 sterol reductase of Danio rerio for cholesterol production in an engineered Y. lipolytica of the invention as illustrated by strain ST11829 described further herein (see Example 4). For this purpose, the heterologous coding sequences for both the delta-7 sterol reductase and delta-24 sterol reductase will desirably be expressed by codon-optimised coding sequences for Y. lipolytica under the control of the PrTEFintron promoter or a functionally equivalent yeast promoter.

Again, preferably, the sterol surrogate may be provided by expression of a squalene-tetrahymanol cyclase under the control of the PrGPAT promoter or a functionally equivalent weak yeast promoter.

As seen in FIG. 7, as the yeast lacks an endogenous C-24 methyltransferase (ERG6) it is incapable of converting zymosterol to fecosterol. Instead zymosterol is converted by endogenous sterol C-8 isomerase (ERG2) to cholesta-7,24-dienol, which is then converted to 7-dehydrodesmosterol by endogenous sterol C-5 desaturase (ERG3). This is then converted to desmosterol by a heterologous delta-7 reductase (DHCR7). Desmosterol is then converted to cholesterol by a heterologous delta-24 sterol reductase (DCHR24). As the yeast lacks endogenous sterol C-22 desaturase (ERG5) it is incapable of acting on the 7-dehydrodesmosterol or desmosterol as well as incapable of producing ergosterol leading to production of cholesterol. Cholesterol is an essential structural component of animal cell membranes as well as being a precursor for the biosynthesis of steroid hormones, bile acid and vitamin D.

D. In a further preferred embodiment a yeast as described above expressing a sterol surrogate and engineered for production of one or more non-native sterols comprising isofucosterol (either the Z isomer or the E-isomer, more commonly distinguished by naming as fucosterol) has:
(i) an attenuated or deleted endogenous sterol C-22 desaturase enzyme (ERG5) and an attenuated or deleted delta-24 sterol reductase enzyme (ERG4);
(ii) a heterologous nucleic acid sequence encoding a delta-7 sterol reductase enzyme as described herein; and
(iii) a heterologous nucleic acid sequence encoding a sterol C-28 methyltransferase enzyme as described herein.

Preferably, the sterol C-28 methyltransferase encoded by a heterologous nucleic acid is a plant sterol C-28 methyl-transferase, for example SMT2 which produces solely isofucosterol as the Z plant isomer. As indicated above, to boost isofucosterol production in some instances it may be found preferable to employ more than one coding sequence for a sterol C-28 methyltransferase, e.g. 1 to 3 such coding sequences which may be the same or different. For production of isofucosterol in an engineered Y. lipolytica of the invention, such coding sequences will desirably be codon optimised for Y. lipolytica and under the control of for example a strong yeast promoter such as the PrTEFintron or PrGPD promoter. Preferably, the delta-7 sterol reductase encoded by a heterologous nucleic acid is a plant delta-7 sterol reductase, for example DWF5. For production of isofucosterol in an engineered Y. lipolytica of the invention, the parent starting strain may for example be a ΔERG5 ΔERG4 Y. lipolytica strain expressing the Tetraselmis sp. GSL018 delta-7 sterol reductase, again from a codon optimised sequence and under control of a PrTEFintron promoter or functionally equivalent promoter as illustrated by strain ST11064 and its further conversion to strains ST11803 and ST12108. For further discussion of such strain development for isofucosterol production, see again Example 4.

Again, preferably, the sterol surrogate may be provided by expression of a squalene-tetrahymanol cyclase under the control of the PrGPAT promoter or a functionally equivalent weak yeast promoter

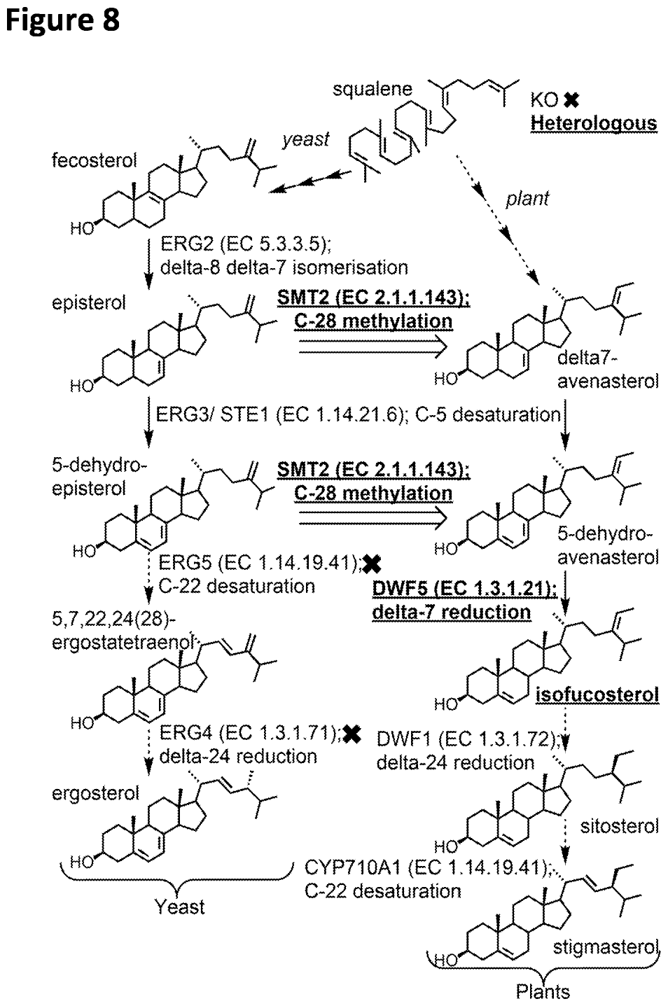

As shown in FIG. 8, squalene is converted to epiestrol and 5-dehydroepisterol by endogenous sterol C-24 methyltransferase (ERG6), sterol C-8 isomerase (ERG2) and sterol C-5 desaturase (ERG3). Epiestrol and 5-dehydroepisterol are then converted to delta 7-avenasterol or 5-dehydroavenasterol respectively by the heterologous sterol C-28 methyltransferase. Delta 7-avenasterol is also converted to 5-dehydroavenasterol by endogenous sterol C-5 desaturase (ERG3), 5-dehydroavenasterol is then converted to isofucosterol by the heterologous delta-7 sterol reductase. As the yeast lacks endogenous sterol C-24 reductase (ERG4) activity it is incapable of converting isofucosterol to sitosterol leading to production of isofucosterol.

Isofucosterol exhibits various biological therapeutic properties, including anticancer, antidiabetic, antioxidant, hepatoprotective, antihyperlipidemic, antifungal, antihistaminic, anticholinergic, antiadipogenic, anti-photodamaging, anti-osteoporotic, blood cholesterol reducing, blood vessel thrombosis preventive and butyrylcholinesterase inhibitory activities. Isofucosterol may also be used in artificial dietary compositions for example for insects such as bees.

E. In a further preferred embodiment a yeast as described above expressing a sterol surrogate and engineered for production of one or more non-native sterols comprising beta-sitosterol has:
(i) an attenuated or deleted endogenous sterol C-22 desaturase enzyme (ERG5):
(ii) a heterologous nucleic acid sequence encoding a delta-7 sterol reductase enzyme as described herein; and
(ii) a heterologous nucleic acid sequence encoding a sterol C-28 methyltransferase enzyme as described herein.

The yeast may further include an attenuated or deleted endogenous delta-24 sterol reductase enzyme (ERG4) and a heterologous nucleic acid encoding a plant delta-24 sterol reductase enzyme (DWF1). That is to say the yeast may have endogenous delta-24 sterol reductase enzyme (ERG4) replaced by a heterologous nucleic acid encoding a plant delta-24 sterol reductase enzyme (DWF1). This provides a yeast that produces beta-sitosterol in the same configuration (epimer) as found in plants.

Preferably, the sterol C-28 methyltransferase encoded by the heterologous nucleic acid is a plant sterol C-28 methyl-transferase, for example SMT2. Again, it may be found favourable to provide more than one sterol C-28 methyltransferase, preferably under the control of a strong yeast promoter, e.g. a PrTEFintron promoter or PrGPD promoter in the case of an engineered Y. lipolytica host.

Preferably, the delta-7 sterol reductase encoded by the heterologous nucleic acid is a plant delta-7 sterol reductase, for example DWF5, e.g. the delta-7 sterol reductase variant of Tetraselmis sp. GSL018. The parent strain may for example conveniently be a campesterol-producing Y. lipolytica AERG 5 strain in which the delta-7 sterol reductase variant of Tetraselmis sp. GSL018 is expressed from a codon-optimised sequence under the control of a PrTEFintron promoter or functionally equivalent promoter.

Again, preferably, the sterol surrogate may be provided by expression of a squalene-tetrahymanol cyclase under the control of the PrGPAT promoter or a functionally equivalent weak yeast promoter

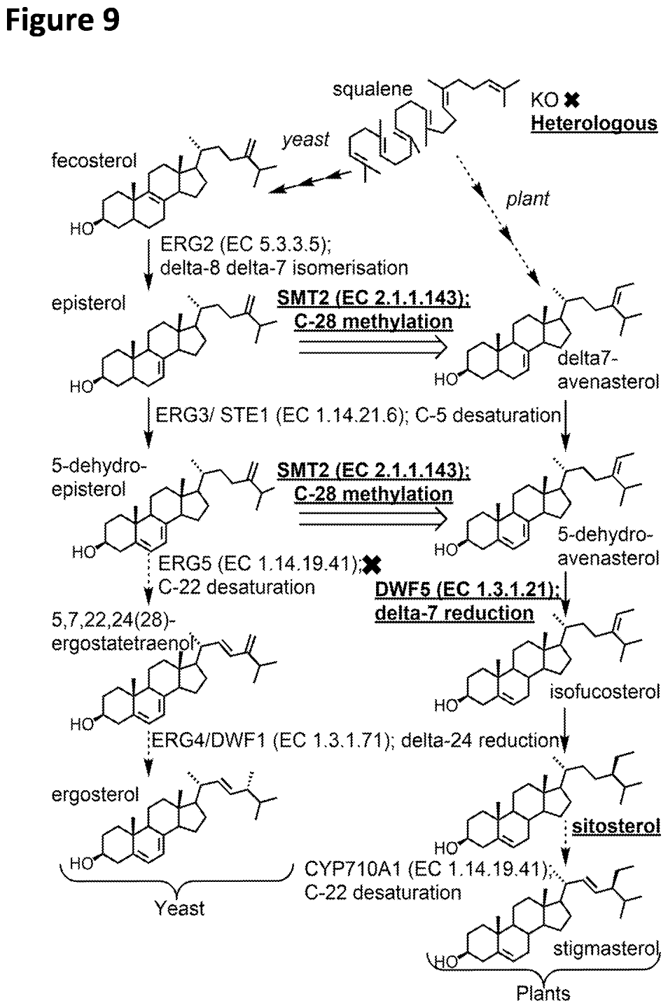

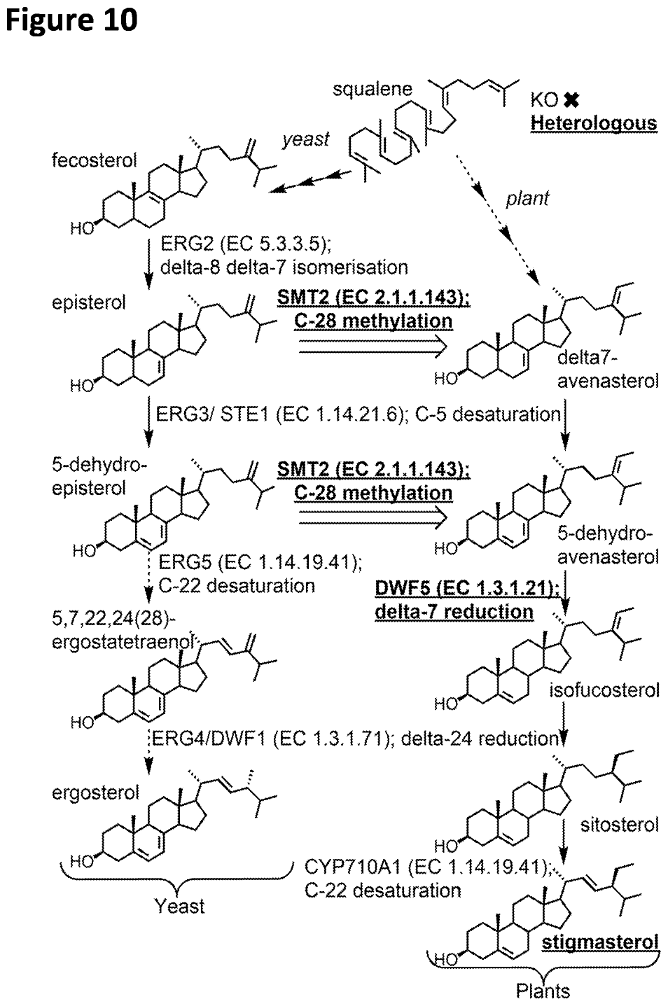

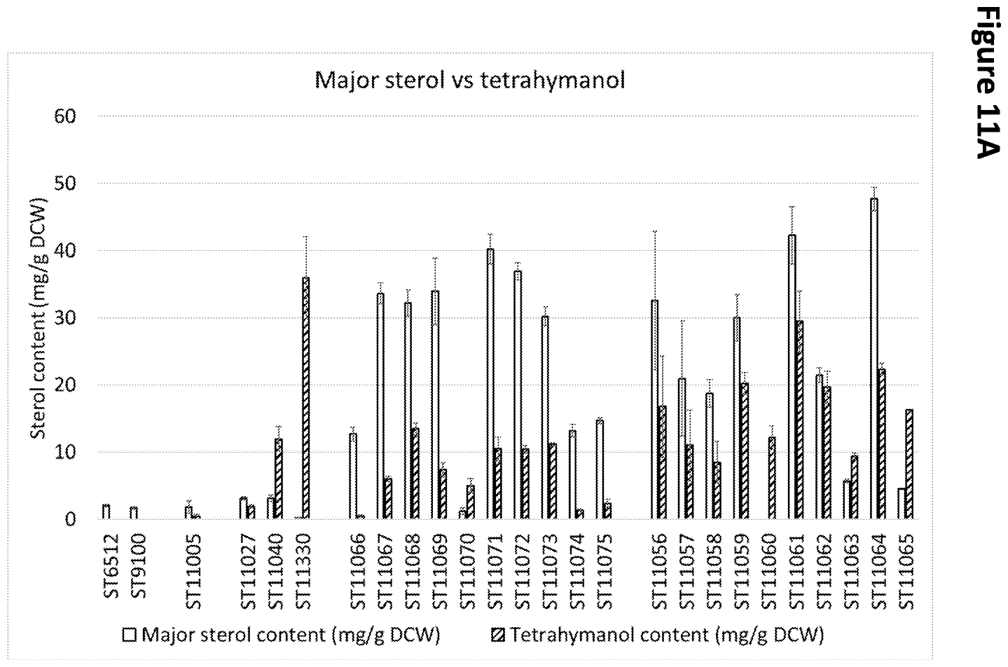

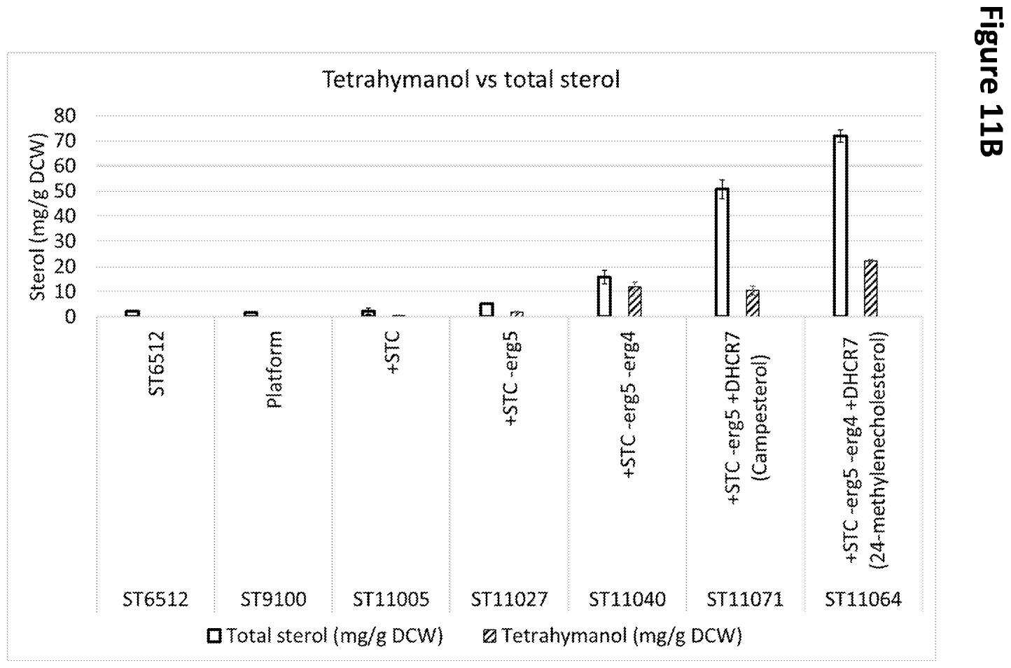

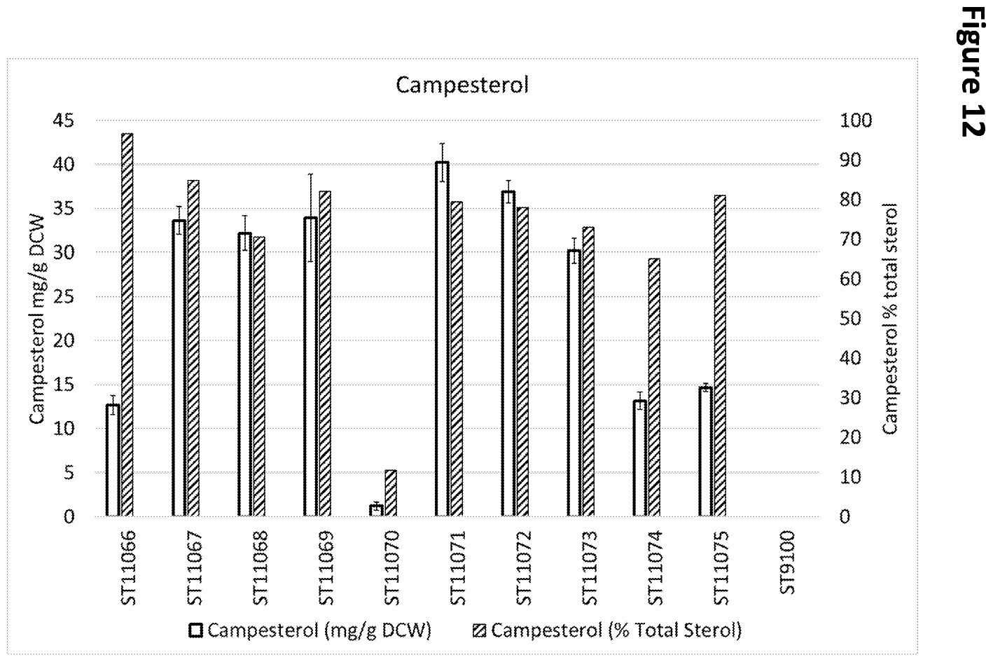

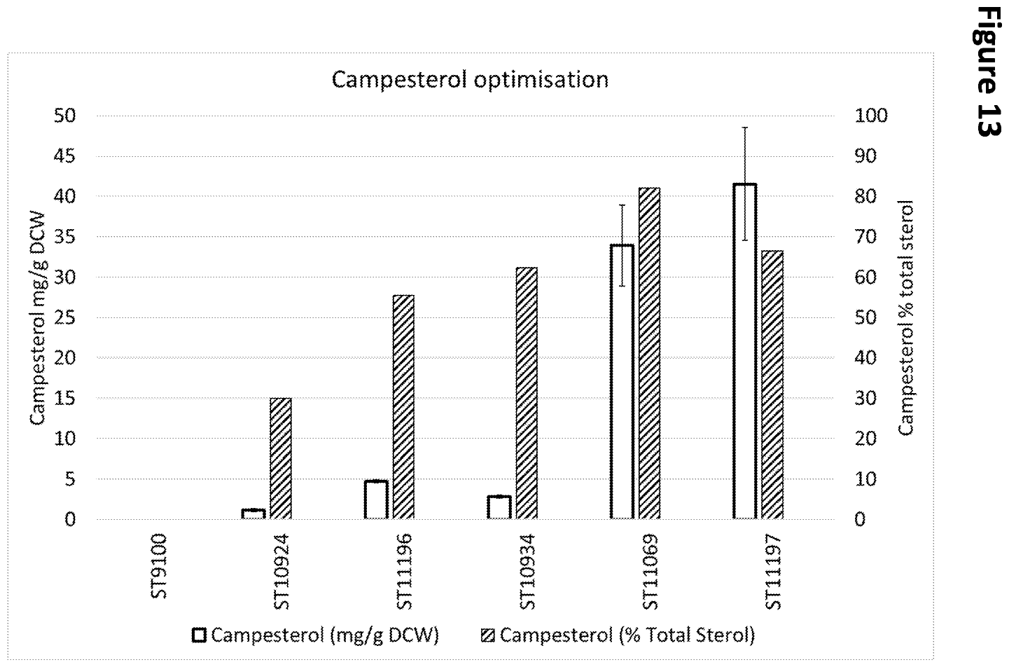

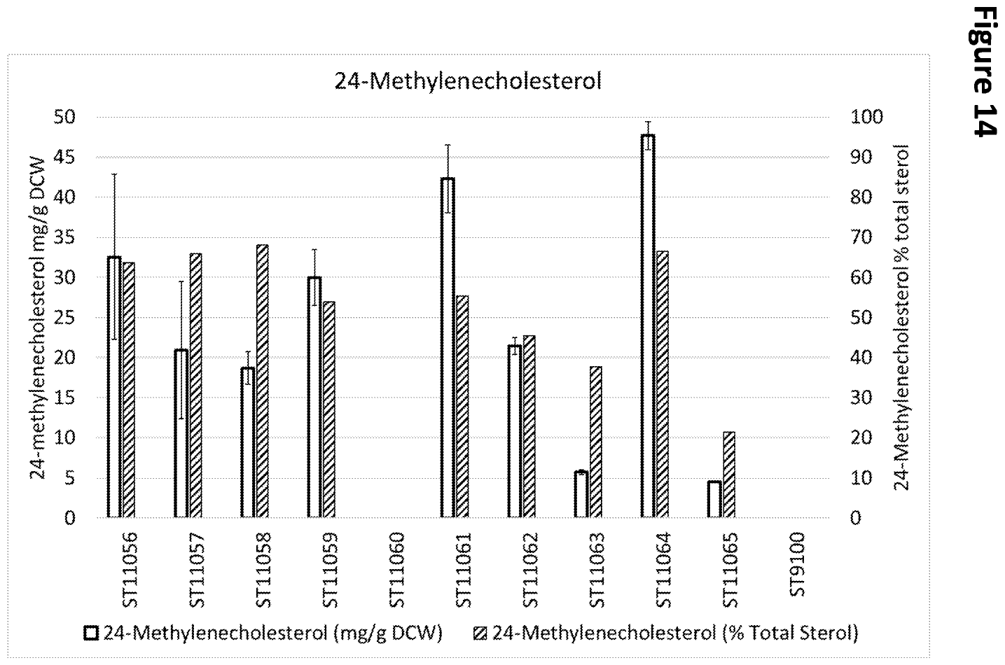

As shown in FIG. 9, squalene is converted to epiestrol and 5-dehydroepisterol by endogenous sterol C-24 methyltransferase (ERG8), sterol C-8 isomerase (ERG2) and sterol C-5 desaturase (ErRG3). Epiestrol and 5-dehydroepisterol are then converted to delta 7-avenasterol or 5-dehydroavenasterol respectively by the heterologous sterol C-28 methyl-transferase. Delta 7-avenasterol is also converted to 5-dehydroavenasterol by endogenous sterol C-5 desaturase (ERG3). 5-dehydroavenasterol is then converted to isofucosterol by heterologous delta-7 reductase. Endogenous delta-24 sterol reductase (ERG4) (or a substituted plant delta-24 sterol reductase) then converts isofucosterol to beta-sitosterol.

Beta-sitosterol is most commonly used for lowering cholesterol levels and improving symptoms of an enlarged prostate (benign prostatic hyperplasia or BPH) In human subjects. As such beta-sitosterol may be used in food compositions. In addition, beta-sitosterol may be used in artificial dietary compositions for example for insects such as bees. Beta-sitosterol is also a precursor for the production of brassinosteroids which may have antiviral, antifungal, antiproliferative, antibacterial, neuroprotective and immunomodulatory properties in animals.

F. As indicated above, of especial importance, for the first time there is now provided engineered yeast for production of 24-methylenecholesterol, either as a single sterol or as a component of a sterol mixture at a measurable amount. Thus, in a preferred embodiment a yeast as described above expressing a sterol surrogate and engineered for production of one or more non-native sterols comprising 24-methylenecholesterol has:
(i) an attenuated or deleted endogenous sterol C-22 desaturase enzyme (ERG5) and an attenuated or deleted delta-24 sterol reductase enzyme (ERG4); and
(ii) a heterologous nucleic acid sequence encoding a delta-7 sterol reductase enzyme as described herein.

Preferably, the delta-7 sterol reductase encoded by the heterologous nucleic acid is a plant delta-7 sterol reductase, for example DWF5.

Again, preferably, the sterol surrogate may be provided by expression of a squalene-tetrahymanol cyclase under the control of the PrGPAT promoter or a functionally equivalent weak yeast promoter

As seen in FIG. 5, if both the ERG5 and ERG4 genes are deleted, the yeast converts squalene to 5-dehydroepisterol using endogenous sterol C-24 methyltransferase (ERG6), sterol C-8 isomerase (ERG2) and sterol C-5 desaturase (ERG3). As the yeast lacks endogenous sterol C-22 desaturase (ERG5), the 5-dehydroepisterol is converted by the heterologous delta-7 reductase (DWFS) to 24-methylenecholesterol. As the yeast lacks sterol C-24 reductase (ERG4) activity, it is incapable of acting on the 24-methylenecholesterol as well as incapable of producing ergosterol leading to production of 24-methylenecholesterol.

24-methylenecholesterol may be used as a precursor for compounds such as withanolides which may have antimicrobial, anti-viral, anti-tumour, anti-arthritic, anti-aging, anti-inflammatory and neuroprotective properties. 24-methylenecholesterol may also be used for artificial dietary compositions, for example for insects such as bees.

24-methylenecholesterol has previously been recognised as an intermediate but has not previously been produced by any engineered yeast in useful manner and quantity. In contrast now provided are engineered yeast strains as indicated above which are able to provide 24-methylenecholesterol at high quantity, either as a single sterol or as part of sterol mixture containing one or more further non-native sterols in measurable amount, e.g. campesterol or campesterol and cholesterol.

By way of example of an especially preferred embodiment, by incorporating into a platform strain as above, e.g. Y. lipolytica ST9100, (i) both ERG5 gene deletion and ERG 4 gene deletion facilitated by expression of a coding sequence for a sterol surrogate such as preferably a codon-optimised coding sequence for squalene-tetrahymanol cyclase of Tetrahymena thermophila (TtSTC) under the control of a weak yeast promoter, e.g. PrGPAT and (ii) a codon-optimised coding sequence for expression of a delta-7 sterol reductase, e.g. Tetraselmis sp. GSL018 delta-7 sterol reductase, under the control of the PrTEFintron promoter or a functionally equivalent yeast promoter, production of 24-methylenecholesterol has been achieved at levels of more than 25 mg/g DCW, e.g. as high as 40-50 mg/g DCW. Using the Tetraselmis sp. GSL018 delta-7 sterol reductase in such an engineered strain derived from ST9100, production of 24-methylenecholesterol has been achieved at about 48 mg/g DCW. Such high 24-methylenecholesterol production was not predictable from any prior art studies with S. cerevisiae and has been achieved by culturing without feeding any sterol precursor but employing a conventional yeast culturing medium (YPD medium) containing glucose at 30° C. See in Example 3 re strain ST11064 and FIG. 14.

G. It will be appreciated however from above that there is equally interest for some applications in obtaining 24-methylenecholesterol as a major component of a sterol mixture. e.g. for incorporation into an artificial dietary composition for bees. In this case a platform strain as above, e.g. Y. lipolytica ST9100, may have an attenuated or deleted ERG5 gene, preferably a deleted ERG5 gene, and activity of ERG4 attenuated. This may be achieved by provision of the same coding sequence under the control of a weak promoter selected from the PrDGA1 promoter and functionally equivalent weak yeast promoters. Alternatively, the ERG4 gene may be substituted by a plant DWF1 coding sequence providing attenuated delta-24(28) sterol reductase activity under the control of a weak promoter such as the PrDGA1 promoter or a functionally equivalent weak promoter. In the presence also of expression of a coding sequence (preferably codon-optimised) for a delta-7 sterol reductase, e.g. Tetraselmis sp. GSL018 delta-7 sterol reductase, under the control of a stronger promoter, e.g. the PrTEFintron promoter or a functionally equivalent promoter, a sterol mixture comprising both 24-methylenecholesterol and campesterol in measurable amount can be attained. See Example 4. Moreover, if the yeast ER4G gene is substituted by a DWF1 gene, the campesterol component may importantly be obtained as the plant epimer. By way of example in providing such an engineered Y. lipolytica strain, expression of a delta-7 sterol reductase, such as the Tetraselmis sp. GSL018 delta-7 reductase under the control of the PrTEFintron promoter or a functionally equivalent strong yeast promoter may be combined with expression of the delta-24(28) sterol reductase gene from Solanum tuberosum (StSSR1) under the control of the PrDGA1 promoter or an equivalent weak yeast promoter as illustrated by strain ST11943 as described herein. By arranging for expression of the heterologous coding sequence for the delta-7 sterol reductase to be controlled by a strong yeast promoter and delta-24(28) sterol reductase expression to be controlled by a weaker promoter, a sterol mixture can be produced in which 24-methylenecholesterol is the dominant sterol, e.g. at above 15 mg/g DCW, e.g. about 18 mg/g DCW, but accompanied by useful, quantifiable campesterol, preferably as the plant epimer

Further incorporating a delta-24(25) sterol reductase under the control of a weak promoter, e.g. the PrGPAT promoter or a functionally equivalent promoter, may be preferred, e.g. introducing a codon-optimised coding sequence for the S. lycopersicum delta-24(25) sterol reductase under the control of the PrGPAT promoter or a functionally equivalent promoter. It has been shown that this can enable some 24-methylenecholesterol to be converted to cholesterol but with maintenance of 24-methylenecholesterol as the primary component of the attained sterol mixture with both measurable cholesterol and campesterol. Indeed, by such engineering of ST9100 as the platform strain 24-methylenecholesterol has still been attained at about 9-10 mg/g DCW in a mixture with both measurable cholesterol and campesterol. See Example 4.

As indicated above, further introduction of a C-28 sterol methyltransferase under the control of a weak promoter, e.g., the C. quinoa C-28 sterol methyltransferase encoded by a codon-optimised nucleic acid sequence under the control of the PrGPAT promoter or a functionally equivalent promoter, may be further considered to obtain a mixture of sterols. Such engineered Y. lipolytica strains are exemplified by strains ST11362 and strains ST11541 as further described in Example 4. As further discussed below, it has been found possible to further engineer these strains to omit expression of the sterol surrogate, thereby providing strains ST11441 and ST11542 (see strain construction tables 19 and 20 in the exemplification).

The genes for expression of a delta-24(25) sterol reductase and C-28 sterol reductase may be introduced in either alternative order as further illustrated by the strain construction table 21 for Y. lipolytica strain ST12178.

Thus as a preferred embodiment of an oleaginous yeast of the invention supplied with a sterol surrogate, there is provided such a yeast capable of producing a mixture of desired sterols comprising 24-methylenecholesterol, campesterol and cholesterol, optionally together with one or more further non-native sterols in detectable amount, wherein the oleaginous yeast comprises:
(i) an attenuated or deleted endogenous sterol C-22 desaturase enzyme (ERG5), preferably deleted ERG5;
(ii) an attenuated delta-24 sterol reductase enzyme (ERG4) or ERG4 substituted by a plant DWF1 enzyme providing attenuated delta-24(28) sterol reductase activity. e.g. where the ERG4 gene coding sequence or plant DWF1 enzyme coding sequence is under the control of the PrDGA1 promoter or a functionally equivalent weak promoter;
(iii) a heterologous nucleic acid sequence encoding a delta-7 sterol reductase enzyme, e.g. the delta-7 sterol reductase variant of Tetraselmis sp. GSL018, preferably under the control of a stronger promoter than employed for (ii), e.g. the PrTEFintron promoter or a functionally equivalent promoter,
(iv) a heterologous nucleic acid sequence encoding a delta-24(25) sterol reductase, e.g. the delta-24(25) sterol reductase of S. lycopersicum, preferably under the control of the PrGPAT promoter or a functionally equivalent weak promoter and optionally
(v) a heterologous nucleic acid sequence encoding a C-28 sterol methyltransferase, e.g. the C-28 sterol methyltransferase of C. quinoa, preferably under the control the PrGPAT promoter or a functionally equivalent promoter,
whereby said mixture of non-native sterols can be produced.

Alternatively, the heterologous nucleic acid sequence encoding the C-28 sterol methyltransferase may be provided and the heterologous nucleic acid sequence encoding the delta-24(25) sterol reductase omitted or introduced subsequently.

By way of a particularly favoured oleaginous yeast of the invention supplied with a sterol surrogate, preferably tetrahymanol from expression of a squalene-tetrahymanol cyclase coding sequence, there is provided such a strain, preferably an engineered Y. lipolytica, which is capable of producing a mixture of non-native sterols comprising 24-methylenecholesterol, campesterol as the 24R plant epimer, cholesterol, isofucosterol and desmosterol, wherein the oleaginous yeast comprises:
(i) a deleted endogenous sterol C-22 desaturase enzyme (ERG5).
(ii) ERG4 substituted by a DWF1 enzyme providing attenuated delta-24(28) sterol reductase activity, e.g. the delta-24(28) sterol reductase (DWF1) of Solanum tuberosum, where said DWF1 enzyme is under the control of the PrDGA1 promoter or a functionally equivalent weak promoter;
(iii) a heterologous nucleic acid sequence encoding a delta-7 sterol reductase enzyme, e.g. the delta-7 sterol reductase of Tetraselmis sp. GSL018, where said delta-7 sterol reductase is under the control of a stronger promoter than employed for (ii), e.g. the PrTEFintron promoter or a functionally equivalent promoter;
(iv) a heterologous nucleic acid sequence encoding a delta-24(25) sterol reductase, e.g. the delta-24(25) sterol reductase of S. lycopersicum, preferably under the control of the PrGPAT promoter or a functionally equivalent weak promoter and
(v) a heterologous nucleic acid sequence encoding a sterol C-28 methyltransferase, e.g. the sterol C-28 methyltransferase of C. quinoa, preferably under the control of the PrGPAT promoter or a functionally equivalent promoter, whereby said mixture of non-native sterols can be produced.

Such an engineered yeast can be obtained starting from Y. lipolytica ST9100 which is capable of producing 24-methylenecholesterol as the dominant sterol. e.g., in an amount of at least about 20 mg/g dry cell weight when cultured at 30° C. in yeast extract peptone dextrose (YPD) medium containing glucose and no sterol precursor, together with all of campesterol as the plant (24R) epimer, cholesterol, isofucosterol and desmosterol in quantifiable amount, preferably additionally with at least detectable beta-sitosterol. As indicated above, Y. lipolytica strain ST12178 is illustrative of such a favoured engineered Y. lipolytica, strain, the development of which from strain ST9100 is detailed in Example 4 and table 21. This exemplifies an engineered strain of the invention which produces both 24-methylenecholesterol and campesterol as the plant epimer together with other non-native sterols favouring use in provision of a sterol mixture for feeding of honeybees. This may be by isolation of sterols from a culture medium or alternatively use of recovered yeast cells as a yeast cell biomass following culturing with subsequent drying and preferably conversion to a powder. To boost p-sitosterol it may be chosen in some instances to add a 6-sitosterol supplement.

It will be appreciated that the strains construction steps set out in tables 19-21 are provided to illustrate production of specific exemplified Y. lipolytica strains. However, by following the same construction steps equivalent strains may be obtained starting from Y. lipolytica ST9100 or another oleaginous strain which shares all or some of the same modified genotype features compared with Y. lipolytica W29 strain Y-63747 as a reference strain with increased synthesis of squalene. Where use of a delta-7 reductase is specified this may be selected from any plant or animal delta-7 reductase that can be expressed in the yeast host. The delta 24(28) reductase which is expressed in strain ST12178 may be substituted by a functionally equivalent enzyme consistent with production of the 24R epimer of campesterol. Similarly the C-28 methyltransferase and delta-24(25) sterol reductase requiring steps may be carried out by providing any enzyme with the required activity. The enzymes employed may be known naturally-occurring enzymes or variants thereof which retain the required functional activity. One or more copies of each heterologous enzyme may be introduced.

Production of a Sterol Mixture without a Sterol Surrogate

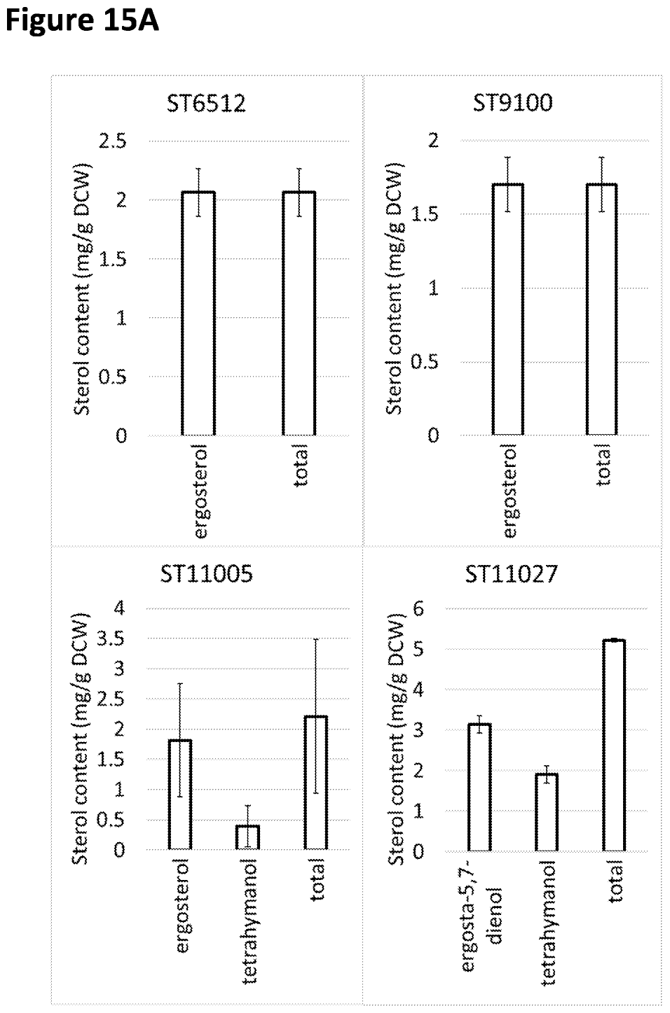

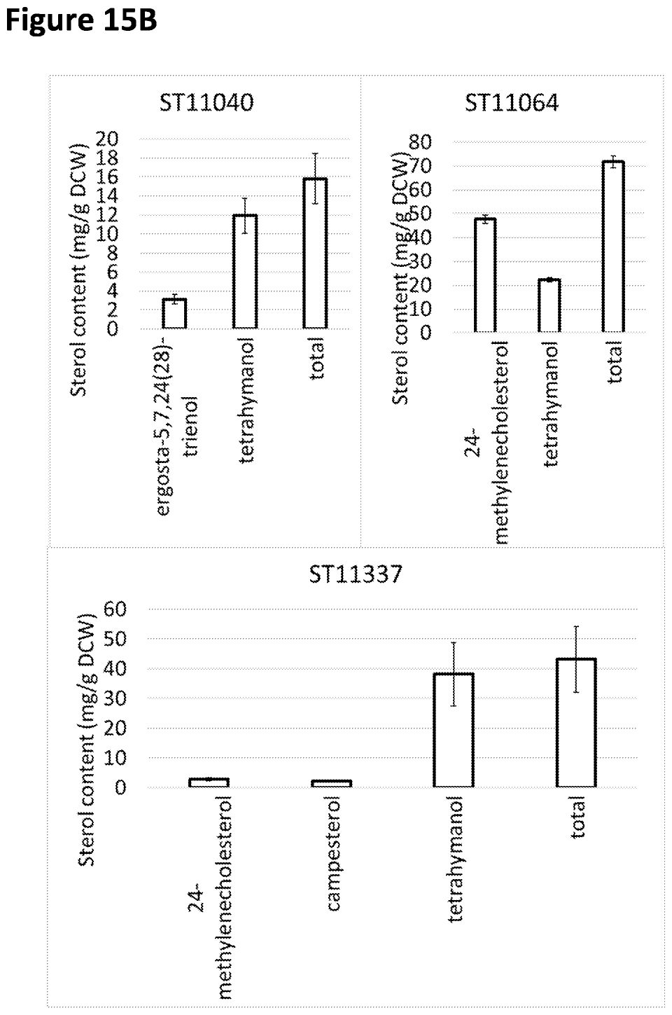

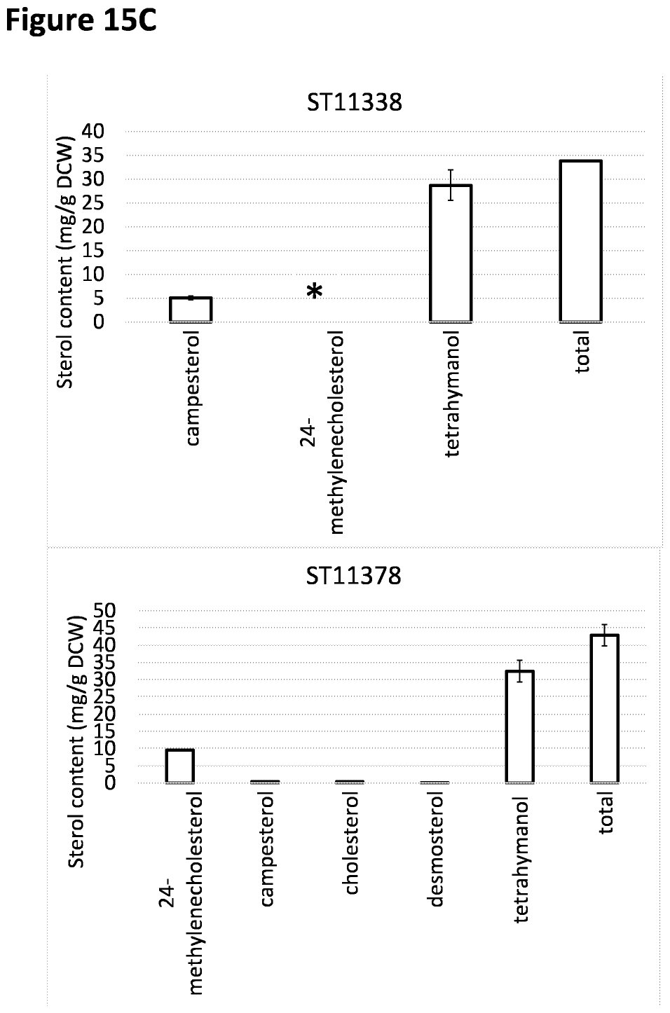

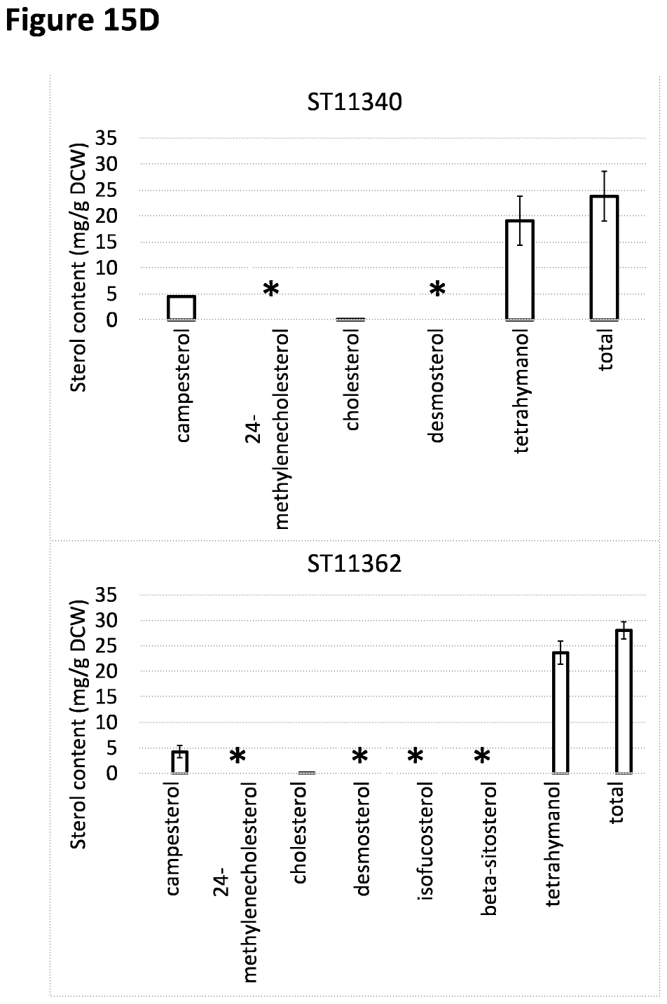

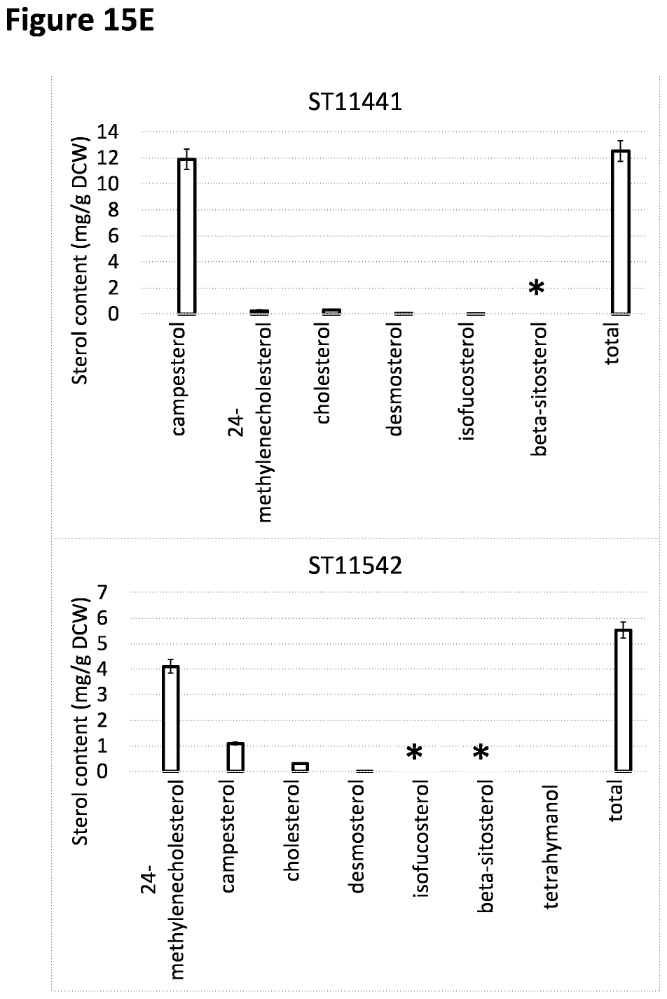

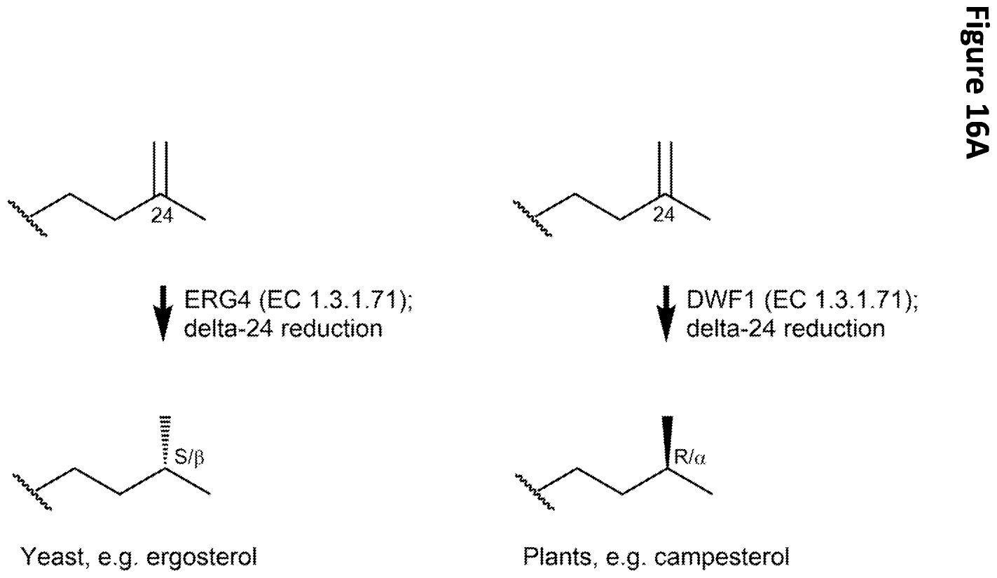

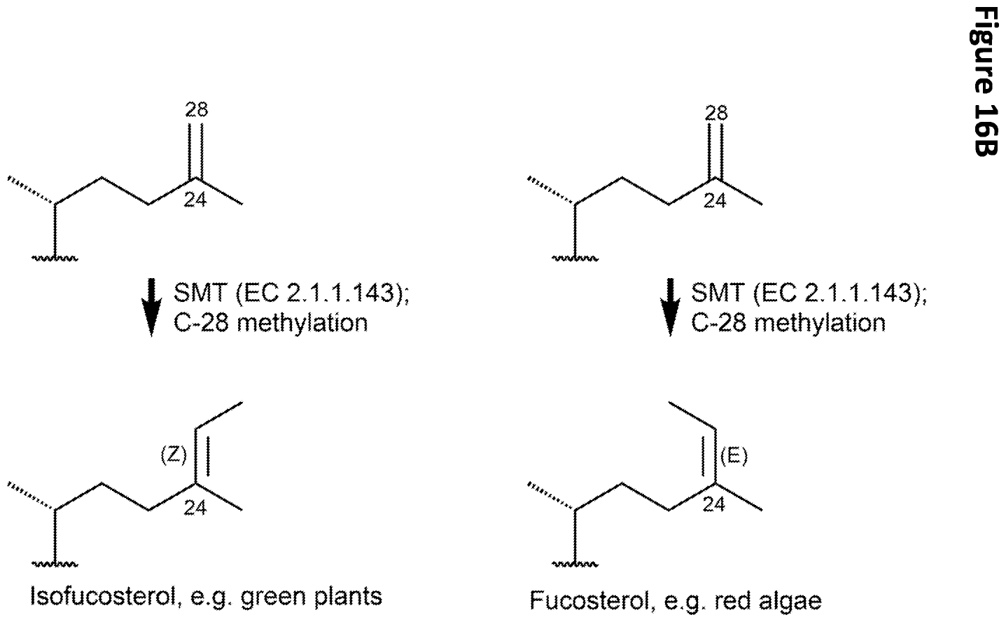

Interestingly, as indicated above, it has been found that the gene for expression of the sterol surrogate can be removed from a strain of the invention as discussed above having a sequence for expression of a C-28 sterol methyltransferase with retention of useful phytosterol mixture production, including campesterol, 24-methylenecholesterol and cholesterol as the major components and including other non-native sterols in detectable amount. Such a strain has been derived from Y. lipolytica strain ST9100 which has been observed to produce campesterol at about 12 mg/g DC with significant 24-methylenecholesterol and cholesterol by conventional culturing in YPD medium including glucose; see discussion of engineered strain ST11441 in Example 4. Furthermore, it has been found possible by such engineering to attain a strain capable of producing a sterol mixture again including all of campesterol, 24-methylenecholesterol and cholesterol but in which 24-methylenecholesterol is the dominant sterol, i.e. present in highest quantity; see additionally the discussion of ST11542 in Example 4 and FIG. 15. It will appreciated that by replacing the yeast ERG4 gene with a plant DWF1 gene, campesterol may be provided more desirably as the plant epimer for some applications.

Thus as a further aspect of the invention, there is provided use of an oleaginous yeast of the invention as discussed above which expresses a sterol surrogate and heterologous genes for production of a sterol mixture including 24-methylenecholesterol as a parent strain to produce a further yeast strain which produces a sterol mixture, where expression of the sterol surrogate is removed from said parent strain simultaneously with expression of all of a delta-7 sterol reductase, a delta 24(25) sterol reductase and a C-28 sterol methyltransferase plus attenuated ERG4 or substitute plant delta-24(28) sterol reductase (DWF1) enzyme. The resulting yeast cells may be cultured and utilised in the same way as other oleaginous yeast cells of the invention. The starting strain may preferably express all of a delta-7 sterol reductase, a delta 24(25) sterol reductase and a C-28 sterol methyltransferase plus DWF1 enzyme whereby a sterol mixture is produced comprising all of 24-methylenecholesterol, epicampesterol, cholesterol, desmosterol and isofucosterol and β-sitosterol. Removal of the sterol surrogate may be achieved with retention of production of the same sterols as illustrated by strains ST11441 and ST1152. See again strain construction tables 19 and 20.

Such a yeast-produced sterol mixture is for example of especial interest for feeding phytosterols via an artificial dietary composition to bees. Moreover, it can be envisaged that such industrially useful sterol compositions may be attained employing a wide variety of oleaginous yeasts, including Y. lipolytica strains without need for provision of any sterol surrogate.

Thus as an especially preferred embodiment of the invention, there is now provided an oleaginous yeast, preferably a Y. lipolytica strain, for producing a mixture of non-native sterols, which comprises: (i) an attenuated or deleted endogenous sterol C-22 desaturase enzyme (ERG5), preferably deleted ERG5;
(ii) an attenuated delta-24 sterol reductase enzyme (ERG4) or ERG4 substituted by an plant delta-24(28) sterol reductase (DWF1) enzyme providing attenuated delta-24(28) sterol reductase activity. e.g. where the ERG4 gene coding sequence or plant DWF1 enzyme coding sequence is under the control of the PrDGA1 promoter or a functionally equivalent weak promoter;
(ii) a heterologous nucleic acid sequence encoding a delta-7 sterol reductase enzyme, e.g. the delta-7 sterol reductase variant of Tetraselmis sp. GSL018, preferably under the control of a stronger promoter than employed for (ii), e.g. the PrTEFintron promoter or a functionally equivalent promoter.
(iv) a heterologous nucleic acid sequence encoding a delta-24(25) sterol reductase, e.g. the delta-24(25) sterol reductase of S. lycopersicum, preferably under the control of the PrGPAT promoter or a functionally equivalent weak promoter and
(v) a heterologous nucleic acid sequence encoding a C-28 sterol methyltransferase, e.g. the C-28 sterol methyltransferase of C. quinoa, preferably under the control the PrGPAT promoter or a functionally equivalent promoter, whereby a mixture of non-native sterols can be produced comprising 24-methylenecholesterol, campesterol and one or more further non-native sterols comprising cholesterol, preferably where 24-methylenecholesterol or campesterol is the dominant sterol of the mixture.

The dominant sterol, either 24-methylenecholesterol or cholesterol, may attain a production level of at least 4 mg/g DCW in YPD medium including glucose and as indicated above possibly higher, e.g. at least 10 mg/g DCW. This combined with measurable cholesterol provides a sterol composition of much interest for many applications, including for example incorporation into an artificial pollen substitute for use in bee feeding.

### Further Modifications

The yeast of the invention described above, either including a sterol surrogate or not including a sterol surrogate, may also include a number of additional genetic modifications. These additional or further genetic modifications may be provided for improving growth or maintenance of the yeast and/or improving the content of exogenous sterol produced.

Sterol content can be increased above the levels of native sterols in wild type or reference strains. For example, the concentration of precursor may be increased. Acetyl CoA is the main precursor to the mevalonate and subsequent pathways, and can be increased by overexpression of ACL (ATP citrate lyase) and/or ACS (acetyl CoA synthetase). Flux through the precursor pathways can be increased by overexpression of the biosynthetic enzymes involved, e.g. HMGR (HMG CoA reductase) and ERG12 (mevalonate kinase). The activity of biosynthetic enzymes in the main sterol biosynthesis pathway can be similarly increased.

As such, yeast of the invention may include further genetic modifications in order to alter and/or improve production of endogenous sterols as described above.

For example, the yeast of the invention may further comprise reduced activity of any one or more of the following enzymes or variants thereof:

PAH1 (Mg2+-dependent phosphatidate phosphatase) which dephosphorylates phosphatidate (PA) to yield diacylglycerol. PAH1 regulates phospholipid synthesis, nuclear/ER membrane growth, lipid droplet formation, triacylglycerol synthesis, vacuolar homeostasis and cell wall integrity. PAH1 also controls transcription of phospholipid biosynthetic genes and nuclear structure by regulating the amount of membrane present at the nuclear envelope;
Bts1 (geranylgeranyl pyrophosphate synthase) which catalyses the trans-addition of the 3 molecules of IPP onto DMAPP to form geranylgeranyl pyrophosphate. Bts1 is required for membrane attachment of YPT1 and SEC4. May be involved in vesicle trafficking and protein sorting;
GDH1 (NADP-specific glutamate dehydrogenase 1) which synthesizes glutamate from ammonia and alpha-ketoglutarate;
Are1 (sterol O-acyltransferase 1) which is an endoplasmic reticulum enzyme that contributes the major sterol esterification activity in the absence of oxygen;
Say1 (stearyl acetyl hydrolase 1) which is required for the deacetylation of acetylated sterols;
Sds23 (NCBI ref XP_504058.1) which is involved in DNA replication and cell separation;
Ins1 (NCBI ref XP_500057.1) which is an INSIG family protein involved in regulation of HMGR activity.
Ku70 (ATP-dependent DNA helicase II subunit 1) which is involved in non-homologous end joining (NHEJ) DNA double strand break repair and telomere maintenance; and/or
Ku80 (ATP-dependent DNA helicase 2 subunit KU80)) which is involved in non-homologous end joining (NHEJ) DNA double strand break repair and telomere maintenance.

Yeast of the invention may comprise increased or introduced activity of any one or more of the following enzymes or variants thereof:
HMG1 (3-hydroxy-3-methylglutaryl-coenzyme A reductase 1) which catalyse the conversion of HMG-CoA to mevalonate;
tHMG1 (truncated 3-hydroxy-3-methylglutaryl-coenzyme A reductase 1) whereby the regulatory domain is removed;
UPC2 (sterol uptake control protein 2) which is a transcription factor involved in activation of anaerobic genes such as DAN/TIR cel wall mannoprotein genes and YML083c;
Ecm22 (sterol regulatory element binding protein) which regulates transcription of sterol biosynthetic genes upon sterol depletion;
Erg1 (squalene epoxidase) which converts squalene into lanosterol; Erg3 (sterol C-5 desaturase) which converts epiestrol to 5, 7, 24(28)-ergostatrienol:
Erg7 (lanosterol synthase) which converts squalene into lanosterol;
Erg8 (phosphomevalonate kinase) which is involved in the formation of farnesyl pyrophosphate;
Erg9 (squalene synthase) which converts farnesyl pyrophosphate to squalene:
Erg10 (acetyl-CoA C-acyltransferase) which forms mevalonate from acetyl-coenzyme A:
Erg11 (lanosterol 14-α-demethylase) which is involved in the conversion of lanosterol to zymosterol;
Erg12 (mevalonate kinase) which is involved in the formation of farnesyl pyrophosphate;
Erg13 (HMG-CoA synthase) which is involved in the formation hydroxy-3-methylglutaryl-coenzyme A;
Erg19 (mevalonate pyrophosphate decarboxylase) which is involved in the formation of farnesyl pyrophosphate;
Erg20 (farnesyl pyrophosphate synthetase) which is involved in the formation of farnesyl pyrophosphate;
Erg25 (sterol C-4 methyloxydase) which is part of the C-4 demethylation complex;
Erg26 (sterol C-3 dehydrogenase) which is part of the C-4 demethylation complex;
Erg27 (sterol C-3 ketoreductase) which is part of the C-4 demethylation complex;
IDI (isopentenyl diphosphate isomerase) which is involved in the formation of farnesyl pyrophosphate;
Acl1 (ATP-citrate lyase subunit 1) which catalyses the formation of cytosolic acetyl-CoA from citrate;
Acl2 (ATP-citrate lyase subunit 2) which catalyses the formation of cytosolic acetyl-CoA from citrate;
Pot1 (3-ketoacyl-CoA thiolase) which cleaves 3-ketoacyl-CoA into acyl-CoA and acetyl-CoA during beta-oxidation of fatty acids:
Pat1 (peroxisomal acetoacetyl-CoA thiolase) which is involved in the last step of beta-oxidation of fatty acids:
Pex10 (Peroxisomal membrane E3 ubiquitin ligase) which is required for Ubc4p-dependent Pex5p ubiquitination and peroxisomal matrix protein import;
GDH2 (NAD(+)-dependent glutamate dehydrogenase) which degrades glutamate to ammonia and alpha-ketoglutarate;
G6PD1 (Glucose-6-phosphate 1-dehydrogenase 1) which provide reducing power (NADPH) and pentose phosphates for fatty acid and nucleic acid synthesis which are involved in membrane synthesis and cell division
Are1 (sterol O-acyltransferase 1) which is an endoplasmic reticulum enzyme that contributes the major sterol esterification activity in the absence of oxygen
Are2 (acyl-CoA:sterol acyltransferase) which is an endoplasmic reticulum enzyme that contributes the major sterol esterification activity in the presence of oxygen
Atf2 (alcohol acyltransferase) which may play a role in steroid detoxification and forms volatile esters during fermentation;
Acs1 (Acetyl-coA synthetase isoform) which catalyses the formation of acetyl-coA from acetate;
SSD1 (protein SSD1) which is a translational repressor with a role in polar growth and wall integrity;
YBP1 (YAP1-binding protein) which is involved in cellular response to oxidative stress and required for oxidation of specific cysteine residues of transcription factor Yap1p, resulting in nuclear localization of Yap1p in response to stress;
Sre1 (sterol regulatory element-binding protein 1) transcriptional activator required for transcription of genes required for adaptation to anaerobic growth like those implicated in the non-respiratory oxygen-consumptive biosynthetic pathways of sterol, heme, sphingolipid, and ubiquinone biosynthesis;
Css1 (secreted protein CSS1) which may be involved in cell wall organization and biosynthesis.

The additional modifications may be used to increase sterol esterification, lipid droplet size and/or intracellular membranes in order improve sterol accumulation in yeast of the invention.

As noted above, the Y. lipolytica strain ST9100 has the genotype:
MATa ku70Δ::PrTEF1→Cas9-TTef12::PrGPD→DsdA-TLip2 IntC_2-HMG1c-PrGPD-PrTeflnt→ERG12 IntC_3-SeACS←PrGPDPrTefint→YIACL1 IntD_1-IDI1←PrGPD-PrTefInt→ERG20 (Arnesen et al (2020) ibid).

Oleaginous yeasts of the invention derived from ST9100 are especially preferred and will desirably retain at least all the genotype features above which contribute to increased squalene synthesis in cultured ST9100 compared to the reference Y. lipolytica W29 Y-63746 (ST4842) as available from the ARS culture collection such as the increased expression of HMG1, ERG12, ACL1, ACs, IDI and ERG20. It will be appreciated however that other oleaginous yeast with the same genotype may be constructed by means of yeast strain development well-known to those skilled in the field.

### Methods to Introduce Genes

One or more of the heterologous nucleic acids encoding any of the enzymes described herein may be chromosomally integrated, or may be expressed on an extrachromosomal vector. Suitable vectors are well known. Similarly, methods of chromosomally inserting a nucleic acid are known.

For example, heterologous nucleic acids as described herein may be introduced into a yeast of the invention by methods such as heterologous recombination, site directed insertion or CRISPR-Cas9 based methods.

The heterologous nucleic acids may be inserted at any location within the yeast's genome. Preferably, the heterologous nucleic acids are inserted at one or more locations in the yeast genome that allow for relatively high expression of the heterologous nucleic acids while not having a deleterious effect on the growth of the yeast. For example, the EasyCloneYALI toolbox (Holkenbrink of at, 2018), defines 11 intergenic sites with high gene expression levels where integration of genes may be made.

Non-limiting examples of suitable promoters may include PrTEFintron, PrGPAT, pCyc, pAdh, pSte5, pPGK1, prGAL1, prENO2, prhp4d and prFIG1.

As indicated above, for expression of a heterologous coding sequence for non-native sterol production where strong expression is desired the yeast promoter PrTEFintron may be considered suitable or a functional equivalent thereof which can direct at least the same expression. Where weaker expression is desired, e.g. for expression of a sterol surrogate, as indicated the yeast promoter PrGPAT may be selected or a functionally equivalent weak yeast promoter. Such functionally equivalent promoters may be natural or synthetic and determined in conventional manner by fluorescent assay of GFP expression in yeast. See Holkenbrink et al (2020) ibid.

Nucleic acid constructs for use in providing a yeast of the invention may also comprise flanking sequences. The phrase “flanking sequence” or “homology arms” refers to a nucleic acid sequence homologous to a chromosomal sequence. A construct comprising a flanking sequence on either side of a construct (i.e., a left flanking sequence and a right flanking sequence) may homologously recombine with the homologous chromosome, thereby integrating the construct between the flanking sequences into the chromosome. Generally speaking, flanking sequences may be of variable length.

Heterologous nucleic acids provided in a yeast of the invention will generally be codon optimised. Codon optimization is a process used to improve gene expression and increase the translational efficiency of a gene of interest by accommodating codon bias of the host organism. Various codon optimization strategies have been developed by using a range of quantitative methods to generate different mRNA sequences, which can result in different levels of final protein expression. Most optimization strategies use codons with host bias to replace less frequently occurring codons.

Examples of codon optimised sequences of heterologous nucleic acids which may be included in oleaginous yeast of the invention are provided in Table 4. The sequences were codon optimised for Y. lipolytica using the GeneArt portal for String DNA fragments by ThermoFisher Scientific.

Nucleic acids employed in constructing yeast of the invention may include selection markers that allow for selection of yeast that include the heterologous nucleic acids of the invention. For example, markers may include antibiotic resistance.

Nucleic acids may be introduced into a yeast of the invention by any known methods, for example, by transformation methods such as lithium based methods, electroporation, biolistic methods, and glass bead methods. For example, it may be chosen to integrate heterologous sequences at intergenic loci in the Y. lipolytica genome as indicated above by introducing purified DNA for integration using the lithium acetate transformation protocol as described by Holkenbrink et al. (2018) Biotech J.

### Production of Sterol Derivatives

A yeast of the invention may be used for production of one or more desired sterols or compounds derived therefrom. Thus, a yeast of the invention may additionally possess genotype features whereby it can convert one or more initially produced non-native sterols to one or more desired sterol derivatives, e.g. sterol esters. Alternatively, one or more non-native sterols (or derivatives thereof) may be isolated from a yeast of the invention and subsequently converted to one or more sterol derivatives, e.g. sterol esters, for various applications. Such further conversion of initially synthesized non-native sterols may be by well-established means of common general knowledge and will be further discussed below.

Culturing and Isolation of Sterols from Yeast of the Invention

In a further aspect, the present invention provides a method for production of one or more non-native sterols which comprises culturing cells of an oleaginous yeast of the invention whereby said one or more desired non-native sterols are synthesised. Culturing may be in any suitable culture medium used for yeast. For example, the culture medium may be yeast extract peptone dextrose (YPD) media. The culture media may include a number of additional additives.

For example the culture medium may further include one or more of citrate, pyruvate, acetate, vegetable oil, glycerol, a beta-cyclodextrin to increase lipid synthesis or accumulation within the yeast, and/or increase the stability of the yeast in culture.

The culture medium may also include a carbon source. An additional carbon source may help to increase sterol production within yeast of the invention. Suitable carbon sources may include one or more of glucose, fructose, sucrose, xylose, mannose, galactose, rhamnose, arabinose, one or more fatty acids, glycerol, acetate, citrate, pyruvate, starch, glycogen, amylopectin, amylose, cellulose, cellulose acetate, cellulose nitrate, hemicellulose, xylan, glucuronoxylan, arabinoxylan, glucomannan, xyloglucan, lignin, lignocellulose and/or vegetable oil. Preferably, the carbon source is glucose.

Yeast of the invention may be cultured with an isotopically labeled substrate. Thus, the culture medium may include an isotopically-labeled carbon source of a type noted above, e.g. uniformly labelled 13C glucose (D-[U-13C]glucose). Use of an isotopically labelled substrate leads to production of isotopically labelled exogenous sterols. The use of isotopic labels may help with recovery of exogenous sterols from the yeast. Isotopes that may be used include ¹⁸O, ²H, ¹⁵N and ¹³C.

The culture medium may also include antibiotics in order to selectively allow culture of those yeast that include and express heterologous nucleic acids of the invention.

Various temperature and duration of culturing may also be used. The culturing may be performed under aerobic conditions, such as by shaking and/or stirring with aeration. The yeast may be cultured at a temperature of about 20 to about 40° C. Preferably, the yeast may be cultured at a temperature of about 30° C.

Yeast of the invention may be cultured for about 12, 24, 38, 48, 80, 72 hours or more. Preferably, the yeast may be cultivated for 72 hours or more. Yeast of the invention may be cultured for about 1, 2, 3, 4, 5, 8, 7, 8, 9.10 or more days.

Cultivation may preferably be by fed-batch culture, e.g. In a 5-L bioreactor, with glucose feed as the carbon source. Such fed-batch culture is well-known for yeast and may be optimised for any yeast strain of the invention to aid required sterol production as illustrated by the exemplification

Exogenous sterols produced by yeast of the invention may be stored in lipid droplets of the yeast and may be stored in esterified form.

The methods of the invention may provide one or more of the exogenous sterols at a titre of at least 0.25 mg/l. For example, the exogenous sterol may be produced by a yeast of the invention at a titre of at least 0.25 mg/l, at least 0.5 mg/ml, at least 1 mg/mi, at least 10 mg/ml, at least 20 mg/ml, at least 25 mg/ml, at least 50 mg/ml, at least 100 mg/ml, at least 150 mg/ml, at least 200 mg/ml, at least 400 mg/ml, at least 500 mg/ml, at least 600 mg/ml, at least 700 mg/ml, at least 800 mg/ml.

The methods of the invention may provide one or more of the exogenous sterols at a dry cell weight (DCW) of at least 0.01-1 mg/g DCW whereby quantification is possible but as illustrated by the exemplification a sterol mixture may include one or more sterols additionally in trace amount. For example, the exogenous sterol may be produced by a yeast of the invention at a DCW of at least 1 mg/g DCW, at least 5 mg/g DCW, at least 10 mg/g DCW, at least 15 mg/g DCW, at least 20 mg/g DCW, at least 25 mg/g DCW, at least 30 mg/g DCW, at least 35 mg/g DCW, at least 40 mg/g DCW. For example, at least 10 mg/g DCW. For example, at least 25 mg/g DCW.

The methods of the invention may provide a yeast that comprises at least 20% of the one or more exogenous sterols compared to the total sterol content of the yeast, for example at least 20%, 21%, 22%, 23%, 24%, 25%, 30%, 35%, 40%, 45%, 50%, 55%, 60%, 65%, 70%, 75%, 80%, 85%, 90%, 95%, 98%, or 99%.

The specific exogenous sterols produced by the combinations of gene attenuations or deletions and the heterologous nucleic acids as described herein, for example as detailed in Table 2 may be the major or dominant exogenous sterol produced. That is to say that the yeast may also include other intermediate sterol compounds that may be produced during synthesis of the major sterol. The term major or dominant sterol is used to describe the main or most abundant sterol produced by a yeast of the invention. For example, the major or dominant sterol may be the sterol produced at the highest percentage of all sterols produced by the yeast i.e. highest percentage sterol of the total sterol content of the yeast. As such, yeast of the invention may produce a composition of sterols which may include one or more intermediates of the biosynthetic pathway for production of the specified sterol.

By way of example Y. lipolytica strain ST12178 when grown on conventional YPD medium at 30° C. with glucose at 80 g/l was observed to produce 22.6 mg/g DCW of 24-methylenecholesterol as the dominant sterol, 2.8.mg/g DCW campesterol as the plant epimer (24R), 0.01 mg/g DCW isofucosterol, 8.5 mg/g DCW cholesterol and 0.8 mg/g DCW desmosterol plus detectable (trace amount) β-sitosterol. For this reason, it was considered of especial interest for larger scale fed-batch culture for production of a sterol mixture for various uses by incorporation into a composition, particularly an artificial dietary composition for use in artificial feeding of managed honeybees. It will be appreciated that other Y. lipolytica strains capable of producing the same combination of sterols (at least 24-methylenecholesterol, campesterol, cholesterol, desmosterol and isofucosterol in measurable amount, preferably with 24-methylenecholesterol as the dominant sterol) may be obtained in accordance with the construction steps shown in table 21. As previously noted, Y. lipolytica ST9100 may be the chosen starting strain but an alternative starting Y. lipolytica strain is not excluded which shares al or some of the same modified genotype features as Y. lipolytica ST9100 compared with Y. lipolytica W29 strain Y-63748.

After culturing, in some instances the yeast may be used as a whole or the desired one or more sterols and/or derivatives may be recovered. Recovery of the sterols may be carried out by saponification which refers to the process of hydrolysis of an ester in the presence of an aqueous solution of hydroxide.

Saponified sterols may then be extracted using a hydrophobic solvent in order to provide free sterols. For example, n-hexane may be preferably employed to facilitate sterol extraction from the cell membrane. Alternatively, exogenous sterols may be recovered in esterified form.

Bi-phasic cultivation with an organic phase such as dodecane may allow for passive extraction of sterols from the cel membrane into a dodecane phase. This may reduce the toxicity of sterol accumulation and allow for greater sterol production. Sterols can be recovered from both phases.

Recovery of desired sterol(s) and/or derivatives from the culture medium simply retained within a yeast cell biomass may in some instances be preferred with subsequent inactivation. In this case, the yeast cell biomass may in some instances preferably be dried, e.g. by heating at no more than 80° C. Such heating will generally be for at least 24 hrs so that the yeast is heat-inactivated but the desired sterol(s) and/or derivatives(s) are maintained. The dried yeast cells may be used directly but more commonly will be converted into a powder for incorporation into a composition such as an artificial dietary composition, a food product, an agricultural composition, a cosmetic composition or pharmaceutical composition. Such processing to form a sterol-comprising dried powder may for example be utilised for incorporation of a sterol mixture produced by an oleaginous yeast of the invention into an artificial dietary composition for bees (see Example 5). For this purpose, the dried yeast powder may be combined with other components to provide a holistic diet at for example 20% w/w. Such a dietary composition may combine such a dried yeast powder with other components commonly considered desirable for bee development in managed honeybee colonies such as protein, lipid, sugar, minerals and vitamins. Where the sterol mixture produced in accordance with the invention is lacking β-sitosterol or has lower than desired β-sitosterol, then as indicated above additional β-sitosterol may be added.

### Examples of Downstream Products Obtainable by Use of Yeast of the Invention for Sterol Production

The exogenous sterols, either within the yeast or recovered from the yeast may be further processed or converted to a compound derived from said sterols. For example, campesterol may be further converted to sterol drugs such as progesterone or hydrocortisone.

Phytosterols (plant sterols) such as 24-methylenecholesterol, campesterol, isofucosterol, beta-sitosterol and/or stigmasterol may be converted to brassinosteroids or withanolides.

Sterols such as cholesterol may be converted to vitamin D (cholecalciferol).

The recovered exogenous one or more sterols and/or sterol derivatives may be further incorporated into an artificial dietary composition, an agricultural composition, a food product, a cosmetic composition or a pharmaceutical composition. As indicated above, recovered cultured yeast cells of the invention may in some instances be directly incorporated into a composition, e.g. an artificial dietary composition for bees.

Artificial dietary compositions refers to a mixture of compounds that may be used for providing nutrition to a subject that are not the natural food of a subject. As indicated above, one especially favoured application of the invention is in enabling convenient production of one or more sterols or cultured yeast cells for use in feeding honeybees as a pollen substitute. One or more sterols or yeast cells may be incorporated into an artificial dietary compositions for this purpose as described in WO 2017/085477.

However, equally sterols or sterol derivatives prepared by culturing yeast cells of the invention may be incorporated into a variety of commercially useful compositions requiring one or more such components such as cosmetic compositions. Cosmetic composition refers to a non-therapeutic composition for care of the skin, hair, nails or other body parts of a subject. Sterols may be used in anti-aging creams and sun-care lotions. Oils and creams containing sterols may exhibit strong UV-protection. Cosmetic compositions may be a makeup product or a makeup-removal product. Cosmetic compositions may be serums, lotions, creams, shampoos, conditioners, oils, milks, ointments, pastes, foams, emulsions, hydrogels, shower gels, masks, lacquers, sprays or waxes.

Food compositions into which sterols prepared in accordance with the invention may be incorporated include yoghurts and milk, cheese, fat spreads, mayonnaise, salad dressing and other dairy products. It is thought that including plant stanols and sterols in the diet reduces the absorption of cholesterol. The unabsorbed cholesterol is excreted via the large bowel and this results in much less cholesterol reaching the blood supply and hence a lower blood cholesterol concentration. Total and LDL (low density lipoprotein) cholesterol levels are lowered without affecting HDL (high density lipoprotein) concentrations. Approximately 3040% of total cholesterol is absorbed from the intestine into the blood; however when plant stanol and sterol esters are present, absorption of cholesterol may fall to approximately 20%.

One or more sterols or sterol-derived compounds obtained by culturing yeast cells of the invention may further be incorporated into a variety of pharmaceutical compositions. Sterol-derived compounds of pharmaceutical interest are well known in the pharmaceutical field, e.g. withanolides and bioactive steroidal lactones, and may be synthesized from sterols produced in accordance with the invention by known methods. Sterol derivatives can also include sterol conjugates such as sterol esters or sterol glycosides. Sterols can be conjugated to compounds such as fatty acids and sugars via the C-3 hydroxyl group.

As precursors to brassinosteroids, as previously indicated production of sterols in accordance with the invention may also find application in the agricultural field.

Similarly to sterols, other valuable compounds may be derived from squalene. Terpenoid compounds, including beta-carotene, beta-caryophyllene, beta-cryptoxanthin, lutein and linalool could be synthesised in an oleaginous yeast optimised for increased squalene production (Arnesen et al. 2020). Synthesis of one or more of these compounds could increase the nutritional value of the oleaginous yeast when used in a food or feed. For example, a yeast strain of the invention capable of producing one or more non-native sterols could be further engineered so that it produces one or more additional desired squalene-derived compounds, for example a terpenoid compound useful as a pigment such as beta-carotene and/or a terpenoid compound useful as a scent such as linalool in an artificial dietary composition. This would increase the suitability of a yeast-based feed for insects such as bees. Such a yeast may also be cultured with subsequent recovery of one or more desired non-native sterols and/or one or more desired sterol-derived compounds and/or one or more compounds derived from increased squalene production.

The Invention is illustrated by the non-limiting exemplification provided below.

### EXAMPLES

TABLE 1
Enzyme function and names in yeast, vertebrates and plants

| | Enzyme name & E.C number | | |
|---|---|---|---|
| Enzyme function | Yeast | Vertebrates | Plants |
| C-24 or C-28 sterol | ERG6 | | SMT1 (2.1.1.41), |
| methyl transferase | (2.1.1.41) | | SMT2 (2.1.1.143) |
| Delta-7 sterol | | DHCR7 | DWF5 (1.3.1.21) |
| reductase | | (1.3.1.21) | |
| Delta-24(25) sterol | | DHCR24 | Solanum tuberosum* |
| reductase | | (1.3.1.72) | SSR2 |
| | | | Solanum lycopersicum |
| | | | SSR2 |
| Delta-24(28) sterol | ERG4 | | DWF1 (1.3.1.72) |
| reductase | (1.3.1.71) | | |
| C-22 sterol | ERG5 | | CYP710A1 |
| desaturase | (1.14.19.41) | | (1.14.19.41) |

*Note:
Solanum tuberosum and Solanum lycopersicum are the only plant species known to have two delta-24 sterol reductase variants. In each case, one variant (‘SSR1’) is a delta-24(28) sterol reductase. This is like all other plant delta-24 reductases (plant DWF1 enzymes). The other variant (‘SSR2’) is a delta-24(25) sterol reductase and hence can be used as an alternative to a vertebrate delta-24 reductase (DHCR24).

TABLE 2
Combinations of genetic modifications for production of specific sterols:

| | Product | Gene deletion(s) | Heterologous gene(s) |
|---|---|---|---|
| 1 | Campesterol | Δerg5 | Delta-7 sterol reductase |
| 2 | 24- | Δerg4Δerg5 | Delta-7 sterol reductase |
| | Methylenecholesterol | | |
| 3 | β-Sitosterol | Δerg5 | Delta-7 sterol reductase |
| | | | C-28 sterol methyl transferase |
| 4 | Isofucosterol | Δerg4Δerg5 | Delta-7 sterol reductase |
| | | | C-28 sterol methyl transferase |
| 5 | Stigmasterol | | Delta-7 sterol reductase |
| | | | C-28 sterol methyl transferase |
| 6 | Cholesterol | Δerg5Δerg6 | Delta-7 sterol reductase |
| | | | Delta-24(25) sterol reductase |
| 7 | Desmosterol | Δerg6 | Delta-7 sterol reductase |
| 8 | Mixed sterol | Δerg5, attenuated or | Delta-7 sterol reductase |
| | composition wherein | reduced activity of | Delta-24(28) sterol reductase |
| | 24- | ERG4 | Optionally delta-24(25) sterol |
| | methylenecholesterol | | reductase |
| | is present, preferably | | and optionally |
| | as the major sterol, | | C-28 sterol methyltransferase |
| | and minor sterols | | |
| | may include one or | | |
| | more of campesterol, | | |
| | cholesterol, | | |
| | isofucosterol, | | |
| | desmosterol, beta- | | |
| | sitosterol and | | |
| | stigmasterol | | |

TABLE 3
Reference sequences of enzymes:

| | | GenBank | GenBank |
|---|---|---|---|
| | | Ascension No. | Ascension No. |
| Enzyme | Species | (amino acid) | (mRNA) |
| ERG4 | Yarrowia lipolytica | XP_503021.1 | XM_503021.1 |
| ERG5 | Yarrowia lipolytica | XP_500188.1 | XM_500188.1 |
| ERG6 | Yarrowia lipolytica | XP_505173.1 | XM_505173.1 |
| ERG1 | Yarrowia lipolytica | XP_503994.1 | XM_503994.1 |
| ERG3 | Yarrowia lipolytica | XP_503090.1 | XM_503090.1 |
| ERG7 | Yarrowia lipolytica | XP_504990.1 | XM_504990.1 |
| ERG8 | Yarrowia lipolytica | XP_503619.1 | XM_503619.1 |
| ERG9 | Yarrowia lipolytica | XP_499929.1 | XM_499929.1 |
| ERG10 | Yarrowia lipolytica | XP_500646.1 | XM_500646.1 |
| ERG11 | Yarrowial lipolytica | XP_500518.1 | XM_500518.1 |
| ERG12 | Yarrowia_lipolytica | XP_500956.1 | XM_500956.1 |
| ERG13 | Yarrowia_lipolytica | XP_506052.1 | XM_506052.1 |
| ERG19 | Yarrowia_lipolytica | XP_505041.1 | XM_505041.1 |
| ERG20 | Yarrowia lipolytica | XP_503599.1 | XM_503599.1 |
| ERG25 | Yarrowia lipolytica | XP_505281.1 | XM_505281.1 |
| ERG26 | Yarrowia_{—}lipolytica | XP_502124.1 | XM_502124.1 |
| ERG27 | Yarrowia lipolytica | XP_501024.1 | XM_501024.1 |
| PAH1 | Yarrowia lipolytica | BAO18767.1 | AB795935.1 |
| BTS1 | Yarrowia lipolytica | XP_502923.1 | XM_502923.1 |
| GDH1 | Yarrowia lipolytica | XP_505553.1 | XM_505553.1 |
| ARE1 | Yarrowia lipolytica | XP_505086.1 | XM_505086.1 |
| SAY1 | Saccharomyces cerevisiae | NP_011779.3 | NM_001181392.3 |
| SDS23 | Yarrowia lipolytica | XP_504058.1 | XM_504058.1 |
| INS1 | Yarrowia lipolytica | XP_500057.1 | XM_500057.1 |
| KU70 | Yarrowia_lipolytica | XP_501610.1 | XM_501610.1 |
| KU80 | Yarrowia lipolytica | XP_503443.1 | XM_503443.1 |
| HMG1 | Yarrowia lipolytica | XP_503558.1 | XM_503558.1 |
| tHMG1 | Yarrowia_lipolytica | XP_503558.1 | XM_503558.1 |
| ECM22 | Yarrowia lipolytica | XP_500947.1 | XM_500947.1 |
| UPC2 | Yarrowia lipolytica | AOW01027.1 | |
| IDI | Yarrowia lipolytica | XP_504974.1 | XM_504974.1 |
| ACL1 | Yarrowia_lipolylica | XP_504787.1 | XM_504787.1 |
| ACL2 | Yarrowia_lipolytica | XP_503231.1 | XM_503231.1 |
| POT1 | Yarrowia_lipolytica | XP_504109.1 | XM_504109.1 |
| PAT1 | Yarrowia_lipolytica | XP_503608.1 | XM_503808.1 |
| PEX10 | Yarrowia lipolytica | XP_501311.1 | XM_501311.1 |
| GDH2 | Yarrowia_lipolytica | XP_503741.1 | XM_503741.1 |
| G6PD1 | Arabadopsis_thaliana | NP_198428.1 | NM_122970.6 |
| ARE2 | Saccharomyces cerevisiae | NP_014416.1 | NM_001183196.1 |
| ATF2 | Saccharomyces cerevisiae | NP_011779.3 | NM_001181392.3 |
| ACS1 | Salmonella enterica | WP_000083882.1 | |
| SSD1 | Yarrowia lipolytica | XP_505385.1 | XM_505385.1 |
| YBP1 | Saccharomyces cerevisiae | NP_009775.3 | NM_001178564.3 |
| SRE1 | Schizosaccharomyces pombe | NP_595694.1 | NM_001021591.2 |
| CSS1 | Saccharomyces cerevisiae | NP_012097.1 | NM_001179517.1 |
| STC | Tetrahymena thermophilia | XP_001026698.2 | XM_001026696.2 |
| DHCR7 (EC | Solanum tuberosum | BAQ55276.1 | AB839751 |
| 1.3.1.21) | Danio rerio | NP_958487.2 | NM_201330.2 |
| | Legionella drancourtii | ACO48440.1 | FJ197317.1 |
| | Ectocarpus siliculosus | CBN77313.1 | |
| | Candidatus Protochlamydia | KIC71363.1 | |
| | amoebophila | | |
| | Coccomyxa subellipsoidea | XP_005650343.1 | XM_005650286.1 |
| | Mortierella verticillata | KFH65691.1 | |
| | Glycine soja | XP_028244742.1 | XM_028388941.1 |
| | Tetraselmis sp. GSL018 | JAC78771.1 | GBEZ01006640.1 |
| | Waddlia_chondrophila | ADI39181.1 | |
| DHCR24 | Danio_rerio | AAH86711.1 | BC086711.1 |
| (EC 1.3.1.72) | Bombyx_mori | XP_004926865.1 | XM_004926808.2 |
| | Penaeus vannamei | XP_027224655.1 | XM_027368854.1 |
| | Aedes aegypti | XP_001655874.2 | XM_001655824.2 |
| | Gallus gallus | NP_001026459.1 | NM_001031288.1 |
| | Mus musculus | NP_444502.2 | NM_053272.2 |
| | Xenopus tropicalis | NP_001016800.1 | NM_001016800.2 |
| | Solanum lycopersicum | BAQ55273.1 | AB83957 |
| | Notechis scutatus | XP_026521714.1 | XM_026665929.1 |
| | Amblyraja radiata | XP_032884658.1 | XM_033028767.1 |
| SMT2 | Cucurbita pepo | XP_023535889.1 | XM_023680121 |
| 2.1.1.143 | Eutrema salsugineum SMT3 | XP_006390239.1 | XM_006390177.2 |
| | Arabadopsis thaliana SMT2 | NP_173458.1 | NM_101884.4 |
| | Morus notabilis | XP_010100118.1 | XM_010101816.2 |
| | Amborella trichopoda | XP_006828830.1 | XM_006828767.3 |
| | Creolimax fragrantissima | | CFRG4515T1 |
| | Ulva mutabilis | | UM052_0056.1 |
| | Rhodamnia argentea | XP_030527248.1 | XM_030671388.1 |
| | Chenopodium quinoa SMT3 | XP_021737090.1 | XM_021881398.1 |
| | Glycine_soja | XP_028234656.1 | XM_028378855.1 |
| DWF1 (EC | Solanum_tuberosum | BAQ55274.1 | AB839749.1 |
| 1.3.1.72) | Arabidopsis thaliana | NP_850616.1 | NM_180285.4 |
| | Arachis duranensis | XP_015952627.2 | XM_016097141.2 |
| | Selaginella moellendorffii | XP_002960921.1 | XM_002960875.2 |
| | Capsicum chinense | PHU28681.1 | MCIT02000001.1 |
| | Artemisia annua | PWA66182.1 | PKPP01004085.1 |
| | Helianthus annuus | XP_022012299.1 | XM_022156607.2 |
| | Cocos nucifera | EHA8587492.1 | VOII01002059.1 |
| | Triticum urartu | EMS57493.1 | KD143832.1 |
| | Gracilariopsis chorda | PXF44537.1 | NBIV01000088.1 |
| | Capsella rubella | XP_006297345.1 | XM_006297283.2 |
| | Ajuga reptans var. atropurpurea | BAS68578.1 | LC070675.1 |

TABLE 4a
Codon optimised heterologous nucleic acid sequences

| | Nucleic acid sequence, codon optimised for Yarrowia |
|---|---|
| Gene | lipolytica |
| Solanum tuberosum delta-7 | ATGGCCGAGTCTCAGCTGGTGCACCCTCCTCTGTTCACCTAC |
| sterol reductase | ATCTCTATGCTGGCCCTGCTGACCCTGGTGCCTCCTTTCGTG |
| | ATCCTGATGTGGTACACCAACGTGCACGCCGACGGCTCTGT |
| | GCTGCAGACCTTCAACTACCTGAAGGAAAACGGCCTGCAGG |
| | GCCTGATCGACATCTGGCCCCGACCTACCGCCATTGCCGGA |
| | AAGATCATCATCTGCTACGCCCTGTTCGAGGCTACCCTGCAG |
| | CTGCTGCTGCCCGGCAAGCGAGTGCAGGGCCCCATCTCTCC |
| | CACCGGCCACCGACCTGTGTACAAGGCCAACGGCATGGCCG |
| | CCTACACCGTGACTCTGATTACCTACCTGTCTCTGTGGTGGT |
| | TCGGCATCTTCAACCCCACCGTGGTGTACGACCACCTGGGG |
| | GAGATCCTGTCTACCCTGAACTTCGGCTCTCTGATCTTCTGC |
| | CTGTTCCTGTACATCAAGGGACACGTGGCTCCCTCTTCTACC |
| | GACCACGGCTCCTCTGGCAACATCATCGTGGACTACTACTGG |
| | GGCATGGAACTGTACCCTCGAATCGGCAAGCACTTCGACATC |
| | AAGGTGTTCACCAACTGTCGATTCGGCATGGTGTCTTGGGGA |
| | CTGCTGCCCATCACCTACTGCATCAAGCAGTACGAGGAATAC |
| | GGATCTCTGTCTGACTCCATGCTGATCCACGCCATCATCACC |
| | CTGGTCTACGTGACCAAGTTCTTCTGGTGGGAGGCCGGCTA |
| | CTGGAACACCATGGACATTGCCCACGACCGAGCCGGCTTCT |
| | ACATCTGCTGGGGCTGCCTGGTGTTCCTGCCTTGCATGTACA |
| | CTTCTCCCGGCATGTACCTGGTGAAGCACCCCGTGAACCTG |
| | GGACCTCAGCTGGCCATCTCCATCCTGGTGGCCGGCATCCT |
| | GTGCGTGTACATTAACTACGACTGCGACCGACAGCGACAAGA |
| | GTTCCGACGAACTAACGGCAAGGCCCTGGTGTGGGGCAAGG |
| | CTCCCTCCAAGATCGTGGCCTCTTACACCACCACCACTGGGG |
| | AGACTAAGTCCTCTCTGCTGCTGACCTCCGGCTGGTGGGGC |
| | CTGTCTCGACACTTCCACTACGTGCCCGAGATTCTGGCCTCT |
| | TTCTTTTGGTCTGTGCCCGCTCTGTTCAACCACATTATGCCCT |
| | ACTTCTACGTGATCTACCTGACCGGCCTGCTGCTGGACCGAG |
| | CCAAGCGAGATGACGAACGATGCAAGTCTAAGTACGGCAAGT |
| | ACTGGAAGAAGTACTGCGAGAAGGTCCCCTACCGAGTGATC |
| | CCCGGCATCTACTAA |
| Danio rerio | ATGATGGCCTCTGACCGAGTTCGAAAGCGACACAAGGGCTCT |
| delta-7 sterol reductase | GCTAACGGTGCTCAGACCGTTGAGAAGGAACCCTCCAAGGA |
| | GCCCGCCCAGTGGGGCCGAGCCTGGGAGGTCGATTGGTTCT |
| | CCCTGTCCGGCGTGATTCTGCTGCTGTGCTTTGCCCCTTTCC |
| | TGGTCTTCTTCTTCATCATGGCTTGTGATCAGTACCAGTGCTC |
| | TATCTCCCATCCCCTTCTGGACCTTTACAACGGTGACGCCAC |
| | TCTGTTCACCATCTGGAACCGAGCCCCCTCCTTCACCTGGGC |
| | CGCTGCCAAGATCTACGCCATCTGGGTCACCTTCCAGGTCGT |
| | TCTGTACATGTGCGTCCCCGACTTCCTGCACAAGATCCTGCC |
| | AGGTTACGTCGGCGGTGTCCAGGACGGAGCTAGAACTCCCG |
| | CCGGCCTGATCAACAAGTATGAGGTTAACGGTCTGCAGTGCT |
| | GGCTCATCACCCACGTGCTCTGGGTGCTGAATGCTCAGCACT |
| | TCCACTGGTTTTCACCCACCATTATCATTGACAACTGGATCCC |
| | CCTGCTGTGGTGCACCAACATTCTGGGCTATGCCGTCTCCAC |
| | CTTCGCTTTCATCAAGGCCTACCTGTTCCCCACCAATCCCGA |
| | GGACTGCAAGTTCACCGGAAACATGTTTTACAATTACATGATG |
| | GGTATTGAGTTCAACCCCCGAATCGGTAAGTGGTTCGACTTC |
| | AAGCTGTTCTTCAACGGTCGGCCTGGCATCGTCGCCTGGAC |
| | CCTCATCAACCTTTCCTACGCTGCTAAGCAGCAGGAGCTGTA |
| | CGGCTACGTCACCAACTCTATGATCCTGGTCAACGTCCTGCA |
| | GGCCGTGTACGTTGTCGACTTCTTCTGGAACGAGGCTTGGTA |
| | CCTGAAAACCATCGACATCTGCCACGACCACTTTGGCTGGTA |
| | CCTGGGATGGGGAGACTGCGTTTGGCTGCCTTTCCTGTACAC |
| | CCTGCAGGGTCTGTACCTGGTCTACAACCCTATCCAGCTGTC |
| | CACTCCCCACGCTGCCGGCGTGCTGATCCTGGGTCTGGTCG |
| | GTTACTACATTTTTCGAGTGACCAACCACCAGAAGGACCTCTT |
| | CCGACGAACTGAGGGCAACTGTTCGATCTGGGGCAAGAAGC |
| | CGACCTTTATCGAGTGCTCCTACCGATCTGCCGACGGCGCCA |
| | TCCACAAGTCCAAGCTTATGACCTCCGGCTTTTGGGGTGTTG |
| | CCCGACACATGAACTACACTGGTGACCTTATGGGTTCCCTGG |
| | CCTACTGTCTGGCCTGTGGTGGTAACCACCTCCTCCCCTACT |
| | TCTACATTGTCTACATGACTATTCTGCTGGTCCACAGATGCAT |
| | TCGAGACGAGCACCGATGCTCCAACAAGTACGGCAAGGATT |
| | GGGAACGATACACCGCCGCCGTCTCTTACCGACTGCTGCCC |
| | AACATCTTCTAA |
| Legionella drancourtii | ATGTACTTCAAGATCCGAAACACCCTGGGACCTCTGCTGCTG |
| delta-7 sterol reductase | ATCCTGTCTTGCCCCATCTTCGTGATGCTGATGTGGTACACC |
| | AACACCGAGCTGAAGGGATCTCTGTCTACCCTGTGGGACCTG |
| | ATCGTGCAGCAGGGCCTGTTCGAGACTACCTACAAGATCTGG |
| | CAGCCCTACTTCTGGGGCTCTGCCCTGGCCTGGAAGGTGAT |
| | CTTCGCCTTCATCATCTTCGAGCTGGCCCTGATGCGACTGCT |
| | GCCCGGCAAGGAATTCACCGGACCTGTGACTCCCAAGGGCA |
| | ACGTGCCCATCTACAAGGAAAACGGACCCCTGGCCTTCATTA |
| | CCACCATGACTACCTTTTGCGTGGCCTCTTTCGGCCTGCATC |
| | TGTTCCCCGCCTCTATCCTGTACGACAACCTGGGCGCCATCC |
| | TGGGAGCCCTGAACGTGTTCTCTCTGATCTTCTGCGCCCTGC |
| | TGTATATCAAGGGCCGATACTTCCCCTCTTCTACCGACTCTG |
| | GCATCACCAACAACATCATTTTCGACTACTACTGGGGAACCG |
| | AGCTGTACCCTCACATCTTCGGATGGTCTATCAAGAAGTTCAT |
| | CACCTGTCGATTCGGCATGATGTCTTGGGGACTGTTCCTGAT |
| | CTCTTACTGCGCCAAGCAGGCCGAGCTGGGCGACCTGGCCA |
| | ACTCTATGCTGATCTCTGTGGCCCTGCAGTTCCTGTACCTGT |
| | CTAAGTTTTACCTGTGGGAGAAGGGCTACCTGCGATCTCTGG |
| | ACATTATGCACGACCGAGCCGGCTTCTACATCTGCTGGGGCT |
| | GCCTGGTGTGGGTGCCCTGCATCTACACTTCTCCCTCTATGT |
| | ACCTGGTGCTGCACCCCATCCACCTGTCTTTCTGGCTGGCCA |
| | CCTCCATTCTGGTGCTGGGAGCCGCTTCCATCCTGATCAACT |
| | ACTTCGCCGACCGACAGCGACTGATGACCCGAGCCACCGAC |
| | GGCGAGTGTAAGATCTGGGGAAAGAAGCCCGTCACCGTTTT |
| | CGCCCAGTACCAGACCACCGAGGGCGACAAGAAGCAGACCA |
| | TCCTGCTGGCCTCTGGCTGGTGGGGCGTCGCCCGACACTTC |
| | CACTACGTGCCCGAGCTGGCTGGAACCTTCTTCTGGTCTGTG |
| | CCCGCTCTGTTTGAGAACTTCTCTCCTTACTTCTACCTGTGCT |
| | TCCTGACCATTCTGCTCGTGGACCGAGCCTTCCGAGATGACC |
| | GACGATGCTCTGACAAGTACGGACAGTACTGGCACAAGTACT |
| | GCGAGCTGGTGCCTTACAAGATCGTGCCCTTCGTGATCTAA |
| Ectocarpus siliculosus | ATGATCGACGGCGCTGCCATCGGACGATCTCCCGTGATCTCT |
| delta-7 sterol reductase | TCTTGGCACGGCTACAACCCCGCCTCTTCGCAGCAGCAGTGT |
| | CTGCAGGCCGTCCAGACCTCTCTGCCCACCACCGACGGCGA |
| | CCGAGAGCGACGACGATCTATGGCCCTGCGAACCTCTACCA |
| | AGCAGGCCTCTGACGCCATGGACATCCGAGAGGCCGTGAAG |
| | GCCTCTGTGGCCGCCGAGTCTAAGGACTCTAAGGTGTGGGG |
| | CTTCGTGCCCAACTGGTTCCGAACTACCGTGGGACCCGTGTT |
| | CCTGATCCTGGTGCCTCCTTTCTTCGTGGTGTTCTGGTGGCA |
| | CCTCCTGGTGTCTCACTCTGGCTCTTGGGTGTCTCTGTGGGG |
| | CGACCTGAAGGCCGCTGGACCCGAGTACGTGCTGGACGTGG |
| | TGCCCTCTCCTGTGGACCCCGCTGCCTGGAAGTACATCCTCG |
| | GCTTCGGCGTGTTCGAGATCCTGCTGATGGTGGGACTGCCC |
| | GGCAAGGCCTTCCGAGCTAACCCCACCGCCACCGGACACAT |
| | CCCCGTGTAGAAGGCCAACGGCATGCTGTCTTACCTGGTGAG |
| | CCTGGCTACCCTGTGCGCCCTGGTCGCCACCGACCGACTGG |
| | ACCCCAAGAACGTGTACGACAAGCTGGGCGAGATCTTCACC |
| | GGCCTGTCTGTGTTCTCTCTGTTCTTCGTGCTGCTGCTGACC |
| | GTGAAGGGCCTGTACTTCCCCTCTACCGACGACTCTGGATCT |
| | AACGGATCTTTCCTGCAGAACTACTGGTGGGGCACCGAGCT |
| | GTACCCTCGAGTGTTCGGAGCCGACGTGAAGATGTTCACCAA |
| | CTGCCGATTCGGCATGATGTACTGGGCCGTGGGCGCCGTGA |
| | TCTACGCTTACACCCAGCAGCAGATGTACGGAAAGCTGTCCT |
| | CTTCTATGGCCGTGTCTGTGATCCTGCAGCTGACCTACATCA |
| | CCAAGTTCTTCCACTGGGAGATGGGCTACATGAACTCTATGG |
| | ACATTCAGCACGACCGAGCCGGCTACTACCTGTGCTGGGGC |
| | TGCCTGGTGTGGGTGCCCGCCGTGTACTCTTCTCCCGGCAT |
| | CTACCTGGTCAAGCACCCCATTTCTCTCGGCTGGTACGGCGC |
| | CTCTGCCATTCTGGCCCTGGGCCTGCTGTCTATCTGGGCCAA |
| | CTTCGACGCTGACCGACAGCGACACGCCTTCCGACAGGCCA |
| | AGGGCGACATCATCGTGTGGGGCAAGCCCGCCAAGTACATC |
| | ACCGCCGGCTACATCAACGCCCGAGGCGAGAAGGCCTCCTC |
| | TCTGCTGCTCTGCACCGGCTGGTGGGGAGTCGCCCGACACT |
| | TCCACTACCTGCCTGAGATTACCGGCGCCTTCTTCTGGACCG |
| | TGCCTGCTCTGTTCGAGACTCCCACTCCTTACTTCTACGTCGT |
| | GTTCCTGGTGCTGCTCCTCACTGACCGAGCTTTCCGAGATGA |
| | CACCCGATGCCGAGGCAAGTACGGCAAGCACTGGGACAAGT |
| | ACTGCGCTCAGGTGCCCTACAAGATCGTGCCCGGCATCCTG |
| | TAA |
| Candidatus Protochlamydia | ATGCTGATCGAGATGCTGTGCATCACCAAGCACATCCTCGAG |
| amoebophila | AAGATCTCTAAGTTCTCTCTGGCTCGACCCCACGTGGCCAAC |
| delta-7 sterol reductase | ACCAACATTTTCTTCAGACAGACCTTCGGACCCCTGTTCCTG |
| | CTGCTGCTGTGCCCTCCTACCGTGTTGGCCTTCTGGTACACC |
| | AACACCTACCTCGAGGGATCTCTGTTCCGATTCACCGAGTTC |
| | GCCTGGCAGCAGGGCTTCCTGTCTACCCTCAAGACTATCTGG |
| | TTCCCCTACTTCTTCGGCACCTCTATCGCCTGGACCATGCTG |
| | GCCATCTTCGCTTCTCTGCAGCTGATCCTGATGCGAATTCTG |
| | CCCGGCGAGTGCTACGAGGGCCCCATCACTCCCACCGGACA |
| | CGTGCCCCTGTACAAGGCCAACCGATTCTCTGCCTTCATCGT |
| | GACCGTGTCTATCTTCCTGATCGCCTCTTGCTACTACCAGCT |
| | GTTCGCTCCCACCATCATCTACGACAACTTCCCCGGCCTGCT |
| | GGGCGCCCTGAACATCTTCTCCCTGTGTTTCTGCTTCTTCCT |
| | GGTGCTGAAGGGCCACTACTTCCCTTCTAACGGCGACGTCG |
| | GCGGCTCTGGCAACATCATCTTCGACTACTACTGGGGCATGG |
| | AACTGTACCCTCGACTGCTCGGCTGGGACATCAAGCAGTTCA |
| | CCAACTGCCGATTCGGCATGATGTCTTGGGCCCTGATCGTGA |
| | TCTCTTTCGCCGCCAAGCAGCAACAGCTGGACGGCCTGTCT |
| | GACTCTATGTTCGTGGCCGTGGCTCTCCAGCTCATCTACATT |
| | ACCAAGTTCTTCATCTGGGAGCCCGGCTACCTGCGATCTCTG |
| | GACATTATGCACGACCGAGCCGGCTACTACATCTGCTGGGG |
| | CTGCCTGGTGTGGGTGCCCGGCATCTACACTTCTCCCACTCT |
| | GTACCTGGTGGATCACCCCAACCACCTGGGACTCGCCGTGT |
| | CCTCTCTGCTGTTCGTGGTGGGCGTGATCGGCATCCTGGTG |
| | AACTACCTGGCCGACCGACAGCGACAGCTCGTGCGAAAGAA |
| | CCAGGGCAACTGTCGAATCTGGGGAAAGGAACCCATCCTGA |
| | CCATTGCCAAGTACACCACTCAGACCGGCGAGACTAAGCAGA |
| | ACCTGCTGCTCGCCTCTGGCTGGTGGGGCCTGTCTCGACAC |
| | TTCCACTACCTGCCTGAGCTGCTGGGAGCCTTCTGCTGGTCT |
| | GCTCCCGCTCTGTTCGAGAACTTCCTGCCTTACTTCTACTTCG |
| | TGTTTCTGACCCTGCTCCTGACCGACCGAGCTTTCCGAGATG |
| | ACCAGAGATGCTCTAAGAAGTACGGCGAGGACTGGAAGATCT |
| | ACTGCCAGCGAGTGCCCTACAAGATCATCCCCTTCGTGATCT |
| | AA |
| Coccomyxa subellipsoidea | ATGGTCACAACCCGAGCCGCTGCTCGAGCTCAGACCCCTCT |
| delta-7 sterol reductase | GGGCAAAGCTCCTCCCTCCGACCTGTCCCACTCCGAACCCT |
| | CCGTCTCTACTCAGAACGGCAAGAAGACATGGGCCGAGACC |
| | GCTGGGGCAGGCGAGCACATCGGCGCCTGGGGTATCGGCG |
| | GCACTGCCGGACACGTCTTGGCCTACCTGGGTACTCTTGCC |
| | CTTATGATCGGTTGCCCCGCCTTTGCAATCTACATGTGGTTTA |
| | CCCTCACCCACTTGGATGGCTCTCTTGTGGAGCTCGTCCAGT |
| | TCGCCCAGAAGGCTGGATTTCAGGGCGTCCGAGCCTCATGG |
| | CCCTGGCCCTCCCAGGAGGCCTGGGCCATCATCGCCTCCTT |
| | CGGCGGTCTTCAGGCCTTCCTGCAGCTTGCCTTGCCCGGTG |
| | CTGTGCACAAGGGCCCAGTCTCTCCCAAGGGTAÅCGTCCCC |
| | GTGTACAAGGCCAACGGTGTCCTCGCCTACTTTACCACCCTG |
| | GCTCTGTTCGTCCTTGGCTGGCAGTTCAAGCTGTTTTCCCCC |
| | GCCAGAGTCTACGACCTGTTCGGCGAGATCCTTTCCGGCCT |
| | GAACATGTTCTCTCTGCTGTTCTGCCTGTTCCTGTACTTCAAG |
| | GGCAAGTACGCCCCCTCATCCTCGGATTCTGGTTCTACCGGC |
| | TCCCTGATGTACGATTATTACTGGGGGATGGAGCTGTACCCT |
| | CGGATTGGACGACACTTTGACCTGAAGACTTGGACTAACTGC |
| | CGAATGGGCATGATGGGCTGGGGAGTTCTGGTCCTGTGTTA |
| | CGCTGTGAAGCAGCACGAGCTGTACGGTTACCTTTCTAATTC |
| | GATGGCTGTTTCTATCCTGCTCATGCATTTATACATTTTCAAG |
| | TTCTTCCTCTGGGAGACTGGTTACTGGGGTACCATGGACATC |
| | GCTCACGACCGCGCTGGATACTACCTCTGCTGGGGATGCCT |
| | GAACTGGGTCCCCGCTATCTACACCTCTCCCGCCCTGTACCT |
| | CGTCGAGAACCCTATTCAGTGGTCTCTGCCCGCCGCCACCG |
| | CTATCGCTGTTGCCGGTACCCTGGCTATCTACATCAACTACG |
| | ACTCCGATCGGCAGCGACAGGTTTTCCGAGCTACCAATGGTA |
| | AGGCCCTGGTGTGGGGCAAGCCTCCCCAAATTATCTCTGCCA |
| | AGTACATCACCGGCGATGGCAAGCAGAAGACCTCCCTGCTC |
| | CTGGCCTCTGGCTGGTGGGGGCTCGCCCGACACTTCCACTA |
| | CCTGCCCGAGATCTTGGCCGCCTTCTTTTGGACCCTCCCCGC |
| | TGGTATCTCCCACGCTCTGCCCTACTTTTACGTCTTCTTCCTG |
| | ACTCTGCTCCTTACCGATCGAGCTTTCCGAGACGACGTCCGA |
| | TGTAGCTCCAAATACGGTGCCTACTGGCAGCAGTACACCAAG |
| | GCCGTTCCCTACAAGATGATCCCTTACATTTTCTAA |
| Mortierella verticillata | ATGGCCGTGCAGCAGCGAAAGACCCCTGCTCAGGTGGACGT |
| delta-7 sterol reductase | GAAGGCCGAGTCTAAGCTGGACGCCCAGGTGGGCAAGACCT |
| | GGGGCCGAGATCGAGATGTGTCTTTCGGCACCATCCTGATCT |
| | CCCTGGGCATCCTGGTGATGTCTCCCATCTGGGTGATGTACA |
| | CCTACATCTCTTGCAACGCCTACCAGTGCGCCATGTCTGCTC |
| | CCGCTCTCGAGATCTACAACTCTCCCGACACTCTGGCCGCCA |
| | TTCAGACCCTGCTGCTGCGAGAGGTGCCCCGATTCTCTCCCT |
| | ACGCCGCTCGACTGTTCTTCACCTGGCTGGCCTTCCAGGCC |
| | GCTCTGTACGCCTTCCTGCCTGCTCAGATCGGCTACGGCCA |
| | GCGAACCCCTGCCGGCCACATTCTGCCCTACAAGGTGAACG |
| | GCCTGCTGGCCTGGTTCATCTCTCACTCTATCTACGCTGCCG |
| | GCGGACTGTACTTCGGCTGGTGGAAGCTGTCTATCATCCACG |
| | ACAACTGGGGCGGACTGCTGGTGGCCGCCAACATGTACGGC |
| | TACTTTCTGACATTCTTCTGCTTTATCAAGGCCTACACCTTTC |
| | CTTCTCACCCCGCCGACCGAAAGTTCTCTGGCTCTTTCATCT |
| | ACGACCTGCTGATGGGCATCGAGTTCAACCCTCGAATCGGCA |
| | AGCTGTTCGACTTCAAGCTGTTTCACAACGGACGACCCGGCA |
| | TCGTGGCCTGGACCATGATCAACCTGTCCTTCGCCGCTGCTC |
| | AGTACGAGAAGATCGGATACGTGACCAACTCTATGATCCTGC |
| | TGAACCTGCTGCATGCTACCTACGTGCTGGACTTCTTCTACA |
| | ACGAGGACTGGTATCTGCGAACCATCGACATTGCCCACGACC |
| | ACTTCGGCTTCTACCTGGCCTGGGGCGACTCCGTGTGGCTG |
| | CCCTGGCTGTACACCCTGCAGTCTCACTACCTGGTGCGAAAC |
| | CCCGTGGACCTGACTCCTGTGCAGTTCGCCTTCGTGTTCACC |
| | GTGGGCTACATCGGCTACTTCATCTTCCGATCTGTGAAGCAC |
| | CAGAAGGACATCGTCCGATCTACCAACGGCGAGTGCATGATC |
| | TGGGGCAAGCCCGCCAAGGTGATCCGAACCTCTTTCGTGAC |
| | CTCTGACGGCAAGACCCACAAGTCTCTGCTGCTGTGCTCTGG |
| | CTACTGGGGCCTGTCTCGACACTTCAACTACGTGGGCGACCT |
| | GCTCATCTCTCTGGCCATGTGCATGACCTGCGGCACCCAGCA |
| | TCTGCTGCCCTACTTCTACATCATCTACATGACCATCCTGCTG |
| | CTCCACCGAATCCAGCGAGATCACACCCGATGCAAGGGCAA |
| | GTACGGAAAGTACTGGGACGAGTACATGAAGGCCGTGCCTT |
| | ACAAGCTGATCCCCTACGTGTACTAA |
| Glycine soja | ATGGGCGCTACCGTGCACTCTCCCCTGGTGACCTACGCCTCT |
| delta-7 sterol reductase | GTGATCTCTCTGCTGACCCTGTGTCCTCCTTTCGTGGTGCTG |
| | CTGTGGTACACCATGACTCTGGCCGACGGCTCTGTGTCTGAG |
| | ACTTTCCACTACCTGCGACAGAACGGCCTGCAGGGCCTGCT |
| | GCACATCTGGCCCACTCCTACTOCTACCGCCTGCAAGATCAT |
| | TGCCGTGTACGCCGCCTTCGAGGCCGCTCTGCAGCTGCTGC |
| | TGCCCGGCAAGACCGTGTACGGCCCCATCTCTCCCACCGGC |
| | CACCGACCTGTGTACAAGGCCAACGGACTGCAGGCCTACTT |
| | CGTGACCCTGATCACCTACTTCGCCGTGTGGTGGTTCGGCAT |
| | CTTCAACCCCACCATCGTGTACCACCACCTGGGCGAGATCTA |
| | CTCTGCCCTGATCTTCGGCTCTTTCCTGTTCTGCGTGTTCCTG |
| | TACATCAAGGGCCATCTGGCTCCCTCTTCTACCGACTCTGGA |
| | TCTTCTGGCAACCTGATCATCGACTTCTACTGGGGCATGGAA |
| | CTGTACCCTCGAATCGGCAAGCACTTCGACATGAAGGTGTTC |
| | ACCAACTGTCGATTCGGCATGATGTCTTGGGCCGTGCTGGCC |
| | CTGACCTACTGCATCAAGCAGTACGAAGAGAACGGCAAGGT |
| | GGCCGACTCTATGCTGGTCAACACCGCTCTGATGCTGGTGTA |
| | CGTGACCAAGTTCTTCTGGTGGGAGGCCGGCTACTGGTCTA |
| | CCATGGACATTGCCCACGACCGAGCCGGCTTCTACATCTGCT |
| | GGGGCTGCCTGGTGTGGGTGCCCTCTGTGTACACCTCTCCT |
| | GGCATGTACCTGGTGAACCATCCTGTGAACCTGGGCATCAAG |
| | CTGGCTCTGTCTATCCTGGTGGCCGGCATCCTGTGCATCTAC |
| | ATCAACTACGACTGCGACCGACAGCGACAAGAGTTCCGACG |
| | AACTAACGGAAAGGGCACCGTGTGGGGCAAGGCCCCTTCTA |
| | AGATCGAGGCCACCTACACCACTACCTCTGGCGAGACTAAGC |
| | GATCCCTGCTGCTGACCTCTGGCTGGTGGGGCCTGTCTCGA |
| | CACTTCCACTACGTGCCCGAGATCCTGGCCGCCTTCTTCTGG |
| | ACCGTGCCTGCTCTGTTCGAGCACTTCCTGCCTTACTTCTAC |
| | GTGATCTTCCTGACCATCCTGCTGTTCGACCGAGCTAAGCGA |
| | GATGACGACCGATGCCGATCTAAGTACGGCAAGTACTGGAA |
| | GCTGTACTGCGACAAGGTGCCCTACCGAATCATCCCCGGCAT |
| | CTACTAA |
| Tetraselmis sp. GSL018 | ATGAAGCGAGCCTCCAAGACCCCCGACACCGCCTCTAAGGG |
| delta-7 sterol reductase | TCGAGAACCCCTTTCTGAGCCTCACACCAACGGTGTCGCTAA |
| | GGCCAGCAACAAGACCTCTTGGGCCGAGTCCAATGGCATCG |
| | GTGATCGAGACGGATTCATGGGCCTGTCCGGTGCCGCCGGC |
| | CATGCTGTGGCCCTTCTGGGCACCGTCGTGCTGCTCGTCGG |
| | TTGCCCCGCCTTTGTCTTCGTGCTCTGGTACATTAATTGTCGA |
| | CTCGACGGCTCCGTCTCCGAGTTCGTCGCCCTCGCCGCTCG |
| | AGAGGGCGCCGTGGGTCTCTGGCAACGATGGCCTACTCCCA |
| | CTGCTGAGGCCTGGGCCATCATTGGCACCTTCGGTGCAGTC |
| | GAGGCCTTCCTGCAGCTGGCTCTCCCTGGCAAGAAGTTCCT |
| | GGGTCCCGTCTCTCCTAAGGGTAACGTCCCCGTCTACAAGG |
| | CCAACGGCATGCAAGCCTACGTGACTACCCTGGTCCTGTTCT |
| | TTGCCGTCTGGGGCTCCGGCATCTACAACCCCGCGCGAGTC |
| | TACGATCTCATGGGTGAGATTCTGGCCGCCCTGAACATGTTT |
| | TCTCTGCTGTTTTGCCTGTTCCTCAACATTAAGGGTCATGTCG |
| | CCCCTTCCTCTACTGACTCCGGCTCTACCGGCTCTTTGCTGT |
| | ACGACTACTACTGGGGCATGGAGCTTTACCCTAGAATTGGCC |
| | GATCCTTCGACATTAAGACCTGGACCAACTGCCGAGTCGGCA |
| | TGATGGGTTGGGGCATCCTGATCCTGTGCTACGCCGCCAAG |
| | CAGGTGGAGGAGGCTGGATTTCTGTCCGACTCCATGGCTGT |
| | GTCCGTCATTCTCATGCACGTTTACATCGCCAAGTTCTTCTGG |
| | TGGGAGACTGGTTACTGGAAGACCATGGACATCATGCACGAT |
| | CGAGCCGGTTACTACATCTGCTGGGGTTGCCTCGTGTGGATC |
| | CCCTCCATGTACACCTCTCCCACCATGTTTCTTGTCAAGCATC |
| | CTATGGTGCTGGGCCCCACCCTCACCGGCGCTGTCCTGGCT |
| | GCTGGCCTGCTGTGTATCTACATCAACTACGACGCCGACCGA |
| | CAGCGACAGGTTTTCCGAGAGTCTAACGGCAAGGCCCTGATT |
| | TGGGGTCGAAAGCCTAAGAAGATCGAGGCCCAGTACACTAC |
| | CGCGGACGGCCAAACTAAGACCTCTCTGCTGCTGGTGTCTG |
| | GTTGGTGGGGTGTCTCCCGACATTTCCACTACCTGCCCGAGA |
| | TACTTGCCTCCGTGTTCTGGTCTGTGCCCGCCCAGACTGATT |
| | ACGCTATGGCCTACCTGTACTCTGCCTACCTCACCATTCTTCT |
| | CGTGGACCGTGCCTTCCGAGACGACCTGCGATGCGCTTCCA |
| | AGTACGGCAAGCACTGGGTCGAGTATTGCCGACAGGTCCCC |
| | CACAAGATCGTGCCCTACATCTTCTAA |
| Waddlia chondrophila | ATGGCCGCCACCACCACCAACGTGCAGACCCGAAACTGGGG |
| delta-7 sterol reductase | CCGAGCCTGGGAGACTACCTGGCTGTCTCTGTTCTCTACCAT |
| | TGCTCTGCTGGCCACCGCTCCTATGATGGTGCTGTACTGCTA |
| | CATTGCCTGCGTGCGATTCCGAGGCTCTCTGATCGGACCCG |
| | CCTACGCTCTGGCCTCTGGCGCCGTGTCTCTGGACTCTCTGT |
| | TCCCCTCGTTCGAGGTGGGCATCTTCGCCCTGTACCTCGGCT |
| | GGTTCGCCTTCCAGCTGCTGCTGTACCTGGGACTGCCCGAC |
| | CTGCTGCACCGAATTCTGCCCCGATACCGAGGGGGCCGACA |
| | AGAGGGCGCTGTGACCCCTGCCGGCAAGCAGCTGGTGTACC |
| | AGATCAACGGACTGCAGGCCTGGCTGATCTCCCACCTGTTCT |
| | TCGGCATCGGCGCCTACGTGCTCGGATGGTTCTCTCCCTCG |
| | ATCATTGCCGAGAACTGGGGAGGCTTCCTGATCGTGACCAAC |
| | GTGATGGGCTACCTGACCGCCATCTTCGTGTACGTGAAGGC |
| | CTACCGATTTCCTTCGAACGCCGAGGACCGAAAGTTCTCTGG |
| | CAACCCTCTGTACGACTTCTTCATGGGCATCGAGTTCAACCC |
| | TCGAATCGGCAAGTTCGACTTCAAGCTGTTCTTCAACGGACG |
| | ACCCGGCATCATTGCTTGGACCCTGATCAACTGGTCTTTCGC |
| | CGCCAAGCAGTACGCCGACCTGGGATACCTGCCTAACTCTAT |
| | GCTGCTGGTGAACGTGCTGCAGGCTATCTACGTGCTGGACTT |
| | CTTCTGGCACGAGACTTGGTATCTCAAGACCATCGACATCTG |
| | CCACGACCACTTCGGCTGGATGCTGTCTTGGGGCGACCTGG |
| | TGTGGCTGCCCTACATGTACACCCTCCAGGGCCTGTACCTGC |
| | TCTACCATCCTGTGGACCTGTCTACCGGCTTCGCTCTGTTCG |
| | TGCTGACCCTGGGCGTCGTGGGCTACGCCATTTTCCGATCTG |
| | CCAACCACCAGAAGGACCACTTCCGAGGAGTGCAAGGCAAG |
| | GAACCCATCTGGGGAAAGATGCCCGAGTTCATCTCTTGCCAG |
| | TATACCGCCGCTGACGGCTCTCTGCACCACACCAAGCTGCTC |
| | CTCTCCGGCTGGTGGGGACGAGCCCGACACATGAACTACAC |
| | CGGCGACCTGATGCTGTCCCTGGCCTACTGCCTGGCCTGCG |
| | GCTTCTCTCACCTCCTGCCTTACTTCTACTTCGTCTACATGAC |
| | CATCCTGCTGGTCAACCGATGCTACCGAGATGAGCACCGATG |
| | CGAGAACAAGTACGGCGACGCCTGGCGAAAGTACTGCCGAC |
| | GAGTCCCCTACCGACTGATCCCCGGCATCTACTAA |
| Tetrahymena thermnophilia | ATGAAGAAGATCCTCATCGGTCTCATCATCGGTCTCTTCCTCT |
| squalene-tetrahymanol | TCTCCTCCGTCAACGCCTCCGTCAACCTCACCGAGGTCCAGA |
| cyclase | ACGCCATCTCCATCCAGCAGGGTATCAACTGGGCTGAGGTC |
| | CACAACAACACCTGGTACTACCCTCCCTACCTCGGTGAGATG |
| | TTCATCTCCGAGTACTACTTCGAGCTCCTCGTCCTCAACTGG |
| | ACCCACAAGTCCGCCTTCAACGCCACCTACTTCACCGAGCGA |
| | CTCCTCCAGACCCAGTTCGAGGACGGTTCCTGGGAGCAGGT |
| | CCGAGAGCAGAACCTCGAGACCGGTCAGCTCGACGCCACCG |
| | TCTTCAACTACTGGTACCTCAAGTCCATCAACAACAACCCCAA |
| | GATCGAGGCTGCCCTCCAGAAGGCCCGAAAGTGGATCGTCG |
| | CTCAGGGTGGTATCGAGGCCACCCAGACCATGACCAAGTTC |
| | AAGCTCGCTGCCTTCGGTCAGTACTCCTGGGAGGACCTCTG |
| | GTACGTCCCTCTCTTCATCTTCAAGCAGAACGGTATCTTCAAG |
| | TACACCTACGTCAAGGACATCGTCGCCCAGTGGGTCTACCCC |
| | CACCTCACCGCTCTCGCCTACCTCCGATACCAGCGAACCGTC |
| | TTCAACGTCCCTGTCGCTGACCTCCGAGAGCTCTGGATCAAC |
| | TACCCCAAGAACGGTATCAAGATCTCCCCCCGAGAGTACTCC |
| | ACCCTCAACCCCGACTCCGACCTCCTCATCCTCATGGACGAG |
| | ATCTTCAAGCTCAAGCAGCCTCTCGGTTCCTTCGGTGCCTAC |
| | ACCATCTCCACCCTCCTCACCCTCATGTCCTTCAAGGACTTC |
| | CAGTCCAAGCACCCCCACCTCTACCAGAACGAGATCCAGAAG |
| | GCCTACGAGGACGGTTACTACTTCGTCGAGTTCAACTACTTC |
| | AACTTCCGAGAGGCCTACCACGGTTCCCTCGACGACGGTCG |
| | ATGGTGGGACACCATCCTCATCTCCTGGGCCATGCTCGAGTC |
| | CGGTCAGGACAAGGAGCGAATCTTCCCCATCGTCCAGAACAT |
| | GGTCAAGGAGGGTCTCCAGCCCAAGAAGGGTATCGGTTACG |
| | GTTACGACTTCGAGTACGCTCCCGACACCGACGACACCGGT |
| | CTCCTTCTCGTCGTCATGTCCTACTACAAGGAGGCCTTCCAG |
| | AAGCAGATCCCCGAGACCATCGAGTGGCTCTTCTCCATGCAG |
| | AACGACGACGGTGGTTACCCCGCCTTCGACAAGGGTAAGAA |
| | CGAGGACAACCTCCTCTTCAAGTTCGCCTTCAACATGGCCGG |
| | TATCGCCAACTCCGCCGAGATCTTCGACCCTTCCTGCCCTGA |
| | CATCACCGGTCACATCATGGAGGGTCTCGGTGAGTTCGGTTA |
| | CCAGGCCAACCACCCCCAGATCCAGAACATGATCAAGTACCA |
| | GCGAAAGACCCAGAACAAGTGGGGTTCCTGGCAGGCTCGAT |
| | GGGGTGTCAACTACATCATGGCTGTCGGTGCTGTCGTTCCTG |
| | GTCTCGCTCGAGTCAACTACGACCTCAACGAGCAGTGGGTC |
| | CAGAACTCCATCAACTACCTCCTCAACAAGCAGAACAAGGAT |
| | GGCGGCTTCGGTGAGTGCGTCCTCTCCTACAACGACCCCGA |
| | GAAGTGGAACGGTATCGGTAAGTCCACCGTCACCCAGACCT |
| | CCTGGGGTCTCCTCGCTCTCCTCGAGGTCTACAACCAGAACG |
| | AGCAGATCAAGCACGCTGCCGATCGAGCTGCCCAGTACCTC |
| | CTCGACCAGTTCAAGCGAGACGACAACACCTTCTACGACCAC |
| | TCCACCATCGGTACCGGTCACCGAGGTCTCCTCTACCTCCAG |
| | TACCCCTCCTACGCCCAGTCCTTCCCTCTCGTCGCCCTCAAC |
| | CGATACCAGAAGATCTCCCAGGGTCAGTACCACTTCTCCAAG |
| | AACCTCTACAACGGTAACGGTGAGCCCGTCCAGAAGCAGAA |
| | CATCTAA |
| Salmonella enterica | ATGTCTCAGACCCACAAGCACGCTATCCCCGCCAACATTGCC |
| acetyl-CoA synthetase | GACCGATGCCTGATCAACCCCGAGCAGTACGAGACTAAGTAC |
| | AAGCAGTCTATCAACGACCCCGACACCTTCTGGGGCGAGCA |
| | GGGCAAGATCCTGGACTGGATCACCCCTTACCAGAAGGTCAA |
| | GAACACCTCTTTCGCTCCCGGCAACGTGTCTATCAAGTGGTA |
| | CGAGGACGGCACCCTGAACCTGGCCGCCAACTGCCTGGACC |
| | GACACCTCCAAGAGAACGGCGACCGAACCGCCATCATCTGG |
| | GAGGGCGACGACACCTCTCAGTCTAAGCACATCTCTTACCGA |
| | GAGCTGCACCGAGATGTGTGCCGATTCGCTAACACCCTGCT |
| | GGACCTGGGCATCAAGAAGGGCGACGTCGTCGCTATCTACA |
| | TGCCCATGGTGCCCGAGGCCGCCGTGGCCATGCTGGCCTGC |
| | GCTCGAATCGGCGCCGTGCACTCTGTGATCTTCGGCGGCTT |
| | CTCGCCCGAGGCTGTGGCCGGACGAATCATCGACTCCTCTT |
| | CTCGACTGGTGATTACCGCCGACGAGGGCGTGCGAGCCGGC |
| | CGATCTATTCCCCTGAAGAAGAACGTCGACGACGCTCTGAAG |
| | AACCCCAACGTGACCTCTGTCGAGCACGTGATCGTGCTGAAG |
| | CGAACCGGCTCTGACATCGACTGGCAAGAGGGCCGAGATCT |
| | GTGGTGGCGAGATCTGATCGAGAAGGCTTCTCCCGAGCACC |
| | AGCCTGAGGCCATGAACGCTGAGGACCCTCTGTTCATCCTGT |
| | ACACCTCTGGCTCTACCGGCAAGCCCAAGGGCGTGCTGCAC |
| | ACCACCGGCGGCTACCTGGTGTACGCCGCCACCACCTTCAA |
| | GTACGTGTTCGACTACCATCCTGGCGACATCTACTGGTGCAC |
| | CGCTGACGTCGGCTGGGTGÅCCGGCCACTCTTACCTGCTGT |
| | ACGGACCCCTGGCCTGTGGCGCTACCACTCTGATGTTCGAG |
| | GGCGTCCCCAACTGGCCCACTCCTGCTCGAATGTGCCAGGT |
| | GGTGGACAAGCACCAGGTGAAGATTCTGTACACCGCTCCTAC |
| | CGCCATTCGAGCCCTGATGGCCGAGGGCGACAAGGCCATCG |
| | AGGGCACCGACCGATCTTCTCTGCGAATCCTGGGCTCTGTG |
| | GGCGAGCCTATTAACCCCGAGGCCTGGGAGTGGTACTGGAA |
| | GAAGATTGGCAAGGAAAAGTGCCCCGTCGTTGACACCTGGT |
| | GGCAGACCGAGACTGGCGGCTTCATGATTACCCCTCTGCCT |
| | GGCGCCATCGAGCTGAAGGCCGGCTCTGCTACCCGACCATT |
| | CTTCGGCGTGCAGCCCGCTCTGGTCGACAACGAGGGACACC |
| | CTCAAGAGGGCGCCACCGAGGGCAACCTGGTCATCACCGAC |
| | TCTTGGCCCGGACAGGCTCGAACCCTGTTGGGGGACCACGA |
| | GCGATTTGAGCAGACCTACTTCTCTACCTTCAAGAACATGTAC |
| | TTCTCTGGCGACGGCGCTCGACGAGATGAGGACGGCTACTA |
| | CTGGATTACCGGCCGAGTGGACGACGTGCTGAACGTGTCTG |
| | GCCACCGACTGGGCACCGCCGAGATCGAGTCTGCCCTGGTG |
| | GCTCACCCCAAGATCGCCGAGGCTGCCGTCGTGGGCATTCC |
| | CCACGCCATCAAGGGCCAAGCCATCTACGCCTACGTGACCC |
| | TCAACCACGGCGAGGAACCCTCGCCTGAGCTGTACGCCGAG |
| | GTGCGAAACTGGGTGCGAAAGGAAATCGGTCCCCTGGCTAC |
| | CCCTGACGTCCTGCATTGGACCGACTCGCTGCCCAAGACAC |
| | GATCTGGAAAGATCATGCGACGAATCCTGCGAAAGATCGCTG |
| | CCGGTGACACCTCTAACCTGGGCGACACTTCTACCCTGGCTG |
| | ACCCTGGCGTGGTCGAGAAGCCCCTGGAAGAGAAGCAGGCC |
| | ATTGCTATGCCCTCTTAA |
| Solanum lycopersicum | ATGTCTGACGCCAAGGCTCCCGTGGCCACTGCTTACCCCAA |
| delta-24 sterol reductase | GCGAAAGATCCAGCTGGTGGACTTCCTGCTGTCTTTCCGATG |
| | GATCATCGTGATTTTCTTCGTGCTGCCCTTCTCTTTCCTGTAC |
| | TACTTCTCTATCTACCTGGGCGACGTGAAGTCTGAGCGAAAG |
| | TCTTACAAGGAGCGACAGATGGAACACGACGAGAACGTGAA |
| | GGAAGTGGTGAAGCGACTGGGCCAGCGAAACGCCGAGAAG |
| | GACGGCCTGGTGTGCACCGCTCGACCTCCATGGGTCGTCGT |
| | GGGCATGCGAAACGTGGACTACAAGCGAGCCCGACACTTCG |
| | AGGTGGACCTGTCTAAGTTCCGAAACATCCTGGACATCGACA |
| | CCGAGCGAATGGTGGCCAAGGTCGAGCCCCTGGTGAACATG |
| | GGCCAGATGTCTCGAGTGACCATTCCTATGAACCTGTCTCTG |
| | GCCGTGCTGGCCGAGCTGGACGACCTGACCGTCGGCGGCC |
| | TGATCAACGGCTTCGGCGTCGAGGGATCTTCTCACATCTTCG |
| | GCCTGTTCTCTGACACCGTGGTGGCCCTCGAGGTGGTGCTG |
| | GCTGACGGCAAGGTGGTGCGAGCCACCAAGGACAACGAGTA |
| | CTCTGACCTGTTCTACGCTATCCCCTGGTCGCAGGGCACCCT |
| | GGGCCTGCTGGTGTCTGCCGAGATCAAGCTGATCCCCGTGG |
| | ACCAGTACGTGAAGCTGACCTACAAGCCCGTGCGAGGCAAC |
| | CTGAAGGAACTGGCCCAGGCCTACGCCGACTCTTTCGCTCC |
| | CAAGGACGGCGACCAGGACAACCCCTCTAAGGTGCCCGAGA |
| | TGGTCGAGGGCATGATCTACGGCCCCACCGAGGGCGTGATG |
| | ATGACCGGCATGTACGCCTCTCGAAACGAGGCCAAGCGACG |
| | AGGCAACGTGATCAACAACTACGGCTGGTGGTTCAAGCCCTG |
| | GTTCTACCAGCACGCTCAGACCGCTCTGAAGCGAGGCGAGT |
| | TCGTCGAGTACATCCCTACTCGAGACTACTACCACCGACACA |
| | CCCGATCTCTGTACTGGGAGGGCAAGCTGATTCTGCCCTTCG |
| | GTGACCAGTTCTGGTTCCGATTCCTGCTCGGCTGGCTGATGC |
| | CTCCTAAGATCGCCCTGCTGAAGGCTACCCAGTCTGAGGCCA |
| | TCCGAAACTACTATCACGACCACCACGTGATCCAGGACCTGC |
| | TCGTGCCCCTGTACAAGGTGGGGGACTGCCTCGAGTGGGTG |
| | CACCGAGAGATGGAAGTGTACCCCATCTGGCTGTGTCCCCA |
| | CCGAATCTACAAGCTGCCTGTGCGACCTATGATCTACCCCGA |
| | GCCTGGCTTCGAGAAGCACAAGCGACAGGGTGACACCGAGT |
| | ACGCCCAGATGTACACCGACGTGGGCGTGTACTACGTGCCC |
| | GGTGCCGTGCTGCGAGGTGAGCCCTTCGACGGCTCTGAGAÅ |
| | GTGCCGACAGCTGGAACTGTGGCTGATCGAGAACCACGGCT |
| | TCCAGGCTCAGTACGCCGTGACCGAGCTGACCGAGAAGAAC |
| | TTCTGGCGAATGTTCGAGAAGGGCCTGTACGAGCAGTGCCG |
| | ACGAAAGTACAAGGCCATCGGCACCTTCATGTCTGTGTACTA |
| | CAAGTCTAAGAAGGGCCGAAAGACTGAGAAGGAAGTTCAAGA |
| | GGCCGAGCAAGAGAAGGCTGAGCAAGAAACCCCTGAGGCCA |
| | ACTAA |
| Chenopodium quinoa C-28 | ATGGACTCTATGGCCCTGATCTGCACCGTGGGCCTGCTGTTC |
| sterol methyltransferase | GGCGGCCTGTACTGGTTCATCTGCATTCACGGACCCGCCGA |
| | GCGAAAGGGCAAGCGAGCCGTGGACCTGTCTGGCGGCTCTA |
| | TCTCTTCTGACAAGGTGCAGGACAAGTACCAGCAGTACTGGT |
| | CGTTCTTCCGACGACCTAAGGAAATCGAGACTGCCGAGAAG |
| | GTGCCCGACTTCGTGGACACCTTCTACAACCTGGTGACCGAC |
| | ATCTACGAGTGGGGCTGGGGCCAGTCTTTCCACTTCTCTCCC |
| | TCGATTCCCGGCAAGTCTCACCGAGATGCTACCCGAATCCAC |
| | GAAGAGATGGCCGTCGACCTGATCAAGGTGTCTCCCGGCCA |
| | AAAGATCCTGGACGTCGGCTGCGGCGTCGGCGGACCCATGC |
| | GAGCCATTGCCGCTCACTCTCGAGCCAAGGTGACCGGCATC |
| | ACCATCAACGAGTACCAGGTGAAGCGAGCCAAGCTGCACAA |
| | CAAGAAGGCCGGACTGGACTCTCTGTGCGAGGTGGTGTGCG |
| | GCAACTTCCTCGAGATGCCCTTCGCCTCTAACACCTTCGACG |
| | GCGCCTACTCTATCGAGGCCACCTGTCACGCTCCCAAGCTG |
| | GACGACGTGTACTCTGAGATCTTCCGAGTGCTGAAGCCCGG |
| | CTCTCTGTACGTGTCTTACGAATGGGTGACCACCGACAAGTT |
| | CAACGGCGACGACTCTGAGCACTGCGACGTGATCCAGGGCA |
| | TCGAGCGAGGCGACGCTCTGCCCGGCCTGCGACGATACGAC |
| | GAGATCTCTGAGGCCGCCAAGAAGGTGGGCTTCGAGATCGT |
| | GGACGAGCGAGATCTGGCCGCTCCTCCTGCTAAGCCCTGGT |
| | GGGACCGACTGAAGATGGGCCGAATCGCCTACTGGCGAAAC |
| | CACATCGTGGTGACCGTGCTGGCCGCCATGGGCGTGGCTCC |
| | CAAGGGCACCGTGGACGTGCACGACATGCTGTTCAAGACCG |
| | CCGACTACCTGACTCGAGGCGGCGAGTCTGGCATTTTCTCTC |
| | CCATGCACATGATCCTGTGTCGAAAGCCCGTGGACGCCAAGT |
| | CTGACTCTTAA |

TABLE 4b
Codon-optimised sequences of heterologous genes

| Gene | Nucleic acid sequence, codon optimised for Yarrowia lipolytica |
|---|---|
| Danio rerio | ATGGACCCTCTGCTGTACCTCGGGGGCCTGGCCGTGCTGTTCCTGATGTGGAT |
| delta- | CAAGGTGAAGGGCCTCGAGTACGTGATCATCCACCAGAGATGGATCTTCGTGT |
| 24(25) | GCCTGTTCCTGCTGCCTCTGTCTGTGGTGTTCGACGTGTACTACCACCTCCGAG |
| sterol | CCTGGATCATCTTCAAGATGTGCTCTGCTCCCAAGCAGCACGACCAGCGAGTG |
| reductase | CGAGACATCCAGCGACAGGTGCGAGAGTGGCGAAAGGACGGCGGCAAGAAGT |
| | ACATGTGCACCGGACGACCCGGCTGGCTGACCGTGTCTCTGCGAGTGGGCAA |
| | GTACAAGAAGACCCACAAGAACATCATGATCAACATGATGGACATCCTCGAGGT |
| | GGACACCAAGCGAAAGGTGGTGCGAGTCGAGCCCCTGGCCAACATGGGCCAA |
| | GTGACCGCTCTGCTGAACTCTATCGGCTGGACCCTGCCTGTGCTGCCCGAGCT |
| | GGACGACCTGACCGTCGGCGGACTGGTGATGGGAACCGGCATCGAGTCCTCT |
| | TCTCACATCTACGGCCTGTTCCAGCACATCTGCGTGGCCTTCGAGCTGGTGCT |
| | GGCCGACGGCTCTCTGGTCCGATGCACCGAGAAGGAAAACTCTGACCTGTTCT |
| | ACGCTGTGCCCTGGTCTTGGGGAACCCTGGGCTTCCTGGTCGCCGCCGAGATC |
| | CGAATCATCCCCGCTCAGAAGTGGGTCAAGCTGCACTACGAGCCCGTGCGAGG |
| | CCTGGACGCCATCTGCAAGAAGTTCGCCGAGGAATCTGCCAACAAGGAAAACC |
| | AGTTCGTCGAGGGCCTGCAGTACTCTCGAGATGAGGCCGTGATCATGACCGGC |
| | GTGATGACCGACCACGCTGAGCCCGACAAGACCAACTGCATCGGCTACTACTA |
| | CAAGCCCTGGTTCTTCCGACACGTCGAGTCTTTTCTGAAGCAGAACCGAGTGG |
| | CCGTCGAGTACATTCCCCTGCGACACTACTACCATCGACACACCCGATCTATTT |
| | TCTGGGAGCTGCAGGACATCATCCCCTTCGGCAACAACCCTCTGTTCCGATAC |
| | GTGTTCGGCTGGATGGTGCCTCCTAAGATCTCCCTGCTGAAGCTGACCCAGGG |
| | CGAGACTATCCGAAAGCTGTACGAGCAGCACCACGTCGTCCAGGACATGCTGG |
| | TGCCCATGAAGGACATCAAGGCCGCCATTCAGCGATTCCACGAGGACATCCAC |
| | GTGTACCCTCTGTGGCTGTGCCCCTTTCTGCTGCCCAACCAGCCTGGCATGGT |
| | GCACCCCAAGGGCGACGAGGACGAGCTGTACGTGGACATCGGCGCCTACGGC |
| | GAGCCCAAGGTCAAGCACTTCGAGGCCACCTCTTCTACCCGACAGCTCGAGAA |
| | GTTCGTGCGAGATGTGCACGGCTTCCAGATGCTGTACGCCGACGTCTACATGG |
| | AACGAAAGGAATTCTGGGAGATGTTCGACGGCACCCTGTACCACAAGCTGCGA |
| | GAGGAACTGGGCTGCAAGGACGCTTTCCCCGAGGTGTTTGACAAGATCTGCAA |
| | GTCTGCCCGACACTAA |
| Mus | ATGGAACCCGCCGTGTCTCTGGCCGTGTGCGCCCTGCTGTTCCTGCTGTGGGT |
| musculus | GCGAGTGAAGGGCCTCGAGTTCGTGCTGATCCACCAGAGATGGGTGTTCGTGT |
| delta-24 | GCCTGTTTCTGCTGCCCCTGTCTCTGATCTTCGACATCTACTACTACGTGCGAG |
| (25) sterol | CCTGGGTCGTGTTCAAGCTGTCCTCTGCTCCCCGACTGCACGAGCAGCGAGTG |
| reductase | CGAGACATCCAAAAGCAGGTCCGAGAGTGGAAGGAAGAGGGCTCTAAGACCTT |
| | CATGTGCACCGGACGACCCGGCTGGCTGACCGTGTCGCTGCGAGTGGGCAAG |
| | TACAAGAAGACCCACAAGAACATCATGATCAACCTGATGGACATCCTCGAGGTG |
| | GACACCAAGAAGCAGATCGTCCGAGTCGAGCCCCTGGTGTCTATGGGCCAAGT |
| | GACCGCTCTGCTGAACTCTATCGGCTGGACCCTGCCTGTGCTGCCCGAGCTGG |
| | ACGACCTGACCGTCGGGGGCCTGATCATGGGAACCGGCATCGAGTCCTCTTCG |
| | CACAAGTACGGCCTGTTCCAGCACATCTGCACCGCCTACGAGCTGATCCTGGC |
| | CGACGGCTCTTTCGTCCGATGCACCCCTTCTGAGAACTCTGACCTGTTCTACGC |
| | TGTGCCCTGGTCTTGCGGAACCCTGGGCTTCCTGGTGGCCGCCGAGATCCGAA |
| | TCATCCCCGCCAAGAAGTACGTCAAGCTGCGATTCGAGCCCGTGCGAGGACTG |
| | GAAGCCATCTGCGAGAAGTTCACCCGAGAGTCTCAGCGACTCGAGAACCACTT |
| | CGTCGAGGGCCTGCTGTACTCTCTGGACGAGGCCGTGATCATGACCGGCGTG |
| | ATGACCGACGACGTCGAGCCCTCTAAGCTGAACTCCATTGGCTCTTACTACAAG |
| | CCCTGGTTCTTCAAGCACGTCGAGAACTACCTCAAGACCAACCGAGAGGGACT |
| | CGAGTACATTCCCCTGCGACACTACTACCACCGACACACCCGATCTATTTTCTG |
| | GGAGCTGCAGGACATCATCCCCTTCGGCAACAACCCCATCTTCCGATACCTGTT |
| | CGGCTGGATGGTGCCTCCTAAGATCTCCCTGCTGAAGCTGACCCAGGGCGAGA |
| | CTCTGCGAAAGCTGTACGAGCAGCACCACGTCGTCCAGGACATGCTGGTGCCC |
| | ATGAAGTGCATGTCTCAGGCCCTGCACACCTTCCAGAACGACATCCACGTGTAC |
| | CCCATCTGGCTGTGCCCCTTCATTCTGCCCTCTCAGCCCGGCCTGGTGCACCC |
| | CAAGGGCGACGAGGCTGAGCTGTACGTGGACATCGGCGCCTACGGCGAGCCC |
| | CGAGTGAAGCACTTCGAGGCCCGATCTTGCATGCGACAGCTCGAGAAGTTTGT |
| | GCGATCTGTGCACGGCTTCCAGATGCTGTACGCCGACTGCTACATGAACCGAG |
| | AAGAGTTCTGGGAGATGTTCGACGGATCTCTGTACCACAAGCTCCGAAAGCAG |
| | CTGGGCTGCCAGGACGCTTTCCCCGAGGTGTACGACAAGATCTGCAAGGCCG |
| | CTCGACACTAA |
| Solanum | ATGACCGACGTCCAGGCCCCTCCTCGACCCAAGCGAAAGAAGAACATCATGGA |
| tuberosum | CCTCCTCGTCCAGTTCCGATGGATCGTCGTCATCTTCGTCGTCCTCCCCCTCTC |
| delta-24(28) | CTTCCTCTACTACTTCTCCATCTACGTCGGTGACGTCCGATCCGAGTGCAAGTC |
| sterol | CTACAAGCAGCGACAGAAGGAGCACGACGAGAACGTCAAGAAGGTCGTCAAGC |
| reductase | GACTCAAGGACCGAAACGCCTCCAAGGACGGTCTGGTCTGCACCGCCCGAAA |
| | GCCCTGGGTCGCTGTCGGTATGCGAAACGTCGACTACAAGCGAGCCCGACACT |
| | TCGAGGTCGACCTCTCCCCCTTCCGAAACGTCCTCAACATCGACACCGAGCGA |
| | ATGATCGCCAAGGTCGAGCCCCTCGTCAACATGGGTCAGATCTCCCGAGTCAC |
| | CGTCCCCATGAACGTCTCCCTCGCCGTCGTCGCTGAGCTCGACGACCTCACCG |
| | TCGGTGGTCTGATCAACGGTTACGGTATCGAGGGTTCCTCCCACATCTACGGT |
| | CTGTTCTCCGACACCGTCGTCTCCTACGAGGTCGTCCTCGCCGACGGTCAGGT |
| | CGTCCGAGCCACCAAGGACAACGAGTACTCCGACCTCTTCTACGCCATCCCCT |
| | GGTCCCAGGGTACCCTCGGTCTGCTCGTCTCCGCCGAGATCAAGCTCATCCCC |
| | ATCAAGGAGTACATGAAGCTCACCTACAAGCCCGTCGTCGGTAACCTCAAGGA |
| | GATCGCCCAGGCCTACATCGACTCCTTCTCCCCCAAGGACGGTGACCAGGACA |
| | ACCGAGAGAAGGTCCCCGACTTCGTCGAGACTATGGTCTACACCCCCACCGAG |
| | GCTGTCTGCATGACCGGTCGATACGCCTCCAAGGAGGAGGCCAAGAAGAAGG |
| | GTAACGTCATCAACAACGTCGGTTGGTGGTTCAAGACCTGGTTCTACCAGCACG |
| | CCCAGACCGCTCTCAAGAAGGGTGAGTTCGTCGAGTACATCCCCACCCGAGAG |
| | TACTACCACCGACACACCCGATGCCTCTACTGGGAGGGTAAGCTCATCCTCCC |
| | CTTCGGTGACCAGTGGTGGTTCCGATTCTTCTTCGGTTGGGCCATGCCCCCTAA |
| | GGTTTCCCTCCTCAAGGCCACCCAGGGTGAGTACATCCGAAACTACTACCAGG |
| | AGAACCACGTCATCCAGGACATGCTCGTCCCCCTCTACAAGGTCGGTGACGCC |
| | CTCGAGTGGGTCAACCGAGAGATGGAGGTCTACCCCCTCTGGCTCTGCCCCCA |
| | CCGACTCTACCGACTCCCCCTCAAGACCATGGTCTACCCCGAGCCTGGTTTCG |
| | AGCTCCACAAGCGACAGGGTGACACCAAGTACGCCCAGATGTACACCGACGTC |
| | GGTGTCTACTACGCCCCCGGTCCCATCCTCCGAGGTGAGGTCTTCGACGGTAT |
| | CGAGGCCGTCCGAAAGCTCGAGTCCTGGCTCATCGAGAACCACGGTTTCCAGC |
| | CCCAGTACGCCGTCTCCGAGCTCACCGAGAAGAACTTCTGGCGAATGTTCGAC |
| | GGTTCCCTCTACGAGAACTGCCGAAAGAAGTACCGAGCCATCGGTACCTTCAT |
| | GTCCGTCTACTACAAGTCCAAGAAGGGTAAGAAGACCGAGAAGGAGGTCCAGG |
| | ACGCCGAGCAGGAGACTGCCGAGGTCGAGACTCCCGAGGTCGACGAGCCCGA |
| | GGACTAA |
| Arabidopsis | ATGGACTCTCTGACCCTGTTCTTCACCGGCGCTCTGGTGGCCGTGGGAATCTA |
| thaliana C- | CTGGTTCGTGTGCGTGCTGGGACCCGCCGAGCGAAAGGGCAAGCGAGCCGTG |
| 26 sterol | GACCTGTCTGGCGGCTCTATCTCTGCCGAGAAGGTGCAGGACAACTACAAGCA |
| methyl- | GTACTGGTCGTTCTTCCGACGACCTAAGGAAATCGAGACTGCTGAGAAGGTCC |
| transferase | CCGACTTCGTGGACACCTTCTACAACCTGGTGACCGACATCTACGAGTGGGGC |
| | TGGGGCCAGTCTTTCCACTTCTCTCCCTCGATTCCCGGCAAGTCTCACAAGGAC |
| | GCTACCCGACTGCACGAAGAGATGGCCGTCGACCTGATCCAGGTGAAGCCCG |
| | GCCAAAAGATCCTGGACGTCGGCTGCGGCGTCGGCGGACCCATGCGAGCCAT |
| | TGCCTCTCACTCTCGAGCCAACGTGGTGGGCATCACCATCAACGAGTACCAGG |
| | TGAACCGAGCCAGACTGCACAACAAGAAGGCCGGACTGGACGCCCTGTGCGÅ |
| | GGTGGTGTGCGGCAACTTCCTGCAGATGCCCTTCGACGACAACTCTTTCGACG |
| | GCGCCTACTCTATCGAGGCCACCTGTCACGCTCCCAAGCTGGAAGAGGTGTAC |
| | GCCGAGATCTACCGAGTGCTGAAGCCTGGCTCTATGTACGTGTCTTACGAATG |
| | GGTGACCACCGAGAAGTTCAAGGCCGAGGACGACGAGCACGTCGAGGTGATC |
| | CAGGGCATCGAGCGAGGCGACGCTCTGCCCGGCCTGCGAGCCTACGTGGACA |
| | TTGCCGAGACAGCCAAGAAGGTGGGCTTCGAGATCGTGAAGGAAAAGGACCTG |
| | GCCTCGCCTCCTGCTGAGCCCTGGTGGACCCGACTGAAGATGGGCCGACTGG |
| | CCTACTGGCGAAACCACATCGTGGTGCAGATCCTGTCTGCCGTGGGCGTCGCC |
| | CCTAAGGGCACCGTGGACGTGCACGAGATGCTGTTCAAGACCGCCGACTACCT |
| | GÅCTCGAGGCGGCGAGACTGGCATTTTCTCTCCCATGCACATGATCCTGTGTC |
| | GAAAGCCCGAGTCTCCCGAGGAATCTTCTTAA |
| Amborella | ATGGAGACTCTGGCCGCCGTTGTCACCCTGGGTCTGCTCGCTGCCGGCCTCTA |
| trichopoda | CTGGTTCGTCTGCCTGCTGGGTTCTGCCGAGCAGAAAGGTAAGCACGCCTCTG |
| C-28 sterol | AGCTGTCTGGAGGCTCTCTGGGCCGAGAGCAGGTCGCCTCCACCTACCGACA |
| methyl- | GTACTGGACCTTCTTCCGAAAGCCCAAGGAAATTGAACACGCCGACCGAGTGC |
| transferase | CCGACCTCGTCGACTCGTTTTACAACCTGGTGACCGACATCTACGAGTGGGGC |
| | TGGGGACAGTCTTTCCACTTCTCTCCTTCTCTGCCCGGACATTCCCATGCCGCC |
| | GCTACCCGAGCTCACGAGGAGATGGCCGCTAACCTTCTGAAGCTTGGCCCCGC |
| | CATGAAGGTCCTTGACGCTGGATGCGGCGTGGGTGGCCCCATGCGAACTATCG |
| | CTACTCACTCCGGCGCTAACGTCGTCGGCATCACCATCAACGAGTACCAGGTC |
| | GGCCGAGCCCGACTGCACAACATCAAGGCCGGTCTGGACAAGCTGTGCGAAG |
| | TCGTTTGCGGTGATTTCCTGCACATGCCCTTCGATGCTGAGTCCTTCGACGCCG |
| | CCTACTCCATCGAGGCCACCTGCCACGCTCCCAAGCTCTCCGAGGTCTACGCC |
| | GAGATTTTCCGGGTTCTGAAGCCCGGTGCCCTCTACGTGACCTACGAATGGGT |
| | CACCACCCCCAAGTTTCAGCCCGACAATTCCGAACATCTGGAGATCGTCCAGG |
| | GCATCGAGCGAGGTAACGCCCTTCCCGGTCTCCGACGACAGGACGAGGTCGC |
| | CGAAATCGCCAAGGGCGTCGGCTTCGAGCTGGTTGAAGAGCGGGACCTGGCC |
| | CTACCCCCTGCCCTGCCTTGGTGGACTCGACTTAAGATGGGTCGAGTCGCCTA |
| | CTTTCGAAACCACGTGGTCGTCTGGGTCCTGACCATGGTCCGAATTGCCCCTAA |
| | GGGCGTGGACGACGTGCACGAGATGCTGTTTCATACCGCCCACCACCTGACTC |
| | GAGGCGGTCAGACCGGCATCTTCACCCCCATGCACATGATTCTGCTTCGAAAA |
| | CCTAACAACGCTGCTCCCGCCTGCTAA |

TABLE 5a
Yeast strains

| | | | Elements used to construct strain | |
|---|---|---|---|---|
| Strain | | Parent strain/ | | Integration |
| name | Genotype | Reference | gRNA vector | vector/BioBrick |
| ST4840 | | Y. lipolytica Y- | | |
| | | 17536 (ATCC © | | |
| | | 34088 ™) from | | |
| | | the ARS culture | | |
| | | collection | | |
| ST4842 | MATa | Y. lipolytica W29 | | |
| | | (MatA, | | |
| | | ATCC ©20460 ™) | | |
| | | strain Y-63746 | | |
| | | from the ARS | | |
| | | culture collection | | |
| ST6512 | MATa ku70Δ::PrTEF1- | ST4842/(Marella | | |
| | >Cas9- TTef12::PrGPD- | et al, 2020) | | |
| | >DsdA-TLip2 | | | |
| ST8980 | ST8980 MATa | ST6512/(Amesen | pCfB6627 | pCfB8822 |
| | ku70Δ::PrTEF1->Cas9- | et al, 2020); | | |
| | TTef12::PrGPD->DsdA- | available from | | |
| | TLip2 IntC_2-HMG1 <- | Euroscarf | | |
| | PrGPD-PrTefInt- >ERG12 | (accession no. | | |
| | | Y41408) | | |
| ST9027 | MATa ku70Δ::PrTEF1- | ST8980/(Arnesen | pCfB8856 | pCfB8823 |
| | >Cas9-TTef12:PrGPD- | et al, 2020) | | |
| | >DsdA-TLip2 IntC_2- | | | |
| | HMG1<-PrGPD-PrTefInt- | | | |
| | >ERG12 IntC_3-SeACS <- | | | |
| | PrGPDPrTefInt->YIACL1 | | | |
| ST9100 | MATa ku70Δ::PrTEF1- | ST9027/(Arnesen | | |
| | >Cas9- TTef12::PrGPD- | et al, 2020) | | |
| | >DsdA-TLip2 IntC_2- | | | |
| | HMG1<-PrGPD-PrTefInt- | | | |
| | >ERG12 IntC_3-SeACS<- | | | |
| | PrGPDPrTefInt->YIACL1 | | | |
| | IntD_1-IDI1<- PrGPD- | | | |
| | PrTefInt->ERG20 | | | |
| ST3683 | mus51Δ, nugm-Htg2, | (Arnesen et al, | | |
| | ndh2i, lys11-, leu2-, ura3-, | 2020: Angerer et | | |
| | MatB | al, 2014) Y. | | |
| | | lipolytica GB20 | | |
| | | was a kind gift | | |
| | | from Volker | | |
| | | Zickermann. | | |
| ST10924 | MATa ku70Δ::PrTEF1- | ST9100 | pCfB8861 | pCfB10252 |
| | Cas9-TTef12::PrGPD- | | (pHyg- | (IntE4_PrTEF- |
| | DsdA-TLip2 IntC_2- | | YLgRNA2_IntE_4) | >DHCR7_Esilicul) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_4-PrTefInt- | | | |
| | >DHCR7_Esiliculosus | | | |
| ST10934 | MATa ku70Δ::PrTEF1- | ST10924 | | pCfB10367 |
| | Cas9-TTef12::PrGPD- | | | (Erg5_tPr48) |
| | DsdA-TLip2 IntC_2- | | | |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_4-PrTefInt- | | | |
| | >DHCR7_Esiliculosus | | | |
| | ERG5_tPr48NatMX | | | |
| ST11005 | MATa ku70Δ::PrTEF1- | ST9100 | | pCfB10494 |
| | Cas9-TTef12::PrGPD- | | | (IntE3_PrGPAT- |
| | DsdA-TLip2 IntC_2- | | | >TtSTC) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 InIE_3-PrGPAT- | | | |
| | >TtSTC | | | |
| ST11014 | MATa ku70Δ::PrTEF1- | ST11005 | | BB5098 |
| | Cas9-TTef12::PrGPD- | | | (Erg5_HphMX_KO) |
| | DsdA-TLip2 IntC_2- | | | |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5_HphMX | | | |
| ST11027 | MATa ku70Δ::PrTEF1- | ST11014 | | pCfB6611 (pNat- |
| | Cas9-TTef12::PrGPD- | | | PrExp-Cre) |
| | DsdA-TLip2 IntC_2- | | | |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 | | | |
| ST11028 | MATa ku70Δ::PrTEF1- | ST11014 | | BB5246 |
| | Cas9-TTef12::PrGPD- | | | (Erg4_NatMX_KO) |
| | DsdA-TLip2 IntC_2- | | | |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 Δerg5_HphMX | | | |
| | Δerg4_NatMX | | | |
| ST11325 | MATa ku70Δ::PrTEF1- | ST11027 | | BB5099 |
| | Cas9-TTef12::PrGPD- | | | (Erg6_HphMX_KO) |
| | DsdA-TLip2 IntC_2- | | | |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC -erg5 - | | | |
| | erg6_HphMX | | | |
| ST11326 | MATa ku70Δ::PrTEF1- | ST6512 | | BB5248 |
| | Cas9-TTef12::PrGPD- | | | (Erg4_NatMX_KO) |
| | DsdA-TLip2 -erg4_NatMX | | | |
| ST11327 | MATa ku70Δ::PrTEF1- | ST6512 | | BB5247 |
| | Cas9-TTef12::PrGPD- | | | (Erg5_NatMX_KO) |
| | DsdA-TLip2 -erg5_NatMX | | | |
| ST11040 | MATa ku70Δ::PrTEF1- | ST11027 | | BB5097 |
| | Cas9-TTef12::PrGPD- | | | (Erg4_HphMX_KO) |
| | DsdA-TLip2 IntC_2- | | | |
| | HMG1<-PrGPD-PrTefInt- | | | |
| | >ERG12 pCfBB823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 | | | |
| | Δerg4_HphMX | | | |
| ST11066 | MATa ku70Δ::PrTEF1- | ST11027 | pCfB6638 | pCfB10249 |
| | Cas9-TTef12::PrGPD- | | (pNat- | (IntE4_PrTEF- |
| | DsdA-TLip2 IntC_2- | | YLgRNA2_IntE_4) | >DHCR7_Stuberosum) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 IntE_4- | | | |
| | PrTEF- | | | |
| | >DHCR7_Stuberosum | | | |
| ST11067 | MATa ku70Δ::PrTEF1- | ST11027 | pCfB6638 | pCfB10250 |
| | Cas9-TTef12::PrGPD- | | (pNat- | (IntE4_PrTEF- |
| | DsdA-TLip2 IntC_2- | | YLgRNA2_IntE_4) | >DHCR7_Drerio) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 IntE_4- | | | |
| | PrTEF->DHCR7_Drerio | | | |
| ST11068 | MATa ku70Δ::PrTEF1- | ST11027 | pCfB6638 | pCfB10251 |
| | Cas9-TTef12::PrGPD- | | (pNat- | (IntE4_PrTEF- |
| | DsdA-TLip2 IntC_2- | | YLgRNA2_IntE_4) | >DHCR7_Ldrancou) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 IntE_4- | | | |
| | PrTEF- | | | |
| | >DHCR7_Ldrancou | | | |
| ST11069 | MATa ku70Δ::PrTEF1- | ST11027 | pCfB6638 | pCfB10252 |
| | Cas9-TTef12::PrGPD- | | (pNat- | (IntE4_PrTEF- |
| | DsdA-TLip2 IntC_2- | | YLgRNA2_IntE_4) | >DHCR7_Esilicul) |
| | HMG1<-PrGPD-PrTefInt- | | | |
| | >ERG12 pCfB8823 | | | |
| | IntC_3-SeAC<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 IntE_4- | | | |
| | PrTEF->DHCR7_Esilicul | | | |
| ST11070 | MATa ku70Δ::PrTEF1- | ST11027 | pCfB6638 | pCfB10253 |
| | Cas9-TTef12::PrGPD- | | (pNat- | (In(E4_PrTEF- |
| | DsdA-TLip2 IntC_2- | | YLgRNA2_IntE_4) | >DHCR7_Cprotoch) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 IntE_4- | | | |
| | PrTEF- | | | |
| | >DHCR7_Cprotoch | | | |
| ST11071 | MATa ku70Δ::PrTEF1- | ST11027 | pCfB6638 | pCfB10254 |
| | Cas9-TTef12::PrGPD- | | (pNat- | (IntE4_PrTEF- |
| | DsdA-TLip2 IntC_2- | | YLgRNA2_IntE_4) | >DHCR7_Csubellip) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 IntE_4- | | | |
| | PrTEF- | | | |
| | >DHCR7_Csubellip | | | |
| ST11072 | MATa ku70Δ::PrTEF1- | ST11027 | pCfB6638 | pCfB10255 |
| | Cas9-TTef12::PrGPD- | | (pNat- | (IntE4 PrTEF- |
| | DsdA-TLip2 IntC_2- | | YLgRNA2_IntE_4) | >DHCR7_Mverticillata) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 IntE_4- | | | |
| | PrTEF- | | | |
| | >DHCR7_Mverticillata | | | |
| ST11073 | MATa ku70Δ::PrTEF1- | ST11027 | pCfB6638 | pCfB10256 |
| | Cas9-TTef12::PrGPD- | | (pNat- | (InIE4_PrTEF- |
| | DsdA-TLip2 IntC_2- | | YLgRNA2_IntE_4) | >DHCR7_Gsoja) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 IntE_4- | | | |
| | PrTEF->DHCR7_Gsoja | | | |
| ST11074 | MATa ku70Δ::PrTEF1- | ST11027 | pCfB6638 | pCfB10257 |
| | Cas9-TTef12::PrGPD- | | (pNat- | (IntE4_PrTEF- |
| | DsdA-TLip2 IntC_2- | | YLgRNA2_IntE_4) | >DHCR7_Tsp) |
| | HMG1<-PrGPD-PrTefInt- | | | |
| | >ERG12 pCfB8823 | | | |
| | IntC_3-SeACS<-PrGPD- | | | |
| | PrTeflat->YIACL1 IntD_1- | | | |
| | IDI1<-PrGPD-PrTefInt- | | | |
| | >ERG20 IntE_3-PrGPAT- | | | |
| | >TtSTC Δerg5 IntE_4- | | | |
| | PrTEF->DHCR7_Tsp | | | |
| ST11075 | MATa ku70A::PrTEF1- | ST11027 | pCfB6638 | pCfB10258 |
| | Cas9-TTef12::PrGPD- | | (pNat- | (IntE4_PrTEF- |
| | DsdA-TLip2 IntC_2- | | YLgRNA2_IntE_4) | >DHCR7_Wchondrophi) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 IntE_4- | | | |
| | PrTEF- | | | |
| | >DHCR7_Wchondrophi | | | |
| ST11056 | MATa ku70A:PrTEF1- | ST11040 | pCf86638 | pCfB10249 |
| | Cas9-TTef12::PrGPD- | | (pNat- | (IntE4_PrTEF- |
| | DsdA-TLip2 IntC_2- | | YLgRNA2_IntE_4) | >DHCR7_Stuberosum) |
| | HMG1<-PrGPD-PrTefInt- | | | |
| | >ERG12 pCf88823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 | | | |
| | Δerg4_HphMX IntE_4- | | | |
| | PrTEF- | | | |
| | >DHCR7_Stuberosum | | | |
| ST11057 | MATa ku70Δ::PrTEF1- | ST11040 | pCfB6638 | pCfB10250 |
| | Cas9-TTef12::PrGPD- | | (pNat- | (IntE4_PrTEF- |
| | DsdA-TLip2 IntC_2- | | YLgRNA2_IntE_4) | >DHCR7_Drerio) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 | | | |
| | Δerg4_HphMX IntE_4- | | | |
| | PrTEF->DHCR7_Drerio | | | |
| ST11058 | MATa ku70Δ::PrTEF1- | ST11040 | pCfB6638 | pCfB10251 |
| | Cas9-TTef12::PrGPD- | | (pNat- | (IntE4_PrTEF- |
| | DsdA-TLip2 IntC_2- | | YLgRNA2_IntE_4) | >DHCR7 Ldrancou) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 | | | |
| | Δerg4_HphMX IntE_4- | | | |
| | PrTEF- | | | |
| | >DHCR7_Ldrancou | | | |
| ST11059 | MATa ku70Δ::PrTEF1- | ST11040 | pCfB6638 | pCfB10252 |
| | Cas9-TTef12::PrGPD- | | (pNat- | (IntE4 PrTEF- |
| | DsdA-TLip2 IntC_2- | | YLgRNA2_IntE_4) | >DHCR7_Esilicul) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 | | | |
| | Δerg4_HphMX IntE_4- | | | |
| | PrTEF->DHCR7_Esilicul | | | |
| ST11060 | MATa ku70Δ::PrTEF1- | ST11040 | pCfB6638 | pCfB10253 |
| | Cas9-TTef12::PrGPD- | | (pNat- | (IntE4_PrTEF- |
| | DsdA-TLip2 IntC_2- | | YLgRNA2_IntE_4) | >DHCR7_Cprotoch) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 | | | |
| | Δerg4_HphMX IntE_4- | | | |
| | PrTEF- | | | |
| | >DHCR7_Cprotoch | | | |
| ST11061 | MATa ku70Δ::PrTEF1- | ST11040 | pCfB6638 | pCfB10254 |
| | Cas9-TTef12::PrGPD- | | (pNat- | (IntE4_PrTEF- |
| | DsdA-TLip2 IntC_2- | | YLgRNA2_IntE_4) | >DHCR7_Csubellip) |
| | HMG1<-PrGPD-PrTefInt- | | | |
| | >ERG12 pCfB8823 | | | |
| | IntC_3-SeACS<-PrGPD- | | | |
| | PrTeflat->YIACL1 IntD_1- | | | |
| | IDI1<-PrGPD-PrTefInt- | | | |
| | >ERG20 IntE_3-PrGPAT- | | | |
| | >TtSTC Δerg5 | | | |
| | Δerg4_HphMX IntE_4- | | | |
| | PrTEF- | | | |
| | >DHCR7_Csubellip | | | |
| ST11062 | MATa ku70Δ::PrTEF1- | ST11040 | pCfB6638 | pCfB10255 |
| | Cas9-TTef12::PrGPD- | | (pNat- | (IntE4_PrTEF- |
| | DsdA-TLip2 IntC_2- | | YLgRNA2_IntE_4) | >DHCR7_Mverticillata) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 | | | |
| | Δerg4_HphMX IntE_4- | | | |
| | PrTEF- | | | |
| | >DHCR7_Mverticillata | | | |
| ST11063 | MATa ku70Δ::PrTEF1- | ST11040 | pCfB6638 | pCfB10256 |
| | Cas9-TTef12::PrGPD- | | (pNat- | (IntE4_PrTEF- |
| | DsdA-TLip2 IntC_2- | | YLgRNA2_IntE_4) | >DHCR7_Gsoja) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 | | | |
| | Δerg4_HphMX IntE_4- | | | |
| | PrTEF->DHCR7_Gsoja | | | |
| ST11064 | MATa ku70Δ::PrTEF1- | ST11040 | pCfB6638 | pCfB10257 |
| | Cas9-TTef12::PrGPD- | | (pNat- | (IntE4_PrTEF- |
| | DsdA-TLip2 IntC_2- | | YLgRNA2_IntE_4) | >DHCR7_Tsp) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 | | | |
| | Δerg4_HphMX IntE_4- | | | |
| | PrTEF->DHCR7_Tsp | | | |
| ST11065 | MATa ku70Δ::PrTEF1- | ST11040 | pCfB6638 | pCfB10258 |
| | Cas9-TTef12::PrGPD- | | (pNat- | (IntE4 PrTEF- |
| | DsdA-TLip2 IntC_2- | | YLgRNA2_IntE_4) | >DHCR7_Wchondrophi) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Δerg5 | | | |
| | Δerg4_HphMX IntE_4- | | | |
| | PrTEF- | | | |
| | >DHCR7_Wchondrophi | | | |
| ST10924 | MATa ku70Δ::PrTEF1- | ST9100 | pCfB8861 | pCfB10252 |
| | Cas9-TTef12::PrGPD- | | (pHyg- | (IntE4_PrTEF- |
| | DsdA-TLip2 IntC_2- | | YLgRNA2_IntE_4) | >DHCR7_Esilicul) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_4-PrTefInt- | | | |
| | >DHCR7_Esiliculosus | | | |
| ST10934 | MATa ku70Δ::PrTEF1- | ST10924 | | pCfB10367 |
| | Cas9-TTef12::PrGPD- | | | (Erg5_trPr48) |
| | DsdA-TLip2 IntC_2- | | | |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_4-PrTefInt- | | | |
| | >DHOR7_Esiliculosus | | | |
| | HygMX-ERG5_tPr48 | | | |
| ST11196 | MATa ku70Δ::PrTEF1- | ST10924 | | pCfB7254 (gRNA |
| | Cas9-TTef12::PrGPD- | | | for PAH1 deletion |
| | DsdA-TLip2 IntC_2- | | | Y.L); BB5314 |
| | HMG1<-PrGPD-PrTefInt- | | | (PAH1 repair |
| | >ERG12 pCfB8823 | | | template) |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_4-PrTefInt- | | | |
| | >DHCR7_Esiliculosus | | | |
| | ΔPAH1 | | | |
| ST11197 | MATa ku70Δ::PrTEF1- | ST11069 | | pCfB7254 (gRNA |
| | Cas9-TTef12::PrGPD- | | | for PAH1 deletion |
| | DsdA-TLip2 IntC_2- | | | Y.L); BB5314 |
| | HMG1<-PrGPD-PrTefInt- | | | (PAH1 repair |
| | >ERG12 pCfB8623 | | | template) |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC IntE_4-PrTEF- | | | |
| | >DHCR7_Esilicul ΔERG5 | | | |
| | ΔPAH1 | | | |
| ST11337 | MATa ku70Δ::PrTEF1- | ST11064 | pBP8003 (pNat- | pCfB10841 |
| | Cas9-TTef12::PrGPD- | | YLgRNA4- | (IntF3_PrDGA1- |
| | DsdA-TLip2 IntC_2- | | IntF_3) | >ERG4) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC -erg5 - | | | |
| | erg4_HphMX IntE_4- | | | |
| | PrTEF->DHCR7_Tsp | | | |
| | IntF3_PrDGA1->ERG4 | | | |
| ST11338 | MATa ku70Δ::PrTEF1- | ST11064 | pBP8003 (pNat- | pCfB10842 |
| | Cas9-TTef12::PrGPD- | | YLgRNA4- | (IntF3_PrGPAT- |
| | DsdA-TLip2 IntC_2- | | IntF_3) | >ERG4) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC -erg5 - | | | |
| | erg4 HphMX IntE_4- | | | |
| | PrTEF->DHCR7_Tsp | | | |
| | IntF3_PrGPAT->ERG4 | | | |
| ST11378 | MATa ku70Δ::PrTEF1- | ST11337 | pCfB10783 | pCfB10853 |
| | Cas9-TTef12::PrGPD- | | (pNatYlgRNA- | (IntE5_PrGPAT- |
| | DsdA-TLip2 IntC_2- | | IntE_5) | >DHCR24_Slycopersic) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC -erg5 - | | | |
| | erg4_HphMX IntE_4- | | | |
| | PrTEF->DHCR7_Tsp | | | |
| | IntF_3-PrDGA1->ERG4 | | | |
| | IntE_5-PrGPAT- | | | |
| | >DHCR24_Slycopersic | | | |
| ST11340 | MATa ku70Δ::PrTEF1- | ST11338 | pCfB10783 | pCfB10853 |
| | Cas9-TTef12:PrGPD- | | (pNatYlgRNA- | (IntE5_PrGPAT- |
| | DsdA-TLip2 IntC_2- | | IntE_5) | >DHCR24_Slycopersic) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC -erg5 - | | | |
| | erg4_HphMX IntE_4- | | | |
| | PrTEF->DHCR7_Tsp | | | |
| | IntF_3-PrGPAT->ERG4 | | | |
| | IntE_5-PrGPAT- | | | |
| | >DHCR24_Slycopersic | | | |
| ST11362 | MATa ku70Δ::PrTEF1- | ST11340 | pCfB6633 | pCfB10851 |
| | Cas9-TTef12::PrGPD- | | (pNat- | (IntE1_PrGPAT- |
| | DsdA-TLip2 IntC_2- | | YLgRNA2_IntE_1) | >SMT2_Cquinoa) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC -erg5 - | | | |
| | erg4_HphMX IntE_4- | | | |
| | PrTEF->DHCR7_Tsp | | | |
| | IntF 3-PrGPAT->ERG4 | | | |
| | IntE_5-PrGPAT- | | | |
| | >DHCR24_Slycopersic | | | |
| | IntE1_PrGPAT- | | | |
| | >SMT2_Cquinoa | | | |
| ST11441 | MATa ku70Δ::PrTEF1- | ST11362 | | pCfB4783 (Int_3 |
| | Cas9-TTef12::PrGPD- | | | NatMX) |
| | DsdA-TLip2 IntC_2- | | | |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-NatMX - | | | |
| | erg5 -erg4_HphMX | | | |
| | IntE_4-PrTEF- | | | |
| | >DHCR7_Tsp IntF_3- | | | |
| | PrGPAT->ERG4 IntE_5- | | | |
| | PrGPAT- | | | |
| | >DHCR24_Slycopersic | | | |
| | IntE1_PrGPAT- | | | |
| | >SMT2_Cquinoa | | | |
| ST11540 | MATa ku70Δ::PrTEF1- | ST11378 | | pCfB6611 (pNat- |
| | Cas9-TTef12::PrGPD- | | | PrExp-Cre |
| | DsdA-TLip2 IntC_2- | | | |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC -erg5 -erg4 | | | |
| | IntE_4-PrTEF- | | | |
| | >DHCR7_Tsp IntF_3- | | | |
| | PrDGA1->ERG4 IntE_5- | | | |
| | PrGPAT- | | | |
| | >DHCR24_Slycopersic | | | |
| ST11541 | MATa ku70Δ::PrTEF1- | ST11540 | | pCfB10919 (Int_3 |
| | Cas9-TTef12::PrGPD- | | | NatMX_PrGPAT- |
| | DsdA-TLip2 IntC_2- | | | >SMT2_Cquinoa) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-NatMX- | | | |
| | PrGPAT->SMT2_Cquinoa | | | |
| | -erg5 -erg4 IntE_4-PrTEF- | | | |
| | >DHCR7_Tsp IntF_3- | | | |
| | PrDGA1->ERG4 IntE_5- | | | |
| | PrGPAT- | | | |
| | >DHCR24_Slycopersic | | | |
| ST11542 | MATa ku70Δ::PrTEF1- | ST11541 | | pCfB6612 (pHph- |
| | Cas9-TTef12::PrGPD- | | | PrExp-Cre) |
| | DsdA-TLip2 IntC_2- | | | |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >SMT2_Cquinoa -erg5 - | | | |
| | erg4 IntE_4-PrTEF- | | | |
| | >DHCR7_Tsp IntF_3- | | | |
| | PrDGA1->ERG4 IntE_5- | | | |
| | PrGPAT- | | | |
| | >DHCR24_Slycopersic | | | |

TABLE 5b
Yeast strains

| | | | Elements used to construct strain | |
|---|---|---|---|---|
| Strain | | Parent strain/ | | Integration |
| name | Genotype | Reference | gRNA vector | vector/BioBrick |
| ST4842 | MATa | Y. lipolytica W29 | | |
| | | (MatA, | | |
| | | ATCC © 20460 ™) | | |
| | | strain Y-63746 | | |
| | | from the ARS | | |
| | | culture collection | | |
| ST6512 | MATa ku70Δ::PrTEF1- | ST4842/(Marella | | |
| | >Cas9- TTef12::PrGPD- | et al. 2020) | | |
| | >DsdA-TLip2 | | | |
| ST9100 | MATa ku70Δ::PrTEF1- | ST6512/(Arnesen | | |
| | >Cas9- TTef12::PrGPD- | et al, 2020) | | |
| | >DsdA-TLip2 IntC_2- | | | |
| | HMG1<-PrGPD-PrTefInt- | | | |
| | >ERG12 IntC_3-SeACS<- | | | |
| | PrGPDPrTefInt->YIACL1 | | | |
| | IntD_1-IDI1<- PrGPD- | | | |
| | PrTefInt->ERG20 | | | |
| ST11005 | MATa ku70Δ::PrTEF1- | ST9100 | pCfB6637 | pCfB10494 |
| | Cas9-TTef12::PrGPD- | | (pNat- | (IntE3_PrGPAT- |
| | DsdA-TLip2 IntC_2- | | YLgRNA3_IntE_3) | >TtSTC) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC | | | |
| ST11014 | MATa ku70Δ::PrTEF1- | ST11005 | | BB5098 |
| | Cas9-TTef12::PrGPD- | | | (Erg5_HphMX_KO) |
| | DsdA-TLip2 IntC_2- | | | |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Derg5_HphMX | | | |
| ST11027 | MATa ku70Δ::PrTEF1- | ST11014 | | pCfB6611 (pNat- |
| | Cas9-TTef12:PrGPD- | | | PrExp-Cre) |
| | DsdA-TLip2 IntC_2- | | | |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Derg5 | | | |
| ST11040 | MATa ku70Δ::PrTEF1- | ST11027 | | BB5097 |
| | Cas9-TTef12::PrGPD- | | | (Erg4_HphMX_KO) |
| | DsdA-TLip2 IntC_2- | | | |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Derg5 | | | |
| | Derg4_HphMX | | | |
| ST11325 | MATa ku70Δ::PrTEF1- | ST11027 | | BB5099 |
| | Cas9-TTef12::PrGPD- | | | (Erg6_HphMX_KO) |
| | DsdA-TLip2 IntC_2- | | | |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC -erg5 - | | | |
| | erg6_HphMX | | | |
| ST11330 | MATa ku70Δ::PrTEF1- | ST11325 | | pCfB6611 (pNat- |
| | Cas9-TTef12::PrGPD- | | | PrExp-Cre) |
| | DsdA-TLip2 IntC_2- | | | |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC -erg5 -erg6 | | | |
| ST11071 | MATa ku70Δ::PrTEF1- | ST11027 | pCfB6638 | pCfB10254 |
| | Cas9-TTef12::PrGPD- | | (pNat- | (IntE4_PrTEF- |
| | DsdA-TLip2 IntC_2- | | YLgRNA2_IntE_4) | >DHCR7_Csubellip) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Derg5 IntE_4- | | | |
| | PrTEF- | | | |
| | >DHCR7_Csubellip | | | |
| ST11064 | MATa ku70Δ::PrTEF1- | ST11040 | pCfB6638 | pCfB10257 |
| | Cas9-TTef12::PrGPD- | | (pNat- | (IntE4_PrTEF- |
| | DsdA-TLip2 IntC_2- | | YLgRNA2_IntE_4) | >DHCR7_Tsp) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC Derg5 | | | |
| | Derg4_HphMX IntE_4- | | | |
| | PrTEF->DHCR7_Tsp | | | |
| ST11804 | MATa ku70Δ::PrTEF1- | ST11071 | pCfB6633 | pCfB10311 |
| | Cas9-TTef12::PrGPD- | | (pNat- | (intE1_PrTEF- |
| | DsdA-TLip2 IntC_2- | | YLgRNA2_IntE_1) | >SMT2_Cquinoa) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC -erg5 IntE_4- | | | |
| | PrTEF- | | | |
| | >DHCR7_Csubellip | | | |
| | IntE_1-PrTEF- | | | |
| | >SMT2_Cquinoa | | | |
| ST12139 | MATa ku70Δ::PrTEF1- | ST11804 | pBP8003 (pNat- | pCfB11486 |
| | Cas9-TTef12::PrGPD- | | YLgRNA4- | (IntF3_SMT_Ath<- |
| | DsdA-TLip2 IntC_2- | | IntF_3) | PrGPD_TEFi- |
| | HMG1<-PrGPD-PrTefInt- | | | >SMT_Atr) |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC -erg5 IntE_4- | | | |
| | PrTEF- | | | |
| | >DHCR7_Csubellip | | | |
| | IntE_1-PrTEF- | | | |
| | >SMT2_Cquinoa | | | |
| | IntF3_SMT_Ath<- | | | |
| | PrGPD_TEFi->SMT_Atr | | | |
| ST11803 | MATa ku70Δ::PrTEF1- | ST11064 | pCfB6633 | pCfB10311 |
| | Cas9-TTef12::PrGPD- | | (pNat- | (intE1_PrTEF- |
| | DsdA-TLip2 IntC_2- | | YLgRNA2_IntE_1) | >SMT2_Cquinoa) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC -erg5 - | | | |
| | erg4_HphMX IntE_4- | | | |
| | PrTEF->DHCR7_Tsp | | | |
| | IntE_1-PrTEF- | | | |
| | >SMT2_Cquinoa | | | |
| ST12108 | MATa ku70Δ::PrTEF1- | ST11803 | pBP8003 (pNat- | pCfB11486 |
| | Cas9-TTef12::PrGPD- | | YLgRNA4- | (IntF3_SMT_Ath<- |
| | DsdA-TLip2 IntC_2- | | IntF_3) | PrGPD_TEFi- |
| | HMG1<-PrGPD-PrTefInt- | | | >SMT_Atr) |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC -erg5 - | | | |
| | erg4_HphMX IntE_4- | | | |
| | PrTEF->DHCR7_Tsp | | | |
| | IntE_1-PrTEF- | | | |
| | >SMT2_Cquinoa | | | |
| | IntF3_SMT_Ath<- | | | |
| | PrGPD_TEFi->SMT_Atr | | | |
| ST11346 | MATa ku70Δ::PrTEF1- | ST11330 | pCfB6638 | pCfB10251 |
| | Cas9-TTef12::PrGPD- | | (pNat- | (intE4_PrTEF- |
| | DsdA-TLip2 IntC_2- | | YLgRNA2_IntE_4) | >DHCR7_Ldrancou) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC -erg5 -erg6 | | | |
| | IntE_4-PrTEF- | | | |
| | >DHCR7_Ldrancou | | | |
| ST11829 | MATa ku70Δ::PrTEF1- | ST11346 | pCfB6633 | pCfB10293 |
| | Cas9-TTef12::PrGPD- | | (pNat- | (intE1_PrTEF- |
| | DsdA-TLip2 IntC_2- | | YLgRNA2_IntE_1) | >DHCR24_Drerio) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC -erg5 -erg6 | | | |
| | IntE_4-PrTEF- | | | |
| | >DHCR7_Ldrancou | | | |
| | IntE1_PrTEF- | | | |
| | >DHCR24_Drerio | | | |
| ST11830 | MATa ku70Δ::PrTEF1- | ST11346 | pCfB6633 | pCfB10298 |
| | Cas9-TTef12::PrGPD- | | (pNat- | (intE1_PrTEF- |
| | DsdA-TLip2 IntC_2- | | YLgRNA2_IntE_1) | >DHCR24_Mmusculus) |
| | HMG1<-PrGPD-PrTefInt- | | | |
| | >ERG12 pCfB8823 | | | |
| | IntC_3-SeACS<-PrGPD- | | | |
| | PrTefInt->YIACL1 IntD_1- | | | |
| | IDI1<-PrGPD-PrTefInt- | | | |
| | >ERG20 IntE_3-PrGPAT- | | | |
| | >TtSTC -erg5 -erg6 | | | |
| | IntE_4-PrTEF- | | | |
| | >DHCR7_Ldrancou | | | |
| | IntE1_PrTEF- | | | |
| | >DHCR24_Mmusculus | | | |

TABLE 5c
Yeast strains

| | | | Elements used to construct strain | |
|---|---|---|---|---|
| Strain | | Parent strain/ | | Integration |
| name | Genotype | Reference | gRNA vector | vector/BioBrick |
| ST4842 | MATa | Y. lipolytica W29 | | |
| | | (MatA, | | |
| | | ATCC ©20460 ™) | | |
| | | strain Y-63746 | | |
| | | from the ARS | | |
| | | culture | | |
| | | collection | | |
| ST6512 | MATa | ST4842/ | | |
| | ku70Δ::PrTEF1- | (Marella et al, | | |
| | >Cas9- | 2020) | | |
| | TTef12::PrGPD- | | | |
| | >DsdA-TLip2 | | | |
| ST9100 | MATa | ST6512/ | | |
| | ku70Δ::PrTEF1- | (Arnesen et al, | | |
| | >Cas9- | 2020) | | |
| | TTef12::PrGPD- | | | |
| | >DsdA-TLip2 IntC_2- | | | |
| | HMG1<-PrGPD- | | | |
| | PrTefInt- >ERG12 | | | |
| | IntC_3-SeACS<- | | | |
| | PrGPDPrTefInt- | | | |
| | >YIACL1 IntD_1- | | | |
| | IDI1<- PrGPD- | | | |
| | PrTefInt->ERG20 | | | |
| ST11005 | MATa | ST9100 | pCfB6637 | pCfB10494 |
| | ku70Δ::PrTEF1- | | (pNat- | (IntE3_PrGPAT->TtSTC) |
| | Cas9- | | YLgRNA3_IntE_3) | |
| | TTef12::PrGPD- | | | |
| | DsdA-TLip2 IntC_2- | | | |
| | HMG1<-PrGPD- | | | |
| | PrTefInt->ERG12 | | | |
| | pCfB8823 IntC_3- | | | |
| | SeACS<-PrGPD- | | | |
| | PrTefInt->YIACL1 | | | |
| | IntD_1-IDI1<-PrGPD- | | | |
| | PrTefInt->ERG20 | | | |
| | IntE_3-PrGPAT- | | | |
| | >TtSTC | | | |
| ST11014 | MATa | ST11005 | | BB5098 |
| | ku70Δ::PrTEF1- | | | (Erg5_HphMX_KO) |
| | Cas9- | | | |
| | TTef12::PrGPD- | | | |
| | DsdA-TLip2 IntC_2- | | | |
| | HMG1<-PrGPD- | | | |
| | PrTefInt->ERG12 | | | |
| | pCfB8823 IntC_3- | | | |
| | SeACS<-PrGPD- | | | |
| | PrTefInt->YIACL1 | | | |
| | IntD_1-IDI1<-PrGPD- | | | |
| | PrTefInt->ERG20 | | | |
| | IntE_3-PrGPAT- | | | |
| | >TtSTC | | | |
| | Derg5_HphMX | | | |
| ST11027 | MATa | ST11014 | | pCfB6611 (pNat-PrExp- |
| | ku70Δ::PrTEF1- | | | Cre) |
| | Cas9- | | | |
| | TTef12::PrGPD- | | | |
| | DsdA-TLip2 IntC_2- | | | |
| | HMG1<-PrGPD- | | | |
| | PrTefInt->ERG12 | | | |
| | pCfB8823 IntC_3- | | | |
| | SeACS<-PrGPD- | | | |
| | PrTefInt->YIACL1 | | | |
| | IntD_1-IDI1<-PrGPD- | | | |
| | PrTefInt->ERG20 | | | |
| | IntE_3-PrGPAT- | | | |
| | >TtSTC Derg5 | | | |
| ST11040 | MATa | ST11027 | | BB5097 |
| | ku70Δ::PrTEF1- | | | (Erg4_HphMX_KO) |
| | Cas9- | | | |
| | TTef12::PrGPD- | | | |
| | DsdA-TLip2 IntC_2- | | | |
| | HMG1<-PrGPD- | | | |
| | PrTefInt->ERG12 | | | |
| | pCfB8823 IntC_3- | | | |
| | SeACS<-PrGPD- | | | |
| | PrTefInt->YIACL1 | | | |
| | IntD_1-IDI1<-PrGPD- | | | |
| | PrTefInt->ERG20 | | | |
| | IntE_3-PrGPAT- | | | |
| | >TtSTC -erg5 - | | | |
| | erg4_HphMX | | | |
| ST11064 | MATa | ST11040 | pCfB6638 | pCfB10257 |
| | ku70Δ::PrTEF1- | | (pNat- | (IntE4_PrTEF- |
| | Cas9- | | YLgRNA2_IntE_4) | >DHCR7_Tsp) |
| | TTef12::PrGPD- | | | |
| | DsdA-TLip2 IntC_2- | | | |
| | HMG1<-PrGPD- | | | |
| | PrTefInt->ERG12 | | | |
| | pCfB8823 IntC_3- | | | |
| | SeACS<-PrGPD- | | | |
| | PrTefInt->YIACL1 | | | |
| | IntD_1-IDI1<-PrGPD- | | | |
| | PrTefInt->ERG20 | | | |
| | IntE_3-PrGPAT- | | | |
| | >TtSTC -erg5 - | | | |
| | erg4_HphMX IntE_4- | | | |
| | PrTEF->DHCR7_Tsp | | | |
| ST11943 | MATa | ST11064 | pBP8003 (pNat- | pCfB10948 |
| | ku70Δ::PrTEF1- | | YLgRNA4- | (IntF3_PrDGA1- |
| | Cas9- | | IntF_3) | >StSSR1) |
| | TTef12::PrGPD- | | | |
| | DsdA-TLip2 IntC_2- | | | |
| | HMG1<-PrGPD- | | | |
| | PrTefInt->ERG12 | | | |
| | pCfB8823 IntC_3- | | | |
| | SeACS<-PrGPD- | | | |
| | PrTefInt->YIACL1 | | | |
| | IntD_1-IDI1<-PrGPD- | | | |
| | PrTefInt->ERG20 | | | |
| | IntE_3-PrGPAT- | | | |
| | >TtSTC -erg5 - | | | |
| | erg4_HphMX IntE_4- | | | |
| | PrTEF->DHCR7_Tsp | | | |
| | IntF3_PrDGA1- | | | |
| | >StSSR1 | | | |
| ST12140 | MATa | ST11943 | pCfB6633 | pCfB10851 |
| | ku70Δ::PrTEF1- | | (pNat- | (IntE1_PrGPAT- |
| | Cas9- | | YLgRNA2_IntE_1) | >SMT2_Cquinoa) |
| | TTef12::PrGPD- | | | |
| | DsdA-TLip2 IntC_2- | | | |
| | HMG1<-PrGPD- | | | |
| | PrTefInt->ERG12 | | | |
| | pCfB8823 IntC_3- | | | |
| | SeACS<-PrGPD- | | | |
| | PrTefInt->YIACL1 | | | |
| | IntD_1-IDI1<-PrGPD- | | | |
| | PrTefInt->ERG20 | | | |
| | IntE_3-PrGPAT- | | | |
| | >TtSTC -erg5 - | | | |
| | erg4_HphMX IntE_4- | | | |
| | PrTEF->DHCR7_Tsp | | | |
| | IntF3_PrDGA1- | | | |
| | >StSSR1 | | | |
| | IntE1_PrGPAT- | | | |
| | >SMT2_Cq | | | |
| ST12178 | MATa | ST12140 | pCfB10783 | pCfB10853 |
| | ku70Δ::PrTEF1- | | (pNatYlgRNA- | (IntE5_PrGPAT- |
| | Cas9- | | IntE_5) | >DHCR24_Slycopersic) |
| | TTef12::PrGPD- | | | |
| | DsdA-TLip2 IntC_2- | | | |
| | HMG1<-PrGPD- | | | |
| | PrTefInt->ERG12 | | | |
| | pCfB8823 IntC_3- | | | |
| | SeACS<-PrGPD- | | | |
| | PrTefInt->YIACL1 | | | |
| | IntD_1-IDI1<-PrGPD- | | | |
| | PrTefInt->ERG20 | | | |
| | IntE_3-PrGPAT- | | | |
| | >TtSTC -erg5 - | | | |
| | erg4_HphMX IntE_4- | | | |
| | PrTEF->DHCR7_Tsp | | | |
| | IntF3_PrDGA1- | | | |
| | >StSSR1 | | | |
| | IntE1_PrGPAT- | | | |
| | >SMT2_Cq | | | |
| | IntE5_PrGPAT- | | | |
| | >DHCR24_Sl | | | |

Foundational Genome Engineering Toolbox for Y. lipolytica

The engineering undertaken for Y. lipolytica strain production as described herein below utilised the EasyClone YALI toolbox with USER® cloning for Y. lipolytica as established by Holkenbrink et al. and described in Holkenbrink et al. EasyCloneYALI:CRISPR/Cas9-Based Synthetic Toolbox for Engineering of the Yeast Yarrowia lipolytica, Biotechnol. J. (2018). This approach relies on a toolbox of genome engineering components all of which are attainable with reference to known construct or sequence information including a set of integrative expression vectors which allow expression of one or two genes per vector and integration into highly expressed intergenic genome sites (see for example Addgene for vector information). The exact genome locations of these integration sites are listed in Table S5 of the supporting information of the Holkenbrink et al. 2018 paper and are also listed in Table 18 below. For the purpose of the Y. lipolytica engineering disclosed herein, reference to integrative expression vectors and other information available from the Holkenbrink et al. 2018 paper is supplemented with information on plasmids required and their construction with reference to biobricks (DNA fragments for cloning) and their derivation by PCR amplification. Heterologous genes for expression in Y. lipolytica were codon-optimised for this yeast and obtained as synthetic DNA fragments (see Table 4 above).

### Example 1—Construction of Yarrowia lipolytica Platform Strain for Sterol Production

### Materials and Methods

### Strains, Culture Conditions and Chemicals

Escherichia coli strain DH5α was used for plasmid construction. E. coli was grown at 37° C. and 300 rpm in Lysogeny Broth (LB) liquid medium and at 37° C. on LB solid medium plates supplemented with 20 g/l agar. Ampicillin was supplemented at a concentration of 100 mg/l for plasmid selection.

The Yarrowia lipolytica W29 derived strain ST6512 (Mata ku70Δ:PrTEF1→Cas9-TTef12:PrGPD→DsdA-TLip2) was obtained from Irina borodina (Novo Nordisk Foundation Centre for Biosustainability, Technical University of Denmark. Copenhagen). The strain may also be requested from Euroscarf (accession no. Y41408). This strain is engineered from the commercially available Y. lipolytica W29 strain Y-63746 (MatA, ATCC® 20460™/ARS Culture Collection. NCAUR. United States) as described fully in Marella et al. (2020) Metabol. Eng. The strain ST8512 was used to construct the platform strain (designated ST9100). All strains are detailed in Table 5.

Y. lipolytica was grown at 30° C. on yeast extract peptone dextrose (YPD) media containing 10 g/l yeast extract, 20 g/l peptone and 20 g/l glucose, supplemented with 20 g/l agar for preparation or solid media. For selection, either nourseothricin (250 mg/l) or hygromycin (400 mg/l) was added to the media.

Cultivation of strains for sterol production was performed in yeast extract peptone medium containing 80 g/l glucose.

Chemicals were obtained, if not indicated otherwise, from Sigma-Aldrich. Nourseothricin was purchased from Jena BioScience GmbH (Germany).

### Plasmid Construction

Native gene sequences were amplified from CLIB122 derived strain ST3883 gDNA. Strain ST9204 as described in Arnesen et al. 2020, is derived from ST9100. Hence, ST9204 includes all the genomic features of ST9100. Sequences required to construct ST9100 can therefore be equivalently amplified from ST9204 gDNA. The strain ST9204 may be requested from Euroscarf (accession no. Y41404).

The gene encoding Salmonella enterica acetyl-CoA synthetase (SeACS. GenBank accession; WP_000083882.1) was codon-optimized for Y. lipolytica and synthesised as GeneArt String DNA fragments by ThermoFisher Scientific (Table 4).

The plasmids, biobricks, and primers used in this study are listed in Tables 8-8, respectively. As indicated above, the biobricks were amplified by PCR. The PCR mix contained 32 μl water, 10 μl high fidelity Phusion® polymerase buffer (5×), 1 μl 10 mM dNTP, 1.5 μl MgCl₂, 2.5 μl 10 μM Fw primer, 2.5 μl 10 μM Rv primer, 0.2 μl Phusion® U polymerase, and 10 ng DNA template. Reactions were multiplexed 8× per BioBrick. The cycling program was: 98° C. for 1 min, 30 cycles of [98° C. for 10 sec, gradient from 50 to 58′C for 20 sec, 72° C. for 30 s/kb], 72° C. for 5 min, pause at 10° C. The gene fragments were purified from agarose gels using the NucleoSpin® Gel and PCR Clean-up kit (Macherey-Nagel). Biobricks were assembled into the EasyCloneYALI vectors with USER cloning (Holkenbrink et al, 2018). The USER reactions were transformed into E. coli and correct assemblies were verified by Sanger sequencing (Eurofins).

TABLE 6
Plasmids for construction of Yarrowia lipolytica platform strain for sterol production

| | Parent plasmid/ | |
|---|---|---|
| Plasmid name | Reference | BioBricks |
| pCfB8843 (pORI1001-Hyg-CEN1-USER) | (Arnesen et al, 2020) | BB3924, BB3925 |
| pCfB6682 (pIntC_2-TPex20-TLip2) | (Holkenbrink et al, 2018) | |
| pCfB6371 (pIntC_3-TPex20-TLip2) | (Holkenbrink et al, 2018) | |
| pCfB6684 (pIntD_1-TPex20-TLip2) | (Holkenbrink et al, 2018) | |
| pCfB6627 (pNat-YLgRNA2_IntC_2) | (Holkenbrink et al, 2018) | |
| pCfB6631 (pNat-YLgRNA2_IntD_1) | (Holkenbrink et al, 2018) | |
| pCfB8856 (pHphMX-YLgRNA2_IntC_3) | pCfB8843/(Arnesen et | BB3927 |
| | al, 2020) | |
| pCfB8822 (IntC_2-HMG1<-PrGPD-PrTefInt- | pCfB6682/(Arnesen et | BB3865, BB3866, |
| >ERG12) | al, 2020) | BB3867 |
| pCfB8823 (IntC_3-SeACS<-PrGPD-PrTefInt- | pCfB6371/(Arnesen et | BB3865, BB3868, |
| >YIACL1) | al, 2020) | BB3869 |
| pCfB8878 (IntD_1-IDI1<-PrGPD-PrTefInt- >ERG20) | pCfB6684/(Arnesen et | BB3865, BB3870, |
| | al, 2020) | BB3871 |
| pCfB6605 (pIntE-4-Hph-PrExp->YISQS1) | (Arnesen et al, 2020) | |
| pCfB6630 (pNat-YLgRNA3_IntC_3) | (Holkenbrink et al, 2018) | |
| pCfB7063 (prDNA-Ura3d1- | (Kildegaard et al, 2017) | |
| TPex20 + PTEfintron + CrtW + TLip2) | | |
| pCfB5119 (pIntB-HphMx-YIHMG1<-PrGPDPrFBA1- | (Kildegaard et al, 2017) | |
| >YIGGS1) | | |
| pCfB6620 (pORI1001-Nat-CEN1-USER-IDI1<- | (Arnesen et al, 2020) | |
| PrEXP-PrGPD->ERG20) | | |

TABLE 7
Biobricks for construction of Yarrowia lipolytica platform strain for sterol production

| | | Forward | Reverse |
|---|---|---|---|
| BioBrick name | Template/Reference | primer | primer |
| BB3924 (Episomal | pCFB3405 | PR-23934 | PR-10593 |
| vector backbone w/o | | | |
| HphMX) | | | |
| BB3925 (HphMX-TTef1 | pCfB6605 | PR-23935 | PR-23936 |
| insert) | | | |
| BB3927 (gRNA-cassette | pCfB6630 | PR-10607 | PR-10604 |
| IntC_3) | | | |
| BB3863 | pCfB7063 | PR-24013 | PR-18214 |
| (Tefint(PrGDPfusion)->) | | | |
| BB3864 (<-PrGDP) | pCfB5119 | PR-15528 | PR-15529 |
| BB3865 (<- | BB3863, BB3864 | PR-15528 | PR-18214 |
| PrGDP_Tefint->) | | | |
| BB3866 (<-HMG1) | pCfB5119 | PR-23753 | PR-23752 |
| BB3867 (ERG12->) | ST3683 gDNA | PR-24014 | PR-24015 |
| BB3868 (<-YIOpSeACS) | Salmonella enterica acetyl-CoA synthetase | PR-24016 | PR-24017 |
| | Y. lipolytica codon optimised | | |
| BB3869 (YIACL1->) | ST3683 gDNA | PR-24018 | PR-24019 |
| BB3870 (<-IDI1) | ST3683 gDNA | PR-24020 | PR-24021 |
| BB3871 (ERG20->) | pCfB6620 | PR-24022 | PR-24023 |

TABLE 8
Primers for construction of Yarrowia lipolytica platform strain for sterol production

| Primer name | Sequence (5′ → 3′) |
|---|---|
| PR-23934 (PrEXP for- | ACCCATTGCTGUAGATATGTCTTGTGTGTAAGGGGG |
| HpHMX_rv) | |
| PR-10593 (Fragment2EpiVecYL_ | AGCAGGCTUGGAGGCGACGTGGCAG |
| fw) | |
| PR-23935 (HphMX_for- | ACAGCAATGGGUAAAAAGCCTGAACTCACCGC |
| PrEXP1_fw) | |
| PR-23936 (TTef1_rv) | AAGCCTGCUGAATTCGGACACGGGCAT |
| PR-10604 (tracrRNA_rev) | CACGCGAUACCGTACCCACACAAAAAAAGCACCACCGACTC |
| PR-10607 (PrtRNAGly_fw) | CGTGCGAUAGTGAATCATTGCTAACAGATC |
| PR-24013 (Tefint_Fuse_fw) | ATCAGTAGCUAGAGACCGGGTTGGCGGCG |
| PR-18214 | AGTACTGCAAAAAGUGCTG |
| (PTEFintron_USER_rv) | |
| PR-15528 (PrYITDH1_rev) | ATGACAGAUTGTTGATGTGTGTTTAATTCAAGAATG |
| PR-15529 (PrYITDH1for | AGCTACTGAUGACGCAGTAGGATGTCCTGCACGG |
| fusion_fw) | |
| PR-23752 (YIHMG1 →_U2_fw) | ATCTGTCAUGCCACAATGCTACAAGCAGCTATTGG |
| PR-23753 (YIHMG1 →_U2_rv) | CACGCGAUCTATGACCGTATGCAAATATTCG |
| PR-24014 (ERG12_fw) | ACTTTTTGCAGTACUAACCGCAGGACTACATCATTTCGGCGCC |
| PR-24015 (ERG12_rev) | CACGCGAUCTAATGGGTCCAGGGACCG |
| PR-24016 (OpSeACS_fw) | CGTGCGAUTTAAGAGGGCATAGCAATGGCC |
| PR-24017 (OpSeACS_rev) | ATCTGTCAUGCCACAATGTCTCAGACCCACAAGCACG |
| PR-24018 (YIACL1_fw) | ACTTTTTGCAGTACUAACCGCAGTCAGCGAAATCCATTCACGAG |
| PR-24019 (YIACL1_rev) | CACGCGAUTTAAACTCCGAGAGGAGTGGAA |
| PR-24020 (YIIDI1_fw) | CGTGCGAUCTACTTGATCCACCGCCGAA |
| PR-24021 (YHIDI1_rev) | ATCTGTCAUGCCACAATGACGACGTCTTACAGCGA |
| PR-24022 (ERG20_fw) | ACTTTTTGCAGTACUAACCGCAGTCCAAGGCGAAATTCGAAAGC |
| PR-24023 (ERG20_rv) | CACGCGAUCTACTTCTGTCGCTTGTAAATCTT |

### Yeast Transformation

The yeast vectors were integrated into different previously characterized intergenic loci in the Y. lipolytica genome as described in Holkenbrink et al, 2018; see also Table 18 below. Integration vectors were digested with Notl enzyme (New England Biolabs) at 37° C. for 1 hr and the digested product purified from solution using the NucleoSpin® Gel and PCR Clean-up kit (Macherey-Nagel). The purified DNA was transformed using the lithium acetate transformation protocol as described by Holkenbrink et al, 2018. Correct integration was verified by colony PCR using Taq DNA Polymerase Master Mix RED (Ampliqon) with vector-specific primers and primers complementary to the genomic region adjacent to the integration site as described by Holkenbrink et al. (2018).

### Results

Yarrowia lipolytica strain ST9100 (MATa ku70Δ::PrTEF1-Cas9-TTef12::PrGPD-DsdA-TLip2 IntC_2-HMG1←PrGPD-PrTeflnt→ERG12 pCfB8823 IntC_3-SeACS←PrGPD-PrTeflnt→YIACL1 IntD_1-IDI1←PrGPD-PrTeflnt→ERG20) was constructed from ST6512 by consecutive rounds of engineering via firstly construction of ST8980 and then ST9027 as indicated in Table 5α above and also described in Arnesen et al. (2020) Front. Bioeng. Biotech. This was chosen as the platform Y. lipolytica strain for further sterol production studies for reasons given below.

Y lipolytica is an oleaginous species with a high acetyl-coenzyme A (CoA) flux, the main precursor to the mevalonate (MVA) and subsequent sterol pathways, which makes it valuable for sterol production. Two key approaches were used to develop the platform strain: improvement of the acetyl-CoA pool and up-regulation of the MVA-pathway to improve the accumulation of isopentyl diphosphate (IPP)/dimethylallyl diphosphate (DMAPP). The acetyl-CoA pool was increased by overexpressing the native ATP citrate lyase 1 (ACL, YALI0_D24431 g, Y. lipolytica CLIB122 genome assembly Dujon et al, 2004) and the Salmonella enterica acetyl-CoA synthetase (SeACS). In Y. lipolytica. Aclp generates acetyl-CoA and oxaloacetate from citrate, whilst SeAcsp produces acetyl-CoA from acetate and COA. Flux through the mevalonate pathway was increased by overexpression of native 3-hydroxy-3-methylglutaryl-CoA reductase (HMG, YALI0_E04807 g), mevalonate kinase (ERG12, YALI0_B16038 g), isopentyl diphosphate isomerase (IDI, YALI0_F004015 g) and farnesyl diphosphate synthase (ERG20, YALI0_E05753 g) genes.

### REFERENCES

Arnesen J A, Kildegaard K R, Cernuda Pastor M, Jayachandran S, Kristensen M & Borodina I (2020) Yarrowia lipolytica Strains Engineered for the Production of Terpenoids. Front. Bioeng. Biotechnol. 8: 945 Available at: www.frontiersin.org
Dujon B, Sherman D, Fischer G, Durrens P, Casaregela S, Lafentaine I, De Montigny J, Marck C, Neuvéglise C, Talia E, Goffard N, Frangeul L, Algie M, Anthouard V. Babour A, Barbe V, Barnay S. Blanchin S, Beckerich J M, Beyne E. et al (2004) Genome evolution in yeasts. Nature 430: 35-44 Available at: https://pubmed.ncbi.nlm.nih.gov/15229592/
Holkenbrink C. Dam M I. Kildegaard K R, Beder J, Dahlin J, Doménech Belda D & Borodina I (2018) EasyCloneYALI: CRISPR/Cas9-Based Synthetic Toolbox for Engineering of the Yeast Yarrowia lipolytica. Biotechnol. J.
Marella Er, Dahlin J, Dam M I, ter Horst J, Christensen H B, Sudarsan S, Wang G, Holkenbrink c & Borodina I (2020) A single-host fermentation process for the production of flavour lactones from non-hydroxylated fatty acids. Metabol. Eng. 61, 427-438

### Example 2—Engineering Yarrowia Lipolytica Strains Incapable of Synthesising Ergosterol

### Materials and Methods

### Strains, Culture Conditions and Chemicals

Escherichia coli strain DH5α was used for plasmid construction as noted in Example 1 above.

The Y. lipolytica strain ST4840 (ATCC® 34088™/Y-17536 ARS Culture Collection, NCAUR, United States), and W29-derived strains ST4842 (MatA, ATCC® 20460™/Y-63746), ST6512 (MatA ku70Δ:PrTEF1→Cas9-TTef12:PrGPD→DsdA-TLip2, Marella et al. 2020) and ST9100 (MatA ku70Δ::PrTEF1-Cas9-TTef12::PrGPD-DsdA-TLip2 IntC_2-HMG1←PrGPD-PrTefInt→ERG12 pCfB8823 IntC_3-SeACS←PrGPD-PrTefInt→YIACL1 IntD_1-IDI1←PrGPD-PrTeflnt→ERG20, Arnesen et al, 2020) were obtained from Irina Borodina (Novo Nordisk Foundation Centre for Biosustainability, Technical University of Denmark, Copenhagen). All strains are detailed in Table 5.

Y. lipolytica was grown at 30° C. on yeast extract peptone dextrose (YPD) media containing 10 g/i yeast extract, 20 g/l peptone, and 20 g/l glucose, supplemented with 20 g/l agar for preparation of solid media. For selection, either nourseothricin (250 mg/l) or hygromycin (400 mg/l) was added to the media. Where indicated, 25 μl of ergosterol dissolved in 1:3 ether-hexane (0.002% w/v) was added to each plate. Solvent was allowed to evaporate prior to use.

### Yeast Cultivation for Sterol Production

Cultivation of strains for sterol production was performed in yeast extract peptone medium containing 80 g/l glucose. Yeast strains were inoculated into 2.5 ml YPD in 24-deepwell plates with air-penetrable lids (EnzyScreen, Netherlands). The plates were incubated at 30° C. with 300 rpm agitation at 5 cm orbit cast for 24 hours. The cultures were then diluted to OD₆₀₀ 0.1 in 2.5 ml fresh YPD-media with 80 g/l glucose and grown for a further 72 hours at 30° C. with 300 rpm agitation. All cultivations were performed in triplicate. Cel dry weight was measured at the end of cultivation: 1 ml of culture broth was transferred into a pre-weighed 2 ml microcentrifuge tube, centrifuged (3000 g, 5 min) and the supernatant was discarded. The cells were washed twice with deionized water (1 ml). The cell pellet was dried at 60° C. for 7 days before the final weight was measured.

Chemicals were obtained, if not indicated otherwise, from Sigma-Aldrich. Nourseothricin was purchased from Jena BioScience GmbH (Germany).

### Plasmid Construction

The plasmids, BioBricks, and primers used in this study are listed in Tables 9-11, respectively.

TABLE 9
Plasmids for engineering Yarrowia lipolytica strains incapable of synthesising
ergosterol

| | Parent plasmid/ | |
|---|---|---|
| Plasmid name | Reference | BioBricks |
| pCfB8861 (pHyg- | (Arnesen et al, | |
| YLgRNA3_IntE_4) | 2020) | |
| pCfB6679 (pintE_4-TPex20-TLip2) | (Holkenbrink et al, | |
| | 2018) | |
| pCfB10252 (IntE4_PrTEF- | pcf86679 | BB3879: BB4879 |
| >DHCR7_Esilicul) | | |
| pCfB10367 (Erg5_tPr48) | | BB1135: BB5040: BB5101: BB5044 |
| pCfB3405 (pORI1001-Nat-CEN1- | (Holkenbrink et al, | |
| USER) | 2018) | |
| pCfB8843 (pORI1001-Hyg-CEN1- | (Arnesen et al, | |
| USER) | 2020) | |
| pCfB10243 | pCfB8843 | BB1635: BB1636: PR-27625: PR-27626 |
| (pERG4_gRNA_KO_Hyg) | (pORI1001-Hyg- | |
| | CEN1-USER) | |
| pCfB10244 | pCFB8843 | BB1635: BB1636: PR-27627: PR-27628 |
| (pERG5_gRNA KO_Hyg) | (pORI1001-Hyg- | |
| | CEN1-USER) | |
| pCfB10245 | pCFB8843 | BB1635: BB1636: PR-27629: PR-27630 |
| (pERG6_gRNA_KO_Hyg) | (pORI1001-Hyg- | |
| | CEN1-USER) | |
| pCfB10246 | pCfB3405 | BB1635: BB1636: PR-27625: PR-27626 |
| (pERG4_gRNA_KO_Nat) | (pORI1001-Nat- | |
| | CEN1-USER) | |
| pCfB10247 | pCfB3405 | BB1635: BB1636: PR-27627: PR-27628 |
| (pERG5_gRNA_KO_Nat) | (pORI1001-Nat- | |
| | CEN1-USER) | |
| pCfB10248 | pCfB3405 | BB1635: BB1636: PR-27629: PR-27630 |
| (pERG6_gRNA_KO_Nat) | (pORI1001-Nat- | |
| | CEN1-USER) | |
| pCfB10347 | pCFB8843 | BB1635: BB1636: PR-27929: PR-27930 |
| (pERG4_gRNA2_KO_Hyg) | (pORI1001-Hyg- | |
| | CEN1-USER) | |
| pCfB10348 | pCF88843 | BB1635: BB1636: PR-27931: PR-27932 |
| (pERGS_gRNA2_KO_Hyg) | (pORI1001-Hyg- | |
| | CEN1-USER) | |
| pCfB10349 | pCF88843 | BB1635: BB1636: PR-27933: PR-27934 |
| (pERG6_gRNA2_KO_Hyg) | (pORI1001-Hyg- | |
| | CEN1-USER) | |
| pCfB10350 | pCfB3405 | BB1635: BB1636: PR-27929: PR-27930 |
| (pERG4_gRNAZ_KO_Nat) | (pORI1001-Nat- | |
| | CEN1-USER) | |
| pCfB10351 | pCfB3405 | BB1635: BB1636: PR-27931: PR-27932 |
| (pERGS_gRNA2_KO_Nat) | (pORI1001-Nat- | |
| | CEN1-USER) | |
| pCfB10352 | pCfB3405 | BB1635: BB1636: PR-27933: PR-27934 |
| (pERG6_gRNA2_KO_Nat) | (pORI1001-Nat- | |
| | CEN1-USER) | |
| pCfBS935 (pintA-1-HphMx- | (Holkenbrink et al, | |
| TPex20-TLip2) | 2018) | |
| pCf84788 (pintA_1-Nat-TPex20- | (Holkenbrink et al, | |
| TLip2) | 2018) | |
| pCf86677 (pintE_1-TPex20-TUp2) | (Holkenbrink et al, | |
| | 2018) | |
| pCfB10494 (IntE3_PrGPAT- | pCfB6681 | BB1617 (PrGPAT): BB5100 (TtSTC_USER) |
| >TISTC) | | |
| pCfB10224 (ERG4_KORep_90 bp) | DNA Duplex | Sequence: ACATAAAGCCATCCACCCTTCCTT |
| | Synthesis from IDT | CGCAACCACACACACCATACAACGATAAGCT |
| | (n.b. NOT a plasmid) | TAGTGAGCGAATGGTGAGGTTACTTAATTGA |
| | | GTGG |
| pCf810225 (ERG5_KORep_90 bp) | DNA Duplex | Sequence: AACTTCTCTCTCTCACACCACCA |
| | Synthesis from IDT | CCACAACACAACTCCGCCCACCGGTATATAG |
| | (n.b. NOT a plasmid) | GTTTGGTAATGTATTAATATTAATGATGGGG |
| | | CGAGG |
| pCfB10226 (ERG6_KORep_90 bp) | DNA Duplex | Sequence: |
| | Synthesis from IDT | GATCTGAATCGCCCTTGTAAACCC |
| | (n.b. NOT a plasmid) | CCCCAAAACACCACATTCAACACTGAGTAACT |
| | | TATAGAGGGAGCCACGGCCCCAAAATTTATA |
| | | ATG |

TABLE 10
Biobricks for engineering Yarrowia lipolytica strains incapable of synthesising ergosterol

| | | Forward | Reverse |
|---|---|---|---|
| BioBrick name | Template/Reference | primer | primer |
| BB1635 (gRNA Pr) | (Holkenbrink et al, 2018) | | |
| BB1636 (gRNA Ter) | (Holkenbrink et al, 2018) | | |
| BB3879 (PrTefInt->) | (Arnesen et al, 2020) | | |
| BB4879 (PrTEF_DHCR7Es) | Ectocarpus siliculosus delta-7 sterol reductase | PR-27571 | PR-27572 |
| | codon optimised | | |
| BB5356 | ST9100 gDNA | PR-27891 | PR-27892 |
| (Erg4_1 kbUP_U2) | | | |
| BB5092 | ST9100 gDNA | PR-27893 | PR-27894 |
| (Erg4_1 kbDOWN_U2) | | | |
| BB5359 | BB5356 + BB5092 | PR-27891 | PR-27894 |
| (Erg4_2 kb_Rtemp) | | | |
| BB5357 | ST9100 gDNA | PR-27895 | PR-27896 |
| (Erg5_1 kbUP_U2) | | | |
| BB5093 | ST9100 gDNA | PR-27897 | PR-27898 |
| (Erg5_1 kbDOWN_U2) | | | |
| BB5360 | BB5357 + BB5093 | PR-27895 | PR-27898 |
| (Erg5_2 kb_Rtemp) | | | |
| BB5358 | ST9100 gDNA | PR-27899 | PR-27900 |
| (Erg6_1 kbUP_U2) | | | |
| BB5094 | ST9100 gDNA | PR-27901 | PR-27902 |
| (Erg6_1 kbDOWN_U2) | | | |
| BB5361 | BB5358 + BB5094 | PR-27899 | PR-27902 |
| (Erg6_2 kb_Rtemp) | | | |
| BB5093 | ST9100 gDNA | PR-27891 | PR-28358 |
| (ERG4_1 kbUP_U1) | | | |
| BB5093 | ST9100 gDNA | PR-27895 | PR-28359 |
| (ERG5_1 kbUP_U1) | | | |
| BB5093 | ST9100 gDNA | PR-27899 | PR-28360 |
| (ERG6_1 kbUP_U1) | | | |
| BB5041 (LoxP_HygMX) | pCfB5935 | PR-27972 | PR-27973 |
| BB5101 (LoxP_NatMX) | pCfB4788 | PR-27972 | PR-27973 |
| BB5097 | BB5091 + BB5092 + BB5041 | PR-27891 | PR-27894 |
| (Erg4_HphMX_KO) | | | |
| BB5098 | BB5093 + BB5094 + BB5041 | PR-27895 | PR-27898 |
| (Erg5_HphMX_KO) | | | |
| BB5099 | BB5095 + BB5096 + BB5041 | PR-27899 | PR-27902 |
| (Erg6_HphMX_KO) | | | |
| BB5246 | BB5091 + BB5092 + BB5101 | PR-27891 | PR-27894 |
| (Erg4_NatMX_KO) | | | |
| BB5247 | BB5093 + BB5094 + BB5101 | PR-27895 | PR-27898 |
| (Erg5_NatMX_KO) | | | |
| BB5248 | BB5095 + BB5096 + BB5101 | PR-27899 | PR-27902 |
| (Erg6_NatMX_KO) | | | |
| BB5040 | ST9100 gDNA | PR-27970 | PR-27971 |
| (Erg5_UPintergenic) | | | |
| BB5044 | ST9100 gDNA | PR-27976 | PR-27969 |
| (Erg5_tPr48_ORF996) | | | |
| BB1135 (Easy Clone | pCfB6677 | PR-11110 | PR-11111 |
| vector backbone) | | | |
| BB1617 (PrGPAT->) | (Holkenbrink et al, 2018) | | |
| BB5100 (TtSTC_USER) | Tetrahymena thermophilia squalene- | PR-28361 | PR-28362 |
| | tetrahymanol cyclase codon optimised | | |

TABLE 11
Primers for engineering Yarrowia lipolytica strains incapable of synthesising
ergosterol

| Primer name | Sequence (5′ → 3′) |
|---|---|
| PR-14617 (vector verification E. | tatccctgtgttgaatc |
| coli cPCR) | |
| PR-14619 (vector verification E. | tatcgacccagttagc |
| coli cPCR) | |
| PR-8859 (integration verification | aagtgtggatggggaagtgag |
| YI cPCR) | |
| PR-14576 (IntE_3 verification YI | cacgcgautgaaggaaatgcctaaaacc |
| cPCR) | |
| PR-14835 (IntE 3 verification YI | cacgcacgccattctataag |
| cPCR) | |
| PR-14592 (IntE_4 verification YI | acgcgauttaacactggaccgtactgc |
| cPCR) | |
| PR-20880 (IntE_4 verification YI | attgctaagcgaccatagac |
| cPCR) | |
| PR-27571 (A_Es_Fw) | actttttgcagtacuaaccgcagatcgacggcgctgccatcg |
| PR-27572 (A_Es Rv) | cacgcgauttacaggatgccgggcacgatc |
| PR-27625 (ERG4_gRNA_Fw) | ggtctcgtactgcttgacagcgggttttagagct |
| PR-27626 (ERG4_gRNA_Rv) | ccgctgtcaagcagtacgagacctaaccaacct |
| PR-27627 (ERG5_gRNA_Pw) | gtagggaaccataatgacagggggttttagagct |
| PR-27628 (ERG5_gRNA_Rv) | cccctgtcattatggttccctactaaccaacct |
| PR-27629 (ERG6_gRNA_Fw) | agaaacaaacagcatcatggggggttttagagct |
| PR-27630 (ERG6_gRNA_Rv) | cccccatgatgctgtttgtttcttaaccaacct |
| PR-27891 (Erg4Rep_P1F) | ctctcaacaccttcaccgc |
| PR-27892 (Erg4Rep_P1R) | acctgcacuacgataagcttagtgagcg |
| PR-27893 (Erg4Rep_P2F) | gtgcaggutgtgtggttgcgaaggaag |
| PR-27894 (Erg4Rep_P2R) | gcactcaaaataccccgttc |
| PR-27895 (Erg5Rep_P1F) | ctcggtttgttgcagcagg |
| PR-27896 (Erg5Rep_P1R) | acctgcacuatggtccgtatcgtgaaatg |
| PR-27897 (Erg5Rep_P2F) | gtgcaggugggggagttgtgttgtg |
| PR-27898 (Erg5Rep_P2R) | ggtcggctatccaatacatctc |
| PR-27899 (Erg6Rep_P1F) | gctacaagccggaggggaac |
| PR-27900 (Erg6Rep_PIR) | acctgcacugttgaatgtggtgttttggg |
| PR-27901 (Erg6Rep_P2F) | gtgcagguactgagtaacttatagaggg |
| PR-27902 (Erg6Rep_P2R) | ctgtaccgtttggaggactc |
| PR-27929 (ERG4_gRNA2_Fw) | atctcgtcgacctactacgagttttagagct |
| PR-27930 (ERG4_gRNA2_Rv) | tcgtagtaggtcgacgagattaaccaacct |
| PR-27931 (ERG5_gRNA2_Fw) | tgagaaatacaaggcccagtgttttagagct |
| PR-27932 (ERG5_gRNA2_Rv) | actgggccttgtatttctcataaccaacct |
| PR-27933 (ERG6_gRNA2_Fw) | ttcccgatactacaagggaggttttagagct |
| PR-27934 (ERG6_gRNA2_Rv) | ctcccttgtagtategggaataaccaacct |
| PR-27969 (Erg5ORF_R_U3) | cacgcgautccgtccgcaacaatctg |
| PR-27970 | cgtgcgautaagcatgcatcggacac |
| (Erg5_UP_WORF_F_U3) | |
| PR-27971 | atcgcacgugatcgtgtgagtcagagg |
| (ErgS_UP_uORF_R_U1) | |
| PR-27976 (ERG5_tPrSO_F_U2) | agtgcaggucacaacttctctctctcacac |
| PR-27972 (Hyg/NatMX_F_U1) | cgtgcgautcagctgaagcttcgtac |
| PR-27973 (Hyg/NatMX_R_U2) | acctgcacugcataggccactagtgg |
| PR-28358 (Erg4Rep_P1R_U1) | atcgcacgugataagcttagtgagcgaatgg |
| PR-28359 (Erg5Rep_PR_U1) | atcgcacgutggtccgtatcgtgaaatgg |
| PR-28360 (Erg6Rep_P1R_U1) | atcgcacgucaagggcgattcagatcagc |
| PR-27631 (ERG4 KOchk_Fw) | cctgatattgatgatcctcc |
| PR-27632 (ERG4 KOchk_Rv1) | agagccttgtttccga |
| PR-27633 (ERG4 KOchk_Rv2) | atacaatcccataggctggc |
| PR-11138 | agcaatggguaaaaagcctgaactcaccgc |
| (HphMX_KOcasette_chkFw) | |
| PR-28001 (Derg4_chkRv) | cgtgcgaugcttgccctggactacatcttg |
| PR-27634 (ERGS_KOchk_Fw) | acttctctctctcacaccacc |
| PR-27635 (ERG5_KOchk_Rv1) | ctgagggctctgttggtgaag |
| PR-27636 (ERG5_KOchk_Rv2) | accagtgtggttgtaaggatg |
| PR-22830 | tcatactcaccgaaacgtg |
| (HphMX_KOcasette_chkRv) | |
| PR-27970 (Derg5_chkRv) | cgtgcgautaagcatgcatggacac |
| PR-27637 (ERG6_KOchk_Fw) | ctcgcatacttcccgtttag |
| PR-27638 (ERG6_KOchk_Rv1) | tgagaccgacaatgttggc |
| PR-27639 (ERG6_KOchk_Rv2) | ccaccgatccttctcagctac |
| PR-26681 | atccgctctaaccgaaaagg |
| (NatMX_KOcasette_chkFw) | |
| PR-28007 (Derg6_chkFw) | cgtgcgaugaaggagatactggtgcc |
| PR-11110 (E. coli backboneUSER_ | atcgcgtgcattcgcggccgcatttaaatcc |
| fw) | |
| PR-11111 (E. coli backboneUSER_ | atcgcacgcattcgcggccgcaaatttaaataaaagatg |
| rev) | |
| PR-28361 (TISTC_F_U3) | atctgtcaugccacaatgaagaagatcctcatcggtc |
| PR-28362 (TISTC_Rv) | cacgcgauttagatgttctgcttctggacg |

BioBricks were again amplified by PCR. The PCR mix contained 32 μl water, 10 μl high fidelity Phusion® polymerase buffer (5×), 1 μl 10 mM dNTP, 1.5 μl MgCl₂, 2.5 μl 10 μM Fw primer, 2.5 μl 10 μM Rv primer, 0.2 μl Phusion® U polymerase and 10 ng DNA template. Reactions were multiplexed 8× per BioBrick. The cycling program was: 98° C. for 1 min, 30 cycles of [98° C. for 10 sec, gradient from 50 to 58° C. for 20 sec. 72° C. for 30 s/kb], 72° C. for 5 min, pause at 10° C. The gene fragments were purified from agarose gels using the NucleoSpin® Gel and PCR Clean-up kit (Macherey-Nagel). Biobricks were assembled into the EasyCloneYALI vectors with USER cloning (Holkenbrink et al, 2018). The USER reactions were transformed into E. coli and correct assemblies were verified by Sanger sequencing (Eurofins).

The delta-7 sterol reductase from Ectocarpus silculosus (GenBank accession: CBN77313.1) and the squalene-tetrahymanol cyclase from Tetrahymena thermophila (accession: XP_001026698.2) were codon-optimized for Y. lipolytica and synthesised as GeneArt String DNA fragments by ThermoFischer Scientific. The codon-optimized sequences are given in Table 4. 90 bp repair templates were synthesised as double-stranded DNA oligos (IDT DNA). Longer repair templates were generated using DNA fragments comprising upstream and downstream homology arms synthesis by PCR amplification from gDNA.

### Yeast Transformation

The yeast vectors and biobricks were transformed into Y. lipolytica using the lithium acetate transformation protocol as described by Holkenbrink et ai, 2016. Vectors were digested prior to transformation with Notl enzyme (New England Biolabs) at 37° C. for 1 hr and the digested product purified from solution using the Nucleospin® Gel and PCR Clean-up kit (Macherey-Nagel). Transformants were selected on antibiotic supplemented plates and correct transformants confirmed by colony PCR using Taq DNA Polymerase Master Mix RED (Ampliqon) with vector-specific primers and primers complementary to the genomic region adjacent to the integration site.

In summary, for marker-mediated gene deletion, Y. lipolytica strains were transformed with biobricks assembled by USER reaction as further detailed below. Transformants were selected on antibiotic supplemented plates and correct transformants confirmed by colony PCR. Marker removal was performed by transformation of the strains with a Cre-recombinase episomal vector. Marker removal was confirmed by colony PCR.

### Results

Unsuccessful ERG4/ERG5/ERG6 Knock-Out Attempts with Guide RNA Mediated Gene Deletion

Guide RNA (gRNA) vectors against ERG4 (YALI1_D24361 g), ERG5 (YALI1_A18344 g) and ERG6 (YALI1_F12138 g) were assembled as described by Holkenbrink et al. 2018. For each of ERG4, ERG5 and ERG6, strain ST9100 was transformed with 500 ng gRNA (pCfB10243 to pCfB10245) and 2 nmole of the corresponding 90 bp repair template. Transformants were selected on YPD plates supplemented with hygromycin. Gene deletion was examined by colony PCR; no colonies contained deletions in the relevant ERG genes.

The transformation was repeated with 1 μg gRNA vector and 4 nmole of the corresponding repair template. Transformants were selected on YPD plates supplemented with hygromycin and ergosterol. No colonies contained deletions in the relevant ERG genes.

Ectocarpus silculosus delta-7 sterol reductase was integrated into the ST9100 genome under the control of the PrTEFintron promoter, generating strain ST10924. Strain ST10924 was transformed with 1 μg ERG5 gRNA vector (pCfB10247) and 4 nmole of repair template. Transformants were selected on YPD plates supplemented with nourseothricin and ergosterol. No colonies contained deletions in the relevant ERG genes.

New repair templates were constructed comprising 1 kb homology arms either side of the ERG4/5/6 coding sequences, amplified from ST9100 gDNA (˜2 kb total length). ST9100 was transformed with 500 ng gRNA (pCfB10243 to pCfB10245) and 500 ng of the corresponding 2 kb repair template. Transformants were selected on YPD plates supplemented with hygromycin and ergosterol. Gene deletion was examined by colony PCR; no colonies contained deletions in the relevant ERG genes.

Strain ST10924 was transformed with 500 ng ERG5 gRNA vector (pCfB10247) and 500 ng of repair template. Transformants were selected on YPD plates supplemented with nourseothricin and ergosterol. No colonies were obtained.

For each of ERG4, ERG5 and ERG8, new gRNAs were designed using the best performing crRNA sequences as identified by Schwartz et al., 2019. Strain ST9100 was transformed with 500 ng gRNA (pCfB10347 to pCfB10352) and 500 ng of the corresponding 2 kb repair template. Transformants were selected on YPD plates supplemented with hygromycin and ergosterol. Gene deletion was examined by colony PCR; no colonies contained deletions in the relevant ERG genes.

Strains ST9100 and ST10924 were transformed with 1 μg ERG5 gRNA (pCfB10244 to pCfB10348) and 500 ng of the corresponding 2 kb repair template. Transformants were selected on YPD plates supplemented with hygromycin and ergosterol. Gene deletion was examined by colony PCR; no colonies contained deletions in the relevant ERG genes.

### Resistance Marker-Mediated Gene Deletion

New ERG4/5/6 repair templates were constructed for marker-mediated knock-out, comprising 1 kb homology arms either side of a hygromycin (HphMX) or nourseothricin (NatMX) resistance marker. Strains ST9100 and ST10924 were transformed with 5 μg of the ERG5 HphMX cassette.

Transformants were selected on YPD plates supplemented with hygromycin and YPD plates supplemented with hygromycin and ergosterol. Gene deletion was examined by colony PCR; no colonies contained deletions in the relevant ERG genes.

Strains ST9100. ST4842 and ST4840 were transformed with 2 μg of the ERG5 HphMX cassette. Transformants were selected on YPD plates supplemented with hygromycin and YPD plates supplemented with hygromycin and ergosterol. Gene deletion was examined by colony PCR; no colonies contained deletions in the relevant ERG genes.

Strains ST9100 and ST4842 were transformed with 5 μg of the ERG4 NatMX cassette. Transformants were selected on YPD plates supplemented with hygromycin and YPD plates supplemented with hygromycin and ergosterol. Gene deletion was examined by colony PCR; no colonies contained deletions in the relevant ERG genes.

The ERG5 promoter was truncated to 48 bp to down-regulate ERG5 expression. A cassette was generated comprising a NatMX resistance marker flanked by two homology arms: the region between 2.5 and 1.5 kb upstream of the ERG5 coding sequence, and the region from 48 bp upstream of the start codon plus 996 bp of the ERG5 open reading frame. The cassette was assembled into a plasmid and transformed into ST10924 to generate ST10934. ERG5 promoter truncation was confirmed by sequencing. Strain ST10934 was transformed with 7.5 μg of either the ERG4 or ERG5 HphMX cassettes. Transformants were selected on YPD plates supplemented with hygromycin. No colonies were obtained.

Efficient ERG4/ERG5/ERG6 Knock-Out with Intracellular Provision of Tetrahymanol

The gene encoding squalene-tetrahymanol cyclase from Tetrahymena thermophila (TtSTC) was integrated into the ST9100 platform strain genome under the control of the PrGPAT promoter, previously characterised as a weak promoter (Holkenbrink et al, 2018). This promoter was selected to drive minimal expression of TtSTC to limit diversion of carbon flux away from the main sterol pathway. [Note: the promoter described as ‘PrGPAT’ in Holkenbrink et al. 2018 and used in this study does not belong to the Y. lipolytica GPAT gene (YALI1_C00230 g, (Magnan et al. 2016), but is instead the sequence corresponding to the promoter region of gene YALI1_C00209 g in Y. lipolytica W29 Y-63746 with genomic location of the promoter being Chromosome 1C20927-22056, and genomic location of the downstream gene being Chromosome 1C: 18,422-20,926.] The resulting strain ST11005 produced 0.4 mg/g dry cell weight (DCW) tetrahymanol, comprising 14.0% of the total sterol fraction.

Construction of Yarrowia lipolytica Strains Incapable of Synthesizing Ergosterol

The tetrahymanol producing strain ST11005 was used for the construction or strains incapable of synthesising ergosterol. Firstly, to create a ΔERG5 strain, the ERG5 gene (sterol C-22 desaturase, YALI1_A18344 g) was deleted by marker-mediated knock out. A knock-out cassette was constructed comprising a hygromycin resistance marker (HphMX) flanked by 1 kb homology arms corresponding to 1 kb upstream and downstream of the ERG5 coding sequence. This construct was used to transform ST11005 thereby generating ST11014.

ST11005 was transformed with 3 μg of the ERG5 HphMX cassette. Transformants were selected on YPD plates supplemented with hygromycin. Gene deletion was examined by colony PCR; all colonies tested contained deletion of ERG5 (ST11014).

The HphMX resistance marker was subsequently looped out by Cre-lox recombination. The resulting strain ST11027 produced 3.1 mg/g DCW ergosta-5,7-dienol, comprising 60.0% of the total sterol fraction. The strain also produced 5.2 mg/g DCW tetrahymanol, comprising 36.7% of the total sterol fraction.

To create a ΔERG4ΔERG5 strain, the ERG4 gene (delta-24 sterol reductase, YALI1_D24361 g) was deleted by HphMX marker-mediated knockout in an analogous manner. The resulting strain ST11040 produced 3.1 mg/g DCW ergosta-5,7,24(28)-trienol, comprising 20.0% of the total sterol fraction. The strain also produced 15.8 mg/g DCW tetrahymanol, comprising 75.2% of the total sterol fraction.

Alternatively, strain ST11014 was transformed with 4 μg of the ERG4 NatMX cassette. Transformants were selected on YPD plates supplemented with nourseothricin. Gene deletion was examined by colony PCR; all colonies tested contained deletion of ERG4 (ST11028).

To create a ΔERG5ΔERG6 strain, the starting yeast strain was ST11027 (the strain obtained following the HphMX resistance marker in the ERG5 locus of ST11014 being looped out by Cre-Lox recombination). The gene encoding ERG6 (sterol methyl transferase, YALI1_F12138 g) was deleted from ST11027 by HphMX marker-mediated knockout in an analogous manner, generating strain ST11325. The HphMX resistance marker was subsequently looped out by Cre-Lox recombination. The resulting strain ST11330 produced 0.2 mg/g DCW zymosterol, comprising 0.6% of the total sterol fraction. The strain also produced 36.0 mg/g DCW tetrahymanol, comprising 99.4% of the total sterol fraction.

Strain ST11027 was transformed with 2 μg of the ERG6 HphMX cassette. Transformants were selected on YPD plates supplemented with hygromycin. Gene deletion was examined by colony PCR; most colonies tested contained deletion of ERG5 (ST11325).

Compared to the wild-type strains ST4840 and ST4842, strain ST6512 is optimised for greater efficiency of homologous recombination. ST6512 was transformed with 8 μg of either the ERG4 or ERG5 NatMX cassettes. Transformants were selected on YPD plates supplemented with nourseothricin. Gene deletion was examined by colony PCR. In each case, only one of the colonies tested contained deletion of the relevant gene (ST11326 and ST11327).

### DISCUSSION

Strains used in this work derived from Y. lipolytica W29 strain Y-63746 (MatA, ARS Culture Collection, NCAUR, United States, ATCC20460™). Strains used in prior work included the E122-derived strain ATCC 201249 (MATA ura3-302 leu2-270 lys8-11 PEX17-HA (Du et al., 2016 & Zhang et al. 2017) and W29-derived strain Po1f (ATCC® MYA-2613™, MATA ura3-302 leu2-270 xpv2-322 axp2-deltaNU49 XPR2::SUC2). It is possible that these different strain backgrounds alter the essentiality of ERG5. ERG5 was identified as non-essential by transposon mutagenesis in strain W29 (CLIB89/ATCC20460™, Patterson et al., 2018) and CRISPR-Cas9 mediated gene disruption in PO1f (MatA, leu2-270, ura3-302, xpr2-322, axp-2, Schwartz et al., 2019). ERG4 was identified as essential by Patterson et al., 2018, but results were inconclusive for Schwartz at al, 2019. ERG8 was classed as essential by both studies. (It is noted that the method employed by Schwartz et al., 2019, also classified genes such as ERG25, ERG28 and ERG8 as non-essential, although these are essential in Saccharomyces cerevisiae. This is unexpected as Yarrowia lacks homologues of the sterol uptake transporters found in Saccharomyces yeasts which allow certain ERG genes to be knocked out when ergosterol is provided in the growth medium).

The platform strain ST9100 is modified for increased squalene synthesis, the precursor to the sterol pathway. This suggests that the sterol pathway may be carefully regulated at multiple levels to prevent build-up of ergosterol. Regulatory mechanisms may include end-product feedback inhibition by ergosterol. This mechanism may be highly specific for ergosterol, to prevent feedback inhibition by structurally similar early intermediates of the sterol pathway. Whilst ergosterol production by this strain is not greater than the unmodified base strain, strains derived from ST9100 that are incapable of ergosterol production show significantly increased sterol titre. For example, flux through the sterol pathway is greater in ST11027 (ΔERG5, total sterol: 5.2 mg/g dry cell weight (DCW) and ST11040 (ΔERG5ΔERG4, total sterol: 15.8 mg/g DCW) than ST9100 (total sterol: 2.1 mg/g DCW). ERG4/5 deletion in a platform strain may therefore be more harmful to the cell due to the accumulation of sterol intermediates which may be toxic in high concentrations, for example due to detrimental effects to the membrane.

The higher efficiency of ERG4/5/6 knock-out in strains capable of synthesising tetrahymanol may indicate that tetrahymanol is able to substitute for ergosterol function in the cell. This may mitigate the detrimental effects of accumulation of sterol intermediates upon ERG gene deletion. Overall, the ergosterol biosynthesis pathway is highly demanding (especially due to oxygen and NADH requirements). However, the prevalence of ergosterol as the dominant sterol across yeast species suggests that ergosterol is superior to the preceding intermediates for cellular function, despite high structural similarity. This may explain the difficulty in obtaining strains incapable of producing ergosterol, in the absence of a suitable surrogate.

### ADDITIONAL REFERENCES

Du H X, Xiao W H, Wang Y. Zhou X, Zhang Y. Liu D & Yuan Y J (2016) Engineering Yarrowia lipolytica for campesterol overproduction. PLoS One
Magnan C. Yu J, Chang I, Jahn E, Kanomata Y, Wu J, Zeller M, Oakes M, Baldi P & Sandmeyer S (2016) Sequence assembly of Yarrowia lipolytica strain W29/CLIB89 shows transposable element diversity. PLoS One 11
Patterson K, Yu J, Landberg J, Chang I, Shavarebi F, Bilanchone V & Sandmeyer S (2018) Functional genomics for the oleaginous yeast Yarrowia lipolytica. Metab. Eng 48:184-196
Schwartz C, Cheng J F, Evans R, Schwartz C A, Wagner J M, Anglin S, Beltz A, Pan W, Lonardi S, Blenner M, Alper H S, Yoshikuni Y & Wheeldon I (2019) Validating genome-wide CRISPR-Cas9 function improves screening in the oleaginous yeast Yarrowia lipolytica. Metab. Eng. 55: 102-110
Zhang Y, Wang Y, Yao M, Liu H, Zhou X. Xiao W & Yuan Y (2017) Improved campesterol production in engineered Yarrowia lipolytica strains. Biotechnol. Lett.

### Example 3—Engineering of Yarrowia lipolytica for Production of Non-Native Sterols

### Materials and Methods

### Strains, Culture Conditions and Chemicals

Escherichia coli strain DH5α was used for plasmid construction as described above in Example 1.

The Y. lipolytica strain ST9100 derived from the Y. lipolytica W29 strain Y-63746 (Mat A. ATCC20460™/ARS Culture Collection, NCAUR, United States) as described above was used to construct all non-native sterol-producing strains. All strains are detailed in Table 5.

Y. lipolytica was grown at 30° C. on yeast extract peptone dextrose (YPD) media containing 10 g/l yeast extract, 20 g/l peptone, and 20 g/l glucose, supplemented with 20 g/l agar for preparation of solid media. For selection, either nourseothricin (250 mg/l) or hygromycin (400 mg/l) was added to the media.

Cultivation of strains for sterol production was performed in yeast extract peptone medium containing 80 g/l glucose as described more fully above.

Chemicals were obtained, if not indicated otherwise, from Sigma-Aldrich. Nourseothricin was purchased from Jena BioScience GmbH (Germany).

### Plasmid Construction

Delta-7 sterol reductase sequences from Solanum tuberosum, (GenBank accession: BAQ55276.1). Danio rerio (accession: NP_958487.2), Legionella drancourtii (accession: FJ197317.1), Ectocarpus siliculosus (accession: CBN77313.1). Candidatus Protochiamydia amoebophila (accession: KIC71363.1), Coccomyxa subellipsoidea (accession: XM_005650286.1), Mortierella verticillata (accession: KFH65691.1), Glycine soja (accession: XP_028244742.1); Tetraselmis sp. GSL018 (accession: JAC78771.1), and Waddlia chondrophila (accession: ADI39181.1), and squalene-tetrahymanol cyclase from Tetrahymena thermophila (accession: XP_001026696.2) were codon-optimized for Y. lipolytica and the codon-optimised sequences synthesised as GeneArt String DNA fragments by ThermoFischer Scientific. The codon-optimized sequences are listed in Table 4a.

Delta-24 sterol reductase sequences from Danio rerio (accession: BC086711.1) and Mus musculus (accession: NM_053272.2) plus C-28 sterol methyl transferase sequences from Arabidopsis thaliana (accession: NM_101884.4). Amborella trichopoda (accession: XP_006828830.1) and Chenopodium quinoa (accession: XP_021737090.1) were codon-optimised for Y. lipolytica (see Tables 4a and 4b). The codon-optimised sequences were again synthesised as above.

The plasmids, biobricks, and primers used in this study are listed in Tables 12-14, respectively. BioBricks were amplified by PCR. The PCR mix contained 32 μl water, 10 μl high fidelity Phusion® polymerase buffer (5×), 1 pd 10 mM dNTP, 1.5 μl MgCl₂, 2.5 μl 10 μM Fw primer, 2.5 μl 10 μM Rv primer, 0.2 μl Phusion® U polymerase, and 10 ng DNA template. Reactions were multiplexed 8× per BioBrick. The cycling program was: 98° C. for 1 min, 30 cycles of [98° C. for 10 sec, gradient from 50 to 58° C. for 20 sec, 72° C. for 30 s/kb], 72° C. for 5 min, pause at 10° C. The gene fragments were purified from agarose gels using the NucleoSpin® Gel and PCR Clean-up kit (Macherey-Nagel). Biobricks were assembled into the EasyCloneYALI vectors with USER cloning (Holkenbuink et al. 2018). The USER reactions were transformed into E. coli and correct assemblies were verified by Sanger sequencing (Eurofins).

TABLE 12a
Plasmids for engineering of Yarrowia lipolytica for production of non-native sterols

| | Parent plasmid/ | |
|---|---|---|
| Plasmid name | Reference | BioBricks |
| pCfB3405 (pORI1001-Nat-CEN1-USER) | (Holkenbrink et al, | |
| | 2018) | |
| pCfB4788 (pIntA_1-Nat-TPex20-TLip2) | (Holkenbrink et al, | |
| | 2018) | |
| pCfB5935 (pIntA-1-HphMx-TPex20- | (Holkenbrink et al, | |
| TLip2) | 2018) | |
| pCFB8843 (pORI1001-Hyg-CEN1-USER) | (Arnesen et al, | |
| | 2020) | |
| pCfB6637 (pNat-YLgRNA3_IntE_3) | (Holkenbrink et al, | |
| | 2018) | |
| pCfB6638 (pNat-YLgRNA2_IntE_4) | (Holkenbrink et al, | |
| | 2018) | |
| pCfB6677 (pIntE_1-TPex20-TLip2) | (Holkenbrink et al, | |
| | 2018) | |
| pCfB6679 (pIntE_4-TPex20-TLip2) | (Holkenbrink et al, | |
| | 2018) | |
| pCfB8861 (pHyg-YLgRNA3_IntE_4) | (Arnesen et al, | |
| | 2020) | |
| pCfB6681 (pIntE_3-TPex20-TLip2) | (Holkenbrink et al, | |
| | 2018) | |
| pCfB6611 (pNat-PrExp-Cre) | pCfB4158 (pPrExp- | |
| | Cre) - (Holkenbrink | |
| | et al, 2018) | |
| pCfB10249 (IntE4_PrTEF- | pCfB6679 | BB3879 (Tefint->):BB4876 |
| >DHCR7_Stuberosum) | | (PrTEF_DHCR7St) |
| pCfB10250 (IntE4_PrTEF- | pCfB6679 | BB3879 (Tefint->):BB4877 |
| >DHCR7_Drerio) | | (PrTEF_DHCR7Dr) |
| pCfB10251 (IntE4_PrTEF- | pCfB6679 | BB3879 (Tefint->):BB4878 |
| >DHCR7_Ldrancou) | | (PrTEF_DHCR7Ld) |
| pCfB10252 (IntE4_PrTEF- | pCfB6679 | BB3879 (Tefint->):BB4879 |
| >DHCR7_Esilicul) | | (PrTEF_DHCR7Es) |
| pCfB10253 (IntE4_PrTEF- | pCfB6679 | BB3879 (Tefint->):BB4880 |
| >DHCR7_Cprotoch) | | (PrTEF_DHCR7Cp) |
| pCfB10254 (IntE4_PrTEF- | pCfB6679 | BB3879 (Tefint->):BB4881 |
| >DHCR7_Csubellip) | | (PrTEF_DHCR7Cs) |
| pCfB10255 (IntE4_PrTEF- | pCfB6679 | BB3879 (Tefint->):BB4882 |
| >DHCR7_Mverticillata) | | (PrTEF_DHCR7Mv) |
| pCfB10256 (IntE4_PrTEF- | pCfB6679 | BB3879 (Tefint->):BB4883 |
| >DHCR7_Gsoja) | | (PrTEF_DHCR7Gs) |
| pCfB10257 (IntE4_PrTEF->DHCR7_Tsp) | pCfB6679 | BB3879 (Tefint->):BB4884 |
| | | (PrTEF_DHCR7Ts) |
| pCfB10258 (IntE4_PrTEF- | pCfB6679 | BB3879 (Tefint->):BB4885 |
| >DHCR7_Wchondrophi) | | (PrTEF_DHCR7Wc) |
| pCfB10494 (IntE3_PrGPAT->TtSTC) | pCfB6681 | BB1617 (PrGPAT):BB5100 |
| | | (TtSTC_USER) |
| pCfB10367 (Erg5_tPr48) | | BB1135:BB5040:BB5101:BB5044 |
| pCfB7254 (gRNA for PAH1 deletion Y.L) | pCfB3405 | BB1635:BB1636:PR-29832-PR29833 |

TABLE 12b
Plasmids for engineering of Yarrowia lipolytica for production of non-native sterols

| | Parent plasmid/ | |
|---|---|---|
| Plasmid name | Reference | BioBricks |
| pCfB6633 (pNat-YLgRNA2_IntE_1) | (Holkenbrink et al, | |
| | 2018) | |
| pCfB6637 (pNat-YLgRNA3_IntE_3) | (Holkenbrink et al, | |
| | 2018) | |
| pCfB6638 (pNat-YLgRNA2_IntE_4) | (Holkenbrink et al, | |
| | 2018) | |
| pCfB6677 (pIntE_1-TPex20-TLip2) | (Holkenbrink et al, | |
| | 2018) | |
| pCfB6681 (pIntE_3-TPex20-TLip2) | (Holkenbrink et al, | |
| | 2018) | |
| pCfB4783 (pIntE_3-Nat-TPex20- | (Holkenbrink et al, | |
| TLip2) | 2018) | |
| pCfB6679 (pIntE_4-TPex20-TLip2) | (Holkenbrink et al, | |
| | 2018) | |
| pBP8009 (pIntF_3-TPex20-TLip2) | Dr. K. R. Kildegaard, | |
| | Biophero ApS, | |
| | Denmark | |
| pBP8003 (pNat-YLgRNA4-IntF_3) | Dr. K. R. Kildegaard, | |
| | Biophero ApS, | |
| | Denmark | |
| pCfB8861 (pHyg-YLgRNA3_IntE_4) | (Arnesen et al, 2020) | |
| pCfB6611 (pNat-PrExp-Cre) | pCfB4158 (pPrExp-Cre) - | |
| | (Holkenbrink et al, | |
| | 2018) | |
| pCfB10251 (IntE4_PrTEF- | pCfB6679 | BB3879 (Tefint->):BB4878 |
| >DHCR7_Ldrancou) | | (PrTEF_DHCR7Ld) |
| pCfB10254 (IntE4_PrTEF- | pCfB6679 | BB3879 (Tefint->):BB4881 |
| >DHCR7_Csubellip) | | (PrTEF_DHCR7Cs) |
| pCfB10257 (IntE4_PrTEF- | pCfB6679 | BB3879 (Tefint->):BB4884 |
| >DHCR7_Tsp) | | (PrTEF_DHCR7Ts) |
| pCfB10293 (IntE1_PrTEF- | pCfB6677 | BB3879 (Tefint->):BB4965 |
| >DHCR24_Drerio) | | (PrTEF_DHCR24Dr) |
| pCfB10298 (IntE1_PrTEF- | pCfB6677 | BB3879 (Tefint->):BB4970 |
| >DHCR24_Mmusculus) | | (PrTEF_DHCR24Mm) |
| pCfB10311 (IntE1_PrTEF- | pCfB6677 | BB3879 (Tefint->):BB4983 |
| >SMT2_Cquinoa) | | (PrTEF_SMT2Cq) |
| pCfB11486 (IntF3_SMT_Ath<- | pBP8009 | BB4900 (PrGPD_SMT2Atr):BB3865 |
| PrGPD_TEFi->SMT_Atr) | | (<-PrGPD_Tefint->):BB4977 |
| | | (PrTEF_SMT2Ath) |
| pCfB10494 (IntE3_PrGPAT->TtSTC) | pCfB6681 | BB1617 (PrGPAT):BB5100 |
| | | (TtSTC_USER) |

TABLE 13a
Biobricks for engineering of Yarrowia lipolytica for production of non-native sterols

| | | Forward | Reverse |
|---|---|---|---|
| BioBrick name | Template/Reference | primer | primer |
| BB1635 | (Holkenbrink et al, 2018) | | |
| BB1636 | (Holkenbrink et al, 2018) | | |
| BB1617 (PrGPAT->) | (Holkenbrink et al, 2018) | | |
| BB3879 (PrTefInt->) | (Arnesen et al, 2020) | | |
| BB4876 | Solanum tuberosum delta-7 sterol reductase codon | PR-27565 | PR-27566 |
| (PrTEF_DHCR7St) | optimised | | |
| BB4877 | Danio rerio delta-7 sterol reductase codon | PR-27567 | PR-27568 |
| (PrTEF_DHCR7Dr) | optimised | | |
| BB4878 | Legionella drancourtii delta-7 sterol reductase | PR-27569 | PR-27560 |
| (PrTEF_DHCR7Ld) | codon optimised | | |
| BB4879 | Ectocarpus siliculosus delta-7 sterol reductase | PR-27571 | PR-27572 |
| (PrTEF_DHCR7Es) | codon optimised | | |
| BB4880 | Candidatus Protochlamydia amoebophila delta-7 | PR-27573 | PR-27574 |
| (PrTEF_DHCR7Cp) | sterol reductase codon optimised | | |
| BB4881 | Coccomyxa subellipsoidea delta-7 sterol reductase | PR-27575 | PR-27576 |
| (PrTEF_DHCR7Cs) | codon optimised | | |
| BB4882 | Mortierella verticillate delta-7 sterol reductase | PR-27577 | PR-27578 |
| (PrTEF_DHCR7Mv) | codon optimised | | |
| BB4883 | Glycine soja delta-7 sterol reductase codon | PR-27579 | PR-27570 |
| (PrTEF_DHCR7Gs) | optimised | | |
| BB4884 | Tetraselmis sp. GSL018 delta-7 sterol reductase | PR-27581 | PR-27582 |
| (PrTEF_DHCR7Ts) | codon optimised | | |
| BB4885 | Waddlia chondrophila delta-7 sterol reductase | PR-27583 | PR-27584 |
| (PrTEF_DHCR7Wc) | codon optimised | | |
| BB5041 (LoxP_HphMX) | pCfB5935 - (Holkenbrink et al, 2018) | PR-27972 | PR-27973 |
| BB5101 (LoxP_NatMX) | pCfB4788 | PR-27972 | PR-27973 |
| BB5091 | ST9100 gDNA | PR-27891 | PR-28358 |
| (erg4_1 kbUP_U1) | | | |
| BB5092 | ST9100 gDNA | PR-27893 | PR-27894 |
| (erg4_1 kbDOWN_U2) | | | |
| BB5093 | ST9100 gDNA | PR-27895 | PR-28359 |
| (erg5_1 kbUP_U1) | | | |
| BB5094 | ST9100 gDNA | PR-27897 | PR-27898 |
| (erg5_1 kbDOWN_U2) | | | |
| BB5095 | ST9100 gDNA | PR-27899 | PR-28360 |
| (erg6_1 kbUP_U1) | | | |
| BB5096 | ST9100 gDNA | PR-27901 | PR-27902 |
| (erg6_1 kbDOWN_U2) | | | |
| BB5097 | BB5091 + BB5092 + BB5041 USER reaction | PR-27891 | PR-27894 |
| (erg4_HphMX_KO) | | | |
| BB5098 | BB5093 + BB5094 + BB5041 USER reaction | PR-27895 | PR-27898 |
| (erg5_HphMX_KO) | | | |
| BB5099 | BB5095 + BB5096 + BB5041 USER reaction | PR-27899 | PR-27902 |
| (erg6_HphMX_KO) | | | |
| BB5100 (TtSTC_USER) | Tetrahymena thermophilia squalene-tetrahymanol | PR-28361 | PR-28362 |
| | cyclase codon optimised | | |
| BB5040 | ST9100 gDNA | PR-27970 | PR-27971 |
| (Erg5_UPintergenic) | | | |
| BB5044 | ST9100 gDNA | PR-27976 | PR-27969 |
| (Erg5_tPr48_ORF996) | | | |
| BB1135 (Easy Clone | pCfB6677 | PR-11110 | PR-11111 |
| vector backbone) | | | |
| BB5312 | ST9100 gDNA | PR-26303 | PR-26304 |
| (YIPAH1_repair-up) | | | |
| BB5313 | ST9100 gDNA | PR-26305 | PR-26306 |
| (YIPAH1_repair-dw) | | | |
| BB5314 | BB5312 + BB5313 USER ligation | PR-26303 | PR-26306 |
| (YIPAH1_repair) | | | |

TABLE 13b
Biobricks for engineering of Yarrowia lipolytica for production of non-native sterols

| | | Forward | Reverse |
|---|---|---|---|
| BioBrick name | Template/Reference | primer | primer |
| BB1616 (PrDGA1->) | (Holkenbrink et al, 2018) | | |
| BB1617 (PrGPAT->) | (Holkenbrink et al, 2018) | | |
| BB3865 ( <- | (Arnesen et al, 2020) | | |
| PrGPD_Tefint->) | | | |
| BB3879 (PrTefint->) | (Arnesen et al, 2020) | | |
| BB5041 (LoxP_HphMX) | pCfB5935 - (Holkenbrink et al, 2018) | PR-27972 | PR-27973 |
| BB5101 (LoxP_NatMX) | pCfB4788 | PR-27972 | PR-27973 |
| BB5091 | ST9100 gDNA | PR-27891 | PR-28358 |
| (erg4_1 kbUP_U1) | | | |
| BB5092 | ST9100 gDNA | PR-27893 | PR-27894 |
| (erg4_1 kbDOWN_U2) | | | |
| BB5093 | ST9100 gDNA | PR-27895 | PR-28359 |
| (erg5_1 kbUP_U1) | | | |
| BB5094 | ST9100 gDNA | PR-27897 | PR-27898 |
| (erg5_1 kbDOWN_U2) | | | |
| BB5095 | ST9100 gDNA | PR-27899 | PR-28360 |
| (erg6_1 kbUP_U1) | | | |
| BB5096 | ST9100 gDNA | PR-27901 | PR-27902 |
| (erg6_1 kbDOWN_U2) | | | |
| BB5097 | BB5091 + BB5092 + BB5041 USER reaction | PR-27891 | PR-27894 |
| (erg4_HphMX_KO) | | | |
| BB5098 | BB5093 + BB5094 + BB5041 USER reaction | PR-27895 | PR-27898 |
| (erg5_HphMX_KO) | | | |
| BB5099 | BB5095 + BB5096 + BB5041 USER reaction | PR-27899 | PR-27902 |
| (erg6_HphMX_KO) | | | |
| BB5100 (TtSTC_USER) | Tetrahymena thermophilia squalene-tetrahymanol | PR-28361 | PR-28362 |
| | cyclase codon optimised | | |
| BB1135 (Easy Clone | pCfB6677 | PR-11110 | PR-11111 |
| vector backbone) | | | |
| BB4878 | Legionella drancourtii delta-7 sterol reductase | PR-27569 | PR-27570 |
| (PrTEF_DHCR7Ld) | codon optimised | | |
| BB4881 | Coccomyxa subellipsoidea delta-7 sterol reductase | PR-27575 | PR-27576 |
| (PrTEF_DHCR7Cs) | codon optimised | | |
| BB4884 | Tetraselmis sp. GSL018 delta-7 sterol reductase | PR-27581 | PR-27582 |
| (PrTEF_DHCR7Ts) | codon optimised | | |
| BB4965 | Danio rerio delta-24 sterol reductase codon | PR-27749 | PR-27750 |
| (PrTEF_DHCR24Dr) | optimised | | |
| BB4970 | Mus musculus delta-24 sterol reductase codon | PR-27759 | PR-27760 |
| (PrTEF_DHCR24Mm) | optimised | | |
| BB4983 | Chenopodium quinoa sterol methyltransferase | PR-27785 | PR-27786 |
| (PrTEF_SMT2Cq) | codon optimised | | |
| BB4900 | Amborella trichopoda sterol methyltransferase | PR-27613 | PR-27614 |
| (PrGPD_SMT2Atr) | codon optimised | | |
| BB4977 | Arabidopsis thaliana sterol methyltransferase | PR-27773 | PR-27774 |
| (PrTEF_SMT2Ath) | codon optimised | | |

TABLE 14a
Primers for engineering of Yarrowia lipolytica for production of non-native sterols

| Primer name | Sequence (5′ → 3′) |
|---|---|
| PR-14617 (vector verification E. | Tatccctgtgttgaatc |
| coli cPCR) | |
| PR-14619 (vector verification E. | Tatcgacccagttagc |
| coli cPCR) | |
| PR-8859 (integration verification | Aagtgtggatggggaagtgag |
| YI cPCR) | |
| PR-14576 (IntE_3 verification YI | Cacgcgautgaaggaaatgcctaaaacc |
| cPCR) | |
| PR-14835 (IntE_3 verification YI | Cacgcacgccattctataag |
| cPCR) | |
| PR-14592 (IntE_4 verification YI | Acgcgauttaacactggaccgtactgc |
| cPCR) | |
| PR-20880 (IntE 4 verification Y]I | Attgctaagcgaccatagac |
| cPCR) | |
| PR-11110 (E. coli backboneUSER_ | Atcgcgtgcattcgcggccgcatttaaatcc |
| fw) | |
| PR-11111 (E. coli backboneUSER_ | Atcgcacgcattcgcggccgcaaatttaaataaaatg |
| rev) | |
| PR-27565 (A_St_Fw) | Actttttgcagtacuaaccgcaggccgagtctcagctggtgcac |
| PR-27566 (A_St_Rv) | Cacgcgauttagtagatgccggggatcac |
| PR-27567 (A_Dr_Fw) | Actttttgcagtacuaaccgcagatggcctctgaccgagttcg |
| PR-27568 (A_Dr_Rv) | Cacgcgauttagaagatgttgggcagcag |
| PR-27569 (A_Ld_Fw) | Actttttgcagtacuaaccgcagtacttcaagatccgaaacac |
| PR-27570 (A_Ld_Rv) | Cacgcgauttagatcacgaagggcacgatc |
| PR-27571 (A_Es_Fw) | Actttttgcagtacuaaccgcagatcgacggcgctgccatcg |
| PR-27572 (A_Es_Rv) | Cacgcgauttacaggatgccgggcacgatc |
| PR-27573 (A_Cp_Fw) | Actttttgcagtacuaaccgcagctgatcgagatgctgtgcatc |
| PR-27574 (A_Cp_Rv) | Cacgcgauttagatcacgaaggggatgatc |
| PR-27575 (A_Cs_Fw) | Actttttgcagtacuaaccgcaggtcacaacccgagccgctg |
| PR-27576 (A_Cs_Rv) | Cacgcgauttagaaaatgtaagggatcatc |
| PR-27577 (A_Mv_Fw) | Actttttgcagtacuaaccgcaggccgtgcagcagcgaaagac |
| PR-27578 (A_Mv_Rv) | Cacgcgauttagtacacgtaggggatcagc |
| PR-27579 (A_Gs_Fw) | Actttttgcagtacuaaccgcagggcgctaccgtgcactctc |
| PR-27580 (A_Gs_Rv) | Cacgcgauttagtagatgccggggatg |
| PR-27581 (A_Ts_Fw) | Actttttgcagtacuaaccgcagaagcgagcctccaagaccc |
| PR-27582 (A_Ts_Rv) | Cacgcgauttagaagatgtagggcacgatc |
| PR-27583 (A_Wc_Fw) | Actttttgcagtacuaaccgcaggccgccaccaccaccaacg |
| PR-27584 (A_Wc_Rv) | Cacgcgauttagtagatgccggggatcag |
| PR-27891 (Erg4Rep_P1F) | Ctctcaacaccttcaccgc |
| PR-28358 (Erg4Rep_P1R_U1) | Atcgcacgugataagcttagtgagcgaatgg |
| PR-27893 (Erg4Rep_P2F) | gtgcaggutgtgtggttgcgaaggaag |
| PR-27894 (Erg4Rep_P2R) | gcactcaaaataccccgttc |
| PR-27895 (Erg5Rep_P1F) | ctcggtttgttgcagcagg |
| PR-28359 (Erg5Rep_P1R_U1) | atcgcacgutggtccgtatcgtgaaatgg |
| PR-27897 (ErgSRep_P2F) | gtgcaggugggcggagttgtgttgtg |
| PR-27898 (Erg5Rep_P2R) | ggtcggctatccaatacatctc |
| PR-27899 (Erg6Rep_P1F) | gctacaagccggaggggaac |
| PR-28360 (Erg6Rep_P1R_U1) | atcgcacgucaagggcgattcagatcagc |
| PR-27901 (Erg6Rep_P2F) | gtgcagguactgagtaacttatagaggg |
| PR-27902 (Erg6Rep_P2R) | ctgtaccgtttggaggactc |
| PR-27972 (HphMX_F_U1) | cgtgcgautcagctgaagcttcgtac |
| PR-27973 (HphMX_R_U2) | acctgcacugcataggccactagtgg |
| PR-28361 (TISTC_F_U3) | atctgtcaugccacaatgaagaagatcctcatcggtc |
| PR-28362 (TtSTC_Rv) | cacgcgauttagatgttctgcttctggacg |
| PR-22830 (Berg5_chk) | tcatactcaccgaaacgtg |
| PR-27961 (Derg5_chk) | gttccaatgcctggcaag |
| PR-27634 (Berg5_chk) | acttctctctctcacaccacc |
| PR-27635 (Berg5_chk) | ctgagggctctgttggtgaag |
| PR-27636 (Berg5_chk) | accagtgtagttgtaaggatg |
| PR-11138 (Berg4_chk) | agcaatggguaaaaagcctgaactcaccgc |
| PR-27631 (Berg4_chk) | cctgatattggtgatcctcc |
| PR-27632 (Berg4_chk) | agagccttgtttccgaggtg |
| PR-27633 (Berg4_chk) | atacaatcccataggctggc |
| PR-28001 (Berg4_chk) | cgtgcgaugcttgccctggactacatcttg |
| PR-22830 (Berg6_chk) | tcatactcaccgaaacgtg |
| PR-28007 (Berg6_chk) | cgtgcgaugaaggagatactggtgcc |
| PR-27637 (Berg6_chk) | ctcgcatacttcccgtttgg |
| PR-27639 (Berg6__chk) | ccaccgatccttctcagctac |
| PR-27969 (ErgSORF_R_U3) | cacgcgautcggtcggcaacaatctg |
| PR-27970 | cgtgcgautaagcatgcatcggacac |
| (ErgS_UP_uORF_F_U3) | |
| PR-27971 | atcgcacgugatcgtgtgagtcagagg |
| (ErgS_UP_UORF_R_U1) | |
| PR-27976 (ERG5_tPrSO_F_U2) | agtgcaggucacaacttctctctctcacac |
| PR-26303 (YIPAH1_repair- | acgtactgcgcccatatt |
| up_fw) | |
| PR-26304 (YIPAH1_repair-up_rv) | aggccacutatggttgtgtggtgatga |
| PR-26305 (YIPAH1_repair- | aggtggccuattccagcccgtttcgt |
| dw_fw) | |
| PR-26306 (YIPAH1_repair- | cttggctttctagcggga |
| dw_rv) | |
| PR-26307 (YIPAH1_check_fw) | cgaacccaaatccggac |
| PR-26308 (YIPAH1_check_rv) | acctgctcctccaccta |
| PR-29832 (PAH1_gRNA_Sense) | ggaaggttagaagaagggaggttttagagct |
| PR-29833 | ctcccttcttctaaccttcctaaccaacct |
| (PAH1_gRNA_AntiSense) | |

TABLE 14b
Additional primers for engineering of Yarrowia lipolytica for production of non-
native sterols

| Primer name | Sequence (5′ → 3′) |
|---|---|
| PR-14442 (IntE_1 verification YI | agttgtgaccaagacaaatg |
| cPCR) | |
| PR-14398 (IntE_1 verification YI | cacgcgaUgttagaagcaattggagaag |
| cPCR) | |
| PR-14837 (IntF_3 verification YI | acatgctcgcgcctcgatag |
| cPCR) | |
| PR-14584 (IntF_3 verificiationYI | cacgcgautttggtcgtcgcccaacaag |
| cPCR) | |
| PR-27749 (B_Dr_Fw) | actttttgcagtacuaaccgcaggaccctctgctgtac |
| PR-27750 (B_Dr_Rv) | cacgcgauttagtgtcgggcagacttg |
| PR-27759 (B_Mm_Fw) | actttttgcagtacuaaccgcaggaacccgccgtgtctc |
| PR-27760 (B_Mm_Rv) | cacgcgauttagtgtcgagcggccttg |
| PR-27785 (C_Cq_Fw) | actttttgcagtacuaaccgcaggactctatggccctg |
| PR-27786 (C_Cq_Rv) | cacgcgauttaagagtcagacttggc |
| PR-27613 (C_At_Pw) | atctgtcaugccacaatggagactctggccgc |
| PR-27614 (C_At_Rv) | cgtgcgauttagcaggcgggagcagc |
| PR-27773 (C_AtSMT2_Fw) | actttttgcagtacuaaccgcaggactctctgaccctg |
| PR-27774 (C_AtSMT2_Rv) | cacgcgauttaagaagattcctcgggag |

### Yeast Transformation

The yeast vectors were integrated into different previously characterized intergenic loci in the Y. lipolytica genome as described in Holkenbrink et al, 2018: see also Table 18 below. Integration vectors were digested with Notl enzyme (New England BioLabs) at 37° C. for 1 hr and the digested product purified from solution using the NucleoSpin® Gel and PCR Clean-up kit (Macherey-Nagel). The purified DNA was transformed using the lithium acetate transformation protocol as described by Holkenbrink et al, 2018. Correct integration was verified by colony PCR using Taq DNA Polymerase Master Mix RED (Ampliqon) with vector-specific primers and primers complementary to the genomic region adjacent to the integration site.

For marker-mediated gene deletion, Y. lipolytica strains were transformed with BioBricks assembled by USER reaction as detailed above. Transformants were selected on antibiotic supplemented plates and correct transformants confirmed by colony PCR. Marker removal was performed by transformation of the strains with a Cre-recombinase episomal vector. Marker removal was confirmed by colony PCR.

### Yeast Cultivation

Yeast strains were inoculated into 2.5 ml YPD in 24-deepwell plates with air-penetrable lids (EnzyScreen, Netherlands). The plates were incubated at 30° C. with 300 rpm agitation at 5 cm orbit cast for 24 hours. The cultures were then diluted to OD600 0.1 in 2.5 ml fresh YPD-media with 80 g/l glucose and grown for a further 72 hours at 30° C. with 300 rpm agitation. All cultivations were performed in triplicate. Dry cell weight (DCV) was measured at the end of cultivation: 1 ml of culture broth was transferred into a pre-weighed 2 ml microcentrifuge tube, centrifuged (3000 g, 5 min) and the supernatant was discarded. The cells were washed twice with deionized water (1 ml). The cell pellet was dried at 0° C. for 7 days before the final weight was measured.

### Sterol Analysis

For sterol extraction, 1 ml of culture broth was transferred into a 2 ml microcentrifuge tube, centrifuged and the supernatant was discarded. The cells were washed twice with deionized water (1 ml). The cell pellet was resuspended in 10% w/v methanolic potassium hydroxide (500 μl) and transferred to a 1 ml glass vial for saponification. The suspension was incubated at 70° C. for 2 hours with vortexing at 15 minute intervals. The saponified samples were then vortexed and spiked with 50 μl of internal standard (1 mg/ml epicoprostanol in absolute ethanol). 500 μl of n-hexane was added to each sample for extraction of the free sterol component. Samples were vortexed and the organic phase transferred to a 2 ml microcentrifuge tube. The extraction step was repeated in a further 500 μl of n-hexane. The combined hexane phases were left overnight at room temperature for evaporation of the solvent. The resulting crystals were resuspended in 50 μl of n-hexane for concentration at the bottom of the tube and left at room temperature overnight for final drying. Samples were then stored at 4° C. prior to analysis.

Sterols were derivatized with 20 μl Tri-Sil (Sigma) and then briefly vortexed before direct injection into an Agilent Technologies (Palo Alto, CA, USA) 7890A gas chromatograph connected to an Agilent Technologies 5975C. MSD mass spectrometer (for gas chromatography-mass spectrometry (GC-MS)) and eluted over an Agilent DB5 column using a splitless injection at 250° C. with a standard GC program at 170° C. for 1 min, ramped to 280° C. at 20° C. mint and monitoring between 50 and 550 amu.

Sterols were identified by comparison of their retention time relative to cholesterol and mass spectra data available from the NIST (National Institute of Standards and Technology) mass spectral library and (Zu et al. 2021). Sterols were quantified by calculating the ratio of the peak area of the targeted sterol to that of the internal standard. The mass of each sterol in the sample was obtained by multiplying the ratio with the mass of the internal standard. Compound identification (using target ions) and quantification were carried out with ChemStation Enhanced Data Analysis (v.E.01.00).

### Results

Construction of a Yarrowia lipolytica Capable of Synthesizing a Sterol Surrogate

The gene encoding squalene-tetrahymanol cyclase from Tetrahymena thermophila (TtSTC) was integrated into the ST9100 platform strain genome under the control of the PrGPAT promoter, previously characterised as a weak promoter (Holkenbrink et al, 2018). This promoter was selected to drive minimal expression of TtSTC to limit diversion of carbon flux away from the main sterol pathway. As noted above, the promoter described as ‘PrGPAT’ in Holkenbrink et al, 2018 (sequence provided in the supplementary information of that paper) and used in this study does not belong to the Y. lipolytica GPAT gene (YALI1_C00230 g, Y. lipolytica W29/CLIB89 genome assembly, Magnan et al, 2016), but is instead the promoter of a putative gene (YALI1C00209 g). The resulting strain ST11005 produced 0.4 mg/g dry cell weight tetrahymanol, comprising 14.0% of the total sterol fraction (FIG. 11).

Construction of Yarrowia lipolytica Strains Incapable of Synthesising Ergosterol

As noted above, the tetrahymanol producing strain ST11005 was used for the construction of strains incapable of synthesising ergosterol. Firstly, to create a ΔERG5 strain, the ERG5 gene (sterol C-22 desaturase. YALI1_A18344 g) was deleted by marker-mediated knock-out. A knock-out cassette was constructed comprising a hygromycin resistance marker (HphMX) flanked by 1 kb homology arms corresponding to 1 kb upstream and downstream of the ERG5 coding sequence. This construct was used to transform ST11005, generating ST11014. The HphMX resistance marker was subsequently looped out by Cre-Lox recombination. The resulting strain ST11027 produced 3.1 mg/g DCW ergosta-5,7-dienol, comprising 60.0% of the total sterol fraction. The strain also produced 5.2 mg/g DCW tetrahymanol, comprising 36.7% of the total sterol fraction.

To create a ΔERG4ΔERG5 strain, the ERG4 gene (delta-24 sterol reductase, YALI1_D24361 g)) was deleted by HphMX marker-mediated knockout in an analogous manner. The resulting strain ST11040 produced 3.1 mg/g DCW ergosta-5,7,24(28)-trienol, comprising 20.0% of the total sterol fraction. The strain also produced 15.8 mg/g DCW tetrahymanol, comprising 75.2% of the total sterol fraction.

To create a ΔERG5ΔERG6 strain, the gene encoding ERG6 (sterol methyl transferase, YALI1_F12138 g) was deleted from ST11027 by HphMX marker-mediated knockout in an analogous manner, generating strain ST11325. The HphMX resistance marker was subsequently looped out by Cre-Lox ecombination. The resulting strain ST11330 produced 0.2 mg/g DCW zymosterol, comprising 0.6% of the total sterol fraction. The strain also produced 36.0 mg/g DCW tetrahymanol, comprising 99.4% of the total sterol fraction.

### Construction of Campesterol-Producing Strains

The ERG5 knock-out strain ST11027 was used for the construction of campesterol-producing strains. Delta-7 sterol reductase gene variants were expressed under the control of the PrTEFintron promoter, generating strains ST11066 to ST11075. The highest campesterol yield, 40.2 mg/g DCW, was obtained in the strain ST11071 expressing the Coccomyxa subellipsoidea delta-7 sterol reductase (FIG. 12).

### Optimisation of Campesterol-Producing Strains

Campesterol can be produced by expressing a delta-7 sterol reductase in a base strain, a strain with down-regulated ERG5 expression, or a strain in which ERG5 is deleted. Ectocarpus siliculosus delta-7 sterol reductase was expressed under the control of the PrTEFintron promoter in strain ST9100 and strain ST11027 (ΔERG5), generating strains ST10924 and ST11069. The ERG5 promoter was truncated to 48 bp in strain ST10924, generating strain ST91034 (ERG5_trPr_48 bp). The PAH1 gene (YALI1_D35593 g) was also knocked out in ST10924 and ST11069 to increase the amount of membranes and capacity for sterol accumulation. Gene deletion was achieved by CRISPR-Cas9 and a 1 kb repair template comprising homology arms corresponding to 500 bp up- and downstream of the PAH1 coding sequence. ERG5 deletion results in the greatest increase in campesterol yield. The highest campesterol yield, 41.5 mg/g DCW, was obtained in the strain ST11197 (FIG. 13).

### Construction of 24-Methylenecholesterol-Producing Strains

The ΔERG5ΔERG4 strain ST11040 was used for the construction of 24-methylenecholesterol-producing strains. Delta-7 sterol reductase gene variants were similarly expressed under the control of the PrTEFintron promoter, generating strains ST11056 to ST11065. All ten variants were tested as the likely substrate of delta-7 reductase is ergosta-5,7,24(28)-trienol in these strains, compared to ergosta-5,7-dienol in the campesterol-producing strains. [Nb. predicted substrates of delta-7 sterol reductase were inferred from parental strains and from the biochemical pathway. As noted above, ergosta-5,7-dienol accumulates in Δerg5 strain ST11027 (from which campesterol strains are derived by addition of delta-7 sterol reductase)]. Ergosta-5,7,24(28)-trienol accumulates in ΔERG4-ΔERG5 strain ST11040 (from which 24-methylenecholesterol strains are derived by addition of delta-7 sterol reductase)]. Thus, the substrate specificity and catalytic activity of the delta-7 sterol reductase variants may differ between backgrounds.

The highest 24-methylenecholesterol yield, 47.7 mg/g DCW, was obtained in the strain ST11064 expressing the Tetraselmis sp. GSL018 delta-7 reductase under the control of the PrTEFintron promoter. (FIG. 14).

### DISCUSSION

The total sterol content is greater in all strains incapable of producing ergosterol than those capable of producing ergosterol. It is interesting to note that the ergosterol contents in the platform strain ST9100 and the strain ST11005 expressing TtSTC are not greater than that of the parent strain ST6512 which does not contain modifications for increased terpenoid production. It is therefore anticipated that the ergosterol pathway is tightly regulated to control ergosterol concentration in the cell. This may partly be achieved by end-product feedback inhibition. This mechanism would be expected to be highly specific to ergosterol, to prevent interference by structurally similar early intermediates of the ergosterol pathway. Hence, sterol accumulation is greater in strains lacking ergosterol, particularly in those producing non-native sterols. These sterols are likely to be esterified and stored in lipid droplets to prevent toxicity.

Not all strains capable of producing campesterol require a tetrahymanol substitute. Campesterol can be produced in strains expressing a heterologous delta-7 sterol reductase alone, or strains expressing a heterologous delta-7 sterol reductase with partial reduction of ERG5 expression. However, a strain capable of producing a small amount of tetrahymanol seems to accumulate more campesterol than a strain that produces a small amount of ergosterol. It is possible that this is because tetrahymanol substitutes for ergosterol function in the membrane but does not down-regulate the pathway by end-product inhibition in the same way that ergosterol might. Increasing the amount of cellular membranes in a strain producing campesterol and tetrahymanol has a greater effect on the accumulation of tetrahymanol than campesterol. This suggests that tetrahymanol accumulates in the membrane. Campesterol is likely esterified and stored in lipid droplets.

Similarly, the tetrahymanol content is greater in all strains incapable of producing ergosterol than those capable of producing ergosterol. Whilst this is expected if overall flux through the ergosterol and preceding pathways is increased, the percentage of tetrahymanol relative to the total sterol component is also increased. This may suggest that whilst ergosterol is the preferred sterol, tetrahymanol may be used in the absence of a suitable alternative.

As such, ERG4 has been previously identified as an essential gene in Y. lipolytica wild-type strain CLIB89 (W29, ATCC® 20460™) by transposon mutagenesis (Patterson et al. 2018). The main Erg4p substrate, ergosta-5,7,22.24(28)-tetraenal, may be unable to support normal cellular function alone, for example due to toxic effects on membrane structure and properties. However, in strains capable of synthesising tetrahymanol. ERG4 is non-essential. In ST11040, ergosta-5.7.24(28)-trienol accumulates, yet tetrahymanol comprises 75.2% of the total sterol fraction. This suggests that tetrahymanol can better substitute for ergosterol function in the cell. Similarly, ERG6 has been classified as an essential gene in Y. lipolytica by transposon mutagenesis (Patterson at al. 2018) and CRISPR-Cas9 mediated gene disruption (Schwartz et al, 2019). However, in strains capable of synthesising tetrahymanol, ERG6 is non-essential. In ST11330, zymosterol accumulates, yet tetrahymanol comprises 99.4% of the total sterol fraction. This again suggests that tetrahymanol can better substitute for ergosterol function in the cell.

Engineered Y. lipolytica strains therefore hold great potential for the synthesis of non-native sterol structures, due to the capacity of this oleaginous yeast to store and accumulate sterols. It is expected that sterol content could be improved by further strain modification and scale-up of cultivations.

### Construction of a β-Sitosterol-Producing Strain

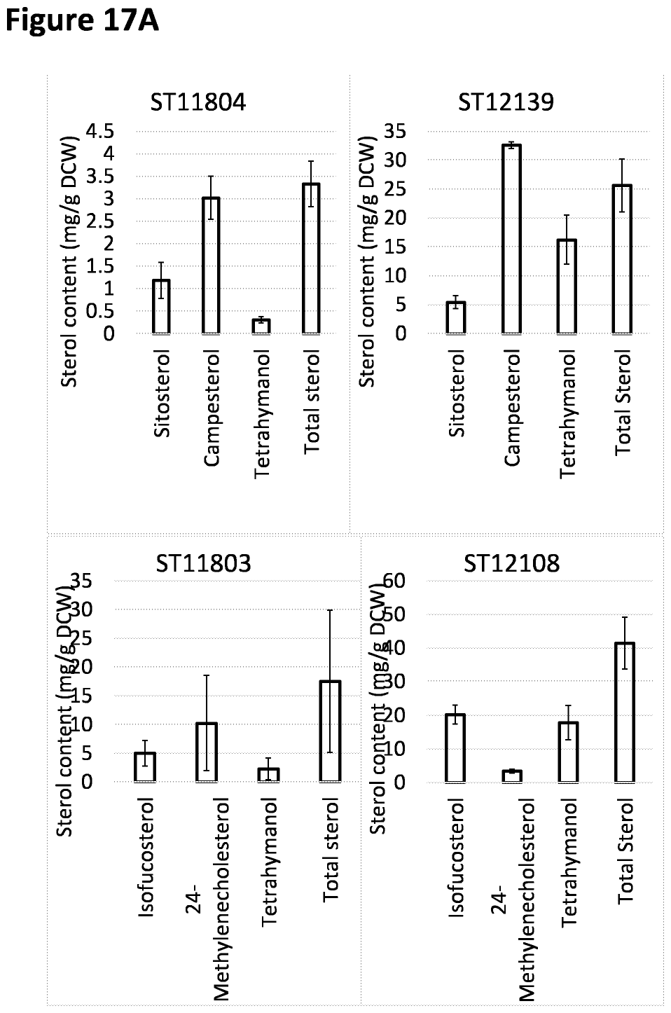

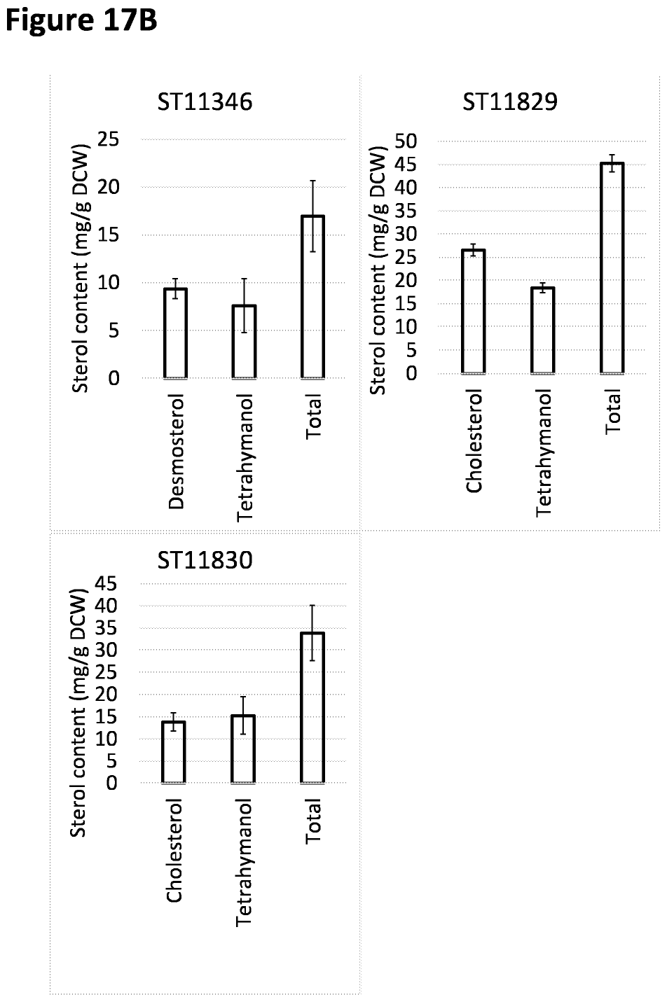

The campesterol-producing strain ST11071 was used for the construction of a β-sitosterol-producing strain (FIG. 17A). The C-28 sterol methyltransferase gene variant from C. quinoa was expressed under the control of the PrTEFintron promoter. The resulting strain ST11804 produced 1.2 mg/g DCW β-sitosterol. To further increase β-sitosterol production, two additional C-28 sterol methyltransferase gene variants from A. thaliana and A. trichopoda were expressed under the control of the PrGPD and PrTEFintron promoters, respectively. The resulting strain ST12139 produced 5.4 mg/g DCW β-sitosterol.

### Construction of an Isofucosterol-Producing Strain

The 24-methylenecholesterol-producing strain ST11064 was used for the construction of an isofucosterol-producing strain (FIG. 17A). The C-28 sterol methyltransferase gene variant from C. quinoa was expressed under the control of the PrTEFintron promoter. The resulting strain ST11803 produced 5.0 mg/g DCW isofucosterol. To further increase isofucosterol production, two additional C-28 sterol methyltransferase gene variants from A. thaliana and A. trichopoda were expressed under the control of the PrGPD and PrTEFintron promoters, respectively. The resulting strain ST12108 produced 20.2 mg/g DCW isofucosterol.

### Construction of a Desmosterol-Producing Strain

The ΔERG5ΔERG6 strain ST11330 was used for the construction of a desmosterol-producing strain (FIG. 17B). The delta-7 sterol reductase gene variant from L. drancourtii was expressed under the control of the PrTEFintron promoter, generating strain ST11348. Strain ST11346 produced 9.4 mg/g DCW desmosterol. Note, as explained in the discussion below, the deletion of ERG5 is not deemed essential.

### Construction of a Cholesterol-Producing Strain

The desmosterol-producing strain ST11346 was used for the construction of a cholesterol-producing strain (FIG. 17B). The delta-24 sterol reductase gene variants from D. rerio and M. musculus were expressed under the control of the PrTEFintron promoter, generating strains ST11829 and ST11830, respectively. Strain ST11829 produced 26.6 mg/g DCW cholesterol and strain ST11830 produced 13.9 mg/g DCW cholesterol.

### DISCUSSION

Again, since tetrahymanol is able to substitute for ergosterol function in the cel, the ergosterol biosynthesis pathway could subsequently be disrupted without compromising cell viability. The native C-22 sterol desaturase gene ERG5 was deleted together with either ERG4 or ERG5 to prevent ergosterol synthesis and allow diversion of flux toward the non-native sterols upon the introduction of additional heterologous genes. The single gene deletion of ERG6 in combination with the expression of a heterologous delta-7 sterol reductase is sufficient for desmosterol production. In the case of strain ST11346, the deletion of ERG5 is not strictly necessary to obtain desmosterol but was included to facilitate the subsequent construction of cholesterol-producing strains.

In some strains, additional intermediate sterols may accumulate together with the main desired sterol. For example, the D. rerio delta-24 sterol reductase gene variant in ST11829 is more efficient at reducing desmosterol to cholesterol than the M. musculus variant in ST11830. Hence, ST11829 contains 0.3 mg/g DCW desmosterol and 26.6 mg/g DCW cholesterol, compared to 4.7 mg/g DCW desmosterol and 13.9 mg/g DCW cholesterol in strain ST11830. The C. quinoa C-28 sterol methyltransferase gene variant appeared to have weak activity and using this methyltransferase the production of 24-ethyl sterols was comparatively low-strain ST11804 produced 1.2 mg/g DCW β-sitosterol and 3.0 mg/g DCW of the precursor campesterol. Strain ST11803 produced 5.0 mg/g DCW isofucosterol and only 10.2 mg/g DCW of the precursor 24-methylenecholesterol. Consequently, two additional sterol methyltransferase gene variants were introduced into these strains to increase the relative content of the 24-ethyl sterols. The difference in the relative content of 24-ethyl sterols in the resulting strains ST12139 and ST12108 may be due to a difference in substrate specificity of the sterol methyltransferase enzymes toward the respective 24-methyl sterol precursors in each strain.

As previously noted above, it is anticipated that the ergosterol pathway is tightly regulated to control ergosterol concentration in the cell. This may partly be achieved by end-product feedback inhibition. This mechanism would be expected to be highly specific to ergosterol, to prevent interference by structurally similar early intermediates of the ergosterol pathway. Hence, sterol accumulation is greater in strains lacking ergosterol, particularly in those producing non-native sterols. These sterols are likely to be esterified and stored in lipid droplets to prevent toxicity. The amount of tetrahymanol remained less than 20 mg/g DCW in the strains producing each of the non-native sterols.

The examples given demonstrate that Y. lipolytica has the capacity to accumulate and store a range of different sterol structures. Engineered Y. lipolytica strains therefore hold great potential for the synthesis of many non-native sterol structures, and the dominant sterol may be tailored to suit different applications. It is expected that sterol content could be improved by further strain modification and scale-up of cultivations.

### ADDITIONAL REFERENCE

Zu P, Koch H, Schwery O. Pironon S, Phillips C, Ondo I, Farrell I W, Nes W D, Moore E. Wright G A, Farman D I & Stevenson P C (2021) Pollen sterols are associated with phylogeny and environment but not with pollinator guilds. New Phytol. 230: 1169-1184

### Example 4—Engineering of Yarrowia lipolytica for Production of a Mixture of Non-Native Sterols

### Materials and Methods

### Strains, Culture Conditions and Chemicals

Escherichia coli strain DH5α was used for plasmid construction as above.

The strain ST9100 was again used to construct all non-native sterol-producing strains. All strains are detailed in Tables 5a and 5b.

Y. lipolytica was grown at 30° C. on yeast extract peptone dextrose (YPD) media containing 10 g/l yeast extract, 20 g/l peptone, and 20 g/l glucose, supplemented with 20 g/l agar for preparation of solid media. For selection, either nourseothricin (250 mg/l) or hygromycin (400 mg/l) was added to the media.

Cultivation of strains for sterol production was again performed in yeast extract peptone medium containing 80 g/l glucose.

Chemicals were obtained, if not indicated otherwise, from Sigma-Aldrich. Nourseothricin was purchased from Jena BioScience GmbH (Germany).

### Plasmid Construction

The coding sequences for delta-7 sterol reductase from Tetraselmis sp. GSL018 (GenBank accession: JAC78771.1), squalene-tetrahymanol cyclase from Tetrahymena thermophila (accession: XP_001026696.2), delta-24(25) sterol reductase from Solanum lycopersicum (accession: BAQ55273.1), C-28 sterol methyltransferase from Chenopodium quinoa (accession: XP_021737090.1) and delta-24(28) sterol reductase from Solanum tuberosum (accession; AB839749.1) were codon-optimized for Y. lipolytica and synthesised as GeneArt String DNA fragments by ThermoFisher Scientific. The codon-optimized sequences are listed in Tables 4a and b.

The plasmids, biobricks, and primers used in this study are listed in Tables 15-17, respectively. BioBricks were again amplified by PCR. The PCR mix contained 32 μl water, 10 μl high fidelity Phusion® polymerase buffer (5×), 1 μl 10 mM dNTP, 1.5 μl MgCl₂, 2.5 μL 10 μM Fw primer, 2.5 μl 10 μM Rv primer, 0.2 μl Phusion® U polymerase, and 10 ng DNA template. Reactions were multiplexed 8× per BioBrick. The cycling program was: 98° C. for 1 min. 30 cycles of [98° C. for 10 sec. gradient from 50 to 58° C. for 20 sec. 72° C. for 30 s/kb], 72° C. for 5 min, pause at 10° C. The gene fragments were purified from agarose gels using the NucleoSpin® Gel and PCR Clean-up kit (Macherey-Nagel). Biobricks were assembled into the EasyCloneYALI vectors with USER cloning (Holkenbrink et al. 2018). The USER reactions were transformed into E. coli and correct assemblies were verified by Sanger sequencing (Eurofins).

TABLE 15
Plasmids for engineering of Yarrowia lipolytica
for production of a mixture of non-native sterols

| | Parent plasmid/ | |
|---|---|---|
| Plasmid name | Reference | BioBricks |
| pCfB5935 (pIntA-1-HphMx-TPex20- | (Holkenbrink et al, | |
| TLip2) | 2018) | |
| pCfB6633 (pNat-YLgRNA2_IntE_1) | (Holkenbrink et al, | |
| | 2018) | |
| pCfB6637 (pNat-YLgRNA3_IntE_3) | (Holkenbrink et al, | |
| | 2018) | |
| pCfB6638 (pNat-YLgRNA2_IntE_4) | (Holkenbrink et al, | |
| | 2018) | |
| pCfB6677 (pIntE_1-TPex20-TLip2) | (Holkenbrink et al, | |
| | 2018 | |
| pCfB6681 (pIntE_3-TPex20-TLip2) | (Holkenbrink et al, | |
| | 2018) | |
| pCfB4783 (pIntE_3-Nat-TPex20- | (Holkenbrink et al, | |
| TLip2) | 2018) | |
| pCfB6679 (pIntE_4-TPex20-TLip2) | (Holkenbrink et al, | |
| | 2018) | |
| pBP8009 (pIntF_3-TPex20-TLip2) | (Sáez-Sáez et al, 2020) | |
| pCfB10783(gRNA_IntE_5) | Dr. J. Dahlin, DTU CfB, | |
| | Denmark | |
| pBP8003 (pNat-YLgRNA4-IntF_3) | (Sáez-Sáez et al, 2020) | |
| pCfB8861 (pHyg-YLgRNA3_IntE_4) | (Arnesen et al, 2020) | |
| pBP8660 (pIntE_5-TPex20-TLip2) | Dr. K. R. Kildegaard, | |
| | Biophero ApS, | |
| | Denmark | |
| pCfB6611 (pNat-PrExp-Cre) | pCfB4158 (pPrExp-Cre) - | |
| | (Holkenbrink et al, | |
| | 2018) | |
| pCfB6612 (pHph-PrExp-Cre) | pCfB6611 (backbone | BB2816 (PrExp-HphMx-Ttef) |
| | amplified with PR- | |
| | 21844 and PR-10593) | |
| pCfB10257 (IntE4_PrTEF- | pCfB6679 | BB3879 (Tefint->):BB4884 |
| >DHCR7_Tsp) | | (PrTEF_DHCR7Ts) |
| pCfB10494 (IntE3_PrGPAT->TtSTC) | pCfB6681 | BB1617 (PrGPAT):BB5100 |
| | | (TtSTC_USER) |
| pCfB10841 (IntF3_PrDGA1->ERG4) | pBP8009 | BB1616 (PrDGA1):BB5362 |
| | | (ERG4_USER) |
| pCfB10842 (IntF3_PrGPAT->ERG4) | pBP8009 | BB1617 (PrGPAT):BB5362 |
| | | (ERG4_USER) |
| pCfB10851 (IntE1_PrGPAT- | pCfB6677 | BB1617 (PrGPAT->):BB5371 |
| >SMT2_Cquinoa) | | (PrGPAT_SMT2Cq) |
| pCfB10853 (IntE5_PrGPAT- | pBP8660 | BB1617 (PrGPAT->):BB5373 |
| >DHCR24_Slycopersic) | | (PrGPAT_DHCR24SI) |
| pCfB10919 (Int_3 NatMX_PrGPAT- | pCfB4783 | BB5371 (PrGPAT_SMT2Cq) |
| >SMT2_Cquinoa) | | |
| pCfB10948 (IntF3_PrDGA1->StSSR1) | pCfB8009 | BB1616 (PrDGA1):BB5456 |
| | | (StSSR1_USER) |

Full reference for Sáez-Sáez et al. (2020): Sáez-Sáez J, Wang G, Marella E R, Sudarsan S, Cemuda Pastor M & Borodina I (2020) Engineering the oleaginous yeast Yarrowia lipolytica for high-level resveratrol production. Metab. Eng. 62: 5

TABLE 16
Biobricks for engineering of Yarrowia lipolytica
for production of a mixture of non-native sterols

| | | Forward | Reverse |
|---|---|---|---|
| Biobrick name | Template/Reference | primer | primer |
| BB1616 (PrDGA1->) | (Holkenbrink et al, 2018) | | |
| BB1617 (PrGPAT->) | (Holkenbrink et al, 2018) | | |
| BB3879 (PrTefint->) | (Arnesen et al, 2020) | | |
| BB4884 | Tetraselmis sp. GSL018 delta-7 | PR- | PR- |
| (PrTEF_DHCR7Ts) | sterol reductase codon | 27581 | 27582 |
| | optimised | | |
| BB2816 (PExp- | pCfB5935 - (Holkenbrink et al, | PR- | PR- |
| HphMx-Ttef) | 2018) | 15604 | 22297 |
| BB5041 | pCfB5935 - (Holkenbrink et al, | PR- | PR- |
| (LoxP_HphMX) | 2018) | 27972 | 27973 |
| BB5101 | | PR- | PR- |
| (LoxP_NatMX) | pCfB4788 | 27972 | 27973 |
| BB5091 | ST9100 gDNA | PR- | PR- |
| (erg4_1 kbUP_U1) | | 27891 | 28358 |
| BB5092 | ST9100 gDNA | PR- | PR- |
| (erg4_1 kbDOWN_U2) | | 27893 | 27894 |
| BB5093 | ST9100 gDNA | PR- | PR- |
| (erg5_1 kbUP_U1) | | 27895 | 28359 |
| BB5094 | ST9100 gDNA | PR- | PR- |
| (erg5_1 kbDOWN_U2) | | 27897 | 27898 |
| BB5097 | BB5091 + BB5092 + BB5041 | PR- | PR- |
| (erg4_HphMX_KO) | USER reaction | 27891 | 27894 |
| BB5098 | BB5093 + BB5094 + BB5041 | PR- | PR- |
| (erg5_HphMX_KO) | USER reaction | 27895 | 27898 |
| BB5100 (TtSTC_USER) | Tetrahymena thermophilia | PR- | PR- |
| | squalene-tetrahymanol cyclase | 28361 | 28362 |
| | codon optimised | | |
| BB1135 (Easy Clone | pCfB6677 | PR- | PR- |
| vector backbone) | | 11110 | 11111 |
| BB5362 (ERG4_USER) | ST9100 gDNA | PR- | PR- |
| | | 29139 | 29140 |
| BB5371 | Chenopodium quinoa sterol | PR- | PR- |
| (PrGPAT_SMT2Cq) | methyltransferase codon | 27621 | 27786 |
| | optimised | | |
| BB5373 | Solanum lycopersicum delta-24 | PR- | PR- |
| (PrGPAT_DHCR24SI) | sterol reductase codon | 27599 | 27764 |
| | optimised | | |
| BB5456 | Solanum tuberosum delta- | PR | PR- |
| (StSSR1_USER) | 24(28) sterol reductase codon | 29404 | 29405 |
| | optimised | | |

TABLE 17
Primers for engineering of Yarrowia lipolytica for production of a mixture of non-
native sterols

| Primer name | Sequence (5′ → 3′) |
|---|---|
| PR-14617 (vector verification E. | tatccctgtgttgaatc |
| coli cPCR) | |
| PR-14619 (vector verification E. | tatcgacccagttagc |
| coli cPCR) | |
| PR-8859 (integration verification | aagtgtggatggggaagtgag |
| YI cPCR) | |
| PR-14442 (IntE_1 verification YI | agttgtgaccaagacaaatg |
| cPCR) | |
| PR-14398 (IntE_1 verification YI | cacgcgaUgttagaagcaattggagaag |
| cPCR) | |
| PR-14576 (IntE_3 verification YI | cacgcgautgaaggaaatgcctaaaacc |
| cPCR) | |
| PR-14835 (IntE_3 verification YI | cacgcacgccattctataag |
| cPCR) | |
| PR-14592 (IntE_4 verification YI | acgcgauttaacactggaccgtactgc |
| cPCR) | |
| PR-20880 (IntE_4 verification YI | attgctaagcgaccatagac |
| cPCR) | |
| PR-14837 (IntF_3 verification YI | acagctcgcgcctcgatag |
| cPCR) | |
| PR-14584 (IntF_3 verificiationYI | cacgcgautttggtcgtcgcccaacaag |
| cPCR) | |
| PR-26834 (IntE_5 verification YI | gggacaagtgcaatcgttcacttg |
| cPCR) | |
| PR-26835 (IntE_5 verification YI | cgttgctgttgccacaatggac |
| cPCR) | |
| PR-27581 (A_Ts_Fw) | actttttgcagtacuaaccgcagaagcgagcctccaagaccc |
| PR-27582 (A_Ts_Rv) | cacgcgauttagaagatgtagggcacgatc |
| PR-27891 (Erg4Rep_P1F) | ctctcaacaccttcaccgc |
| PR-28358 (Erg4Rep_P1R_U1) | atcgcacgugataagcttagtgagcgaatgg |
| PR-27893 (Erg4Rep_P2F) | gtgcaggutgtgtggttgcgaaggaag |
| PR-27894 (Erg4Rep_P2R) | gcactcaaaataccccgttc |
| PR-27895 (ErgSRep_P1F) | ctcggtttgttgcagcagg |
| PR-28359 (Erg5Rep_P1R_UI) | atcgcacgutggtccgtatcgtgaaatgg |
| PR-27897 (Erg5Rep_P2F) | gtgcaggugggcggagttgtgttgtg |
| PR-27898 (Erg5Rep_P2R) | ggtcggctatccaatacatctc |
| PR-27972 (HphMX_ F_U1) | cgtgcgautcagctgaagcttcgtac |
| PR-27973 (HphMX_R_U2) | acctgcacugcataggccactagtgg |
| PR-15604 (PrExp_for fusion- | agctactgauaaggagtttggcgcccgtt |
| >_U2_fw) | |
| PR-22297 (Ttef_U_EpiVecYL_rv) | aagcctgcugaattcggacacgggcat |
| PR-21844 (gRNA_opened_rv) | atcagtagcuctactgtatattcaagcaag |
| PR-10593 (Fragment2EpiVecYL_ | agcaggctuggaggcgacgtggcag |
| fw) | |
| PR-28361 (TISTC_F_U3) | atctgtcaugccacaatgaagaagatcctcatcggtc |
| PR-28362 (TtSTC_Rv) | cacgcgauttagatgttctgcttctggacg |
| PR-22830 (Derg5_chk) | tcatactcaccgaaacgtg |
| PR-27961 (Derg5_chk) | gttccaatgcctggcaag |
| PR-27634 (Derg5_chk) | acttctctctctcacaccacc |
| PR-27635 (Derg5_chk) | ctgagggctctgttggtgang |
| PR-27636 (Derg5_chk) | accagtgtggttgtaaggatg |
| PR-11138 (Derg4_chk) | agcaatggguaaaaagcctgaactcaccgc |
| PR-27631 (Derg4_chk) | cctgatattggtgatcctcc |
| PR-27632 (Derg4_chk) | agagccttgtttccgaggtg |
| PR-27633 (Derg4_chk) | atacaatcccatagcctggc |
| PR-28001 (Derg4_chk) | cgtgcgaugcttgccctggactacatcttg |
| PR-27599 (B_SI_Fw) | atctgtcaugccacaatgtctgacgccaaggctc |
| PR-27764 (B_SI_Rv) | cacgcgauttagttggcctcagggg |
| PR-27621 (C_Cq_Fw) | atctgtcaugccacaatggactctatggccctg |
| PR-27786 (C_Cq_Rv) | cacgcgauttaagagtcagacttggc |
| PR-29139 (ERG4_ORF_F_U3) | atctgtcaugccacaatgtctgctgttcgacaacg |
| PR-29140 (ERG4_ORF_Rv) | cacgcgauttagaaaacataagggatgaagatcc |
| PR-28981 | atcgcgtgcautccttctgttcggaatcaacct |
| (Backbone_amp_Tlip_F) | |
| PR-29404 (StSSR1_F_U3) | atctgtcaugccacaatgaccgacgtccagg |
| PR-29405 (StSSR1_Rv) | cacgcgauttagtcctcgggctcgtc |

Yeast Transformation Again, the yeast vectors were integrated into different previously characterized intergenic loci in the Y. lipolytica genome as described in Holkenbrink et al, 2018; see also Table 18 below. Integration vectors were digested with Notl enzyme (New England Biolabs) at 37° C. for 1 hr and the digested product purified from solution using the NucleoSpin® Gel and PCR Clean-up kit (Macherey-Nagel). The purified DNA was transformed using the lithium acetate transformation protocol as described by Holkenbrink et al, 2018. Correct integration was verified by colony PCR using Taq DNA Polymerase Master Mix RED (Ampliqon) with vector-specific primers and primers complementary to the genomic region adjacent to the integration site.

For marker-mediated gene deletion, Y. lipolytica strains were transformed with biobricks assembled by USER reaction as detailed in Table 18. Transformants were selected on antibiotic supplemented plates and correct transformants confirmed by colony PCR. Marker removal was performed by transformation of the strains with a Cre-recombinase episomal vector. Marker removal was confirmed by colony PCR.

### Yeast Cultivation

Yeast strains were inoculated into 2.5 ml YPD in 24-deepwell plates with air-penetrable lids (EnzyScreen, Netherlands). The plates were incubated at 30° C. with 300 rpm agitation at 5 cm orbit cast for 24 hours. The cultures were then diluted to OD600 0.1 in 2.5 ml fresh YPD-medium with 80 g/l glucose and grown for a further 72 hours at 30° C. with 300 rpm agitation. All cultivations were performed in triplicate. Dry cell weight was measured at the end of cultivation: 1 ml of culture broth was transferred into a pre-weighed 2 mi microcentrifuge tube, centrifuged (3000 g, 5 min) and the supernatant was discarded. The cells were washed twice with deionized water (1 ml). The con pellet was dried at 0° C. for 7 days before the final weight was measured.

### Sterol Analysis

For sterol extraction, 1 ml of culture broth was transferred into a 2 ml microcentrifuge tube, centrifuged and the supernatant was discarded. The cells were washed twice with deionized water (1 ml). The cell pellet was resuspended in 10% w/v methanolic potassium hydroxide (500 μl) and transferred to a 1 ml glass vial for saponification. The suspension was incubated at 70° C. for 2 hours with vortexing at 15 minute intervals. The saponified samples were then vortexed and spiked with 50 μl of internal standard (1 mg/mi epicoprostanol in absolute ethanol). 500 μl of n-hexane was added to each sample for extraction of the free sterol component. Samples were vortexed and the organic phase transferred to a 2 ml microcentrifuge tube. The extraction step was repeated in a further 500 μl of n-hexane. The combined hexane phases were left overnight at room temperature for evaporation of the solvent. The resulting crystals were resuspended in 50 μl of n-hexane for concentration at the bottom of the tube and left at room temperature overnight for final drying. Samples were then stored at 4° C. prior to analysis.

Sterols were derivatized with 20 μl Tri-Sil (Sigma) and then briefly vortexed before direct injection into an Agilent Technologies (Palo Alto, CA, USA) 7890A gas chromatograph connected to an Agilent Technologies 5975° C. MSD mass spectrometer (for gas chromatography-mass spectrometry (GC-MS)) and eluted over an Agilent DB5 column using a splitless injection at 250° C. with a standard GC program at 170° C. for 1 min, ramped to 280° C. at 20° C. min⁻¹ and monitoring between 50 and 550 amu.

Sterols were identified by comparison of their retention time relative to cholesterol and mass spectra data available from the NIST (National Institute of Standards and Technology) mass spectral library and Zu et al. 2021. Sterols were quantified by calculating the ratio of the peak area of the targeted sterol to that of the internal standard. The mass of each sterol in the sample was obtained by multiplying the ratio with the mass of the internal standard. Compound identification (using target ions) and quantification were carried out with ChemStation Enhanced Data Analysis (v.E.01.00).

### Bioreactor Fed-Batch Cultivation

The Ambr® 250 system (Sartorius Stedim Biotech, Göttingen, Germany) was used to carry out 250 ml fed-batch fermentation in duplicate. Strain ST11064 was re-streaked from glycerol stocks onto a YPD agar plate and incubated at 30° C. for 48 h. The preculture was prepared by inoculating strain ST11064 biomass from the plate into 50 ml YPD medium in a 250 ml shake flask and incubating at 30° C. for 24 hours with 250 rpm agitation. Five ml of preculture was used to inoculate 115 ml batch medium, to a starting OD₆₀₀ of 0.25. For Ambr® 250 cultivation, the batch medium comprised mineral medium supplanted with yeast extract (10 g/l) and citric acid (20 g/l). The mineral medium was prepared with 0.5 g/l MgSO₄·7H₂O, 14.4 g/KH₂PO₄, 0.1% (v/v) vitamin solution, 0.2% (v/v) trace metal solution as described in (Jensen et al, 2014), but with 3.4 g/l NH₄Cl and glycerol as the carbon source (40 g/l) Temperature was held constant at 30° C. Dissolved oxygen was maintained above 20% by using a cascade of stirring speed ranging from 600 to 3000 rpm and aeration up to 1 v/v. The pH was maintained at 5.5 by the automatic addition of 1 M NaOH and 2.8 M H₃PO₄ phosphoric acid. Antifoam 204 (Sigma) was added automatically. The feed medium comprised 250 g/l glycerol. Feeding was automatically initiated once CER dropped below 50% at the end of the batch phase. Feeding was set to a constant rate of 0.9 m/h. Samples were taken every 8 h for the first 24 hours, and then every 12 hours, and immediately frozen until preparation for analysis. CDW and sterol content were determined from 1 ml samples as described above for small scale cultivation.

For larger scale fermentation, strains were cultivated by fed-batch fermentation in a 5 L bioreactor (BIOSTAT® B-DCU, Sartorius, Goettingen, Germany). All fermentations were carried out in duplicate. For each of strains ST4842. ST11005 and ST12178, the strain was re-streaked from glycerol stocks onto a YPD agar plate and incubated at 30° C. for 24 h. The preculture was prepared by inoculating strain biomass from the plate into 50 ml YPD medium in a 250 ml shake flask and incubating at 30° C. for 24 hours with 250 rpm agitation. The volume of pre-culture required to inoculate 2 L batch medium to a starting OD₆₀₀ of 2.5 was centrifuged for 10 min at 4000×g and concentrated in 10 ml volume.

This cell suspension was used to inoculate the bioreactors. The bioreactors were equipped with pH, pO2 and temperature probes. Temperature was held constant at 30° C. Dissolved oxygen was maintained above 20% by adjusting stirring between 800 and 1200 rpm and aeration (via a horseshoe sparger) between 0.5 and 3 standard-litre per min (SPLM). The pH was kept at 5.5 by automatic addition of 5 M NaOH. Antifoam A (Sigma) was added as required.

The batch media comprised of mineral medium supplanted with yeast extract (20 g/l) and peptone (40 g/l). The mineral medium was prepared with 0.5 g/l MgSO₄·7H₂O, 14.4 g/l KH₂PO₄, 0.1% (v/v) vitamin solution, 0.2% (v/v) trace metal solution as described in (Jensen et al, 2014), 40 g/l glucose and 1 ml/l antifoam A (Sigma). The feed contained 5 g/l MgSO₄·7H₂O, 30 g/l KH₂PO₄, 1% (v/v) vitamin solution, 2% (v/v) trace metal solution as described in (Sáez-Sáez et al, 2020), with 300 g/l glucose. An exponential feeding profile was programmed, and feeding was initiated 24 h after inoculation. Feed rate, F (ml/h) followed the profile F=10*e\hat{ }(0.05*t), where t=time (h) from the start of feeding. After 36 h of exponential feeding, the feed was switch to a constant rate of 75 m/h until the end of fermentation.

Samples were taken every 8 hours for the first 24 hours, and then every 12 hours, to measure CDW, sterol content, OD600 and glucose concentration. CDW and sterol content were determined from 1 ml samples as described above for small scale cultivation. OD800 was measured with a VWR NanoPhotometer 7122. During fermentation, 1 ml culture broth was centrifuged and the supernatant was used to measure glucose concentration via a glucose HK assay kit (Sigma). The supernatant was then filtered and frozen until further analysis. Glucose was later quantified on a Dionex Ultimate 3000 HPLC system equipped with a RI-101 Refractive Index Detector (Dionex) refractive index detector. An Aminex HPX-87H column 7.8 mm×300 mm (Bio-Rad) with a Micro-Guard Cation H+ guard column 4.6 mm×30 mm heated to 30° C. was injected with 10 μl sample. The mobile phase consisted of 5 mM H₂SO₄ with an isocratic flow rate of 0.8 ml/min, which was held for 15 min. HPLC data were processed using Chromeleon 7.2.9 software (Thermo Fisher Scientific). Glucose was identified and quantified using authentic standards. Glucose concentrations were calculated from peak area by extrapolation from a six-point calibration curve regression.

### Results

Construction of a Yarrowia lipolytica Strain Capable of Synthesising a Sterol Surrogate

The gene encoding squalene-tetrahymanol cyclase from Tetrahymena thermophila (TtSTC) was integrated into the ST9100 platform strain genome under the control of the PrGPAT promoter as previously described above.

Construction of Yarrowia lipolytica Strains Incapable of Synthesising Ergosterol

The tetrahymanol producing strain ST11005 was used for the construction of strains incapable of synthesising ergosterol. Firstly, to create a erg5 knock-out strain, the ERG5 gene (sterol C-22 desaturase, YALI1_A18344 g) was deleted by marker-mediated knock-out as described above to generate ST11014. The HphMX resistance marker was subsequently looped out by Cre-Lox recombination to provide strain ST11027 (produced 3.1 mg/g DCW ergosta-5,7-dienol, comprising 60.0% of the total sterol fraction; the strain also produced 5.2 mg/g DCW tetrahymanol, comprising 36.7% of the total sterol fraction).

To create a ΔERG5ΔERG4 strain, the ERG4 gene (delta-24 sterol reductase, YALI1_D24361 g) was deleted by HphMX marker-mediated knockout in an analogous manner. As noted above, the resulting strain ST11040 produced 3.1 mg/g DCW ergosta-5,7,24(28)-trienol, comprising 20.0% of the total sterol fraction. The strain also produced 15.8 mg/g DCW tetrahymanol, comprising 75.2% of the total sterol fraction.

### Construction of Strains Capable of Producing One or More Exogenous Sterols

The ΔERG5ΔERG4 strain ST11040 was used for the construction of 24-methylenecholesterol-producing strains. The delta-7 sterol reductase gene variant from Tetraselmis sp. GSL018 was expressed under the control of the PrTEFintron promoter, generating strain ST11064 as described above (produced 47.7 mg/g DCW 24-methylenecholesterol).

Strain ST11064 was then used to create strains capable of producing 24-methylenecholesterol and campesterol. The native ERG4 gene was re-introduced, under the control of previously characterised weak promoters (Holkenbrink et al, 2018). ERG4 was expressed under the control of the PrDGA1 promoter in ST11064, generating ST11337 which produced 2.8 mg/g DCW 24-methylenecholesterol and 2.2 mg/g DCW campesterol. ERG4 was expressed under the control of the PrGPAT promoter in ST11064, generating ST11338 which produced 5.0 mg/g DCW campesterol and trace 24-methylenecholesterol.

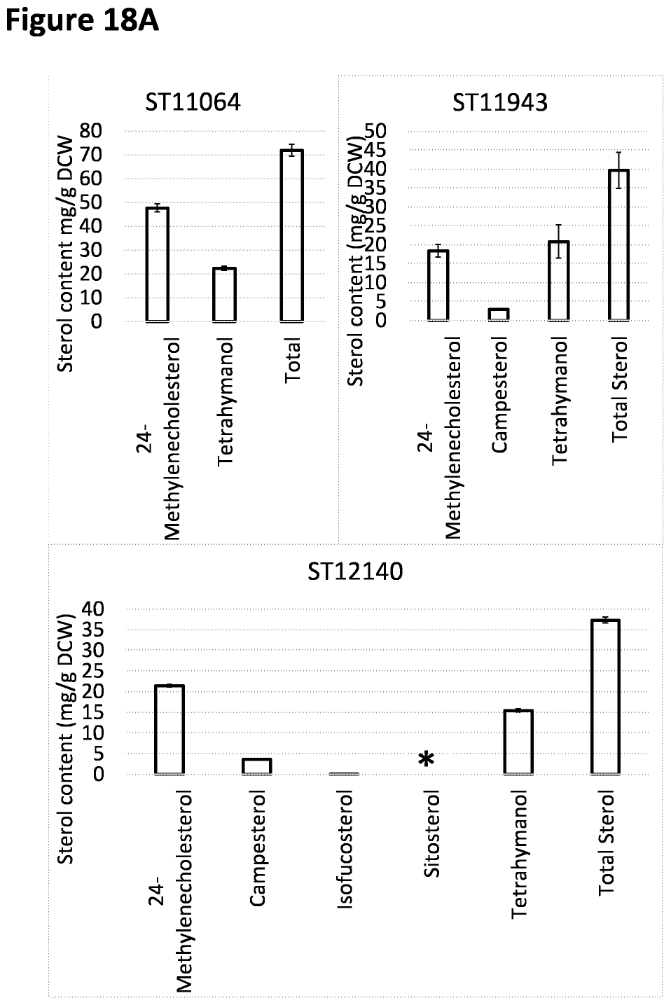

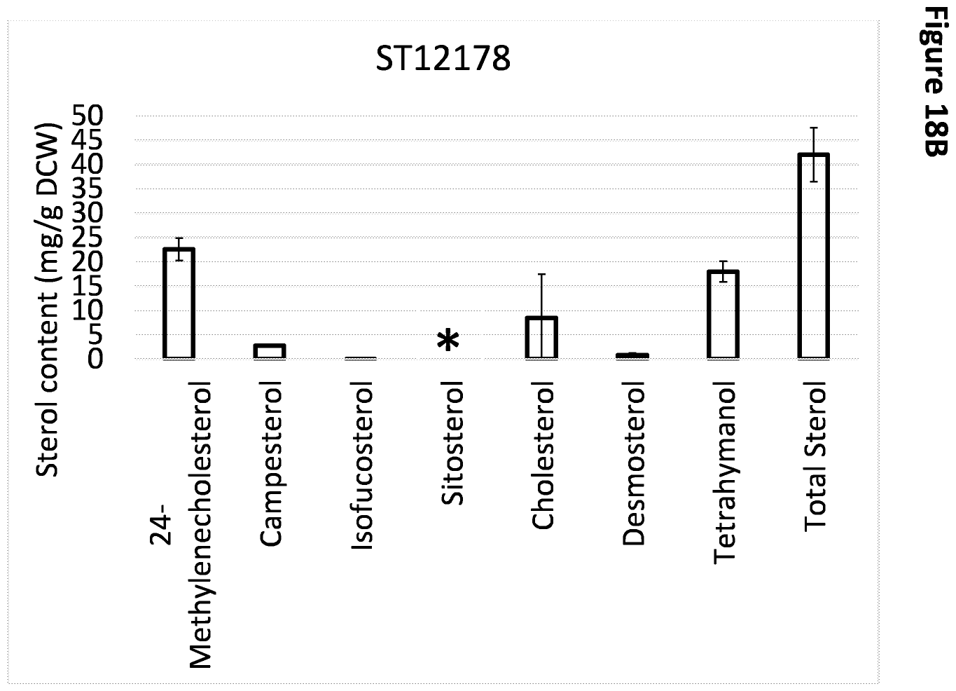

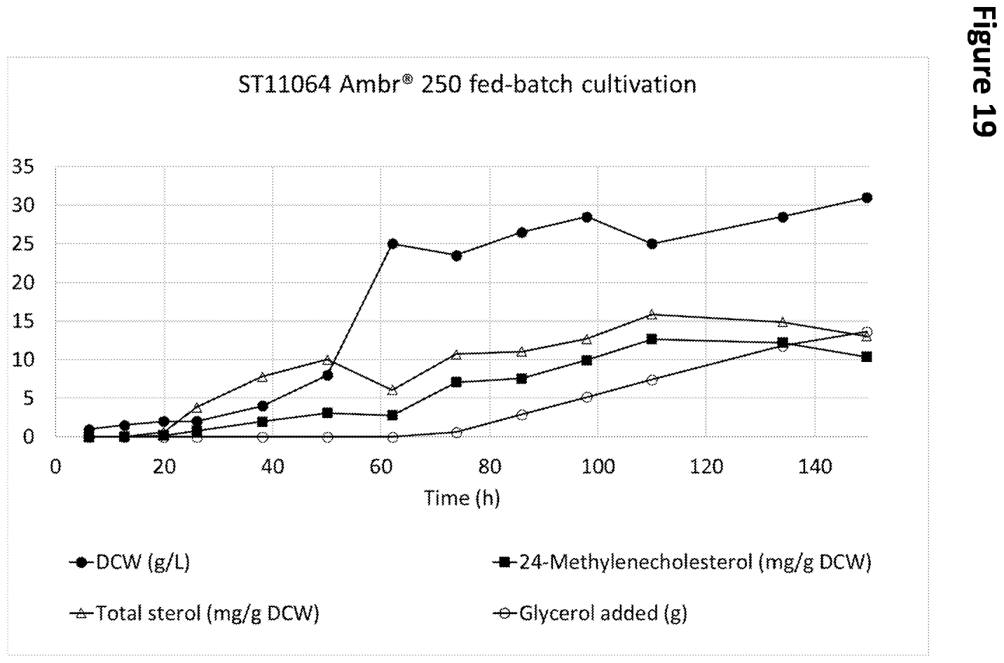

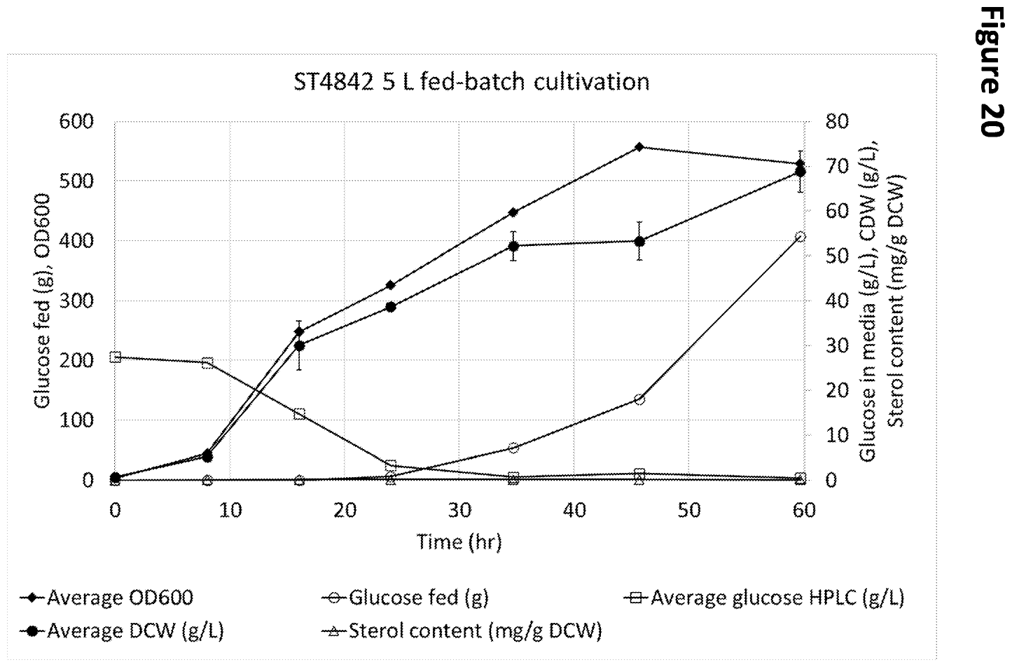

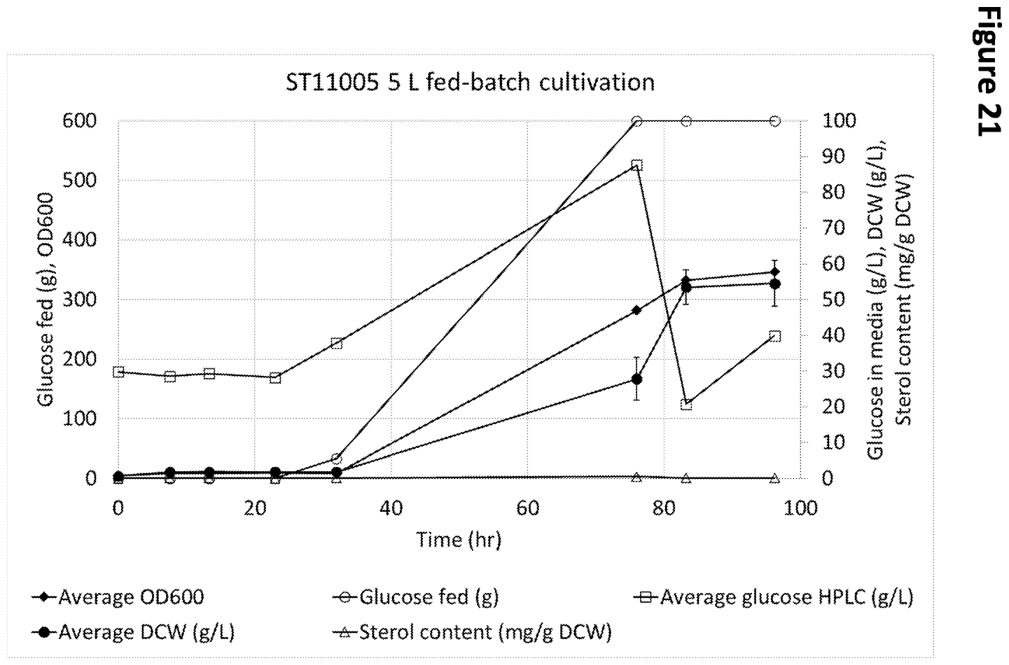

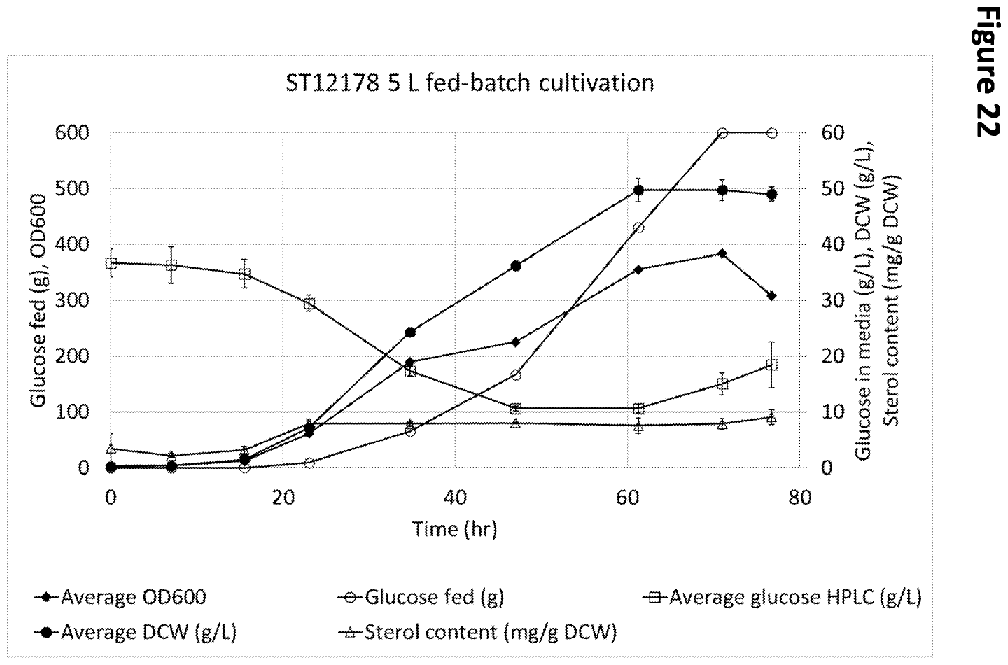

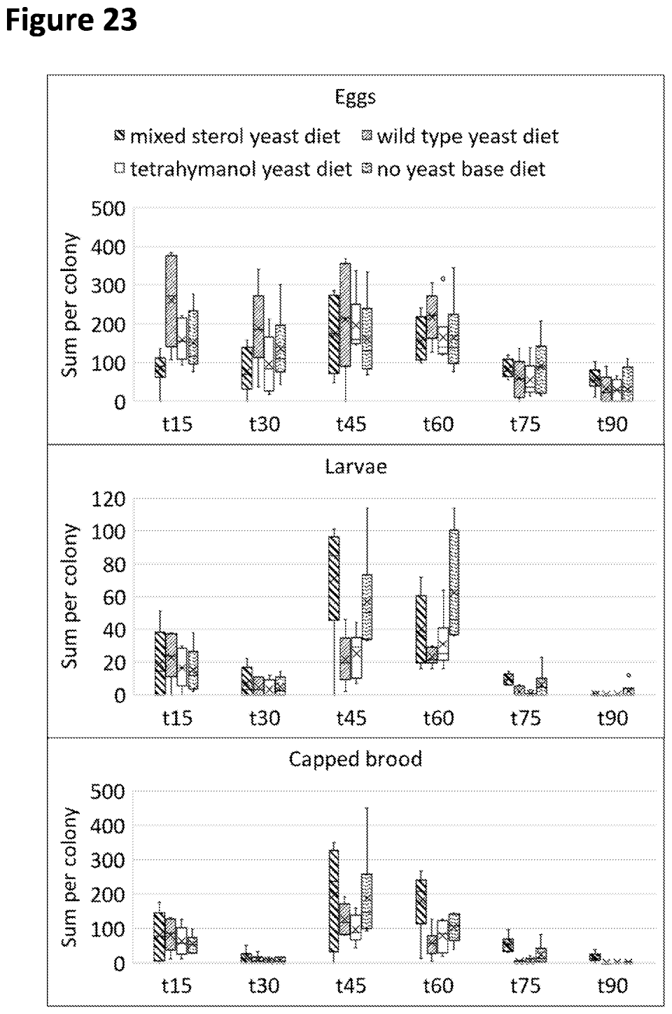

Strain ST11064 was also used to create a strain capable of producing 24-methylenecholesterol and campesterol by introducing the optimised coding sequence for the delta-24(28) sterol reductase of S. tuberosum (StSSR1) under the control of the PrDGA1 promoter. This resulted in strain ST11943 which produced 18.4.mg/g DCW 24-methylenecholesterol and 3.0 mg/g DCW campesterol (see FIG. 18A). Importantly, as further discussed below the choice of a plant delta-24(28) sterol reductase (DWF1) for expression in this strain ensured the production of the plant epimer of campesterol. Strain ST11943 was further engineered to attain additional non-native sterol mixtures.

The C-28 sterol methyl transferase from C. quinoa was expressed under the control of the PrGPAT promoter in strain ST11943 to enable the production of C-24 ethyl sterols (FIG. 18A). The resulting strain ST12140 produced 21.4 mg/g DCW 24-methylenecholesterol, 3.7 mg/g DCW campesterol, 0.02 mg/g DCW isofucosterol, and trace amounts of beta-sitosterol.

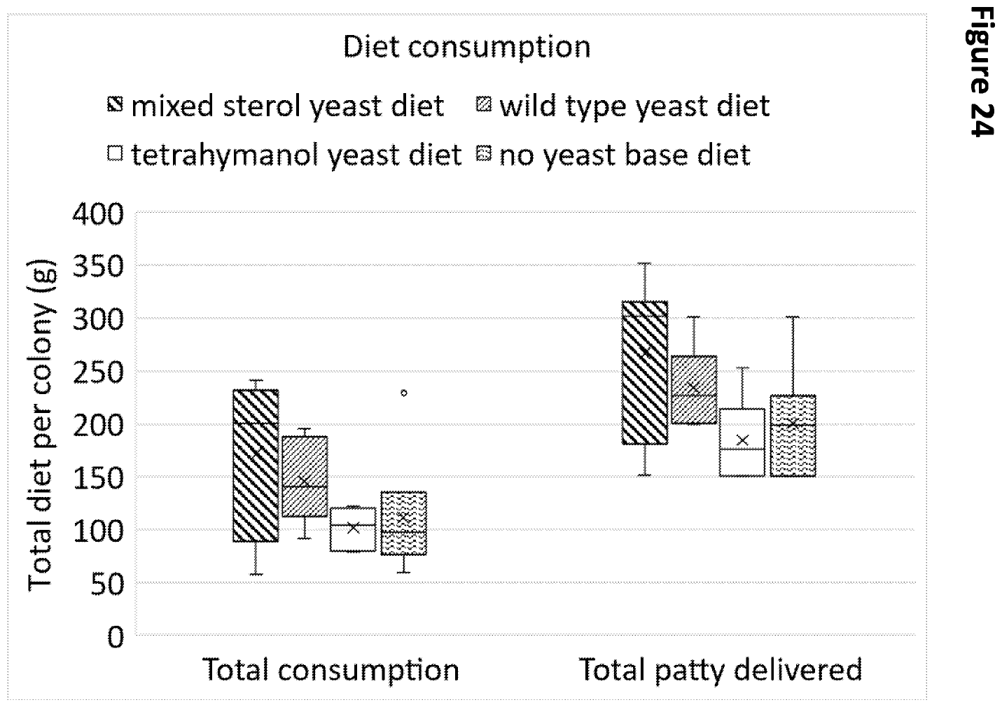

An optimised coding sequence for the delta-24(25) sterol reductase of S. lycopersicum was introduced, under the control of the PrGPAT promoter, into strain ST12140 to generate strain ST12178 (FIG. 188). Strain ST12178 produced 22.6 mg/g DCW 24-methylenecholesterol, 2.8 mg/g DCW campesterol, 0.01 mg/g DCW isofucosterol, trace amounts of beta-sitosterol, 8.5 mg/g DCW cholesterol and 0.8 mg/g DCW desmosterol. Again importantly production of the plant epimer of campesterol was ensured but with 24-methylenecholesterol as the dominant non-native sterol. The mix of non-native sterols observed led to this strain being further tested as a dried yeast powder for feeding honeybees following larger scale fermentation as further described below.

The same optimised coding sequence for the delta-24(25) sterol reductase gene of S. lycopersicum was also introduced, under the control of the PrGPAT promoter, into strains ST11337 and ST11338 to generate strains ST11378 and ST11340, respectively. The S. lycopersicum delta-24(25) sterol reductase enables the production of cholesterol from 24-methylenecholesterol (Sawai et al., (2014) The Plant Cell 26: 3763-3774) as shown also by strain ST12178. Strain ST11378 produced 9.6 mg/g DCW 24-methylenecholesterol, 0.4 mg/g DCW campesterol and 0.4 mg/g DCW cholesterol. Unexpectedly, strain ST11378 also produced 0.1 mg/g DCW desmosterol. This is likely to be an intermediate or side-product arising from activity of the S. lycopersicum delta-24(25) sterol reductase. Strain ST11340 produced 4.6 mg/g DCW campesterol, trace 24-methylenecholesterol, 0.1 mg/g DCW cholesterol and trace desmosterol.

To create strains of mixed composition wherein campesterol is the dominant sterol, SMT (C-28 sterol methyltransferase) was expressed in ST11340, followed by deletion of TtSTC to remove tetrahymanol production. The C-28 sterol methyltransferase from C. quinoa was expressed under the control of the PrGPAT promoter in strain ST11340 to enable the production of C-24 ethyl sterols. The resulting strain. ST11362, produced 4.3 mg/g DCW campesterol and 0.1 mg/g DCW cholesterol as well as trace amounts of 24-methylenecholesterol, isofucosterol and beta-sitosterol. It was found possible to remove the TtSTC gene from that strain to create strain ST11441. The resulting strain can produce a mixture of phytosterols without a sterol surrogate. Strain ST11441 produced 11.9 mg/g DCW campesterol, 0.2 mg/g DCW 24-methylenecholesterol, 0.3 mg/g DCW cholesterol and trace amounts of desmosterol, isofucosterol and beta-sitosterol (see FIG. 15)

To create strains of mixed composition wherein 24-methylenecholesterol is the dominant sterol, TtSTC was deleted from ST11378 to remove tetrahymanol production and SMT was simultaneously introduced. The HphMX resistance marker was first removed from the ERG4 locus by Cre-Lox recombination. The TtSTC gene was then removed from strain ST11378 by homologous recombination with a cassette containing the C-28 sterol methyltransferase from C. quinoa under the control of the PrGPAT promoter and a NatMX resistance marker. The NatMX resistance marker was subsequently looped out by Cre-Lox recombination. The resulting strain ST11542 can produce a mixture of phytosterols without a sterol surrogate. Strain ST11542 produced 4.1 mg/g DCW 24-methylenecholesterol, 1.1 mg/g DCW campesterol, 0.3 mg/g DCW cholesterol, 0.01 mg/g DCW desmosterol and trace amounts of isofucosterol and beta-sitosterol (see FIG. 15).

### Cultivation in Bioreactors

The temporal growth and sterol production of strain ST11064 was investigated by fed-batch cultivation in 250-ml Ambr® bioreactors (FIG. 19). DO was maintained above 20%. pH kept at 5.5 and temperature at 30° C. Fermentations were carried out in duplicate. Defined medium has previously been used to reach high biomass concentrations in Y. lipolytica cultivation (Sáez-Sáez et al, 2020). Hence, the batch medium comprised mineral medium supplemented with yeast extract and citric acid. The addition of citric acid has been shown to increase production of the monoterpenoid linalool, which may be due to the concurrent increase in cytosolic acetyl-CoA and flux though the mevalonate pathway (Cao at al, 2017), which may also benefit sterol production. Yeast extract was used as an organic nitrogen source. Inorganic nitrogen was provided in the form of ammonium chloride since ammonium chloride was found to be better than ammonium sulphate for lipid accumulation when using glycerol as a carbon source (Back et al, 2016). The biomass concentration increased for the first 60 hrs of fermentation, at which point 24-methylenecholesterol accumulated during the stationary phase until ˜130 hrs of cultivation. After 150 hours of cultivation, the final biomass concentration was 31 g/l, with a 24-methylenecholesterol content of 10.4 mg/g DCW.

Strains ST4842, ST11005 and ST12178 were cultivated in duplicate in 5 L bioreactors by fed-batch fermentation (FIGS. 20-22). DO was maintained above 20%. pH kept at 5.5 and temperature at 30° C. The batch medium comprised mineral media (without inorganic nitrogen) supplemented with yeast extract and peptone to create a rich medium to support biomass production. The feed was also enriched to maximise biomass production. The feed contained 300 g/l glucose with additional 5 g/l MgSO₄·7H₂O, 30 g/l KH₂PO₄, 1% (v/v) vitamin solution, 2% (v/v) trace metal solution as described in (Sáez-Sáez et al, 2020). After a 24 hour batch phase, exponential feeding was initiated. After 80 hours of cultivation, the final biomass concentration was 68.8 g/l, with a total sterol content of 0.11 mg/g DCW for ST4842. For ST11005, fermentation ran for 72 hours and the final biomass concentration was 54.5 g/l, with a sterol content of 0.15 mg/g DCW. Strain ST12178 was cultivated for 84 hours, with a final biomass concentration was 49.0 g/l, with a total sterol content of 9.1 mg/g DCW comprising 6.0 mg/g DCW 24-methylenecholesterol, 0.4 mg/g DCW campesterol, 0.5 mg/g DCW isofucosterol, trace amounts of beta-sitosterol, 0.3 mg/g DCW cholesterol, 0.04 mg/g DCW desmosterol and 1.8 mg/g DCW tetrahymanol.

### DISCUSSION

Since tetrahymanol is able to substitute for ergosterol function in the cell, as previously the ergosterol biosynthesis pathway could subsequently be disrupted without compromising cell viability. The native genes ERG4 and ERG5 were deleted to prevent ergosterol synthesis and allow diversion of flux toward the non-native sterol structure 24-methylenecholesterol, upon introduction of a heterologous delta-7 sterol reductase. As noted above, it is anticipated that the ergosterol pathway is tightly regulated to control ergosterol concentration in the cell. This may partly be achieved by end-product feedback inhibition. This mechanism would be expected to be highly specific to ergosterol, to prevent interference by structurally similar early intermediates of the ergosterol pathway. Hence, sterol accumulation is greater in strains lacking ergosterol, particularly in those producing non-native sterols. These sterols are likely to be esterified and stored in lipid droplets to prevent toxicity.

24-methylenecholesterol is converted to campesterol by the delta-24 sterol reductase DWF1 in plants. ERG4 catalyses sterol delta-24 reduction in yeasts. Hence, the native ERG4 was re-introduced under the control of weak promoters to enable partial conversion of 24-methylenecholesterol to campesterol.

However, DWF1 In plants and ERG4 generate different epimers. The reduced 24-methyl sterol produced by a DWF1 is the C-24R epimer. ERG4 produces the C-24S epimer, as seen in ergosterol or epi-campesterol produced previously in Y. lipolytica (Du et al., 2016; Zhang et al., 2017. Qian et al. 2020).

To ensure production of the C-24R configuration of campesterol, a plant DWF1 was introduced under the control of a weak promoter in strain ST11064 in which the native ERG4 was deleted. This enabled partial conversion of 24-methylenecholesterol to campesterol, rather than to C-24S epi-campesterol. Similarly, the C-24R epimer of sitosterol is produced (0-sitosterol) rather than the C-24S epimer (clionasterol). It is important to distinguish these isomers as the honeybee ecdysteroid hormone makisterone A is derived from campesterol. The stereochemistry at C-24 is conserved during this conversion, and the molecular implications of an alternative stereochemistry at this position are unknown.

Strains were further engineered to incorporate a C-28 sterol methyltransferase. The broad specificity of this enzyme enabled production of the 24-ethyl sterol isofucosterol. β-sitosterol is produced from isofucosterol by DWF1. A weak promoter was chosen to ensure low C-28 sterol methyl transferase activity and the production of a small amount of isofucosterol relative to 24-methylenecholesterol.

As noted above, the S. lycopersicum delta-24(25) sterol reductase (SSR2) has been shown to convert 24-methylenecholesterol to cholesterol (Sawai et al. 2014). Hence, ΔERG5ΔERG4 Y. lipolytica yeast strains further engineered to express this plant delta-24 sterol reductase plus a delta-7 sterol reductase permit the production of a small amount of cholesterol. This is exemplified by strain ST12178 which was derived from strain ST12140 and hence additionally expressed a C-28 sterol methyltransferase. Delta-24(25) sterol reductase expression was controlled by use of a weak promoter to allow the production of a small amount of cholesterol relative to 24-methylenecholesterol. In addition, trace amounts of desmosterol were detected. This was unexpected but is expected to be an intermediate or side-product arising from activity of the S. lycopersicum delta-24(25) sterol reductase upon 24-methylenecholesterol.

Hence, ST12178 was considered a favoured strain for further study in the context of providing a mixed sterol containing artificial dietary composition for honeybees. It contained 24-methylenecholesterol as the dominant sterol, some campesterol as the plant epimer plus cholesterol together with low amounts of all of isofucosterol, β-sitosterol and desmosterol.

Not all strains capable of producing exogenous sterols require a tetrahymanol substitute. The TtSTC gene could be removed from strains ST11362 and ST11378, which could produce a mixture of exogenous sterols. Total sterol content in the resulting strains ST11441 and ST11542 is still six times greater than in the wild-type strains, suggesting that one or more of the exogenous sterols is capable of substituting for ergosterol function in the yeast cel.

Sterols accumulate during the stationary phase in yeast (Rychtera et al. 2010). The total nitrogen was reduced compared to standard Delft mineral medium (Jensen at al, 2014) in order to increase the C/N ratio, since a high C/N ratio appears to be beneficial for lipid production, (Kerkhoven et al, 2018; Bellou et al, 2016) and may therefore support the accumulation of hydrophobic sterol compounds. The C/N of the batch media in the Ambr bioreactors was approximately 15. Hence, increasing the C/N may be expected to promote lipid accumulation and increase sterol content.

For stains ST4842, ST11005 and ST12178, the final biomasses achieved in 5-L fermentation were 68.8, 54.5 and 49.0 g/L respectively. The corresponding total sterol contents were 0.1, 0.2 and 9.1 mg/g DCW. The sterol content was significantly higher in the ST12178 than either the wild-type strain or the tetrahymanol producing strain ST11005. This suggests that the ergosterol present in ST4842 and ST11005 may lead to feedback inhibition on the sterol pathway. This effect may be reduced in ST12178 that lacks the native ergosterol, allowing greater total sterol content. The biomass specific yield for the ST12178 fermentation was consistent with that obtained in small scale cultivation. However, the sterol content of ST12178 was not increased beyond the level obtained in small scale cultivation, despite the increase in biomass concentration. Higher sterol accumulation may be achieved by increasing the lipid content, although this may be at the cost of biomass production. The C/N ratio of the batch media was roughly 6. Hence, increasing the C/N may again be expected to promote lipid accumulation and increase sterol content.

A similar platform strain has also been used to produce the sesquiterpenoid abscisic acid (Arnesen et al, 2022). The biomass yield and abscisic acid titre obtained during bioreactor cultivation were both lower than achieved in small-scale cultivation. However, a higher specific yield was obtained in bioreactor cultivation by altering conditions such as pH and dissolved oxygen. This suggests that terpenoid production in such strains is sensitive to fermentation conditions, and these can be fine-tuned to optimise production.

The engineered Y. lipolytica strains discussed herein demonstrate the potential for the synthesis of a tailored mixture of non-native sterol structures with specific stereochemistry. It is expected that sterol content could be improved by further strain modification and optimisation of fermentation conditions.

### ADDITIONAL REFERENCES

Arnesen J A, Jacobsen I H, Dyekjaer J D, Rago D, Kristensen M, Klitgaard A K, Randelovic M, Martinez J L & Borodina 1(2022) Production of abscisic acid in the oleaginous yeast Yarrowia lipolytica. FEMS Yeast Res 22: 1-10
Back A, Rossignol T, Krier F, Nicaud J M & Dhulster P (2016) High-throughput fermentation screening for the yeast Yarrowia lipolytica with real-time monitoring of biomass and lipid production. Microb Cell Fact 15: 1-12
Bellou S, Triantaphyllidou I E, Mizerakis P & Aggelis G (2016) High lipid accumulation in Yarrowia lipolytica cultivated under double limitation of nitrogen and magnesium. J Biotechnol 234: 116-126
Cao X, Wei L J, Un J Y & Hua Q (2017) Enhancing linalool production by engineering oleaginous yeast Yarrowia lipolytica. Bioresour Technol
Jensen N B, Strucko T, Kildegaard K R, David F, Maury J, Mortensen U H, Forster J, Nielsen J & Borodina 1(2014) EasyClone: method for iterative chromosomal integration of multiple genes Saccharomyces cerevisise. FEMS Yeast Res 14:238-248
Kerkhoven E J. Pomraning K R, Baker S E & Nielsen J (2016) Regulation of amino-acid metabolism controls flux to lipid accumulation in Yarrowia lipolytica, npj Systems Biology and Appkcations 2016 2:12: 1-7
Rychtera M, Cermak J, Votruba J, Nahlik J, Melzoch K. A. Kent C & Estela Escalante W (2010) Optimization of feeding strategy for the ergosterol production by yeasts Saccharomyces cerevisiae. Rev Colomb Biotechnol
Sáez-Sáez J. Wang G, Marela E R, Sudarsan S, Cernuda Pastor M & Borodina 1(2020) Engineering the oleaginous yeast Yarrowia lipolytica for high-level resveratrol production. Metab Eng 62: 51-81
Sawai S, Ohyama K. Yasumoto S. Seki H, Sakuma T, Yamamoto T. Takebayashi Y, Kojima M, Sakakibara H, Aoki T, et al (2014) Sterol side chain reductase 2 is a key enzyme in the biosynthesis of cholesterol, the common precursor of toxic steroidal glycoalkaloids in potato. Plant Cell

TABLE 18
Genomic location of integration sites (based
on CLIB122 genome sequence, ASM252v1)

| Name of integration site | Chromosome | Genome region (bp) |
|---|---|---|
| IntA_1 | A | 1556243-1557282 |
| IntC_1 | C | 1793168-1797643 |
| IntC_2 | C | 1842294-1843343 |
| IntC_3 | C | 568316-569321 |
| IntD_1 | D | 2192680-2193710 |
| IntE_1 | E | 1722042-1723055 |
| IntE_2 | E | 3758687-3759710 |
| IntE_3 | E | 1845309-1846364 |
| IntE_4 | E | 2881519-2882566 |
| IntF_2 | F | 110095-111114 |
| IntF_3 | F | 795904-796943 |

TABLE 19
Construction of Y. lipolytica strains ST11362 and ST11441

| Strain | Sterol composition (dominant sterol underlined) |
|---|---|
| ST9100 | Ergosterol |
| ST11005 | Ergosterol, tetrahymanol |
| ST11027 | Ergsota-5,7-dienol, tetrahymanol |
| ST11040 | Ergosta-5,7,24(28)-trienol, tetrahymanol |
| ST11064 | 24-methylenecholesterol, tetrahymanol |
| ↓ | +ERG4 delta-24 reductase |
| ST11338 | 24-methylenecholesterol, epicampesterol, |
| | tetrahymanol |
| ↓ | +DHCR24 delta-24(25) sterol reductase |
| ST11340 | 24-methylenecholesterol, epicampesterol, |
| | cholesterol, desmosterol, tetrahymanol |
| ↓ | +SMT C-28 sterol methyltransferase |
| ST11362 | 24-methylenecholesterol, epicampesterol, |
| | isofucosterol, sitosterol, cholesterol, |
| | desmosterol, tetrahymanol |
| ↓ | −TtSTC squalene-tetrahymanol cyclase |
| ST11441 | 24-methylenecholesterol, epicampesterol, |
| | isofucosterol, sitosterol, cholesterol, |
| | desmosterol |

TABLE 20
Construction of Y. lipolytica strains ST11541 and ST11542

| Strain | Sterol composition (dominant sterol underlined) |
|---|---|
| ST9100 | Ergosterol |
| ST11005 | Ergosterol, tetrahymanol |
| ST11027 | Ergsota-5,7-dienol, tetrahymanol |
| ST11040 | Ergosta-5,7,24(28)-trienol, tetrahymanol |
| ST11064 | 24-methylenecholesterol, tetrahymanol |
| ↓ | +ERG4 delta-24 reductase |
| ST11337 | 24-methylenecholesterol, epicampesterol, |
| | tetrahymanol |
| ↓ | +DHCR24 delta-24(25) sterol reductase |
| ST11378 | 24-methylenecholesterol, epicampesterol, |
| | cholesterol, desmosterol, tetrahymanol |
| ↓ | +SMT C-28 sterol methyltransferase |
| ST11541 | 24-methylenecholesterol, epicampesterol, |
| | isofucosterol, sitosterol, cholesterol, |
| | desmosterol, tetrahymanol |
| ↓ | −TtSTC squalene-tetrahymanol cyclase |
| ST11542 | 24-methylenecholesterol, epicampesterol, |
| | isofucosterol, sitosterol, cholesterol, |
| | desmosterol |

TABLE 21
Construction of Y. lipolytica strain ST12178

| Strain | Sterol composition (dominant sterol underlined) |
|---|---|
| ST9100 | Ergosterol |
| ↓ | +TtSTC squalene-tetrahymanol cyclase |
| ST11005 | Ergosterol, tetrahymanol |
| ↓ | −ERG5 C-22 desaturase |
| ST11027 | Ergsota-5,7-dienol, tetrahymanol |
| ↓ | −ERG4 delta-24 reductase |
| ST11040 | Ergosta-5,7,24(28)-trienol, tetrahymanol |
| ↓ | +DHCR7 delta-7 reductase |
| ST11064 | 24-methylenecholesterol, tetrahymanol |
| ↓ | +StSSR1 delta-24(28) reductase |
| ST11943 | 24-methylenecholesterol, campesterol, |
| | tetrahymanol |
| ↓ | +SMT C-28 sterol methyltransferase |
| ST12140 | 24-methylenecholesterol, campesterol, |
| | isofucosterol, sitosterol, tetrahymanol |
| ↓ | +DHCR24 delta-24(25) sterol reductase |
| ST12178 | 24-methylenecholesterol, campesterol, |
| | isofucosterol, sitosterol, cholesterol, |
| | desmosterol, tetrahymanol |

### Example 6—Testing of Engineered Yarrowia lipolytica Strains in Artificial Diets for Managed Honeybee Colonies

### Materials and Methods

### Diet Preparation

Yeast strains ST4842 (wild-type, containing ergosterol), ST11005 (platform strain engineered to produce tetrahymanol, containing ergosterol and tetrahymanol) and ST12178 (mixed sterol producing strain producing desired phytosterols and tetrahymanol) were cultivated by 5-L fed-batch fermentation as described above. At the end of fermentation, the yeast biomass was recovered from the culture by centrifugation (4000×g, 20 min) and washed with deionized water. The biomass was heat-inactivated and dried (60° C. for a minimum of 24 h). The dried material was ground to a fine powder and stored at −20° C. until further use.

The yeast biomass cannot be subject to inactivation by autoclave or chemical treatment, as this will degrade the sterols present in the yeast. Incubation at 60° C. is commonly deemed sufficient for irreversible inactivation of the yeast and heat-inactivation of GMO yeast followed by feeding the inactivated yeast to live animals is a method that has been used previously in the UK (e.g. see Goh et al., 2021). Irreversible inactivation of the yeast was confirmed by a standard colony forming unit assay. The heat-inactivated dried yeast was dissolved at 10 mg/ml in water. The suspension was plated in serial dilution (100 μl plated, of 10, 1, 0.1, 0.01 and 0.001 mg/ml suspensions) on YPD-agar (yeast extract 10 g/l, peptone 20 g/l, glucose 20 g/l. Bacto agar 24 g/l) and the plates were incubated at 30° C. for at least seven days. No growth of Yarrowia lipolytica colonies was observed. The detection limit is 1 organism/mg material or 10⁶ viable organisms/kg material.

The yeast biomass was then incorporated at 20% w/w into a holidic diet designed to provide additional protein, lipid, sugar, vitamins and minerals.

Since the mixed sterol yeast strain only contained trace β-sitosterol, a small amount of commercially available phytosterol mixture including β-sitosterol (β-sitosterol, BulkSupplements) was added (0.125% w/w). This commercially available phytosterol mixture comprised 40% β-sitosterol, 27% campesterol, 10% stigmasterol and 10% brassicasterol. Assuming 1% w/w sterol content in the mixed sterol yeast dry powder, the sterol provided by the yeast together with the β-sitosterol provided by the commercial phytosterol mixture equated to 0.25% w/w sterol in the final diet. In the base diet control treatment, additional commercial phytosterol mixture was added to match the total level of relevant sterol in the mixed sterol yeast treatment. The phytosterol mixture was added at 0.625% w/w such that final base diet contained 0.25% w/w β-sitosterol. This allowed the effects of total sterol concentration in the diet to be distinguished from the effects of sterol composition, as the sterol content of the mixed sterol yeast ST12178 is higher than that of the wild-type or tetrahymanol producing yeast strains ST4842 and ST11005 (FIGS. 20-22).

Thus four diet types were prepared: a ‘mixed sterol yeast’ diet containing strain ST12178, a ‘wild-type yeast’ diet containing strain ST4842, a ‘platform yeast’ diet containing strain ST11005, and a base diet control without yeast supplementation. The yeast strains were mixed with a holidic diet with added protein, oil, sugar, minerals and vitamins. The base diet control was formulated to maintain total protein, sugar, sterol and fat content at the same level as in the yeast supplemented diets.

The diets were supplied as 50 g patties to APIDEA honeybee colonies managed within a glasshouse and stored at −20° C. until use.

### Honeybee Colonies

Honeybee colonies were maintained for three months in APIDEA hives (Styrofoam queen mating mini nucleus hives, (Paynes Southdown Bee Farms Ltd, West Sussex. UK) made up of one brood box with five frames and a top feeder with a hole for petty delivery. Hives were maintained within a closed glasshouse environment designed to negate bee escape. Hives were distributed across two rooms of the glasshouse, with varying orientation. Feeders with 30% w/v sugar solution and water were distributed inside the glasshouse and replenished as required. Regular temperature and humidity measurements were recorded on a datalogger. A misting system was installed to cool the temperatures in the glasshouse.

Initially, each APIDEA hive contained 400-500 adult bees, 2-3 frames brood/larvae/eggs and 1-2 frames of honey stores, but no bee bread. Mated queens were introduced in cages with fondant three days prior to the start of the experiment. If necessary, queens were manually released from the cages on the first day of the experiment.

At the start of the experiment, diet patties were added via the top feeder, and replaced throughout the experiment as required. Six hives were randomly assigned to each treatment group (n=6). A full assessment of the hives was conducted every 15 days. Hive weight, patty weight and ‘bee seams’ (one seam defined as a continuous line of bees between adjacent frames, observed upon initial hive opening) were measured. Presence of the mated queen, honey stores, eggs, larvae and capped brood were checked, and brood frames were photographed for subsequent counting. Eggs, larvae and capped brood were counted in Adobe Photoshop. Statistical analysis was performed in Microsoft Excel and R (v4.2.1).

Six days after each full assessment, hives were partially assessed with minimal disruption to the colony. Hive weight, patty weight and bee seams' were measured. Presence of the mated queen, honey stores, eggs, larvae and capped brood were briefly checked. On days 21 and 45, hives with low populations (less than four bee seams) were topped up with orphanized bees.

### Results

Diet consumption, colony size, brood production and development were monitored over the course of three months for each treatment group. Brood at egg, larval and capped stages in each hive were counted every 15 days, to assess the ability of each treatment to support both brood production and proper development (FIGS. 23 and 24).

No consistent trends in egg or larvae production were found to differ between treatment groups. As such, the mixed sterol diet does not appear to confer altered laying behaviour in the queen.

Although the number of eggs and larvae did not differ significantly between treatment groups, the number of capped brood cells was significantly higher in the mixed sterol yeast diet by day 60.

Although brood production declined in all treatment groups in the last month at the end of summer, the mixed sterol yeast diet conferred greater capped brood than all other treatment groups by the end of the experiment. This demonstrates that the colonies can support continued brood development later in the season. The base diet control does not support brood development to the same extent. This diet contained 0.25% b-sitosterol, to match the total sterol in the mixed sterol yeast treatment, as well as other phytosterols including campesterol and stigmasterol. This suggests that the specific type and composition of sterols in the mixed sterol yeast diet, such as 24-methylenecholesterol, isofucosterol, cholesterol and desmosterol are necessary for proper brood development.

As well as sterols, the yeast provides beneficial nutrients such as protein, amino acids and lipids, mostly oleic acid (D9C 18:1), palmitic acid (C16:0), stearic acid (C18:0), and linoleic acid (D9,12 C18:2, Athenstaedt et al., 2006). The wild-type yeast diet containing small amounts of ergosterol did not result in reduced brood production, development or colony health compared to the base diet control. This suggests additional compounds present in the yeast powder, as prepared by the method described, do not confer toxicity to the honeybee. The tetrahymanol-producing platform strain containing small amounts of both ergosterol and tetrahymanol did not result in reduced brood production, development or colony health compared to the wild-type yeast diet. This suggests that the presence of tetrahymanol does not have adverse effects, for example by competitively inhibiting sterol uptake from the gut.

There were no significant differences in the total amount of diet delivered or consumed between treatment groups. This suggests that the effects observed were not attributed to different intake of other macronutrients such as fats or protein. The presence of yeast powder or different sterol compositions also does not prompt a change in feeding behaviour. However, there may be additional nutrients provided by the mixed sterol yeast that differ from the wild-type yeast or tetrahymanol platform strains, that contribute to the effects observed.

Overall, the results demonstrate that Yarrowia lipolytica engineered by the methods described to produce a plurality of key phytosterols is a suitable dietary supplement for managed honeybee colonies. The provision of a phytosterol mixture including 24-methylenecholesterol, campesterol, cholesterol, desmosterol, isofucosterol and β-sitosterol can improve the duration of brood production, as well as proper brood development, compared to a diet containing sub-optimal sterol composition.

Improving the sterol content in the dry yeast powder or increasing the percentage of yeast in the final diet may further support brood production and development

### REFERENCES

Athenstaedt, K., Jolivet, P., Boulard, C., Zivy, M., Negroni, L., Nicaud, J. M., & Chardot, T. (2006). Lipid particle composition of the yeast Yarrowia lipolytica depends on the carbon source. Proteomics.
Goh, S., Kolakowski, J., Holder, A., Pfuhl, M., Ngugi, D., Ballingall, K., Tombacz, K., & Werling, D. (2021). Development of a Potential Yeast-Based Vaccine Platform for Theileria parva Infection in Cattle. Frontiers in Immunology, 12, 2511.

1. An oleaginous yeast for expression of one or more heterologous genes for production of one or more desired non-native sterols or compounds derived therefrom, wherein
(i) the yeast has reduced production of ergosterol compared with a wild-type oleaginous yeast or is incapable of producing ergosterol; and
(ii) is provided with a sterol surrogate to aid cell growth.

2. An oleaginous yeast as claimed in claim 1, wherein the sterol surrogate is tetrahymanol or a hopanoid.

3. An oleaginous yeast as claimed in claim 1, wherein the oleaginous yeast comprises a heterologous nucleic acid sequence encoding a squalene-tetrahymanol cyclase or a squalene-hopene cyclase for providing the sterol surrogate, preferably wherein said heterologous nucleic acid sequence is under the control of the PrGPAT promoter or a functionally equivalent weak yeast promoter.

4. An oleaginous yeast as claimed in claim 1, wherein the heterologous nucleic acid sequence encodes Tetrahymena thermophila squalene-tetrahymanol cyclase or a functional variant thereof or Schizosaccharomyces japonicus squalene-hopene cyclase or a functional variant thereof.

5. An oleaginous yeast as claimed in claim 1, wherein the oleaginous yeast has an attenuated or deleted endogenous sterol C-22 desaturase (ERG5) or an attenuated or deleted endogenous sterol C-24 methyltransferase (ERG6).

6. An oleaginous yeast as claimed in claim 1, wherein the oleaginous yeast has an attenuated or deleted endogenous sterol C-22 desaturase (ERG5) and further comprises an attenuated or deleted endogenous delta-24 sterol reductase (ERG4) and/or sterol C-24 methyltransferase (ERG6).

7. An oleaginous yeast as claimed in claim 1, wherein the oleaginous yeast further comprises one or more heterologous nucleic acid sequences capable of expression to provide one or more of:
a. a delta-7 sterol reductase enzyme;
b. a delta-24(28) sterol reductase enzyme;
c. a delta-24(25) sterol reductase enzyme;
d. a sterol C-28 methyltransferase enzyme and
e. a sterol C-22 desaturase enzyme,
whereby production of said one or more desired non-native sterols or compounds derived therefrom can be achieved.

8. An oleaginous yeast as claimed in claim 1,
a. wherein the oleaginous yeast comprises:
(i) an attenuated or deleted endogenous sterol C-22 desaturase enzyme (ERG5);
(ii) a heterologous nucleic acid sequence encoding a delta-7 sterol reductase enzyme,
whereby one or more non-native sterols can be produced comprising campesterol;
b. wherein the oleaginous yeast comprises:
(i) an attenuated or deleted endogenous sterol C-22 desaturase enzyme (ERG5) and an attenuated or deleted delta-24 sterol reductase enzyme (ERG4);
(ii) a heterologous nucleic acid sequence encoding a delta-7 sterol reductase enzyme,
whereby one or more non-native sterols can be produced comprising 24-methylenecholesterol;
c. wherein the oleaginous yeast comprises:
(i) an attenuated or deleted endogenous sterol C-24 methyltransferase (ERG6), optionally an attenuated or deleted endogenous sterol C-22 desaturase enzyme (ERG5) and/or optionally an attenuated or deleted delta-24 sterol reductase enzyme (ERG4) and;
(ii) a heterologous nucleic acid sequence encoding a delta-7 sterol reductase enzyme,
whereby one or more non-native sterols can be produced comprising desmosterol;
d. wherein the oleaginous yeast comprises:
(i) an attenuated or deleted endogenous sterol C-22 desaturase enzyme (ERG5) and an attenuated or deleted sterol C-24 methyltransferase (ERG6);
(ii) a heterologous nucleic acid sequence encoding a delta-7 sterol reductase enzyme; and
(iii) a heterologous nucleic acid sequence encoding a delta-24 sterol reductase enzyme,
whereby one or more non-native sterols can be produced comprising cholesterol;
e. wherein the oleaginous yeast comprises:
(i) an attenuated or deleted endogenous sterol C-22 desaturase enzyme (ERG5) and an attenuated or deleted delta-24 sterol reductase enzyme (ERG4);
(ii) a heterologous nucleic acid sequence encoding a delta-7 sterol reductase enzyme and
(iii) a heterologous nucleic acid sequence encoding a sterol C-28 methyltransferase enzyme,
whereby one or more non-native sterols can be produced comprising isofucosterol (delta-24(28)-Z isomer) or fucosterol (delta-24(28)-E isomer);
f. wherein the oleaginous yeast comprises:
(i) an attenuated or deleted endogenous sterol C-22 desaturase enzyme (ERG5);
(ii) a heterologous nucleic acid sequence encoding a delta-7 sterol reductase enzyme and
(iii) a heterologous nucleic acid sequence encoding a sterol C-28 methyltransferase enzyme,
whereby one or more non-native sterols can be produced comprising beta-sitosterol; or
g. wherein the oleaginous yeast further comprises:
(i) a heterologous nucleic acid sequence encoding a delta-7 sterol reductase enzyme and
(ii) a heterologous nucleic acid sequence encoding a sterol C-28 methyltransferase enzyme
whereby one or more non-native sterols can be produced comprising stigmasterol, optionally
wherein the oleaginous yeast has an attenuated or deleted endogenous sterol C-22 desaturase enzyme (ERG5) and/or delta-24 sterol reductase enzyme (ERG4) and optionally additionally one or more further heterologous nucleic acid sequences are provided to express a plant delta-24(28) sterol reductase (DWF1) enzyme and/or sterol C-22 desaturase enzyme;
h. wherein the oleaginous yeast is capable of producing a mixture of desired non-native sterols and comprises:
(i) an attenuated or deleted endogenous sterol C-22 desaturase enzyme (ERG5) and an attenuated delta-24 sterol reductase enzyme (ERG4), preferably where the ERG5 gene is deleted and the activity of ERG4 is attenuated by provision of the same encoding sequence, or a corresponding plant delta-24(28) sterol reductase (DWF1) coding sequence, under the control of a weak promoter selected from PrDGA1 and functionally equivalent weak yeast promoters;
(ii) a heterologous nucleic acid sequence encoding a delta-7 sterol reductase enzyme, optionally a heterologous nucleic acid sequence encoding a delta-24(25) sterol reductase and/or optionally additionally a heterologous nucleic acid sequence encoding a C-28 sterol methyltransferase, whereby said non-native sterol mixture can be produced.

9. An oleaginous yeast according to claim 1 which is capable of producing a mixture of desired sterols comprising 24-methylenecholesterol, campesterol and cholesterol, optionally together with one or more further non-native sterols in detectable amount, wherein the oleaginous yeast comprises:
(i) an attenuated or deleted endogenous sterol C-22 desaturase enzyme (ERG5), preferably deleted ERG5;
(ii) an attenuated delta-24 sterol reductase enzyme (ERG4) or ERG4 substituted by a plant DWF1 enzyme providing attenuated delta-24(28) sterol reductase activity, e.g. where the ERG4 gene coding sequence or plant DWF1 enzyme coding sequence is under the control of the PrDGA1 promoter or a functionally equivalent weak promoter;
(iii) a heterologous nucleic acid sequence encoding a delta-7 sterol reductase enzyme, e.g. the delta-7 sterol reductase variant of Tetraselmis sp. GSL018, preferably under the control of a stronger promoter than employed for (ii), e.g. the PrTEFintron promoter or a functionally equivalent promoter;
(iv) a heterologous nucleic acid sequence encoding a delta-24(25) sterol reductase, e.g. the delta-24(25) sterol reductase of S. lycopersicum, preferably under the control of the PrGPAT promoter or a functionally equivalent weak promoter and optionally
(v) a heterologous nucleic acid sequence encoding a sterol C-28 sterol methyltransferase, e.g. the sterol C-28 sterol methyltransferase of C. quinoa, preferably under the control of the PrGPAT promoter or a functionally equivalent promoter,
whereby said mixture of non-native sterols can be produced.

10. An oleaginous yeast as claimed in claim 1 which is capable of producing 24-methylenecholesterol in an amount of at least about 2-3 mg/g, preferably at least about 9-10 mg/g dry cell weight, when cultured at 30° C. in yeast extract peptone dextrose (YPD) medium containing glucose and no sterol precursor.

11. An oleaginous yeast according to claim 1 wherein the yeast delta-24 sterol reductase enzyme (ERG4) is additionally substituted by a plant delta-24(28) sterol reductase enzyme (DWF1 enzyme) or an oleaginous yeast according to claim 8(h) or claim 9 wherein a plant DWF1 enzyme is expressed such that the oleaginous yeast can produce the plant epimer (24R) of campesterol.

12. An oleaginous yeast as claimed in claim 1 which is an engineered Yarrowia lipolytica strain where provision of said sterol surrogate is required to facilitate cell growth in the face of deletion of one or more of the ERG 4, ERG5 and ERG6 genes, preferably where said strain is engineered from Y. lipolytica ST9100 or another Y. lipolytica which shares all or some of the same modified genotype features as Y. lipolytica ST9100 compared with Y. lipolytica W29 strain Y-63746 (available from the ARS culture collection, NAUR and the ATCC) as the reference strain with increased synthesis of squalene or another sterol precursor or sterol pathway intermediate compared to that reference strain.

13. An oleaginous yeast according to claim 1 which is an engineered Y. lipolytica strain capable of producing a mixture of non-native sterols comprising 24-methylenecholesterol, campesterol as the 24R plant epimer, cholesterol, isofucosterol and desmosterol, wherein the oleaginous yeast comprises:
(i) a deleted endogenous sterol C-22 desaturase enzyme (ERG5),
(ii) ERG4 substituted by a DWF1 enzyme providing attenuated delta-24(28) sterol reductase activity, e.g. the delta-24(28) sterol reductase (DWF1) of Solanum tuberosum, where said DWF1 enzyme is under the control of the PrDGA1 promoter or a functionally equivalent weak promoter;
(iii) a heterologous nucleic acid sequence encoding a delta-7 sterol reductase enzyme, e.g. the delta-7 sterol reductase of Tetraselmis sp. GSL018, where said delta-7 sterol reductase is under the control of a stronger promoter than employed for (ii), e.g. the PrTEFintron promoter or a functionally equivalent promoter;
(iv) a heterologous nucleic acid sequence encoding a delta-24(25) sterol reductase, e.g. the delta-24(25) sterol reductase of S. lycopersicum, preferably under the control of the PrGPAT promoter or a functionally equivalent weak promoter and
(v) a heterologous nucleic acid sequence encoding a sterol C-28 methyltransferase, e.g. the sterol C-28 methyltransferase of C. quinoa, preferably under the control of the PrGPAT promoter or a functionally equivalent promoter,
whereby said mixture of non-native sterols can be produced.

14. An engineered yeast as claimed in claim 1 which is capable of producing 24-methylenecholesterol as the dominant sterol, e.g. in an amount of at least about 20 mg/g dry cell weight when cultured at 30° C. in yeast extract peptone dextrose (YPD) medium containing glucose and no sterol precursor, together with all of campesterol as the plant (24R) epimer, cholesterol, isofucosterol and desmosterol in quantifiable amount, preferably additionally with at least detectable beta-sitosterol

15. An oleaginous yeast for production of at least one non-native sterol, wherein the at least one non-native sterol comprises:
a. 24-methylenecholesterol or a derivative thereof, the oleaginous yeast comprising:
i. an attenuated or deleted endogenous sterol C-22 desaturase enzyme (ERG5) and an attenuated or deleted delta-24 sterol reductase enzyme (ERG4); and
ii. a heterologous nucleic acid sequence encoding a delta-7 sterol reductase enzyme; or
b. desmosterol or a derivative thereof, the oleaginous yeast comprising:
i. an attenuated or deleted endogenous sterol C-24 methyltransferase (ERG6), optionally an attenuated or deleted endogenous sterol C-22 desaturase enzyme (ERG5) and/or optionally an attenuated or deleted delta-24 sterol reductase enzyme (ERG4) and
ii. a heterologous nucleic acid sequence encoding a delta-7 sterol reductase enzyme; or
c. isofucosterol (delta-24(28)-Z isomer) and/or fucosterol (delta-24(28)-E isomer) or a derivative thereof, the oleaginous yeast comprising:
i. an attenuated or deleted endogenous sterol C-22 desaturase enzyme (ERG5) and an attenuated or deleted delta-24 sterol reductase enzyme (ERG4);
ii. a heterologous nucleic acid sequence encoding a delta-7 sterol reductase enzyme and
iii. a heterologous gene nucleic acid sequence encoding a sterol C-28 methyltransferase enzyme; or
d. cholesterol or a derivative thereof, the oleaginous yeast comprising:
i. an attenuated or deleted endogenous sterol C-22 desaturase enzyme (ERG5), and an attenuated or deleted sterol C-24 methyltransferase (ERG6);
ii. a heterologous nucleic acid sequence encoding a delta-7 sterol reductase enzyme and
iii. a heterologous nucleic acid encoding a delta-24 sterol reductase enzyme; or
e. beta-sitosterol or a derivative thereof, the oleaginous yeast comprising:
i. an attenuated or deleted endogenous sterol C-22 desaturase enzyme (ERG5);
ii. a heterologous nucleic acid sequence encoding a delta-7 sterol reductase enzyme and
iii. a heterologous nucleic acid sequence encoding a sterol C-28 methyltransferase enzyme; or
f. stigmasterol or a derivative thereof, the oleaginous yeast comprising:
i. a heterologous nucleic acid sequence encoding a delta-7 sterol reductase enzyme and
ii. a heterologous nucleic acid encoding a sterol C-28 methyltransferase enzyme; optionally
wherein the oleaginous yeast comprises an attenuated or deleted endogenous sterol C-22 desaturase enzyme (ERG5) and/or delta-24 sterol reductase enzyme (ERG4) and a heterologous nucleic acid encoding a sterol C-22 desaturase enzyme and optionally one or more further heterologous nucleic acid sequences are provided to express a plant delta-24(28) sterol reductase (DWF1) enzyme and/or sterol C-22 desaturase enzyme;
g. a sterol mixture, the oleaginous yeast comprising:
i. an attenuated or deleted endogenous sterol C-22 desaturase enzyme (ERG5) and an attenuated delta-24 sterol reductase enzyme (ERG4), preferably where the ERG5 gene is deleted and the activity of ERG4 is attenuated by provision of the same encoding sequence, or a corresponding plant delta-24(28) sterol reductase (DWF1) enzyme coding sequence, under the control of a weak promoter selected from PrDGA1 and functionally equivalent weak yeast promoters;
ii. a heterologous nucleic acid encoding a delta-7 sterol reductase enzyme,
optionally a heterologous nucleic acid encoding a delta-24(25) sterol reductase and/or
optionally additionally a heterologous nucleic acid encoding a C-28 sterol methyltransferase,
whereby a non-native sterol mixture can be produced, preferably such that a sterol mixture is produced comprising both 24-methylenecholesterol and campesterol, optionally together with one or more further non-native sterols, e.g. cholesterol;
h. a sterol mixture, the oleaginous yeast comprising:
i. an attenuated or deleted endogenous sterol C-22 desaturase enzyme (ERG5), preferably deleted ERG5;
ii. an attenuated delta-24 sterol reductase enzyme (ERG4) or ERG4 substituted by a plant delta-24(28) sterol reductase (DWF1) enzyme providing attenuated delta-24 sterol reductase activity, e.g. where the ERG4 gene coding sequence or plant delta-24(28) sterol reductase enzyme (DWF1) coding sequence is under the control of the PrDGA1 promoter or a functionally equivalent weak promoter;
iii. a heterologous nucleic acid sequence encoding a delta-7 sterol reductase enzyme, e.g. the delta-7 sterol reductase variant of Tetraselmis sp. GSL018, preferably under the control of a stronger promoter than employed for (ii), e.g. the PrTEFintron promoter or a functionally equivalent promoter;
iv. a heterologous nucleic acid sequence encoding a delta-24(25) sterol reductase, e.g. the delta-24(25) sterol reductase of S. lycopersicum, preferably under the control of the PrGPAT promoter or a functionally equivalent weak promoter;
v. a heterologous nucleic acid sequence encoding a C-28 sterol methyltransferase, e.g. the C-28 sterol methyltransferase of C. quinoa, preferably under the control the PrGPAT promoter or a functionally equivalent promoter,
whereby a mixture of non-native sterols can be produced comprising 24-methylenecholesterol, campesterol and one or more further non-native sterols comprising cholesterol, preferably where 24-methylenecholesterol or campesterol is the dominant sterol of the mixture.

16. An oleaginous yeast as claimed in claim 15 which is an engineered Yarrowia lipolytica.

17. (canceled)

18. An oleaginous yeast as claimed in claim 15 where a heterologous nucleic acid coding sequence for a delta-24(28) sterol reductase is required for production of said one or more non-native sterols and said heterologous nucleic acid sequence is selected to encode a plant DWF1 enzyme

19. An oleaginous yeast as claimed in claim 15, which is engineered so that it produces one or more additional desired squalene-derived compounds, e.g. a terpenoid compound useful as pigment and/or a terpenoid compound useful as a scent in an artificial dietary composition e.g. an insect feed such as a bee feed.

20. A method for production of one or more desired non-native sterols which comprises culturing cells of an oleaginous yeast as claimed in claim 15, under conditions whereby said one or more desired non-native sterols are synthesized.

21. A method as claimed in claim 20 wherein the culture medium comprises a carbon source, optionally isotopically-labelled, selected from one or more of:
glucose, fructose, sucrose, xylose, mannose, galactose, rhamnose, arabinose, one or more fatty acids, glycerol, acetate, citrate, pyruvate, starch, glycogen, amylopectin, amylose, cellulose, cellulose acetate, cellulose nitrate, hemicellulose, xylan, glucuronoxylan, arabinoxylan, glucomannan, xyloglucan, lignin, lignocellulose and/or vegetable oil, preferably glucose, and no sterol precursor is provided in the culture medium.

22. A method as claimed in claim 19, wherein said one or more non-native sterols are further converted in the same cells to one or more desired sterol-derived compounds, for example sterol esters.

23. A method as claimed in claim 19 which further comprises the step of recovering from the cell culture said one or more desired non-native sterols and/or one or more desired sterol-derived compounds and/or one or more compounds derived from increased squalene production such as terpenoid compounds, including beta-carotene, beta-caryophyllene, beta-cryptoxanthin and linalool.

24. A method as claimed in claim 19 which further comprises converting one or more sterols thus recovered to one or more sterol-derived compounds.

25. A method as claimed in claim 19, wherein a hydrophobic solvent is employed to extract the one or more desired sterols or one or more sterol-derivatives from cells following saponification and/or dodecane is employed as an additive in the culture medium to facilitate sterol extraction from the cell membrane.

26. A method as claimed in claim 19 which further comprises incorporating one or more recovered sterols and/or one or more recovered sterol-derived compounds, optionally together with one or more recovered additional compounds derived from increased squalene production, into a composition selected from an artificial dietary composition, a food product, an agricultural composition, a cosmetic composition or pharmaceutical composition.

27. A method as claimed in claim 19 wherein said one or more recovered sterols incorporated into said composition comprise isofucosterol (delta-24(28)-Z isomer) or fucosterol (delta-24(28)-E isomer), preferably isofucosterol (delta-24(28)-Z isomer), said one or more recovered sterols optionally additionally comprising 24-methylenecholesterol and campesterol as the 24R plant epimer.

28. A method as claimed in claim 19 which further comprises following said culturing recovering yeast cells from the culture medium as a yeast cell biomass and inactivating the yeast cells.

29. A method as claimed in claim 19 wherein said yeast cell biomass is heated at no more than 60° C. to heat inactivate and dry the yeast cells.

30. A method as claimed in claim 19 which further comprises converting the dried yeast cell biomass to a powder.

31. A method as claimed in claim 19 which further comprises incorporating said yeast cells or said dried yeast cell powder into a composition selected from an artificial dietary composition, a food product, an agricultural composition, a cosmetic composition or pharmaceutical composition.

32. A method as claimed in claim 19 wherein there is provided in said composition by said yeast cells or said dried yeast cell powder one or more sterols comprising isofucosterol (delta-24(28)-Z isomer) or fucosterol (delta-24(28)-E isomer), preferably isofucosterol (delta-24(28)-Z isomer), optionally together with 24-methylenecholesterol and campesterol as the 24R plant epimer.

33. A method as claimed in claim 19 wherein said composition is an artificial dietary composition for bees or other insects or animals.

34. A method as claimed in claim 19 wherein one or more sterols are incorporated into said artificial dietary composition for bees or other insects or animals, said one or more sterols being selected from 24-methylenecholesterol or a sterol mixture comprising 24-methylenecholesterol, preferably a sterol mixture comprising both 24-methylenecholesterol and the plant epimer of campesterol, optionally together with one or more further sterols in detectable amount.

35. A method as claimed in claim 19 wherein a sterol mixture is incorporated into said artificial dietary composition which comprises 24-methylenecholesterol and isofucosterol (delta-24(28)-Z isomer), optionally together with one or more further sterols in detectable amount.

36. An artificial dietary composition for bees or other insects or animals which comprises oleaginous yeast cells as claimed in claim 15, preferably wherein said yeast cells are yeast cells capable of producing 24-methylenecholesterol, isofucosterol and/or fucosterol, or cholesterol.

37. (canceled)

38. Artificial dietary composition for animals or insects, in particular bees, a food product, an agricultural composition, a cosmetic composition or pharmaceutical composition,
wherein the artificial dietary composition for animals or insects, in particular bees, the food product, the agricultural composition, the cosmetic composition or the pharmaceutical composition comprises isofucosterol and/or fucosterol; and
wherein the artificial dietary composition for animals or insects, in particular bees, the food product, the agricultural composition, the cosmetic composition or the pharmaceutical composition is obtainable by a method comprising culturing cells of an oleaginous yeast, preferably Yarrowia lipolytica, under conditions whereby isofucosterol and/or fucosterol are synthesized and wherein the oleaginous yeast comprises:
i. an attenuated or deleted endogenous sterol C-22 desaturase enzyme (ERG5) and an attenuated or deleted delta-24 sterol reductase enzyme (ERG4);
ii. a heterologous nucleic acid sequence encoding a delta-7 sterol reductase enzyme; and
iii. a heterologous gene nucleic acid sequence encoding a sterol C-28 methyltransferase enzyme.

39. Artificial dietary composition for animals or insects, in particular bees, a food product, an agricultural composition, a cosmetic composition or pharmaceutical composition according to claim 38,
wherein the artificial dietary composition for animals or insects, in particular bees, the food product, the agricultural composition, the cosmetic composition or the pharmaceutical composition is free or essentially free of ergosterol.

40. Artificial dietary composition for animals or insects, in particular bees, a food product, an agricultural composition, a cosmetic composition or pharmaceutical composition according to claim 38,
wherein the artificial dietary composition for animals or insects, in particular bees, the food product, the agricultural composition, the cosmetic composition or the pharmaceutical composition further comprises 24-methylene cholesterol.

41. Artificial dietary composition for animals or insects, in particular bees, a food product, an agricultural composition, a cosmetic composition or pharmaceutical composition according to claim 38, wherein the artificial dietary composition for animals or insects, in particular bees, the food product, the agricultural composition, the cosmetic composition or the pharmaceutical composition further comprises cholesterol.

42. Artificial dietary composition for animals or insects, in particular bees, a food product, an agricultural composition, a cosmetic composition or pharmaceutical composition according to claim 38, wherein the isofucosterol and/or fucosterol is delivered without an extraction step.

43. Artificial dietary composition for animals or insects, in particular bees, a food product, an agricultural composition, a cosmetic composition or pharmaceutical composition according to claim 38, wherein the isofucosterol and/or fucosterol is delivered in the form of a dried yeast cell biomass attained following recovery of yeast cells from a culture medium.

44. A method for producing isofucosterol and/or fucosterol, wherein the method comprises culturing cells of an oleaginous yeast, in particular Yarrowia lipolytica, under conditions whereby isofucosterol is synthesized in a desired amount, and wherein the oleaginous yeast, in particular Yarrowia lipolytica, comprises:
i. an attenuated or deleted endogenous sterol C-22 desaturase enzyme (ERG5) and an attenuated or deleted delta-24 sterol reductase enzyme (ERG4);
ii. a heterologous nucleic acid sequence encoding a delta-7 sterol reductase enzyme; and
iii. a heterologous gene nucleic acid sequence encoding a sterol C-28 methyltransferase enzyme.

45. The method of claim 44, which further comprises following said culturing recovering yeast cells from the culture medium as a yeast cell biomass and inactivating the yeast cells.

46. The method of claim 44, wherein said yeast cell biomass is heated at no more than 60° C. to heat inactivate and dry the yeast cells.

47. The method of claim 44, which further comprises converting the dried yeast cell biomass to a powder or an extract.

48. The method of claim 44, which further comprises incorporating said yeast cells or said dried yeast cell powder or extract into a composition selected from an artificial dietary composition, a food product, an agricultural composition, a cosmetic composition or pharmaceutical composition.


SEQUENCE LISTING (236 sequences)

SEQ ID NO: 1 - DNA, 1305 bp, synthetic construct
  misc_feature 1..1305: note = Solanum tuberosum delta-7 sterol reductase codon optimised for Yarrowia lipolytica
  source 1..1305: mol_type = other DNA
atggccgagtctcagctggtgcaccctcctctgttcacctacatctctatgctggccctgctgaccctggtgcctcctttcgtgatcctgatgtggtacaccaacgtgcacgccgacggctctgtgctgcagaccttcaactacctgaaggaaaacggcctgcagggcctgatcgacatctggccccgacctaccgccattgccggaaagatcatcatctgctacgccctgttcgaggctaccctgcagctgctgctgcccggcaagcgagtgcagggccccatctctcccaccggccaccgacctgtgtacaaggccaacggcatggccgcctacaccgtgactctgattacctacctgtctctgtggtggttcggcatcttcaaccccaccgtggtgtacgaccacctgggcgagatcctgtctaccctgaacttcggctctctgatcttctgcctgttcctgtacatcaagggacacgtggctccctcttctaccgaccacggctcctctggcaacatcatcgtggactactactggggcatggaactgtaccctcgaatcggcaagcacttcgacatcaaggtgttcaccaactgtcgattcggcatggtgtcttggggactgctgcccatcacctactgcatcaagcagtacgaggaatacggatctctgtctgactccatgctgatccacgccatcatcaccctggtctacgtgaccaagttcttctggtgggaggccggctactggaacaccatggacattgcccacgaccgagccggcttctacatctgctggggctgcctggtgttcctgccttgcatgtacacttctcccggcatgtacctggtgaagcaccccgtgaacctgggacctcagctggccatctccatcctggtggccggcatcctgtgcgtgtacattaactacgactgcgaccgacagcgacaagagttccgacgaactaacggcaaggccctggtgtggggcaaggctccctccaagatcgtggcctcttacaccaccaccactggcgagactaagtcctctctgctgctgacctccggctggtggggcctgtctcgacacttccactacgtgcccgagattctggcctctttcttttggtctgtgcccgctctgttcaaccacattatgccctacttctacgtgatctacctgaccggcctgctgctggaccgagccaagcgagatgacgaacgatgcaagtctaagtacggcaagtactggaagaagtactgcgagaaggtcccctaccgagtgatccccggcatctactaa

SEQ ID NO: 2 - DNA, 1437 bp, synthetic construct
  misc_feature 1..1437: note = Danio rerio delta-7 sterol reductase codon optimised for Yarrowia lipolytica
  source 1..1437: mol_type = other DNA
atgatggcctctgaccgagttcgaaagcgacacaagggctctgctaacggtgctcagaccgttgagaaggaaccctccaaggagcccgcccagtggggccgagcctgggaggtcgattggttctccctgtccggcgtgattctgctgctgtgctttgcccctttcctggtcttcttcttcatcatggcttgtgatcagtaccagtgctctatctcccatccccttctggacctttacaacggtgacgccactctgttcaccatctggaaccgagccccctccttcacctgggccgctgccaagatctacgccatctgggtcaccttccaggtcgttctgtacatgtgcgtccccgacttcctgcacaagatcctgccaggttacgtcggcggtgtccaggacggagctagaactcccgccggcctgatcaacaagtatgaggttaacggtctgcagtgctggctcatcacccacgtgctctgggtgctgaatgctcagcacttccactggttttcacccaccattatcattgacaactggatccccctgctgtggtgcaccaacattctgggctatgccgtctccaccttcgctttcatcaaggcctacctgttccccaccaatcccgaggactgcaagttcaccggaaacatgttttacaattacatgatgggtattgagttcaacccccgaatcggtaagtggttcgacttcaagctgttcttcaacggtcggcctggcatcgtcgcctggaccctcatcaacctttcctacgctgctaagcagcaggagctgtacggctacgtcaccaactctatgatcctggtcaacgtcctgcaggccgtgtacgttgtcgacttcttctggaacgaggcttggtacctgaaaaccatcgacatctgccacgaccactttggctggtacctgggatggggagactgcgtttggctgcctttcctgtacaccctgcagggtctgtacctggtctacaaccctatccagctgtccactccccacgctgccggcgtgctgatcctgggtctggtcggttactacatttttcgagtgaccaaccaccagaaggacctcttccgacgaactgagggcaactgttcgatctggggcaagaagccgacctttatcgagtgctcctaccgatctgccgacggcgccatccacaagtccaagcttatgacctccggcttttggggtgttgcccgacacatgaactacactggtgaccttatgggttccctggcctactgtctggcctgtggtggtaaccacctcctcccctacttctacattgtctacatgactattctgctggtccacagatgcattcgagacgagcaccgatgctccaacaagtacggcaaggattgggaacgatacaccgccgccgtctcttaccgactgctgcccaacatcttctaa

SEQ ID NO: 3 - DNA, 1296 bp, synthetic construct
  misc_feature 1..1296: note = Legionella drancourtii delta-7 sterol reductase codon optimised for Yarrowia lipolytica
  source 1..1296: mol_type = other DNA
atgtacttcaagatccgaaacaccctgggacctctgctgctgatcctgtcttgccccatcttcgtgatgctgatgtggtacaccaacaccgagctgaagggatctctgtctaccctgtgggacctgatcgtgcagcagggcctgttcgagactacctacaagatctggcagccctacttctggggctctgccctggcctggaaggtgatcttcgccttcatcatcttcgagctggccctgatgcgactgctgcccggcaaggaattcaccggacctgtgactcccaagggcaacgtgcccatctacaaggaaaacggacccctggccttcattaccaccatgactaccttttgcgtggcctctttcggcctgcatctgttccccgcctctatcctgtacgacaacctgggcgccatcctgggagccctgaacgtgttctctctgatcttctgcgccctgctgtatatcaagggccgatacttcccctcttctaccgactctggcatcaccaacaacatcattttcgactactactggggaaccgagctgtaccctcacatcttcggatggtctatcaagaagttcatcacctgtcgattcggcatgatgtcttggggactgttcctgatctcttactgcgccaagcaggccgagctgggcgacctggccaactctatgctgatctctgtggccctgcagttcctgtacctgtctaagttttacctgtgggagaagggctacctgcgatctctggacattatgcacgaccgagccggcttctacatctgctggggctgcctggtgtgggtgccctgcatctacacttctccctctatgtacctggtgctgcaccccatccacctgtctttctggctggccacctccattctggtgctgggagccgcttccatcctgatcaactacttcgccgaccgacagcgactgatgacccgagccaccgacggcgagtgtaagatctggggaaagaagcccgtcaccgttttcgcccagtaccagaccaccgagggcgacaagaagcagaccatcctgctggcctctggctggtggggcgtcgcccgacacttccactacgtgcccgagctggctggaaccttcttctggtctgtgcccgctctgtttgagaacttctctccttacttctacctgtgcttcctgaccattctgctcgtggaccgagccttccgagatgaccgacgatgctctgacaagtacggacagtactggcacaagtactgcgagctggtgccttacaagatcgtgcccttcgtgatctaa

SEQ ID NO: 4 - DNA, 1539 bp, synthetic construct
  misc_feature 1..1539: note = Ectocarpus siliculosus delta-7 sterol reductase codon optimised for Yarrowia lipolytica
  source 1..1539: mol_type = other DNA
atgatcgacggcgctgccatcggacgatctcccgtgatctcttcttggcacggctacaaccccgcctcttcgcagcagcagtgtctgcaggccgtccagacctctctgcccaccaccgacggcgaccgagagcgacgacgatctatggccctgcgaacctctaccaagcaggcctctgacgccatggacatccgagaggccgtgaaggcctctgtggccgccgagtctaaggactctaagctgtggggcttcgtgcccaactggttccgaactaccgtgggacccctgttcctgatcctggtgcctcctttcttcgtggtgttctggtggcacctcctggtgtctcactctggctcttgggtgtctctgtggggcgacctgaaggccgctggacccgagtacgtgctggacgtggtgccctctcctgtggaccccgctgcctggaagtacatcctcggcttcggcgtgttcgagatcctgctgatggtgggactgcccggcaaggccttccgagctaaccccaccgccaccggacacatccccgtgtacaaggccaacggcatgctgtcttacctggtgaccctggctaccctgtgcgccctggtcgccaccgaccgactggaccccaagaacgtgtacgacaagctgggcgagatcttcaccggcctgtctgtgttctctctgttcttcgtgctgctgctgaccgtgaagggcctgtacttcccctctaccgacgactctggatctaacggatctttcctgcagaactactggtggggcaccgagctgtaccctcgagtgttcggagccgacgtgaagatgttcaccaactgccgattcggcatgatgtactgggccgtgggcgccgtgatctacgcttacacccagcagcagatgtacggaaagctgtcctcttctatggccgtgtctgtgatcctgcagctgacctacatcaccaagttcttccactgggagatgggctacatgaactctatggacattcagcacgaccgagccggctactacctgtgctggggctgcctggtgtgggtgcccgccgtgtactcttctcccggcatctacctggtcaagcaccccatttctctcggctggtacggcgcctctgccattctggccctgggcctgctgtctatctgggccaacttcgacgctgaccgacagcgacacgccttccgacaggccaagggcgacatcatcgtgtggggcaagcccgccaagtacatcaccgccggctacatcaacgcccgaggcgagaaggcctcctctctgctgctctgcaccggctggtggggagtcgcccgacacttccactacctgcctgagattaccggcgccttcttctggaccgtgcctgctctgttcgagactcccactccttacttctacgtcgtgttcctggtgctgctcctcactgaccgagctttccgagatgacacccgatgccgaggcaagtacggcaagcactgggacaagtactgcgctcaggtgccctacaagatcgtgcccggcatcctgtaa

SEQ ID NO: 5 - DNA, 1380 bp, synthetic construct
  misc_feature 1..1380: note = Candidatus Protochlamydia amoebophila delta-7 sterol reductase codon optimised for Yarrowia lipolytica
  source 1..1380: mol_type = other DNA
atgctgatcgagatgctgtgcatcaccaagcacatcctcgagaagatctctaagttctctctggctcgaccccacgtggccaacaccaacattttcttcagacagaccttcggacccctgttcctgctgctgctgtgccctcctaccgtgttcgccttctggtacaccaacacctacctcgagggatctctgttccgattcaccgagttcgcctggcagcagggcttcctgtctaccctcaagactatctggttcccctacttcttcggcacctctatcgcctggaccatgctggccatcttcgcttctctgcagctgatcctgatgcgaattctgcccggcgagtgctacgagggccccatcactcccaccggacacgtgcccctgtacaaggccaaccgattctctgccttcatcgtgaccgtgtctatcttcctgatcgcctcttgctactaccagctgttcgctcccaccatcatctacgacaacttccccggcctgctgggcgccctgaacatcttctccctgtgtttctgcttcttcctggtgctgaagggccactacttcccttctaacggcgacgtcggcggctctggcaacatcatcttcgactactactggggcatggaactgtaccctcgactgctcggctgggacatcaagcagttcaccaactgccgattcggcatgatgtcttgggccctgatcgtgatctctttcgccgccaagcagcaacagctggacggcctgtctgactctatgttcgtggccgtggctctccagctcatctacattaccaagttcttcatctgggagcccggctacctgcgatctctggacattatgcacgaccgagccggctactacatctgctggggctgcctggtgtgggtgcccggcatctacacttctcccactctgtacctggtggatcaccccaaccacctgggactcgccctgtcctctctgctgttcgtggtgggcgtgatcggcatcctggtgaactacctggccgaccgacagcgacagctcgtgcgaaagaaccagggcaactgtcgaatctggggaaaggaacccatcctgaccattgccaagtacaccactcagaccggcgagactaagcagaacctgctgctcgcctctggctggtggggcctgtctcgacacttccactacctgcctgagctgctgggagccttctgctggtctgctcccgctctgttcgagaacttcctgccttacttctacttcgtgtttctgaccctgctcctgaccgaccgagctttccgagatgaccagagatgctctaagaagtacggcgaggactggaagatctactgccagcgagtgccctacaagatcatccccttcgtgatctaa

SEQ ID NO: 6 - DNA, 1452 bp, synthetic construct
  misc_feature 1..1452: note = Coccomyxa subellipsoidea delta-7 sterol reductase codon optimised for Yarrowia lipolytica
  source 1..1452: mol_type = other DNA
atggtcacaacccgagccgctgctcgagctcagacccctctgggcaaagctcctccctccgacctgtcccactccgaaccctccgtctctactcagaacggcaagaagacatgggccgagaccgctggggcaggcgagcacatcggcgcctggggtatcggcggcactgccggacacgtcttggcctacctgggtactcttgcccttatgatcggttgccccgcctttgcaatctacatgtggtttaccctcacccacttggatggctctcttgtggagctcgtccagttcgcccagaaggctggatttcagggcgtccgagcctcatggccctggccctcccaggaggcctgggccatcatcgcctccttcggcggtcttcaggccttcctgcagcttgccttgcccggtgctgtgcacaagggcccagtctctcccaagggtaacgtccccgtgtacaaggccaacggtgtcctcgcctactttaccaccctggctctgttcgtccttggctggcagttcaagctgttttcccccgccagagtctacgacctgttcggcgagatcctttccggcctgaacatgttctctctgctgttctgcctgttcctgtacttcaagggcaagtacgccccctcatcctcggattctggttctaccggctccctgatgtacgattattactgggggatggagctgtaccctcggattggacgacactttgacctgaagacttggactaactgccgaatgggcatgatgggctggggagttctggtcctgtgttacgctgtgaagcagcacgagctgtacggttacctttctaattcgatggctgtttctatcctgctcatgcatttatacattttcaagttcttcctctgggagactggttactggggtaccatggacatcgctcacgaccgcgctggatactacctctgctggggatgcctgaactgggtccccgctatctacacctctcccgccctgtacctcgtcgagaaccctattcagtggtctctgcccgccgccaccgctatcgctgttgccggtaccctggctatctacatcaactacgactccgatcggcagcgacaggttttccgagctaccaatggtaaggccctggtgtggggcaagcctccccaaattatctctgccaagtacatcaccggcgatggcaagcagaagacctccctgctcctggcctctggctggtgggggctcgcccgacacttccactacctgcccgagatcttggccgccttcttttggaccctccccgctggtatctcccacgctctgccctacttttacgtcttcttcctgactctgctccttaccgatcgagctttccgagacgacgtccgatgtagctccaaatacggtgcctactggcagcagtacaccaaggccgttccctacaagatgatcccttacattttctaa

SEQ ID NO: 7 - DNA, 1404 bp, synthetic construct
  misc_feature 1..1404: note = Mortierella verticillate delta-7 sterol reductase codon optimised for Yarrowia lipolytica
  source 1..1404: mol_type = other DNA
atggccgtgcagcagcgaaagacccctgctcaggtggacgtgaaggccgagtctaagctggacgcccaggtgggcaagacctggggccgagatcgagatgtgtctttcggcaccatcctgatctccctgggcatcctggtgatgtctcccatctgggtgatgtacacctacatctcttgcaacgcctaccagtgcgccatgtctgctcccgctctcgagatctacaactctcccgacactctggccgccattcagaccctgctgctgcgagaggtgccccgattctctccctacgccgctcgactgttcttcacctggctggccttccaggccgctctgtacgccttcctgcctgctcagatcggctacggccagcgaacccctgccggccacattctgccctacaaggtgaacggcctgctggcctggttcatctctcactctatctacgctgccggcggactgtacttcggctggtggaagctgtctatcatccacgacaactggggcggactgctggtggccgccaacatgtacggctactttctgacattcttctgctttatcaaggcctacacctttccttctcaccccgccgaccgaaagttctctggctctttcatctacgacctgctgatgggcatcgagttcaaccctcgaatcggcaagctgttcgacttcaagctgtttcacaacggacgacccggcatcgtggcctggaccatgatcaacctgtccttcgccgctgctcagtacgagaagatcggatacgtgaccaactctatgatcctgctgaacctgctgcatgctacctacgtgctggacttcttctacaacgaggactggtatctgcgaaccatcgacattgcccacgaccacttcggcttctacctggcctggggcgactccgtgtggctgccctggctgtacaccctgcagtctcactacctggtgcgaaaccccgtggacctgactcctgtgcagttcgccttcgtgttcaccgtgggctacatcggctacttcatcttccgatctgtgaaccaccagaaggacatcgtccgatctaccaacggcgagtgcatgatctggggcaagcccgccaaggtgatccgaacctctttcgtgacctctgacggcaagacccacaagtctctgctgctgtgctctggctactggggcctgtctcgacacttcaactacgtgggcgacctgctcatctctctggccatgtgcatgacctgcggcacccagcatctgctgccctacttctacatcatctacatgaccatcctgctgctccaccgaatccagcgagatcacacccgatgcaagggcaagtacggaaagtactgggacgagtacatgaaggccgtgccttacaagctgatcccctacgtgtactaa

SEQ ID NO: 8 - DNA, 1299 bp, synthetic construct
  misc_feature 1..1299: note = Glycine soja delta-7 sterol reductase codon optimised for Yarrowia lipolytica
  source 1..1299: mol_type = other DNA
atgggcgctaccgtgcactctcccctggtgacctacgcctctgtgatctctctgctgaccctgtgtcctcctttcgtggtgctgctgtggtacaccatgactctggccgacggctctgtgtctgagactttccactacctgcgacagaacggcctgcagggcctgctgcacatctggcccactcctactcctaccgcctgcaagatcattgccgtgtacgccgccttcgaggccgctctgcagctgctgctgcccggcaagaccgtgtacggccccatctctcccaccggccaccgacctgtgtacaaggccaacggactgcaggcctacttcgtgaccctgatcacctacttcgccctgtggtggttcggcatcttcaaccccaccatcgtgtaccaccacctgggcgagatctactctgccctgatcttcggctctttcctgttctgcgtgttcctgtacatcaagggccatctggctccctcttctaccgactctggatcttctggcaacctgatcatcgacttctactggggcatggaactgtaccctcgaatcggcaagcacttcgacatcaaggtgttcaccaactgtcgattcggcatgatgtcttgggccgtgctggccctgacctactgcatcaagcagtacgaagagaacggcaaggtggccgactctatgctggtcaacaccgctctgatgctggtgtacgtgaccaagttcttctggtgggaggccggctactggtctaccatggacattgcccacgaccgagccggcttctacatctgctggggctgcctggtgtgggtgccctctgtgtacacctctcctggcatgtacctggtgaaccatcctgtgaacctgggcatcaagctggctctgtctatcctggtggccggcatcctgtgcatctacatcaactacgactgcgaccgacagcgacaagagttccgacgaactaacggaaagggcaccgtgtggggcaaggccccttctaagatcgaggccacctacaccactacctctggcgagactaagcgatccctgctgctgacctctggctggtggggcctgtctcgacacttccactacgtgcccgagatcctggccgccttcttctggaccgtgcctgctctgttcgagcacttcctgccttacttctacgtgatcttcctgaccatcctgctgttcgaccgagctaagcgagatgacgaccgatgccgatctaagtacggcaagtactggaagctgtactgcgacaaggtgccctaccgaatcatccccggcatctactaa

SEQ ID NO: 9 - DNA, 1443 bp, synthetic construct
  misc_feature 1..1443: note = Tetraselmis sp. GSL018 delta-7 sterol reductase codon optimised for Yarrowia lipolytica
  source 1..1443: mol_type = other DNA
atgaagcgagcctccaagacccccgacaccgcctctaagggtcgagaacccctttctgagcctcacaccaacggtgtcgctaaggccagcaacaagacctcttgggccgagtccaatggcatcggtgatcgagacggattcatgggcctgtccggtgccgccggccatgctgtggcccttctgggcaccgtcgtgctgctcgtcggttgccccgcctttgtcttcgtgctctggtacattaattgtcgactcgacggctccgtctccgagttcgtcgccctcgccgctcgagagggcgccgtgggtctctggcaacgatggcctactcccactgctgaggcctgggccatcattggcaccttcggtgcagtcgaggccttcctgcagctggctctccctggcaagaagttcctgggtcccgtctctcctaagggtaacgtccccgtctacaaggccaacggcatgcaagcctacgtgactaccctggtcctgttctttgccgtctggggctccggcatctacaaccccgcgcgagtctacgatctcatgggtgagattctggccgccctgaacatcttttctctgctgttttgcctgttcctcaacattaagggtcatgtcgccccttcctctactgactccggctctaccggctctttgctgtacgactactactggggcatggagctttaccctagaattggccgatccttcgacattaagacctggaccaactgccgagtcggcatgatgggttggggcatcctgatcctgtgctacgccgccaagcaggtggaggaggctggatttctgtccgactccatggctgtgtccgtcattctcatgcacgtttacatcgccaagttcttctggtgggagactggttactggaagaccatggacatcatgcacgatcgagccggttactacatctgctggggttgcctcgtgtggatcccctccatgtacacctctcccaccatgtttcttgtcaagcatcctatggtgctgggccccaccctcaccggcgctgtcctggctgctggcctgctgtgtatctacatcaactacgacgccgaccgacagcgacaggttttccgagagtctaacggcaaggccctgatttggggtcgaaagcctaagaagatcgaggcccagtacactaccgcggacggccaaactaagacctctctgctgctggtgtctggttggtggggtgtctcccgacatttccactacctgcccgagatacttgcctccgtgttctggtctgtgcccgcccagactgattacgctatggcctacctgtactctgcctacctcaccattcttctcgtggaccgtgccttccgagacgacctgcgatgcgcttccaagtacggcaagcactgggtcgagtattgccgacaggtcccccacaagatcgtgccctacatcttctaa

SEQ ID NO: 10 - DNA, 1368 bp, synthetic construct
  misc_feature 1..1368: note = Waddlia chondrophila delta-7 sterol reductase codon optimised for Yarrowia lipolytica
  source 1..1368: mol_type = other DNA
atggccgccaccaccaccaacgtgcagacccgaaactggggccgagcctgggagactacctggctgtctctgttctctaccattgctctgctggccaccgctcctatgatggtgctgtactgctacattgcctgcgtgcgattccgaggctctctgatcggacccgcctacgctctggcctctggcgccgtgtctctggactctctgttcccctcgttcgaggtgggcatcttcgccctgtacctcggctggttcgccttccagctgctgctgtacctgggactgcccgacctgctgcaccgaattctgccccgataccgaggcggccgacaagagggcgctgtgacccctgccggcaagcagctggtgtaccagatcaacggactgcaggcctggctgatctcccacctgttcttcggcatcggcgcctacgtgctcggatggttctctccctcgatcattgccgagaactggggaggcttcctgatcgtgaccaacgtgatgggctacctgaccgccatcttcgtgtacgtgaaggcctaccgatttccttcgaacgccgaggaccgaaagttctctggcaaccctctgtacgacttcttcatgggcatcgagttcaaccctcgaatcggcaagttcgacttcaagctgttcttcaacggacgacccggcatcattgcttggaccctgatcaactggtctttcgccgccaagcagtacgccgacctgggatacctgcctaactctatgctgctggtgaacgtgctgcaggctatctacgtgctggacttcttctggcacgagacttggtatctcaagaccatcgacatctgccacgaccacttcggctggatgctgtcttggggcgacctggtgtggctgccctacatgtacaccctccagggcctgtacctgctctaccatcctgtggacctgtctaccggcttcgctctgttcgtgctgaccctgggcgtcgtgggctacgccattttccgatctgccaaccaccagaaggaccacttccgacgagtgcaaggcaaggaacccatctggggaaagatgcccgagttcatctcttgccagtataccgccgctgacggctctctgcaccacaccaagctgctcctctccggctggtggggacgagcccgacacatgaactacaccggcgacctgatgctgtccctggcctactgcctggcctgcggcttctctcacctcctgccttacttctacttcgtctacatgaccatcctgctggtcaaccgatgctaccgagatgagcaccgatgcgagaacaagtacggcgacgcctggcgaaagtactgccgacgagtcccctaccgactgatccccggcatctactaa

SEQ ID NO: 11 - DNA, 1968 bp, synthetic construct
  misc_feature 1..1968: note = Tetrahymena thermophilia squalene-tetrahymanol cyclase codon optimised for Yarrowia lipolytica
  source 1..1968: mol_type = other DNA
atgaagaagatcctcatcggtctcatcatcggtctcttcctcttctcctccgtcaacgcctccgtcaacctcaccgaggtccagaacgccatctccatccagcagggtatcaactgggctgaggtccacaacaacacctggtactaccctccctacctcggtgagatgttcatctccgagtactacttcgagctcctcgtcctcaactggacccacaagtccgccttcaacgccacctacttcaccgagcgactcctccagacccagttcgaggacggttcctgggagcaggtccgagagcagaacctcgagaccggtcagctcgacgccaccgtcttcaactactggtacctcaagtccatcaacaacaaccccaagatcgaggctgccctccagaaggcccgaaagtggatcgtcgctcagggtggtatcgaggccacccagaccatgaccaagttcaagctcgctgccttcggtcagtactcctgggaggacctctggtacgtccctctcttcatcttcaagcagaacggtatcttcaagtacacctacgtcaaggacatcgtcgcccagtgggtctacccccacctcaccgctctcgcctacctccgataccagcgaaccgtcttcaacgtccctgtcgctgacctccgagagctctggatcaactaccccaagaacggtatcaagatctccccccgagagtactccaccctcaaccccgactccgacctcctcatcctcatggacgagatcttcaagctcaagcagcctctcggttccttcggtgcctacaccatctccaccctcctcaccctcatgtccttcaaggacttccagtccaagcacccccacctctaccagaacgagatccagaaggcctacgaggacggttactacttcgtcgagttcaactacttcaacttccgagaggcctaccacggttccctcgacgacggtcgatggtgggacaccatcctcatctcctgggccatgctcgagtccggtcaggacaaggagcgaatcttccccatcgtccagaacatggtcaaggagggtctccagcccaagaagggtatcggttacggttacgacttcgagtacgctcccgacaccgacgacaccggtctccttctcgtcgtcatgtcctactacaaggaggccttccagaagcagatccccgagaccatcgagtggctcttctccatgcagaacgacgacggtggttaccccgccttcgacaagggtaagaacgaggacaacctcctcttcaagttcgccttcaacatggccggtatcgccaactccgccgagatcttcgacccttcctgccctgacatcaccggtcacatcatggagggtctcggtgagttcggttaccaggccaaccacccccagatccagaacatgatcaagtaccagcgaaagacccagaacaagtggggttcctggcaggctcgatggggtgtcaactacatcatggctgtcggtgctgtcgttcctggtctcgctcgagtcaactacgacctcaacgagcagtgggtccagaactccatcaactacctcctcaacaagcagaacaaggatggcggcttcggtgagtgcgtcctctcctacaacgaccccgagaagtggaacggtatcggtaagtccaccgtcacccagacctcctggggtctcctcgctctcctcgaggtctacaaccagaacgagcagatcaagcacgctgccgatcgagctgcccagtacctcctcgaccagttcaagcgagacgacaacaccttctacgaccactccaccatcggtaccggtcaccgaggtctcctctacctccagtacccctcctacgcccagtccttccctctcgtcgccctcaaccgataccagaagatctcccagggtcagtaccacttctccaagaacctctacaacggtaacggtgagcccgtccagaagcagaacatctaa

SEQ ID NO: 12 - DNA, 1959 bp, synthetic construct
  misc_feature 1..1959: note = Salmonella enterica acetyl-CoA synthetase codon optimised for Yarrowia lipolytica
  source 1..1959: mol_type = other DNA
atgtctcagacccacaagcacgctatccccgccaacattgccgaccgatgcctgatcaaccccgagcagtacgagactaagtacaagcagtctatcaacgaccccgacaccttctggggcgagcagggcaagatcctggactggatcaccccttaccagaaggtcaagaacacctctttcgctcccggcaacgtgtctatcaagtggtacgaggacggcaccctgaacctggccgccaactgcctggaccgacacctccaagagaacggcgaccgaaccgccatcatctgggagggcgacgacacctctcagtctaagcacatctcttaccgagagctgcaccgagatgtgtgccgattcgctaacaccctgctggacctgggcatcaagaagggcgacgtcgtcgctatctacatgcccatggtgcccgaggccgccgtggccatgctggcctgcgctcgaatcggcgccgtgcactctgtgatcttcggcggcttctcgcccgaggctgtggccggacgaatcatcgactcctcttctcgactggtgattaccgccgacgagggcgtgcgagccggccgatctattcccctgaagaagaacgtcgacgacgctctgaagaaccccaacgtgacctctgtcgagcacgtgatcgtgctgaagcgaaccggctctgacatcgactggcaagagggccgagatctgtggtggcgagatctgatcgagaaggcttctcccgagcaccagcctgaggccatgaacgctgaggaccctctgttcatcctgtacacctctggctctaccggcaagcccaagggcgtgctgcacaccaccggcggctacctggtgtacgccgccaccaccttcaagtacgtgttcgactaccatcctggcgacatctactggtgcaccgctgacgtcggctgggtgaccggccactcttacctgctgtacggacccctggcctgtggcgctaccactctgatgttcgagggcgtccccaactggcccactcctgctcgaatgtgccaggtggtggacaagcaccaggtgaacattctgtacaccgctcctaccgccattcgagccctgatggccgagggcgacaaggccatcgagggcaccgaccgatcttctctgcgaatcctgggctctgtgggcgagcctattaaccccgaggcctgggagtggtactggaagaagattggcaaggaaaagtgccccgtcgttgacacctggtggcagaccgagactggcggcttcatgattacccctctgcctggcgccatcgagctgaaggccggctctgctacccgaccattcttcggcgtgcagcccgctctggtcgacaacgagggacaccctcaagagggcgccaccgagggcaacctggtcatcaccgactcttggcccggacaggctcgaaccctgttcggcgaccacgagcgatttgagcagacctacttctctaccttcaagaacatgtacttctctggcgacggcgctcgacgagatgaggacggctactactggattaccggccgagtggacgacgtgctgaacgtgtctggccaccgactgggcaccgccgagatcgagtctgccctggtggctcaccccaagatcgccgaggctgccgtcgtgggcattccccacgccatcaagggccaagccatctacgcctacgtgaccctcaaccacggcgaggaaccctcgcctgagctgtacgccgaggtgcgaaactgggtgcgaaaggaaatcggtcccctggctacccctgacgtcctgcattggaccgactcgctgcccaagacacgatctggaaagatcatgcgacgaatcctgcgaaagatcgctgccggtgacacctctaacctgggcgacacttctaccctggctgaccctggcgtggtcgagaagcccctggaagagaagcaggccattgctatgccctcttaa

SEQ ID NO: 13 - DNA, 1701 bp, synthetic construct
  misc_feature 1..1701: note = Solanum lycopersicum delta-24 sterol reductase codon optimised for Yarrowia lipolytica
  source 1..1701: mol_type = other DNA
atgtctgacgccaaggctcccgtggccactgcttaccccaagcgaaagatccagctggtggacttcctgctgtctttccgatggatcatcgtgattttcttcgtgctgcccttctctttcctgtactacttctctatctacctgggcgacgtgaagtctgagcgaaagtcttacaagcagcgacagatggaacacgacgagaacgtgaaggaagtggtgaagcgactgggccagcgaaacgccgagaaggacggcctggtgtgcaccgctcgacctccatgggtcgtcgtgggcatgcgaaacgtggactacaagcgagcccgacacttcgaggtggacctgtctaagttccgaaacatcctggacatcgacaccgagcgaatggtggccaaggtcgagcccctggtgaacatgggccagatgtctcgagtgaccattcctatgaacctgtctctggccgtgctggccgagctggacgacctgaccgtcggcggcctgatcaacggcttcggcgtcgagggatcttctcacatcttcggcctgttctctgacaccgtggtggccctcgaggtggtgctggctgacggcaaggtggtgcgagccaccaaggacaacgagtactctgacctgttctacgctatcccctggtcgcagggcaccctgggcctgctggtgtctgccgagatcaagctgatccccgtggaccagtacgtgaagctgacctacaagcccgtgcgaggcaacctgaaggaactggcccaggcctacgccgactctttcgctcccaaggacggcgaccaggacaacccctctaaggtgcccgagatggtcgagggcatgatctacggccccaccgagggcgtgatgatgaccggcatgtacgcctctcgaaacgaggccaagcgacgaggcaacgtgatcaacaactacggctggtggttcaagccctggttctaccagcacgctcagaccgctctgaagcgaggcgagttcgtcgagtacatccctactcgagactactaccaccgacacacccgatctctgtactgggagggcaagctgattctgcccttcggtgaccagttctggttccgattcctgctcggctggctgatgcctcctaagatcgccctgctgaaggctacccagtctgaggccatccgaaactactatcacgaccaccacgtgatccaggacctgctcgtgcccctgtacaaggtgggcgactgcctcgagtgggtgcaccgagagatggaagtgtaccccatctggctgtgtccccaccgaatctacaagctgcctgtgcgacctatgatctaccccgagcctggcttcgagaagcacaagcgacagggtgacaccgagtacgcccagatgtacaccgacgtgggcgtgtactacgtgcccggtgccgtgctgcgaggtgagcccttcgacggctctgagaagtgccgacagctggaactgtggctgatcgagaaccacggcttccaggctcagtacgccgtgaccgagctgaccgagaagaacttctggcgaatgttcgacaacggcctgtacgagcagtgccgacgaaagtacaaggccatcggcaccttcatgtctgtgtactacaagtctaagaagggccgaaagactgagaaggaagttcaagaggccgagcaagagaaggctgagcaagaaacccctgaggccaactaa

SEQ ID NO: 14 - DNA, 1086 bp, synthetic construct
  misc_feature 1..1086: note = Chenopodium quinoa C-28 sterol methyltransferase codon optimised for Yarrowia lipolytica
  source 1..1086: mol_type = other DNA
atggactctatggccctgatctgcaccgtgggcctgctgttcggcggcctgtactggttcatctgcattcacggacccgccgagcgaaagggcaagcgagccgtggacctgtctggcggctctatctcttctgacaaggtgcaggacaagtaccagcagtactggtcgttcttccgacgacctaaggaaatcgagactgccgagaaggtgcccgacttcgtggacaccttctacaacctggtgaccgacatctacgagtggggctggggccagtctttccacttctctccctcgattcccggcaagtctcaccgagatgctacccgaatccacgaagagatggccgtcgacctgatcaaggtgtctcccggccaaaagatcctggacgtcggctgcggcgtcggcggacccatgcgagccattgccgctcactctcgagccaaggtgaccggcatcaccatcaacgagtaccaggtgaagcgagccaagctgcacaacaagaaggccggactggactctctgtgcgaggtggtgtgcggcaacttcctcgagatgcccttcgcctctaacaccttcgacggcgcctactctatcgaggccacctgtcacgctcccaagctggacgacgtgtactctgagatcttccgagtgctgaagcccggctctctgtacgtgtcttacgaatgggtgaccaccgacaagttcaacggcgacgactctgagcactgcgacgtgatccagggcatcgagcgaggcgacgctctgcccggcctgcgacgatacgacgagatctctgaggccgccaagaaggtgggcttcgagatcgtggacgagcgagatctggccgctcctcctgctaagccctggtgggaccgactgaagatgggccgaatcgcctactggcgaaaccacatcgtggtgaccgtgctggccgccatcggcgtggctcccaagggcaccgtggacgtgcacgacatgctgttcaagaccgccgactacctgactcgaggcggcgagtctggcattttctctcccatgcacatgatcctgtgtcgaaagcccgtggacgccaagtctgactcttaa

SEQ ID NO: 15 - DNA, 36 bp, synthetic construct
  misc_feature 1..36: note = PR-23934 (PrEXP_for-HpHMX_rv)
  source 1..36: mol_type = other DNA
  modified_base 12: mod_base = OTHER; note = uracil
acccattgctgtagatatgtcttgtgtgtaaggggg

SEQ ID NO: 16 - DNA, 25 bp, synthetic construct
  misc_feature 1..25: note = PR-10593 (Fragment2EpiVecYL _fw)
  source 1..25: mol_type = other DNA
  modified_base 9: mod_base = OTHER; note = uracil
agcaggcttggaggcgacgtggcag

SEQ ID NO: 17 - DNA, 32 bp, synthetic construct
  misc_feature 1..32: note = PR-23935 (HphMX_for-PrEXP1_fw)
  source 1..32: mol_type = other DNA
  modified_base 12: mod_base = OTHER; note = uracil
acagcaatgggtaaaaagcctgaactcaccgc

SEQ ID NO: 18 - DNA, 27 bp, synthetic construct
  misc_feature 1..27: note = PR-23936 (TTef1_rv)
  source 1..27: mol_type = other DNA
  modified_base 9: mod_base = OTHER; note = uracil
aagcctgctgaattcggacacgggcat

SEQ ID NO: 19 - DNA, 41 bp, synthetic construct
  misc_feature 1..41: note = PR-10604 (tracrRNA _rev)
  source 1..41: mol_type = other DNA
  modified_base 8: mod_base = OTHER; note = uracil
cacgcgataccgtacccacacaaaaaaagcaccaccgactc

SEQ ID NO: 20 - DNA, 30 bp, synthetic construct
  misc_feature 1..30: note = PR-10607 (PrtRNAGly _fw)
  source 1..30: mol_type = other DNA
  modified_base 8: mod_base = OTHER; note = uracil
cgtgcgatagtgaatcattgctaacagatc

SEQ ID NO: 21 - DNA, 29 bp, synthetic construct
  misc_feature 1..29: note = PR-24013 (Tefint_Fuse_fw)
  source 1..29: mol_type = other DNA
  modified_base 10: mod_base = OTHER; note = uracil
atcagtagctagagaccgggttggcggcg

SEQ ID NO: 22 - DNA, 19 bp, synthetic construct
  misc_feature 1..19: note = PR-18214 (PTEFintron_USER_rv)
  source 1..19: mol_type = other DNA
  modified_base 15: mod_base = OTHER; note = uracil
agtactgcaaaaagtgctg

SEQ ID NO: 23 - DNA, 36 bp, synthetic construct
  misc_feature 1..36: note = PR-15528 (PrYlTDH1 _rev)
  source 1..36: mol_type = other DNA
  modified_base 9: mod_base = OTHER; note = uracil
atgacagattgttgatgtgtgtttaattcaagaatg

SEQ ID NO: 24 - DNA, 34 bp, synthetic construct
  misc_feature 1..34: note = PR-15529 (PrYlTDH1for fusion_fw)
  source 1..34: mol_type = other DNA
  modified_base 10: mod_base = OTHER; note = uracil
agctactgatgacgcagtaggatgtcctgcacgg

SEQ ID NO: 25 - DNA, 35 bp, synthetic construct
  misc_feature 1..35: note = PR-23752 (YlHMG1 ->_U2_fw)
  source 1..35: mol_type = other DNA
  modified_base 9: mod_base = OTHER; note = uracil
atctgtcatgccacaatgctacaagcagctattgg

SEQ ID NO: 26 - DNA, 31 bp, synthetic construct
  misc_feature 1..31: note = PR-23753 (YlHMG1 ->_U2_rv)
  source 1..31: mol_type = other DNA
  modified_base 8: mod_base = OTHER; note = uracil
cacgcgatctatgaccgtatgcaaatattcg

SEQ ID NO: 27 - DNA, 43 bp, synthetic construct
  misc_feature 1..43: note = PR-24014 (ERG12_fw)
  source 1..43: mol_type = other DNA
  modified_base 15: mod_base = OTHER; note = uracil
actttttgcagtactaaccgcaggactacatcatttcggcgcc

SEQ ID NO: 28 - DNA, 27 bp, synthetic construct
  misc_feature 1..27: note = PR-24015 (ERG12_rev)
  source 1..27: mol_type = other DNA
  modified_base 8: mod_base = OTHER; note = uracil
cacgcgatctaatgggtccagggaccg

SEQ ID NO: 29 - DNA, 30 bp, synthetic construct
  misc_feature 1..30: note = PR-24016 (OpSeACS_fw)
  source 1..30: mol_type = other DNA
  modified_base 8: mod_base = OTHER; note = uracil
cgtgcgatttaagagggcatagcaatggcc

SEQ ID NO: 30 - DNA, 37 bp, synthetic construct
  misc_feature 1..37: note = PR-24017 (OpSeACS_rev)
  source 1..37: mol_type = other DNA
  modified_base 9: mod_base = OTHER; note = uracil
atctgtcatgccacaatgtctcagacccacaagcacg

SEQ ID NO: 31 - DNA, 44 bp, synthetic construct
  misc_feature 1..44: note = PR-24018 (YlACL1_fw)
  source 1..44: mol_type = other DNA
  modified_base 15: mod_base = OTHER; note = uracil
actttttgcagtactaaccgcagtcagcgaaatccattcacgag

SEQ ID NO: 32 - DNA, 30 bp, synthetic construct
  misc_feature 1..30: note = PR-24019 (YlACL1_rev)
  source 1..30: mol_type = other DNA
  modified_base 8: mod_base = OTHER; note = uracil
cacgcgatttaaactccgagaggagtggaa

SEQ ID NO: 33 - DNA, 28 bp, synthetic construct
  misc_feature 1..28: note = PR-24020 (YlIDI1_fw)
  source 1..28: mol_type = other DNA
  modified_base 8: mod_base = OTHER; note = uracil
cgtgcgatctacttgatccaccgccgaa

SEQ ID NO: 34 - DNA, 35 bp, synthetic construct
  misc_feature 1..35: note = PR-24021 (YlIDI1_rev)
  source 1..35: mol_type = other DNA
  modified_base 9: mod_base = OTHER; note = uracil
atctgtcatgccacaatgacgacgtcttacagcga

SEQ ID NO: 35 - DNA, 44 bp, synthetic construct
  misc_feature 1..44: note = PR-24022 (ERG20_fw)
  source 1..44: mol_type = other DNA
  modified_base 15: mod_base = OTHER; note = uracil
actttttgcagtactaaccgcagtccaaggcgaaattcgaaagc

SEQ ID NO: 36 - DNA, 32 bp, synthetic construct
  misc_feature 1..32: note = PR-24023 (ERG20_rv)
  source 1..32: mol_type = other DNA
  modified_base 8: mod_base = OTHER; note = uracil
cacgcgatctacttctgtcgcttgtaaatctt

SEQ ID NO: 37 - DNA, 90 bp, synthetic construct
  misc_feature 1..90: note = pCfB10224 (ERG4_KORep_90bp)
  source 1..90: mol_type = other DNA
acataaagccatccacccttccttcgcaaccacacacaccatacaacgataagcttagtgagcgaatggtgaggttacttaattgagtgg

SEQ ID NO: 38 - DNA, 90 bp, synthetic construct
  misc_feature 1..90: note = pCfB10225 (ERG5_KORep_90bp)
  source 1..90: mol_type = other DNA
aacttctctctctcacaccaccaccacaacacaactccgcccaccggtatataggtttggtaatgtattaatattaatgatggggcgagg

SEQ ID NO: 39 - DNA, 90 bp, synthetic construct
  misc_feature 1..90: note = pCfB10226 (ERG6_KORep_90bp)
  source 1..90: mol_type = other DNA
gatctgaatcgcccttgtaaacccccccaaaacaccacattcaacactgagtaacttatagagggagccacggccccaaaatttataatg

SEQ ID NO: 40 - DNA, 17 bp, synthetic construct
  misc_feature 1..17: note = PR-14617 (vector verification E. coli cPCR)
  source 1..17: mol_type = other DNA
tatccctgtgttgaatc

SEQ ID NO: 41 - DNA, 16 bp, synthetic construct
  misc_feature 1..16: note = PR-14619 (vector verification E. coli cPCR)
  source 1..16: mol_type = other DNA
tatcgacccagttagc

SEQ ID NO: 42 - DNA, 21 bp, synthetic construct
  misc_feature 1..21: note = PR-8859 (integration verification Yl cPCR)
  source 1..21: mol_type = other DNA
aagtgtggatggggaagtgag

SEQ ID NO: 43 - DNA, 28 bp, synthetic construct
  misc_feature 1..28: note = PR-14576 (IntE_3 verification Yl cPCR)
  source 1..28: mol_type = other DNA
  modified_base 8: mod_base = OTHER; note = uracil
cacgcgattgaaggaaatgcctaaaacc

SEQ ID NO: 44 - DNA, 20 bp, synthetic construct
  misc_feature 1..20: note = PR-14835 (IntE_3 verification Yl cPCR)
  source 1..20: mol_type = other DNA
cacgcacgccattctataag

SEQ ID NO: 45 - DNA, 27 bp, synthetic construct
  misc_feature 1..27: note = PR-14592 (IntE_4 verification Yl cPCR)
  source 1..27: mol_type = other DNA
  modified_base 7: mod_base = OTHER; note = uracil
acgcgatttaacactggaccgtactgc

SEQ ID NO: 46 - DNA, 20 bp, synthetic construct
  misc_feature 1..20: note = PR-20880 (IntE_4 verification Yl cPCR)
  source 1..20: mol_type = other DNA
attgctaagcgaccatagac

SEQ ID NO: 47 - DNA, 42 bp, synthetic construct
  misc_feature 1..42: note = PR-27571 (A_Es_Fw)
  source 1..42: mol_type = other DNA
  modified_base 15: mod_base = OTHER; note = uracil
actttttgcagtactaaccgcagatcgacggcgctgccatcg

SEQ ID NO: 48 - DNA, 30 bp, synthetic construct
  misc_feature 1..30: note = PR-27572 (A_Es_Rv)
  source 1..30: mol_type = other DNA
  modified_base 8: mod_base = OTHER; note = uracil
cacgcgatttacaggatgccgggcacgatc

SEQ ID NO: 49 - DNA, 34 bp, synthetic construct
  misc_feature 1..34: note = PR-27625 (ERG4_gRNA_Fw)
  source 1..34: mol_type = other DNA
ggtctcgtactgcttgacagcgggttttagagct

SEQ ID NO: 50 - DNA, 33 bp, synthetic construct
  misc_feature 1..33: note = PR-27626 (ERG4_gRNA_Rv)
  source 1..33: mol_type = other DNA
ccgctgtcaagcagtacgagacctaaccaacct

SEQ ID NO: 51 - DNA, 34 bp, synthetic construct
  misc_feature 1..34: note = PR-27627 (ERG5_gRNA_Fw)
  source 1..34: mol_type = other DNA
gtagggaaccataatgacagggggttttagagct

SEQ ID NO: 52 - DNA, 33 bp, synthetic construct
  misc_feature 1..33: note = PR-27628 (ERG5_gRNA_Rv)
  source 1..33: mol_type = other DNA
cccctgtcattatggttccctactaaccaacct

SEQ ID NO: 53 - DNA, 34 bp, synthetic construct
  misc_feature 1..34: note = PR-27629 (ERG6_gRNA_Fw)
  source 1..34: mol_type = other DNA
agaaacaaacagcatcatggggggttttagagct

SEQ ID NO: 54 - DNA, 33 bp, synthetic construct
  misc_feature 1..33: note = PR-27630 (ERG6_gRNA_Rv)
  source 1..33: mol_type = other DNA
cccccatgatgctgtttgtttcttaaccaacct

SEQ ID NO: 55 - DNA, 19 bp, synthetic construct
  misc_feature 1..19: note = PR-27891 (Erg4Rep_P1F)
  source 1..19: mol_type = other DNA
ctctcaacaccttcaccgc

SEQ ID NO: 56 - DNA, 28 bp, synthetic construct
  misc_feature 1..28: note = PR-27892 (Erg4Rep_P1R)
  source 1..28: mol_type = other DNA
  modified_base 9: mod_base = OTHER; note = uracil
acctgcactacgataagcttagtgagcg

SEQ ID NO: 57 - DNA, 27 bp, synthetic construct
  misc_feature 1..27: note = PR-27893 (Erg4Rep_P2F)
  source 1..27: mol_type = other DNA
  modified_base 8: mod_base = OTHER; note = uracil
gtgcaggttgtgtggttgcgaaggaag

SEQ ID NO: 58 - DNA, 20 bp, synthetic construct
  misc_feature 1..20: note = PR-27894 (Erg4Rep_P2R)
  source 1..20: mol_type = other DNA
gcactcaaaataccccgttc

SEQ ID NO: 59 - DNA, 19 bp, synthetic construct
  misc_feature 1..19: note = PR-27895 (Erg5Rep_P1F)
  source 1..19: mol_type = other DNA
ctcggtttgttgcagcagg

SEQ ID NO: 60 - DNA, 29 bp, synthetic construct
  misc_feature 1..29: note = PR-27896 (Erg5Rep_P1R)
  source 1..29: mol_type = other DNA
  modified_base 9: mod_base = OTHER; note = uracil
acctgcactatggtccgtatcgtgaaatg

SEQ ID NO: 61 - DNA, 26 bp, synthetic construct
  misc_feature 1..26: note = PR-27897 (Erg5Rep_P2F)
  source 1..26: mol_type = other DNA
  modified_base 8: mod_base = OTHER; note = uracil
gtgcaggtgggcggagttgtgttgtg

SEQ ID NO: 62 - DNA, 22 bp, synthetic construct
  misc_feature 1..22: note = PR-27898 (Erg5Rep_P2R)
  source 1..22: mol_type = other DNA
ggtcggctatccaatacatctc

SEQ ID NO: 63 - DNA, 20 bp, synthetic construct
  misc_feature 1..20: note = PR-27899 (Erg6Rep_P1F)
  source 1..20: mol_type = other DNA
gctacaagccggaggggaac

SEQ ID NO: 64 - DNA, 29 bp, synthetic construct
  misc_feature 1..29: note = PR-27900 (Erg6Rep_P1R)
  source 1..29: mol_type = other DNA
  modified_base 9: mod_base = OTHER; note = uracil
acctgcactgttgaatgtggtgttttggg

SEQ ID NO: 65 - DNA, 28 bp, synthetic construct
  misc_feature 1..28: note = PR-27901 (Erg6Rep_P2F)
  source 1..28: mol_type = other DNA
  modified_base 8: mod_base = OTHER; note = uracil
gtgcaggtactgagtaacttatagaggg

SEQ ID NO: 66 - DNA, 20 bp, synthetic construct
  misc_feature 1..20: note = PR-27902 (Erg6Rep_P2R)
  source 1..20: mol_type = other DNA
ctgtaccgtttggaggactc

SEQ ID NO: 67 - DNA, 31 bp, synthetic construct
  misc_feature 1..31: note = PR-27929 (ERG4_gRNA2_Fw)
  source 1..31: mol_type = other DNA
atctcgtcgacctactacgagttttagagct

SEQ ID NO: 68 - DNA, 30 bp, synthetic construct
  misc_feature 1..30: note = PR-27930 (ERG4_gRNA2_Rv)
  source 1..30: mol_type = other DNA
tcgtagtaggtcgacgagattaaccaacct

SEQ ID NO: 69 - DNA, 31 bp, synthetic construct
  misc_feature 1..31: note = PR-27931 (ERG5_gRNA2_Fw)
  source 1..31: mol_type = other DNA
tgagaaatacaaggcccagtgttttagagct

SEQ ID NO: 70 - DNA, 30 bp, synthetic construct
  misc_feature 1..30: note = PR-27932 (ERG5_gRNA2_Rv)
  source 1..30: mol_type = other DNA
actgggccttgtatttctcataaccaacct

SEQ ID NO: 71 - DNA, 31 bp, synthetic construct
  misc_feature 1..31: note = PR-27933 (ERG6_gRNA2_Fw)
  source 1..31: mol_type = other DNA
ttcccgatactacaagggaggttttagagct

SEQ ID NO: 72 - DNA, 30 bp, synthetic construct
  misc_feature 1..30: note = PR-27934 (ERG6_gRNA2_Rv)
  source 1..30: mol_type = other DNA
ctcccttgtagtatcgggaataaccaacct

SEQ ID NO: 73 - DNA, 26 bp, synthetic construct
  misc_feature 1..26: note = PR-27969 (Erg5ORF_R_U3)
  source 1..26: mol_type = other DNA
  modified_base 8: mod_base = OTHER; note = uracil
cacgcgattcggtcggcaacaatctg

SEQ ID NO: 74 - DNA, 26 bp, synthetic construct
  misc_feature 1..26: note = PR-27970 (Erg5_UP_uORF_F_U3)
  source 1..26: mol_type = other DNA
  modified_base 8: mod_base = OTHER; note = uracil
cgtgcgattaagcatgcatcggacac

SEQ ID NO: 75 - DNA, 27 bp, synthetic construct
  misc_feature 1..27: note = PR-27971 (Erg5_UP_uORF_R_U1)
  source 1..27: mol_type = other DNA
  modified_base 9: mod_base = OTHER; note = uracil
atcgcacgtgatcgtgtgagtcagagg

SEQ ID NO: 76 - DNA, 30 bp, synthetic construct
  misc_feature 1..30: note = PR-27976 (ERG5_tPr50_F_U2)
  source 1..30: mol_type = other DNA
  modified_base 9: mod_base = OTHER; note = uracil
agtgcaggtcacaacttctctctctcacac

SEQ ID NO: 77 - DNA, 26 bp, synthetic construct
  misc_feature 1..26: note = PR-27972 (Hyg/NatMX_F_U1)
  source 1..26: mol_type = other DNA
  modified_base 8: mod_base = OTHER; note = uracil
cgtgcgattcagctgaagcttcgtac

SEQ ID NO: 78 - DNA, 26 bp, synthetic construct
  misc_feature 1..26: note = PR-27973 (Hyg/NatMX_R_U2)
  source 1..26: mol_type = other DNA
  modified_base 9: mod_base = OTHER; note = uracil
acctgcactgcataggccactagtgg

SEQ ID NO: 79 - DNA, 31 bp, synthetic construct
  misc_feature 1..31: note = PR-28358 (Erg4Rep_P1R_U1)
  source 1..31: mol_type = other DNA
  modified_base 9: mod_base = OTHER; note = uracil
atcgcacgtgataagcttagtgagcgaatgg

SEQ ID NO: 80 - DNA, 29 bp, synthetic construct
  misc_feature 1..29: note = PR-28359 (Erg5Rep_P1R_U1)
  source 1..29: mol_type = other DNA
  modified_base 9: mod_base = OTHER; note = uracil
atcgcacgttggtccgtatcgtgaaatgg

SEQ ID NO: 81 - DNA, 29 bp, synthetic construct
  misc_feature 1..29: note = PR-28360 (Erg6Rep_P1R_U1)
  source 1..29: mol_type = other DNA
  modified_base 9: mod_base = OTHER; note = uracil
atcgcacgtcaagggcgattcagatcagc

SEQ ID NO: 82 - DNA, 20 bp, synthetic construct
  misc_feature 1..20: note = PR-27631 (ERG4_KOchk_Fw)
  source 1..20: mol_type = other DNA
cctgatattggtgatcctcc

SEQ ID NO: 83 - DNA, 20 bp, synthetic construct
  misc_feature 1..20: note = PR-27632 (ERG4_KOchk_Rv1)
  source 1..20: mol_type = other DNA
agagccttgtttccgaggtg

SEQ ID NO: 84 - DNA, 20 bp, synthetic construct
  misc_feature 1..20: note = PR-27633 (ERG4_KOchk_Rv2)
  source 1..20: mol_type = other DNA
atacaatcccataggctggc

SEQ ID NO: 85 - DNA, 30 bp, synthetic construct
  misc_feature 1..30: note = PR-11138 (HphMX_KOcasette_chkFw)
  source 1..30: mol_type = other DNA
  modified_base 10: mod_base = OTHER; note = uracil
agcaatgggtaaaaagcctgaactcaccgc

SEQ ID NO: 86 - DNA, 30 bp, synthetic construct
  misc_feature 1..30: note = PR-28001 (Derg4_chkRv)
  source 1..30: mol_type = other DNA
  modified_base 8: mod_base = OTHER; note = uracil
cgtgcgatgcttgccctggactacatcttg

SEQ ID NO: 87 - DNA, 21 bp, synthetic construct
  misc_feature 1..21: note = PR-27634 (ERG5_KOchk_Fw)
  source 1..21: mol_type = other DNA
acttctctctctcacaccacc

SEQ ID NO: 88 - DNA, 21 bp, synthetic construct
  misc_feature 1..21: note = PR-27635 (ERG5_KOchk_Rv1)
  source 1..21: mol_type = other DNA
ctgagggctctgttggtgaag

SEQ ID NO: 89 - DNA, 21 bp, synthetic construct
  misc_feature 1..21: note = PR-27636 (ERG5_KOchk_Rv2)
  source 1..21: mol_type = other DNA
accagtgtggttgtaaggatg

SEQ ID NO: 90 - DNA, 19 bp, synthetic construct
  misc_feature 1..19: note = PR-22830 (HphMX_KOcasette_chkRv)
  source 1..19: mol_type = other DNA
tcatactcaccgaaacgtg

SEQ ID NO: 91 - DNA, 26 bp, synthetic construct
  misc_feature 1..26: note = PR-27970 (Derg5_chkRv)
  source 1..26: mol_type = other DNA
  modified_base 8: mod_base = OTHER; note = uracil
cgtgcgattaagcatgcatcggacac

SEQ ID NO: 92 - DNA, 20 bp, synthetic construct
  misc_feature 1..20: note = PR-27637 (ERG6_KOchk_Fw)
  source 1..20: mol_type = other DNA
ctcgcatacttcccgtttgg

SEQ ID NO: 93 - DNA, 19 bp, synthetic construct
  misc_feature 1..19: note = PR-27638 (ERG6_KOchk_Rv1)
  source 1..19: mol_type = other DNA
tgagaccgacaatgttggc

SEQ ID NO: 94 - DNA, 21 bp, synthetic construct
  misc_feature 1..21: note = PR-27639 (ERG6_KOchk_Rv2)
  source 1..21: mol_type = other DNA
ccaccgatccttctcagctac

SEQ ID NO: 95 - DNA, 20 bp, synthetic construct
  misc_feature 1..20: note = PR-26681 (NatMX_KOcasette_chkFw)
  source 1..20: mol_type = other DNA
atccgctctaaccgaaaagg

SEQ ID NO: 96 - DNA, 26 bp, synthetic construct
  misc_feature 1..26: note = PR-28007 (Derg6_chkFw)
  source 1..26: mol_type = other DNA
  modified_base 8: mod_base = OTHER; note = uracil
cgtgcgatgaaggagatactggtgcc

SEQ ID NO: 97 - DNA, 31 bp, synthetic construct
  misc_feature 1..31: note = PR-11110 (E.coli backboneUSER _fw)
  source 1..31: mol_type = other DNA
atcgcgtgcattcgcggccgcatttaaatcc

SEQ ID NO: 98 - DNA, 37 bp, synthetic construct
  misc_feature 1..37: note = PR-11111 (E.coli backboneUSER _rev)
  source 1..37: mol_type = other DNA
atcgcacgcattcgcggccgcaaatttaaataaaatg

SEQ ID NO: 99 - DNA, 37 bp, synthetic construct
  misc_feature 1..37: note = PR-28361 (TtSTC_F_U3)
  source 1..37: mol_type = other DNA
  modified_base 9: mod_base = OTHER; note = uracil
atctgtcatgccacaatgaagaagatcctcatcggtc

SEQ ID NO: 100 - DNA, 30 bp, synthetic construct
  misc_feature 1..30: note = PR-28362 (TtSTC_Rv)
  source 1..30: mol_type = other DNA
  modified_base 8: mod_base = OTHER; note = uracil
cacgcgatttagatgttctgcttctggacg

SEQ ID NO: 101 - DNA, 17 bp, synthetic construct
  misc_feature 1..17: note = PR-14617 (vector verification E. coli cPCR)
  source 1..17: mol_type = other DNA
tatccctgtgttgaatc

SEQ ID NO: 102 - DNA, 16 bp, synthetic construct
  misc_feature 1..16: note = PR-14619 (vector verification E. coli cPCR)
  source 1..16: mol_type = other DNA
tatcgacccagttagc

SEQ ID NO: 103 - DNA, 21 bp, synthetic construct
  misc_feature 1..21: note = PR-8859 (integration verification Yl cPCR)
  source 1..21: mol_type = other DNA
aagtgtggatggggaagtgag

SEQ ID NO: 104 - DNA, 28 bp, synthetic construct
  misc_feature 1..28: note = PR-14576 (IntE_3 verification Yl cPCR)
  source 1..28: mol_type = other DNA
  modified_base 8: mod_base = OTHER; note = uracil
cacgcgattgaaggaaatgcctaaaacc

SEQ ID NO: 105 - DNA, 20 bp, synthetic construct
  misc_feature 1..20: note = PR-14835 (IntE_3 verification Yl cPCR)
  source 1..20: mol_type = other DNA
cacgcacgccattctataag

SEQ ID NO: 106 - DNA, 27 bp, synthetic construct
  misc_feature 1..27: note = PR-14592 (IntE_4 verification Yl cPCR)
  source 1..27: mol_type = other DNA
  modified_base 7: mod_base = OTHER; note = uracil
acgcgatttaacactggaccgtactgc

SEQ ID NO: 107 - DNA, 20 bp, synthetic construct
  misc_feature 1..20: note = PR-20880 (IntE_4 verification Yl cPCR)
  source 1..20: mol_type = other DNA
attgctaagcgaccatagac

SEQ ID NO: 108 - DNA, 31 bp, synthetic construct; identical to SEQ ID NO: 97
  misc_feature 1..31: note = PR-11110 (E.coli backboneUSER _fw)
  source 1..31: mol_type = other DNA

SEQ ID NO: 109 - DNA, 37 bp, synthetic construct; identical to SEQ ID NO: 98
  misc_feature 1..37: note = PR-11111 (E.coli backboneUSER _rev)
  source 1..37: mol_type = other DNA

SEQ ID NO: 110 - DNA, 44 bp, synthetic construct
  misc_feature 1..44: note = PR-27565 (A_St_Fw)
  source 1..44: mol_type = other DNA
  modified_base 15: mod_base = OTHER; note = uracil
actttttgcagtactaaccgcaggccgagtctcagctggtgcac

SEQ ID NO: 111 - DNA, 29 bp, synthetic construct
  misc_feature 1..29: note = PR-27566 (A_St_Rv)
  source 1..29: mol_type = other DNA
  modified_base 8: mod_base = OTHER; note = uracil
cacgcgatttagtagatgccggggatcac

SEQ ID NO: 112 - DNA, 43 bp, synthetic construct
  misc_feature 1..43: note = PR-27567 (A_Dr_Fw)
  source 1..43: mol_type = other DNA
  modified_base 15: mod_base = OTHER; note = uracil
actttttgcagtactaaccgcagatggcctctgaccgagttcg

SEQ ID NO: 113 - DNA, 29 bp, synthetic construct
  misc_feature 1..29: note = PR-27568 (A_Dr_Rv)
  source 1..29: mol_type = other DNA
  modified_base 8: mod_base = OTHER; note = uracil
cacgcgatttagaagatgttgggcagcag

SEQ ID NO: 114 - DNA, 43 bp, synthetic construct
  misc_feature 1..43: note = PR-27569 (A_Ld_Fw)
  source 1..43: mol_type = other DNA
  modified_base 15: mod_base = OTHER; note = uracil
actttttgcagtactaaccgcagtacttcaagatccgaaacac

SEQ ID NO: 115 - DNA, 30 bp, synthetic construct
  misc_feature 1..30: note = PR-27570 (A_Ld_Rv)
  source 1..30: mol_type = other DNA
  modified_base 8: mod_base = OTHER; note = uracil
cacgcgatttagatcacgaagggcacgatc

SEQ ID NO: 116 - DNA, 42 bp, synthetic construct; identical to SEQ ID NO: 47
  misc_feature 1..42: note = PR-27571 (A_Es_Fw)
  source 1..42: mol_type = other DNA
  modified_base 15: mod_base = OTHER; note = uracil

SEQ ID NO: 117 - DNA, 30 bp, synthetic construct; identical to SEQ ID NO: 48
  misc_feature 1..30: note = PR-27572 (A_Es_Rv)
  source 1..30: mol_type = other DNA
  modified_base 8: mod_base = OTHER; note = uracil

SEQ ID NO: 118 - DNA, 44 bp, synthetic construct
  misc_feature 1..44: note = PR-27573 (A_Cp_Fw)
  source 1..44: mol_type = other DNA
  modified_base 15: mod_base = OTHER; note = uracil
actttttgcagtactaaccgcagctgatcgagatgctgtgcatc

SEQ ID NO: 119 - DNA, 30 bp, synthetic construct
  misc_feature 1..30: note = PR-27574 (A_Cp_Rv)
  source 1..30: mol_type = other DNA
  modified_base 8: mod_base = OTHER; note = uracil
cacgcgatttagatcacgaaggggatgatc

SEQ ID NO: 120 - DNA, 42 bp, synthetic construct
  misc_feature 1..42: note = PR-27575 (A_Cs_Fw)
  source 1..42: mol_type = other DNA
  modified_base 15: mod_base = OTHER; note = uracil
actttttgcagtactaaccgcaggtcacaacccgagccgctg

SEQ ID NO: 121 - DNA, 30 bp, synthetic construct
  misc_feature 1..30: note = PR-27576 (A_Cs_Rv)
  source 1..30: mol_type = other DNA
  modified_base 8: mod_base = OTHER; note = uracil
cacgcgatttagaaaatgtaagggatcatc

SEQ ID NO: 122 - DNA, 43 bp, synthetic construct
  misc_feature 1..43: note = PR-27577 (A_Mv_Fw)
  source 1..43: mol_type = other DNA
  modified_base 15: mod_base = OTHER; note = uracil
actttttgcagtactaaccgcaggccgtgcagcagcgaaagac

SEQ ID NO: 123 - DNA, 30 bp, synthetic construct
  misc_feature 1..30: note = PR-27578 (A_Mv_Rv)
  source 1..30: mol_type = other DNA
  modified_base 8: mod_base = OTHER; note = uracil
cacgcgatttagtacacgtaggggatcagc

SEQ ID NO: 124 - DNA, 42 bp, synthetic construct
  misc_feature 1..42: note = PR-27579 (A_Gs_Fw)
  source 1..42: mol_type = other DNA
  modified_base 15: mod_base = OTHER; note = uracil
actttttgcagtactaaccgcagggcgctaccgtgcactctc

SEQ ID NO: 125 - DNA, 27 bp, synthetic construct
  misc_feature 1..27: note = PR-27580 (A_Gs_Rv)
  source 1..27: mol_type = other DNA
  modified_base 8: mod_base = OTHER; note = uracil
cacgcgatttagtagatgccggggatg

SEQ ID NO: 126 - DNA, 42 bp, synthetic construct
  misc_feature 1..42: note = PR-27581 (A_Ts_Fw)
  source 1..42: mol_type = other DNA
  modified_base 15: mod_base = OTHER; note = uracil
actttttgcagtactaaccgcagaagcgagcctccaagaccc

SEQ ID NO: 127 - DNA, 30 bp, synthetic construct
  misc_feature 1..30: note = PR-27582 (A_Ts_Rv)
  source 1..30: mol_type = other DNA
  modified_base 8: mod_base = OTHER; note = uracil
cacgcgatttagaagatgtagggcacgatc

SEQ ID NO: 128 - DNA, 42 bp, synthetic construct
  misc_feature 1..42: note = PR-27583 (A_Wc_Fw)
  source 1..42: mol_type = other DNA
  modified_base 15: mod_base = OTHER; note = uracil
actttttgcagtactaaccgcaggccgccaccaccaccaacg

SEQ ID NO: 129 - DNA, 29 bp, synthetic construct
  misc_feature 1..29: note = PR-27584 (A_Wc_Rv)
  source 1..29: mol_type = other DNA
  modified_base 8: mod_base = OTHER; note = uracil
cacgcgatttagtagatgccggggatcag

SEQ ID NO: 130 - DNA, 19 bp, synthetic construct
  misc_feature 1..19: note = PR-27891 (Erg4Rep_P1F)
  source 1..19: mol_type = other DNA
ctctcaacaccttcaccgc

SEQ ID NO: 131 - DNA, 31 bp, synthetic construct; identical to SEQ ID NO: 79
  misc_feature 1..31: note = PR-28358 (Erg4Rep_P1R_U1)
  source 1..31: mol_type = other DNA
  modified_base 9: mod_base = OTHER; note = uracil

SEQ ID NO: 132 - DNA, 27 bp, synthetic construct
  misc_feature 1..27: note = PR-27893 (Erg4Rep_P2F)
  source 1..27: mol_type = other DNA
  modified_base 8: mod_base = OTHER; note = uracil
gtgcaggttgtgtggttgcgaaggaag

SEQ ID NO: 133 - DNA, 20 bp, synthetic construct
  misc_feature 1..20: note = PR-27894 (Erg4Rep_P2R)
  source 1..20: mol_type = other DNA
gcactcaaaataccccgttc

SEQ ID NO: 134 - DNA, 19 bp, synthetic construct
  misc_feature 1..19: note = PR-27895 (Erg5Rep_P1F)
  source 1..19: mol_type = other DNA
ctcggtttgttgcagcagg

SEQ ID NO: 135 - DNA, 29 bp, synthetic construct
  misc_feature 1..29: note = PR-28359 (Erg5Rep_P1R_U1)
  source 1..29: mol_type = other DNA
  modified_base 9: mod_base = OTHER; note = uracil
atcgcacgttggtccgtatcgtgaaatgg

SEQ ID NO: 136 - DNA, 26 bp, synthetic construct
  misc_feature 1..26: note = PR-27897 (Erg5Rep_P2F)
  source 1..26: mol_type = other DNA
  modified_base 8: mod_base = OTHER; note = uracil
gtgcaggtgggcggagttgtgttgtg

SEQ ID NO: 137 - DNA, 22 bp, synthetic construct
  misc_feature 1..22: note = PR-27898 (Erg5Rep_P2R)
  source 1..22: mol_type = other DNA
ggtcggctatccaatacatctc

SEQ ID NO: 138 - DNA, 20 bp, synthetic construct
  misc_feature 1..20: note = PR-27899 (Erg6Rep_P1F)
  source 1..20: mol_type = other DNA
gctacaagccggaggggaac

SEQ ID NO: 139 - DNA, 29 bp, synthetic construct
  misc_feature 1..29: note = PR-28360 (Erg6Rep_P1R_U1)
  source 1..29: mol_type = other DNA
  modified_base 9: mod_base = OTHER; note = uracil
atcgcacgtcaagggcgattcagatcagc

SEQ ID NO: 140 - DNA, 28 bp, synthetic construct
  misc_feature 1..28: note = PR-27901 (Erg6Rep_P2F)
  source 1..28: mol_type = other DNA
  modified_base 8: mod_base = OTHER; note = uracil
gtgcaggtactgagtaacttatagaggg

SEQ ID NO: 141 - DNA, 20 bp, synthetic construct
  misc_feature 1..20: note = PR-27902 (Erg6Rep_P2R)
  source 1..20: mol_type = other DNA
ctgtaccgtttggaggactc

SEQ ID NO: 142 - DNA, 26 bp, synthetic construct
  misc_feature 1..26: note = PR-27972 (HphMX_F_U1)
  source 1..26: mol_type = other DNA
  modified_base 8: mod_base = OTHER; note = uracil
cgtgcgattcagctgaagcttcgtac

SEQ ID NO: 143 - DNA, 26 bp, synthetic construct
  misc_feature 1..26: note = PR-27973 (HphMX_R_U2)
  source 1..26: mol_type = other DNA
  modified_base 9: mod_base = OTHER; note = uracil
acctgcactgcataggccactagtgg

SEQ ID NO: 144 - DNA, 37 bp, synthetic construct; identical to SEQ ID NO: 99
  misc_feature 1..37: note = PR-28361 (TtSTC_F_U3)
  source 1..37: mol_type = other DNA
  modified_base 9: mod_base = OTHER; note = uracil

SEQ ID NO: 145 - DNA, 30 bp, synthetic construct; identical to SEQ ID NO: 100
  misc_feature 1..30: note = PR-28362 (TtSTC_Rv)
  source 1..30: mol_type = other DNA
  modified_base 8: mod_base = OTHER; note = uracil

SEQ ID NO: 146 - DNA, 19 bp, synthetic construct
  misc_feature 1..19: note = PR-22830 (delta erg5_chk)
  source 1..19: mol_type = other DNA
tcatactcaccgaaacgtg

SEQ ID NO: 147 - DNA, 18 bp, synthetic construct
  misc_feature 1..18: note = PR-27961 (delta erg5_chk)
  source 1..18: mol_type = other DNA
gttccaatgcctggcaag

SEQ ID NO: 148 - DNA, 21 bp, synthetic construct
  misc_feature 1..21: note = PR-27634 (delta erg5_chk)
  source 1..21: mol_type = other DNA
acttctctctctcacaccacc

SEQ ID NO: 149 - DNA, 21 bp, synthetic construct
  misc_feature 1..21: note = PR-27635 (delta erg5_chk)
  source 1..21: mol_type = other DNA
ctgagggctctgttggtgaag

SEQ ID NO: 150 - DNA, 21 bp, synthetic construct
  misc_feature 1..21: note = PR-27636 (delta erg5_chk)
  source 1..21: mol_type = other DNA
accagtgtggttgtaaggatg

SEQ ID NO: 151 - DNA, 30 bp, synthetic construct; identical to SEQ ID NO: 85
  misc_feature 1..30: note = PR-11138 (delta erg4_chk)
  source 1..30: mol_type = other DNA
  modified_base 10: mod_base = OTHER; note = uracil

SEQ ID NO: 152 - DNA, 20 bp, synthetic construct
  misc_feature 1..20: note = PR-27631 (delta erg4_chk)
  source 1..20: mol_type = other DNA
cctgatattggtgatcctcc

SEQ ID NO: 153 - DNA, 20 bp, synthetic construct
  misc_feature 1..20: note = PR-27632 (delta erg4_chk)
  source 1..20: mol_type = other DNA
agagccttgtttccgaggtg

SEQ ID NO: 154 - DNA, 20 bp, synthetic construct
  misc_feature 1..20: note = PR-27633 (delta erg4_chk)
  source 1..20: mol_type = other DNA
atacaatcccataggctggc

SEQ ID NO: 155 - DNA, 30 bp, synthetic construct; identical to SEQ ID NO: 86
  misc_feature 1..30: note = PR-28001 (delta erg4_chk)
  source 1..30: mol_type = other DNA
  modified_base 8: mod_base = OTHER; note = uracil

SEQ ID NO: 156 - DNA, 19 bp, synthetic construct
  misc_feature 1..19: note = PR-22830 (delta erg6_chk)
  source 1..19: mol_type = other DNA
tcatactcaccgaaacgtg

SEQ ID NO: 157 - DNA, 26 bp, synthetic construct
  misc_feature 1..26: note = PR-28007 (delta erg6_chk)
  source 1..26: mol_type = other DNA
  modified_base 8: mod_base = OTHER; note = uracil
cgtgcgatgaaggagatactggtgcc

SEQ ID NO: 158 - DNA, 20 bp, synthetic construct
  misc_feature 1..20: note = PR-27637 (delta erg6_chk)
  source 1..20: mol_type = other DNA
ctcgcatacttcccgtttgg

SEQ ID NO: 159 - DNA, 21 bp, synthetic construct
  misc_feature 1..21: note = PR-27639 (delta erg6_chk)
  source 1..21: mol_type = other DNA
ccaccgatccttctcagctac

SEQ ID NO: 160 - DNA, 26 bp, synthetic construct
  misc_feature 1..26: note = PR-27969 (Erg5ORF_R_U3)
  source 1..26: mol_type = other DNA
  modified_base 8: mod_base = OTHER; note = uracil
cacgcgattcggtcggcaacaatctg

SEQ ID NO: 161 - DNA, 26 bp, synthetic construct
  misc_feature 1..26: note = PR-27970 (Erg5_UP_uORF_F_U3)
  source 1..26: mol_type = other DNA
  modified_base 8: mod_base = OTHER; note = uracil
cgtgcgattaagcatgcatcggacac

SEQ ID NO: 162 - DNA, 27 bp, synthetic construct
  misc_feature 1..27: note = PR-27971 (Erg5_UP_uORF_R_U1)
  source 1..27: mol_type = other DNA
  modified_base 9: mod_base = OTHER; note = uracil
atcgcacgtgatcgtgtgagtcagagg

SEQ ID NO: 163 - DNA, 30 bp, synthetic construct; identical to SEQ ID NO: 76
  misc_feature 1..30: note = PR-27976 (ERG5_tPr50_F_U2)
  source 1..30: mol_type = other DNA
  modified_base 9: mod_base = OTHER; note = uracil

SEQ ID NO: 164 - DNA, 18 bp, synthetic construct
  misc_feature 1..18: note = PR-26303 (YlPAH1_repair-up_fw)
  source 1..18: mol_type = other DNA
acgtactgcgcccatatt

SEQ ID NO: 165 - DNA, 27 bp, synthetic construct
  misc_feature 1..27: note = PR-26304 (YlPAH1_repair-up_rv)
  source 1..27: mol_type = other DNA
  modified_base 8: mod_base = OTHER; note = uracil
aggccacttgtggttgtgtggtgatga

SEQ ID NO: 166 - DNA, 25 bp, synthetic construct
  misc_feature 1..25: note = PR-26305 (YlPAH1_repair-dw_fw)
  source 1..25: mol_type = other DNA
  modified_base 8: mod_base = OTHER; note = uracil
agtggcctattccagcccgtttcgt

SEQ ID NO: 167 - DNA, 18 bp, synthetic construct
  misc_feature 1..18: note = PR-26306 (YlPAH1_repair-dw_rv)
  source 1..18: mol_type = other DNA
cttggctttctagcggga

SEQ ID NO: 168 - DNA, 17 bp, synthetic construct
  misc_feature 1..17: note = PR-26307 (YlPAH1_check_fw)
  source 1..17: mol_type = other DNA
cgaacccaaatccggac

SEQ ID NO: 169 - DNA, 17 bp, synthetic construct
  misc_feature 1..17: note = PR-26308 (YlPAH1_check_rv)
  source 1..17: mol_type = other DNA
acctgctcctccaccta

SEQ ID NO: 170 - DNA, 31 bp, synthetic construct
  source 1..31: mol_type = other DNA
  misc_feature 1..31: note = PR-29832 (PAH1_gRNA_Sense)
ggaaggttagaagaagggaggttttagagct

SEQ ID NO: 171 - DNA, 30 bp, synthetic construct
  source 1..30: mol_type = other DNA
  misc_feature 1..30: note = PR-29833 (PAH1_gRNA_AntiSense)
ctcccttcttctaaccttcctaaccaacct

SEQ ID NO: 172 - DNA, 17 bp, synthetic construct
  misc_feature 1..17: note = PR-14617 (vector verification E. coli cPCR)
  source 1..17: mol_type = other DNA
tatccctgtgttgaatc

SEQ ID NO: 173 - DNA, 16 bp, synthetic construct
  misc_feature 1..16: note = PR-14619 (vector verification E. coli cPCR)
  source 1..16: mol_type = other DNA
tatcgacccagttagc

SEQ ID NO: 174 - DNA, 21 bp, synthetic construct
  misc_feature 1..21: note = PR-8859 (integration verification Yl cPCR)
  source 1..21: mol_type = other DNA
aagtgtggatggggaagtgag

SEQ ID NO: 175 - DNA, 20 bp, synthetic construct
  misc_feature 1..20: note = PR-14442 (IntE_1 verification Yl cPCR)
  source 1..20: mol_type = other DNA
agttgtgaccaagacaaatg

SEQ ID NO: 176 - DNA, 28 bp, synthetic construct
  misc_feature 1..28: note = PR-14398 (IntE_1 verification Yl cPCR)
  source 1..28: mol_type = other DNA
  modified_base 8: mod_base = OTHER; note = uracil
cacgcgatgttagaagcaattggagaag

SEQ ID NO: 177 - DNA, 28 bp, synthetic construct
  misc_feature 1..28: note = PR-14576 (IntE_3 verification Yl cPCR)
  source 1..28: mol_type = other DNA
  modified_base 8: mod_base = OTHER; note = uracil
cacgcgattgaaggaaatgcctaaaacc

SEQ ID NO: 178 - DNA, 20 bp, synthetic construct
  misc_feature 1..20: note = PR-14835 (IntE_3 verification Yl cPCR)
  source 1..20: mol_type = other DNA
cacgcacgccattctataag

SEQ ID NO: 179 - DNA, 27 bp, synthetic construct
  misc_feature 1..27: note = PR-14592 (IntE_4 verification Yl cPCR)
  source 1..27: mol_type = other DNA
  modified_base 7: mod_base = OTHER; note = uracil
acgcgatttaacactggaccgtactgc

SEQ ID NO: 180 - DNA, 20 bp, synthetic construct
  misc_feature 1..20: note = PR-20880 (IntE_4 verification Yl cPCR)
  source 1..20: mol_type = other DNA
attgctaagcgaccatagac

SEQ ID NO: 181 - DNA, 20 bp, synthetic construct
  misc_feature 1..20: note = PR-14837 (IntF_3 verification Yl cPCR)
  source 1..20: mol_type = other DNA
acatgctcgcgcctcgatag

SEQ ID NO: 182 - DNA, 28 bp, synthetic construct
  misc_feature 1..28: note = PR-14584 (IntF_3 verificiationYl cPCR)
  source 1..28: mol_type = other DNA
  modified_base 8: mod_base = OTHER; note = uracil
cacgcgattttggtcgtcgcccaacaag

SEQ ID NO: 183 - DNA, 24 bp, synthetic construct
  misc_feature 1..24: note = PR-26834 (IntE_5 verification Yl cPCR)
  source 1..24: mol_type = other DNA
gggacaagtgcaatcgttcacttg

SEQ ID NO: 184 - DNA, 22 bp, synthetic construct
  misc_feature 1..22: note = PR-26835 (IntE_5 verification Yl cPCR)
  source 1..22: mol_type = other DNA
cgttgctgttgccacaatggac

SEQ ID NO: 185 - DNA, 42 bp, synthetic construct; identical to SEQ ID NO: 126
  misc_feature 1..42: note = PR-27581 (A_Ts_Fw)
  source 1..42: mol_type = other DNA
  modified_base 15: mod_base = OTHER; note = uracil

SEQ ID NO: 186 - DNA, 30 bp, synthetic construct; identical to SEQ ID NO: 127
  misc_feature 1..30: note = PR-27582 (A_Ts_Rv)
  source 1..30: mol_type = other DNA
  modified_base 8: mod_base = OTHER; note = uracil

SEQ ID NO: 187 - DNA, 19 bp, synthetic construct
  misc_feature 1..19: note = PR-27891 (Erg4Rep_P1F)
  source 1..19: mol_type = other DNA
ctctcaacaccttcaccgc

SEQ ID NO: 188 - DNA, 31 bp, synthetic construct; identical to SEQ ID NO: 79
  misc_feature 1..31: note = PR-28358 (Erg4Rep_P1R_U1)
  source 1..31: mol_type = other DNA
  modified_base 9: mod_base = OTHER; note = uracil

SEQ ID NO: 189 - DNA, 27 bp, synthetic construct
  misc_feature 1..27: note = PR-27893 (Erg4Rep_P2F)
  source 1..27: mol_type = other DNA
  modified_base 8: mod_base = OTHER; note = uracil
gtgcaggttgtgtggttgcgaaggaag

SEQ ID NO: 190 - DNA, 20 bp, synthetic construct
  misc_feature 1..20: note = PR-27894 (Erg4Rep_P2R)
  source 1..20: mol_type = other DNA
gcactcaaaataccccgttc

SEQ ID NO: 191 - DNA, 19 bp, synthetic construct
  misc_feature 1..19: note = PR-27895 (Erg5Rep_P1F)
  source 1..19: mol_type = other DNA
ctcggtttgttgcagcagg

SEQ ID NO: 192 - DNA, 29 bp, synthetic construct
  misc_feature 1..29: note = PR-28359 (Erg5Rep_P1R_U1)
  source 1..29: mol_type = other DNA
  modified_base 9: mod_base = OTHER; note = uracil
atcgcacgttggtccgtatcgtgaaatgg

SEQ ID NO: 193 - DNA, 26 bp, synthetic construct
  misc_feature 1..26: note = PR-27897 (Erg5Rep_P2F)
  source 1..26: mol_type = other DNA
  modified_base 8: mod_base = OTHER; note = uracil
gtgcaggtgggcggagttgtgttgtg

SEQ ID NO: 194 - DNA, 22 bp, synthetic construct
  misc_feature 1..22: note = PR-27898 (Erg5Rep_P2R)
  source 1..22: mol_type = other DNA
ggtcggctatccaatacatctc

SEQ ID NO: 195 - DNA, 26 bp, synthetic construct
  misc_feature 1..26: note = PR-27972 (HphMX_F_U1)
  source 1..26: mol_type = other DNA
  modified_base 8: mod_base = OTHER; note = uracil
cgtgcgattcagctgaagcttcgtac

SEQ ID NO: 196 - DNA, 26 bp, synthetic construct
  misc_feature 1..26: note = PR-27973 (HphMX_R_U2)
  source 1..26: mol_type = other DNA
  modified_base 9: mod_base = OTHER; note = uracil
acctgcactgcataggccactagtgg

SEQ ID NO: 197 - DNA, 29 bp, synthetic construct
  misc_feature 1..29: note = PR-15604 (PrExp_for fusion->_U2_fw)
  source 1..29: mol_type = other DNA
  modified_base 10: mod_base = OTHER; note = uracil
agctactgataaggagtttggcgcccgtt

SEQ ID NO: 198 - DNA, 27 bp, synthetic construct
  misc_feature 1..27: note = PR-22297 (Ttef_U_EpiVecYL_rv)
  source 1..27: mol_type = other DNA
  modified_base 9: mod_base = OTHER; note = uracil
aagcctgctgaattcggacacgggcat

SEQ ID NO: 199 - DNA, 30 bp, synthetic construct
  misc_feature 1..30: note = PR-21844 (gRNA_opened_rv)
  source 1..30: mol_type = other DNA
  modified_base 10: mod_base = OTHER; note = uracil
atcagtagctctactgtatattcaagcaag

SEQ ID NO: 200 - DNA, 25 bp, synthetic construct
  misc_feature 1..25: note = PR-10593 (Fragment2EpiVecYL _fw)
  source 1..25: mol_type = other DNA
  modified_base 9: mod_base = OTHER; note = uracil
agcaggcttggaggcgacgtggcag

SEQ ID NO: 201 - DNA, 37 bp, synthetic construct; identical to SEQ ID NO: 99
  misc_feature 1..37: note = PR-28361 (TtSTC_F_U3)
  source 1..37: mol_type = other DNA
  modified_base 9: mod_base = OTHER; note = uracil

SEQ ID NO: 202 - DNA, 30 bp, synthetic construct; identical to SEQ ID NO: 100
  misc_feature 1..30: note = PR-28362 (TtSTC_Rv)
  source 1..30: mol_type = other DNA
  modified_base 8: mod_base = OTHER; note = uracil

SEQ ID NO: 203 - DNA, 19 bp, synthetic construct
  misc_feature 1..19: note = PR-22830 (delta erg5_chk)
  source 1..19: mol_type = other DNA
tcatactcaccgaaacgtg

SEQ ID NO: 204 - DNA, 18 bp, synthetic construct
  misc_feature 1..18: note = PR-27961 (delta erg5_chk)
  source 1..18: mol_type = other DNA
gttccaatgcctggcaag

SEQ ID NO: 205 - DNA, 21 bp, synthetic construct
  misc_feature 1..21: note = PR-27634 (delta erg5_chk)
  source 1..21: mol_type = other DNA
acttctctctctcacaccacc

SEQ ID NO: 206 - DNA, 21 bp, synthetic construct
  misc_feature 1..21: note = PR-27635 (delta erg5_chk)
  source 1..21: mol_type = other DNA
ctgagggctctgttggtgaag

SEQ ID NO: 207 - DNA, 21 bp, synthetic construct
  misc_feature 1..21: note = PR-27636 (delta erg5_chk)
  source 1..21: mol_type = other DNA
accagtgtggttgtaaggatg

SEQ ID NO: 208 - DNA, 30 bp, synthetic construct; identical to SEQ ID NO: 85
  misc_feature 1..30: note = PR-11138 (delta erg4_chk)
  source 1..30: mol_type = other DNA
  modified_base 10: mod_base = OTHER; note = uracil

SEQ ID NO: 209 - DNA, 20 bp, synthetic construct
  misc_feature 1..20: note = PR-27631 (delta erg4_chk)
  source 1..20: mol_type = other DNA
cctgatattggtgatcctcc

SEQ ID NO: 210 - DNA, 20 bp, synthetic construct
  misc_feature 1..20: note = PR-27632 (delta erg4_chk)
  source 1..20: mol_type = other DNA
agagccttgtttccgaggtg

SEQ ID NO: 211 - DNA, 20 bp, synthetic construct
  misc_feature 1..20: note = PR-27633 (delta erg4_chk)
  source 1..20: mol_type = other DNA
atacaatcccataggctggc

SEQ ID NO: 212 - DNA, 30 bp, synthetic construct; identical to SEQ ID NO: 86
  misc_feature 1..30: note = PR-28001 (delta erg4_chk)
  source 1..30: mol_type = other DNA
  modified_base 8: mod_base = OTHER; note = uracil

SEQ ID NO: 213 - DNA, 30 bp, synthetic construct; identical to SEQ ID NO: 86
  misc_feature 1..30: note = PR-28001 (delta erg4_chk)
  source 1..30: mol_type = other DNA
  modified_base 8: mod_base = OTHER; note = uracil

SEQ ID NO: 214 - DNA, 34 bp, synthetic construct
  misc_feature 1..34: note = PR-27599 (B_Sl_Fw)
  source 1..34: mol_type = other DNA
  modified_base 9: mod_base = OTHER; note = uracil
atctgtcatgccacaatgtctgacgccaaggctc

SEQ ID NO: 215 - DNA, 25 bp, synthetic construct
  misc_feature 1..25: note = PR-27764 (B_Sl_Rv)
  source 1..25: mol_type = other DNA
  modified_base 8: mod_base = OTHER; note = uracil
cacgcgatttagttggcctcagggg

SEQ ID NO: 216 - DNA, 33 bp, synthetic construct
  misc_feature 1..33: note = PR-27621 (C_Cq_Fw)
  source 1..33: mol_type = other DNA
  modified_base 9: mod_base = OTHER; note = uracil
atctgtcatgccacaatggactctatggccctg

SEQ ID NO: 217 - DNA, 26 bp, synthetic construct
  misc_feature 1..26: note = PR-27786 (C_Cq_Rv)
  source 1..26: mol_type = other DNA
  modified_base 8: mod_base = OTHER; note = uracil
cacgcgatttaagagtcagacttggc

SEQ ID NO: 218 - DNA, 35 bp, synthetic construct
  misc_feature 1..35: note = PR-29139 (ERG4_ORF_F_U3)
  source 1..35: mol_type = other DNA
  modified_base 9: mod_base = OTHER; note = uracil
atctgtcatgccacaatgtctgctgttcgacaacg

SEQ ID NO: 219 - DNA, 34 bp, synthetic construct
  misc_feature 1..34: note = PR-29140 (ERG4_ORF_Rv)
  source 1..34: mol_type = other DNA
  modified_base 8: mod_base = OTHER; note = uracil
cacgcgatttagaaaacataagggatgaagatcc

SEQ ID NO: 220 - DNA, 33 bp, synthetic construct
  misc_feature 1..33: note = PR-28981 (Backbone_amp_Tlip_F)
  source 1..33: mol_type = other DNA
  modified_base 11: mod_base = OTHER; note = uracil
atcgcgtgcattccttctgttcggaatcaacct

SEQ ID NO: 221 - DNA, 31 bp, synthetic construct
  source 1..31: mol_type = other DNA
  misc_feature 1..31: note = PR-29404 (StSSR1_F_U3)
  modified_base 9: mod_base = OTHER; note = uracil
atctgtcatgccacaatgaccgacgtccagg

SEQ ID NO: 222 - DNA, 26 bp, synthetic construct
  source 1..26: mol_type = other DNA
  misc_feature 1..26: note = PR-29405 (StSSR1_Rv)
  modified_base 8: mod_base = OTHER; note = uracil
cacgcgatttagtcctcgggctcgtc

SEQ ID NO: 223 - DNA, 1551 bp, synthetic construct
  source 1..1551: mol_type = other DNA
  misc_feature 1..1551: note = Danio rerio delta-24 sterol reductase codon optimised for Yarrowia lipolytica
atggaccctctgctgtacctcggcggcctggccgtgctgttcctgatctggatcaaggtgaagggcctcgagtacgtgatcatccaccagagatggatcttcgtgtgcctgttcctgctgcctctgtctgtggtgttcgacgtgtactaccacctccgagcctggatcatcttcaagatgtgctctgctcccaagcagcacgaccagcgagtgcgagacatccagcgacaggtgcgagagtggcgaaaggacggcggcaagaagtacatgtgcaccggacgacccggctggctgaccgtgtctctgcgagtgggcaagtacaagaagacccacaagaacatcatgatcaacatgatggacatcctcgaggtggacaccaagcgaaaggtggtgcgagtcgagcccctggccaacatgggccaagtgaccgctctgctgaactctatcggctggaccctgcctgtgctgcccgagctggacgacctgaccgtcggcggactggtgatgggaaccggcatcgagtcctcttctcacatctacggcctgttccagcacatctgcgtggccttcgagctggtgctggccgacggctctctggtccgatgcaccgagaaggaaaactctgacctgttctacgctgtgccctggtcttgcggaaccctgggcttcctggtcgccgccgagatccgaatcatccccgctcagaagtgggtcaagctgcactacgagcccgtgcgaggcctggacgccatctgcaagaagttcgccgaggaatctgccaacaaggaaaaccagttcgtcgagggcctgcagtactctcgagatgaggccgtgatcatgaccggcgtgatgaccgaccacgctgagcccgacaagaccaactgcatcggctactactacaagccctggttcttccgacacgtcgagtcttttctgaagcagaaccgagtggccgtcgagtacattcccctgcgacactactaccatcgacacacccgatctattttctgggagctgcaggacatcatccccttcggcaacaaccctctgttccgatacgtgttcggctggatggtgcctcctaagatctccctgctgaagctgacccagggcgagactatccgaaagctgtacgagcagcaccacgtcgtccaggacatgctggtgcccatgaaggacatcaaggccgccattcagcgattccacgaggacatccacgtgtaccctctgtggctgtgcccctttctgctgcccaaccagcctggcatggtgcaccccaagggcgacgaggacgagctgtacgtggacatcggcgcctacggcgagcccaaggtcaagcacttcgaggccacctcttctacccgacagctcgagaagttcgtgcgagatgtgcacggcttccagatgctgtacgccgacgtctacatggaacgaaaggaattctgggagatgttcgacggcaccctgtaccacaagctgcgagaggaactgggctgcaaggacgctttccccgaggtgtttgacaagatctgcaagtctgcccgacactaa

SEQ ID NO: 224 - DNA, 1551 bp, synthetic construct
  source 1..1551: mol_type = other DNA
  misc_feature 1..1551: note = Mus musculus delta-24 sterol reductase codon optimised for Yarrowia lipolytica
atggaacccgccgtgtctctggccgtgtgcgccctgctgttcctgctgtgggtgcgagtgaagggcctcgagttcgtgctgatccaccagagatgggtgttcgtgtgcctgtttctgctgcccctgtctctgatcttcgacatctactactacgtgcgagcctgggtcgtgttcaagctgtcctctgctccccgactgcacgagcagcgagtgcgagacatccaaaagcaggtccgagagtggaaggaacagggctctaagaccttcatgtgcaccggacgacccggctggctgaccgtgtcgctgcgagtgggcaagtacaagaagacccacaagaacatcatgatcaacctgatggacatcctcgaggtggacaccaagaagcagatcgtccgagtcgagcccctggtgtctatgggccaagtgaccgctctgctgaactctatcggctggaccctgcctgtgctgcccgagctggacgacctgaccgtcggcggcctgatcatgggaaccggcatcgagtcctcttcgcacaagtacggcctgttccagcacatctgcaccgcctacgagctgatcctggccgacggctctttcgtccgatgcaccccttctgagaactctgacctgttctacgctgtgccctggtcttgcggaaccctgggcttcctggtggccgccgagatccgaatcatccccgccaagaagtacgtcaagctgcgattcgagcccgtgcgaggactggaagccatctgcgagaagttcacccgagagtctcagcgactcgagaaccacttcgtcgagggcctgctgtactctctggacgaggccgtgatcatgaccggcgtgatgaccgacgacgtcgagccctctaagctgaactccattggctcttactacaagccctggttcttcaagcacgtcgagaactacctcaagaccaaccgagagggactcgagtacattcccctgcgacactactaccaccgacacacccgatctattttctgggagctgcaggacatcatccccttcggcaacaaccccatcttccgatacctgttcggctggatggtgcctcctaagatctccctgctgaagctgacccagggcgagactctgcgaaagctgtacgagcagcaccacgtcgtccaggacatgctggtgcccatgaagtgcatgtctcaggccctgcacaccttccagaacgacatccacgtgtaccccatctggctgtgccccttcattctgccctctcagcccggcctggtgcaccccaagggcgacgaggctgagctgtacgtggacatcggcgcctacggcgagccccgagtgaagcacttcgaggcccgatcttgcatgcgacagctcgagaagtttgtgcgatctgtgcacggcttccagatgctgtacgccgactgctacatgaaccgagaagagttctgggagatgttcgacggatctctgtaccacaagctccgaaagcagctgggctgccaggacgctttccccgaggtgtacgacaagatctgcaaggccgctcgacactaa

SEQ ID NO: 225 - DNA, 1704 bp, synthetic construct
  source 1..1704: mol_type = other DNA
  misc_feature 1..1704: note = Solanum tuberosum delta-24(28) sterol reductase codon optimised for Yarrowia lipolytica
atgaccgacgtccaggcccctcctcgacccaagcgaaagaagaacatcatggacctcctcgtccagttccgatggatcgtcgtcatcttcgtcgtcctccccctctccttcctctactacttctccatctacgtcggtgacgtccgatccgagtgcaagtcctacaagcagcgacagaaggagcacgacgagaacgtcaagaaggtcgtcaagcgactcaaggaccgaaacgcctccaaggacggtctggtctgcaccgcccgaaagccctgggtcgctgtcggtatgcgaaacgtcgactacaagcgagcccgacacttcgaggtcgacctctcccccttccgaaacgtcctcaacatcgacaccgagcgaatgatcgccaaggtcgagcccctcgtcaacatgggtcagatctcccgagtcaccgtccccatgaacgtctccctcgccgtcgtcgctgagctcgacgacctcaccgtcggtggtctgatcaacggttacggtatcgagggttcctcccacatctacggtctgttctccgacaccgtcgtctcctacgaggtcgtcctcgccgacggtcaggtcgtccgagccaccaaggacaacgagtactccgacctcttctacgccatcccctggtcccagggtaccctcggtctgctcgtctccgccgagatcaagctcatccccatcaaggagtacatgaagctcacctacaagcccgtcgtcggtaacctcaaggagatcgcccaggcctacatcgactccttctcccccaaggacggtgaccaggacaaccgagagaaggtccccgacttcgtcgagactatggtctacacccccaccgaggctgtctgcatgaccggtcgatacgcctccaaggaggaggccaagaagaagggtaacgtcatcaacaacgtcggttggtggttcaagacctggttctaccagcacgcccagaccgctctcaagaagggtgagttcgtcgagtacatccccacccgagagtactaccaccgacacacccgatgcctctactgggagggtaagctcatcctccccttcggtgaccagtggtggttccgattcttcttcggttgggccatgccccctaaggtttccctcctcaaggccacccagggtgagtacatccgaaactactaccacgagaaccacgtcatccaggacatgctcgtccccctctacaaggtcggtgacgccctcgagtgggtcaaccgagagatggaggtctaccccctctggctctgcccccaccgactctaccgactccccctcaagaccatggtctaccccgagcctggtttcgagctccacaagcgacagggtgacaccaagtacgcccagatgtacaccgacgtcggtgtctactacgcccccggtcccatcctccgaggtgaggtcttcgacggtatcgaggccgtccgaaagctcgagtcctggctcatcgagaaccacggtttccagccccagtacgccgtctccgagctcaccgagaagaacttctggcgaatgttcgacggttccctctacgagaactgccgaaagaagtaccgagccatcggtaccttcatgtccgtctactacaagtccaagaagggtaagaagaccgagaaggaggtccaggacgccgagcaggagactgccgaggtcgagactcccgaggtcgacgagcccgaggactaa

SEQ ID NO: 226 - DNA, 1086 bp, synthetic construct
  source 1..1086: mol_type = other DNA
  misc_feature 1..1086: note = Arabidopsis thaliana C-28 sterol methyl-transferase codon optimised for Yarrowia lipolytica
atggactctctgaccctgttcttcaccggcgctctggtggccgtgggaatctactggttcctgtgcgtgctgggacccgccgagcgaaagggcaagcgagccgtggacctgtctggcggctctatctctgccgagaaggtgcaggacaactacaagcagtactggtcgttcttccgacgacctaaggaaatcgagactgctgagaaggtccccgacttcgtggacaccttctacaacctggtgaccgacatctacgagtggggctggggccagtctttccacttctctccctcgattcccggcaagtctcacaaggacgctacccgactgcacgaagagatggccgtcgacctgatccaggtgaagcccggccaaaagatcctggacgtcggctgcggcgtcggcggacccatgcgagccattgcctctcactctcgagccaacgtggtgggcatcaccatcaacgagtaccaggtgaaccgagccagactgcacaacaagaaggccggactggacgccctgtgcgaggtggtgtgcggcaacttcctgcagatgcccttcgacgacaactctttcgacggcgcctactctatcgaggccacctgtcacgctcccaagctggaagaggtgtacgccgagatctaccgagtgctgaagcctggctctatgtacgtgtcttacgaatgggtgaccaccgagaagttcaaggccgaggacgacgagcacgtcgaggtgatccagggcatcgagcgaggcgacgctctgcccggcctgcgagcctacgtggacattgccgagacagccaagaaggtgggcttcgagatcgtgaaggaaaaggacctggcctcgcctcctgctgagccctggtggacccgactgaagatgggccgactggcctactggcgaaaccacatcgtggtgcagatcctgtctgccgtgggcgtcgcccctaagggcaccgtggacgtgcacgagatgctgttcaagaccgccgactacctgactcgaggcggcgagactggcattttctctcccatgcacatgatcctgtgtcgaaagcccgagtctcccgaggaatcttcttaa

SEQ ID NO: 227 - DNA, 1086 bp, synthetic construct
  source 1..1086: mol_type = other DNA
  misc_feature 1..1086: note = Amborella trichopoda C-28 sterol methyl-transferase codon optimised for Yarrowia lipolytica
atggagactctggccgccgttgtcaccctgggtctgctcgctgccggcctctactggttcgtctgcctgctgggttctgccgagcagaaaggtaagcacgcctctgagctgtctggaggctctctgggccgagagcaggtcgcctccacctaccgacagtactggaccttcttccgaaagcccaaggaaattgaacacgccgaccgagtgcccgacctcgtcgactcgttttacaacctggtgaccgacatctacgagtggggctggggacagtctttccacttctctccttctctgcccggacattcccatgccgccgctacccgagctcacgaggagatggccgctaaccttctgaagcttggccccgccatgaaggtccttgacgctggatgcggcgtgggtggccccatgcgaactatcgctactcactccggcgctaacgtcgtcggcatcaccatcaacgagtaccaggtcggccgagcccgactgcacaacatcaaggccggtctggacaagctgtgcgaagtcgtttgcggtgatttcctgcacatgcccttcgatgctgagtccttcgacgccgcctactccatcgaggccacctgccacgctcccaagctctccgaggtctacgccgagattttccgggttctgaagcccggtgccctctacgtgacctacgaatgggtcaccacccccaagtttcagcccgacaattccgaacatctggagatcgtccagggcatcgagcgaggtaacgcccttcccggtctccgacgacaggacgaggtcgccgaaatcgccaagggcgtcggcttcgagctggttgaagagcgggacctggccctaccccctgccctgccttggtggactcgacttaagatgggtcgagtcgcctactttcgaaaccacgtggtcgtctgggtcctgaccatggtccgaattgcccctaagggcgtggacgacgtgcacgagatgctgtttcataccgcccaccacctgactcgaggcggtcagaccggcatcttcacccccatgcacatgattctgcttcgaaaacctaacaacgctgctcccgcctgctaa

SEQ ID NO: 228 - DNA, 38 bp, synthetic construct
  source 1..38: mol_type = other DNA
  misc_feature 1..38: note = PR-27749 (B_Dr_Fw)
  modified_base 15: mod_base = OTHER; note = uracil
actttttgcagtactaaccgcaggaccctctgctgtac

SEQ ID NO: 229 - DNA, 27 bp, synthetic construct
  source 1..27: mol_type = other DNA
  misc_feature 1..27: note = PR-27750 (B_Dr_Rv)
  modified_base 8: mod_base = OTHER; note = uracil
cacgcgatttagtgtcgggcagacttg

SEQ ID NO: 230 - DNA, 39 bp, synthetic construct
  source 1..39: mol_type = other DNA
  misc_feature 1..39: note = PR-27759 (B_Mm_Fw)
  modified_base 15: mod_base = OTHER; note = uracil
actttttgcagtactaaccgcaggaacccgccgtgtctc

SEQ ID NO: 231 - DNA, 27 bp, synthetic construct
  source 1..27: mol_type = other DNA
  misc_feature 1..27: note = PR-27760 (B_Mm_Rv)
  modified_base 8: mod_base = OTHER; note = uracil
cacgcgatttagtgtcgagcggccttg

SEQ ID NO: 232 - DNA, 38 bp, synthetic construct
  source 1..38: mol_type = other DNA
  misc_feature 1..38: note = PR-27785 (C_Cq_Fw)
  modified_base 15: mod_base = OTHER; note = uracil
actttttgcagtactaaccgcaggactctatggccctg

SEQ ID NO: 233 - DNA, 32 bp, synthetic construct
  source 1..32: mol_type = other DNA
  misc_feature 1..32: note = PR-27613 (C_At_Fw)
  modified_base 9: mod_base = OTHER; note = uracil
atctgtcatgccacaatggagactctggccgc

SEQ ID NO: 234 - DNA, 26 bp, synthetic construct
  source 1..26: mol_type = other DNA
  misc_feature 1..26: note = PR-27614 (C_At_Rv)
  modified_base 8: mod_base = OTHER; note = uracil
cgtgcgatttagcaggcgggagcagc

SEQ ID NO: 235 - DNA, 38 bp, synthetic construct
  source 1..38: mol_type = other DNA
  misc_feature 1..38: note = PR-27773 (C_AtSMT2_Fw)
  modified_base 15: mod_base = OTHER; note = uracil
actttttgcagtactaaccgcaggactctctgaccctg

SEQ ID NO: 236 - DNA, 28 bp, synthetic construct
  source 1..28: mol_type = other DNA
  misc_feature 1..28: note = PR-27774 (C_AtSMT2_Rv)
  modified_base 8: mod_base = OTHER; note = uracil
cacgcgatttaagaagattcctcgggag